SEQUENCE LISTING (33 sequences)

SEQ ID NO: 1 - protein, 157 aa, Pseudomonas aeruginosa
MKLDAYDHRILAALQRDGRLSNVQLAEEIGLSPSPCLRRVRLLEEAGVIRGYQASIDRDEVGLGLTVFVGIKVERHRQEQAEAFHRAVVDLPEVISVHLVSGESDFLLQVVVPDLRAYERLLSETLLRLPGVSDIRSNFAIKTVKAPAPLPLGHLAG

SEQ ID NO: 2 - protein, 159 aa, Pseudomonas aeruginosa
MNKLDRYDLRILEELQRDARISNQELAERIGLSPSPCSRRVKQLEDDGYIVRQVALLDRKKLGLSLTAFVLIGMDRHTPERFEHFQEVIGKCPEVLECSLVTGMDADYQLKVVVPDMDHYQKLLLGTLTRIEGVSSVRSSFVLNQVLASTELPLEHLRD

SEQ ID NO: 3 - protein, 153 aa, Pseudomonas aeruginosa
MAELDRIDLKILRALADDGRLSWRDLAQKVGLSLTPTLRRVRRLEEEHYIQGYFARLDEERLSGAMSVFVSVSLEKQTGDYLARFEERIVDAPQVMSCFQMTGDADYMLRVVVKDLAAYQAFLTNTLTCIPGVAGIKSAFALKSVMLRSAPPL

SEQ ID NO: 4 - protein, 160 aa, Pseudomonas aeruginosa
LHMSLDAIDLRILRHLQQDGRMSNQDLAEKVSLSPSACLRRLRLLESEGIIRGYSVELDAERLGVELEAIVHVSLRQDVEGWHEAFIARVRDWPEVVTAYVVTGATNYVLRVRARNLKHYSDFIVNKLNRAAGVTDIRSEIVLQEIKAGADLLDLVNLKS

SEQ ID NO: 5 - protein, 157 aa, Pseudomonas aeruginosa
MISPLDIYDQRILALLQEESSLSTAEIAERIGLSQSPCWRRIQRLKEEGVIRKQVTLLDRKKIGLNTQVFAQIKLNAHGRKNLTDFAEAMHQFPEVLECHVLMGAVDFMLRIVTRDIESYERFFFEKLSAVPGIQEVNSIVALSEIKSTTTLPLLAP

SEQ ID NO: 6 - protein, 145 aa, Pseudomonas aeruginosa
MTDDIDQRLINALMADSRLSLKALAQVSGLSAPSVAERLRKLEERGVIRGYTLDVDPRCFGYQFQAIVRIRPLPGRLHEVERQIQDIAEFTECDKVTGDDCFIARLQVRSMEQLDTLLDRINGYAETNTAIVKKSPVKRRLPPMT

SEQ ID NO: 7 - protein, 155 aa, Pseudomonas aeruginosa
MDKYDRALLAALIDNGRLTFAELARRINLSPPAVADRVAKLEAEGVITGYHASVNLAKLGLPIECVIELRITEKDCQSVLDELARIPQLTLCHRITGDACVLMKAAVASMPELQDLIDRLMKFGISKTSMILSTPFADRQPASLRGQDGNGGCAA

SEQ ID NO: 8 - protein, 162 aa, Pseudomonas aeruginosa
MRTQHQSKRELDKIDRNILRILQEEGRISFTELGERVGLSTTPCTERVRRLEREGLIMGYHARLNPQHLKASLLVFVEISLDYKSGDTFEEFRRAVLKLPHVLECHLVSGHFDYLVKARISEMASYRKLLGDILLKLPHVRESKSYIVMEEVKESLDLPVPD

SEQ ID NO: 9 - protein, 164 aa, Pseudomonas aeruginosa
MVDSKKRPGKDLDRIDRNILNELQKDGRISNVELSKRVGLSPTPCLERVRRLERQGFIQGYTALLNPHYLDASLLVFVEITLNRGAPDVFEQFNTAVQKLEEIQECHLVSGDFDYLLKTRVPDMSAYRKLLGETLLRLPGVNDTRTYVVMEEVKQSNRLVIKTR

SEQ ID NO: 10 - protein, 152 aa, Escherichia coli
MENYLIDNLDRGILEALMGNARTAYAELAKQFGVSPGTIHVRVEKMKQAGIITGARIDVSPKQLGYDVGCFIGIILKSAKDYPSALAKLESLDEVTEAYYTTGHYSIFIKVMCRSIDALQHVLINKIQTIDEIQSTETLIVLQNPIMRTIKP

SEQ ID NO: 11 - protein, 152 aa, Escherichia coli
MLDKIDRKLLALLQQDCTLSLQALAEAVNLTTTPCWKRLKRLEDDGILIGKVALLDPEKIGLGLTAFVLIKTQHHSSEWYCRFVTVVTEMPEVLGFWRMAGEYDYLMRVQVADMKRYDEFYKRLVNSVPGLSDVTSSFAMEQIKYTTSLPIE

SEQ ID NO: 12 - protein, 77 aa, Pyrococcus sp. OT
VMEVFILLVVRPGLENEVYEKLKDRPEVKEIYRLYGDYDIIIRLSVEGIKALDEFHDKVLRKLRGIEISETLIASSY

SEQ ID NO: 13 - protein, 75 aa, Pyrococcus sp. OT
MVTAFILMVTAAGKEREVMEKLLAMPEVKEAYVVYGEYDLIVKVETDTLKDLDQFITEKIRKMPEIQMTSTMIAI

SEQ ID NO: 14 - protein, 80 aa, Pyrococcus sp. OT
MVRAYVLLTIEIGKVEKVIEELKKIPGVTKADAVTGPYDAIVHIEANDLGELTRKILHDIHNIDGVIDTTTAIVVEIEES

SEQ ID NO: 15 - protein, 158 aa, Pyrococcus sp. OT
VITVPHSRKVELDEIDRAILRLLQEDGRMSYSEISRRINVPESTVRARVNRLVKEGVIRKFAALINPFKAGYEIVAFIAVDAEPAKVKQVVEELAKFPEVDVLGIVTGAHDIFMQVTVKDLQELERFILEKMAKIDGIKSTETSILTSVKKWGYARVF

SEQ ID NO: 16 - protein, 155 aa, Pyrococcus sp. OT
MTRERVELTNRQIELLRKLYREGKTIEVHTVEKTQDELAQELGITRQALSNHLKILKELGYIRTGRGFIDLTDKALELLGEKRGDVFIFVKIEPTKRKQVYDTIRKLRVKKIYRVTGDIDLIIEADKSKLDEILEEIAALDGVKETITHVVLGVL

SEQ ID NO: 17 - protein, 170 aa, Pyrococcus sp. OT
MNDDPETAVLKKVFKGKLDDLDIMIYKMLREDGRISDSRIAERLGVSVTTVRRHRLKLQREGILQVIGLLLLRAADVAYADVLVKFSKDATPEEIRKFVNEAVENPRIYEVTEYIGNGYDLMLRFFESNLESLKYHIDRFLRSKTIVEKYEVFTAIGSPKAWYKSLKLKF

SEQ ID NO: 18 - protein, 162 aa, Pyrococcus sp. OT
VLKMKKRKLSKKDWEIIKLLKKDARMSDAEIGRRIGLSKSAVRWRRINLQKRGYLLISAYLRFDKLGYTYAFVLVKIKPDTPRNEILKFKKALMENEHTFEIYEVLGDYNVLIGVFGEDVSELKRNIQELIIGQKCVQEYKVLLGAKSLKGLEVPFWDALED

SEQ ID NO: 19 - protein, 155 aa, Pyrococcus sp. OT
MAGIDEIDEVIVRELRKNSRITLTELGRKVGLTASAVKNRIEKLEKLGVIKGYSAVVDPSFFGEFLTAVIEIELIDPDSPDLPRILTPILKMRNISDVYKKTGEFHLVIRGTFRDVESLNTFLKDLKRNYLRNLARRTRISIVLENFKEAGVTLK

SEQ ID NO: 20 - protein, 162 aa, Pyrococcus sp. OT
MISTPLDDLDRAILKLLKKDARLTIAEISNQLKKPESTVHFRIKKLQERGVIEKYTIILGEPIRPRELALVVLEVDKPIIEDFLDRYMEYVTKTLSGFPEVLFVAKSGKEKIVALVGGEDRDKLLKFIEENIESIPTLRSVQVLPISEIKKGDEISGFLAEV

SEQ ID NO: 21 - protein, 151 aa, Pyrococcus sp. OT
VDCMRKLDRVDMQLVKILSENSRLTYRELADILNTTRQRIARRIDKLKKLGIIRKFTIIPDIDKLGYMYAIVLIKSKVPSDADKVISEISDIEYVKSVEKGVGRYNIIVRLLLPKDIKDAENLISEFLQRIKNAENVEVILISEVRKFEII

SEQ ID NO: 22 - protein, 151 aa, Pyrococcus sp. OT
VLKMELDALDRKILEILLKDSRTSYREIAKDLNVAVGTIYNRIKKLEDSGVIQAFTVKLNYESIGYDLTAIIGIKAQGKKIREIERIIAKDKRVTCVYDVTGEYDIIVIAKFRNREDMNRFVKGVLSIDGVEKTNTHVVLEIVKEDFRLEP

SEQ ID NO: 23 - protein, 160 aa, Pyrococcus sp. OT
LKNFLRGGKVRVPLDEIDKKIIKILQNDGKAPLREISKITGLAESTIHERIRKLRESGVIKKFTAIIDPEALGYSMLAFILVKVKAGKYSEVASNLAKYPEIVEVYETTGDYDMVVKIRTKNSEELNNFLDLIGSIPGVEGTHTMIVLKTHKETTELPIK

SEQ ID NO: 24 - protein, 150 aa, Pyrococcus sp. OT
MPEILDKVDRRLLEELKNNARENIATLSKKLGIPRTTVHYRIKRLVEEGIIEKFTIKPNYKKLNLGTTAFILIRYDPDSGLTQREVAEQIARIPGVYEVHLVAGEWDLLLKVRASNAEEIGRIVIDKLREIRGVGQTVTMVSFVTVKEEI

SEQ ID NO: 25 - protein, 141 aa, Pyrococcus sp. OT
MIDERDKIILEILSKDARTPFTEIAKKLGISETAVRKRVKALEEKGIIEGYTIRINPKKLGYSLVTITGVDTRPEKLFEVAEKLKEFEFVRELYLSSGDHMIMAVIWARDGEDLADIISNKIGKIDGVTKVCPAIILEKLK

SEQ ID NO: 26 - protein, 77 aa, Thermoplasma volcanium
LSIAFVLISTVPGKEHEVYNKVLKINGVVEAHPLFGEYDIIVKLDMKDFTEIGEVVIEKIRTIDGVIDTKTLTGIKL

SEQ ID NO: 27 - protein, 175 aa, Thermoplasma volcanium
MHPEKRNLSNLESRVLRELIDDSRSSVEEISERTGIGRNTVSKIIKDLERNGIIKKYTIKTSYEESEKKIIALTPDDVHIPDDLIYYNFKLSNGKRILVLSKSALDYDFQYDQVWISDTVEEGKAILSNITLYCDYCGNQIHGDPISVKWKNRTYLVCCPNCEKDMLKRLEKDNS

SEQ ID NO: 28 - protein, 139 aa, Thermoplasma volcanium
IDRLDLDIVEMLETDCSLTYEEIAKMTGKSLWTIRDRILALKKRGVIKSCRAEIDYDKLGLPCRAIIGFNVPPDKIDDFVEQAKKDKRIKKFIITTGSRRFHIKIIGKQCGEIREYARTILPKFGVFDVDFEVVLDEII

SEQ ID NO: 29 - protein, 177 aa, Thermoplasma volcanium
MEKKDISDQYLEGYSCDVAIRWSKIDPSLLPVDELDKQIICFLRYNARISNAEIAKALNTSEATVRRRINELVKKKIIIGFSALVDLQAVENSIKAYIIIDVDPNRMDEIAGAISDNINVLSLYRRRGKNQLIAETLFLNLEAIENFEAFISKLNGVKNFETMIISKAYRKNPWIGI

SEQ ID NO: 30 - protein, 130 aa, Thermoplasma volcanium
VDEKDRDILNLLIDNSRISNTEIAKVLNVSEGTVRKRIKKMIDEGIIKRFTVETSDNTIDALILVKIDNKKYKEVLQRLRARYYEIYEFSGRIDVAIRIKTDSTDNLNRIVDQIREIEGVVDTDTLIRLQ

SEQ ID NO: 31 - protein, 143 aa, Thermoplasma volcanium
MDEKDLQILNYLMENGRDKIATLSKKLGIPRTTVHYRIKRLVESGIIKKFTIVPDYKALGIPVVAFILVAYTPKDGISQRDLAAKIATFKEVENVYIIAGEWDLLIKVRESSTEAIGNFVLDKLREMPGIERAQTITVFSEVK

SEQ ID NO: 32 - DNA, 33 bp, Artificial Sequence
  primer
tggtgatgacatatggtgacggcctttatcctg

SEQ ID NO: 33 - DNA, 36 bp, Artificial Sequence
  primer
gaacggatccatcaaattgctatcatagtcgaggtc

(19) United States
(12) Patent Application Publication
(10) Pub. No.: US 2005/0202522 A1
(43) Pub. Date: Sep. 15, 2005

(54) Method of screening for antimicrobial agents

(75) Inventors: Masashi Suzuki, Ibaraki-ken (JP); Hideaki Koike, Ibaraki-ken (JP)

Correspondence Address: SUGHRUE MION, PLLC, 2100 PENNSYLVANIA AVENUE, N.W., SUITE 800, WASHINGTON, DC, 20037, US

(73) Assignee: NATIONAL INSTITUTE OF ADVANCED INDUSTRIAL SCIENCE AND TECHNOLOGY

(21) Appl. No.: 10/793,746

(22) Filed: Mar. 8, 2004

(30) Foreign Application Priority Data
Jun. 20, 2003 (JP) 2003-176921

(51) Int. Cl. G06F 19/00; G01N 33/48; G01N 33/50; C12Q 1/18; G01N 33/543

(52) U.S. Cl. 435/32; 436/518; 702/19

(57) ABSTRACT

[Object] To identify FFRPs which are prospective targets of antimicrobial agents, and by using the FFRPs, to provide a pharmaceutical agent and a novel method for screening for a pharmaceutical agent, in particular, an agent that acts against P. aeruginosa.

[Solving Means] Using the genomic sequence of a bacterium, FFRPs coded therein are identified on the basis of correlation between FFRPs summarized in a multiple alignment shown in FIG. 1. By specifying chemical compounds that can specifically interact with the identified FFRPs or their assemblies, thereby changing their 3D structures, candidates for antimicrobial agents are screened.

### TECHNICAL FIELD OF THE INVENTION

The present invention relates to a method of identifying proteins that can be used for searching for antimicrobial agents and to a method of screening for agents by targeting these proteins.

### DESCRIPTION OF THE RELATED ART

Metabolism, growth, and environmental adaptation of many bacteria are regulated by a group of genes closely related with each other. The products of these genes, which are referred to as “feast/famine regulatory proteins (FFRPs)”, bind to promoter regions in genomic DNAs and efficiently regulate transcription of genes positioned downstream. The term “FFRPs” was proposed by the present inventors by referring to “feast/famine regulation”, an expression (Nonpatent Document 1) used to summarize the function of the leucine-responsive regulatory protein (Lrp) of Escherichia coli, a protein in this group. When sensing an increase in the nutrition outside through a high concentration of leucine, E. coli pauses its motility, initiates active transport of nutrients from outside, shifts its metabolism to a more heterotrophic mode, and initiates its growth, eventually changing its infectivity. Another E. coli protein, the asparagine synthesis C gene product (AsnC) senses an increase in the nutrition outside through high concentration of asparagine and regulates biosynthesis of asparagine and growth of the bacterium. The amino acid sequence of AsnC resembles that of Lrp, and thus AsnC too is classified into the group of FFRPs. E. coli has a third FFRP whose function is yet unknown.

Pseudomonas aeruginosa opportunistically infects people whose defensibility is weakened, i.e., those who are suffering from cancer, burn injuries, immune disorder, or the like, and causes serious diseases, such as sepsis and cystic fibrosis, sometimes leading to death of the patient. P. aeruginosa adopts abilities to resist various antibiotics and disinfectants. The multi-drug resistancy of P. aeruginosa has caused serious inside hospital infections not only in developing countries but also in advanced countries. In an attempt to unravel the infection mechanism of P. aeruginosa, the genomic sequence of its standard strain PAO1 has been determined and published under international collaboration (Nonpatent Document 2). However, to date, this has not lead to development of effective agents.

Whereas most other pathogenic bacteria are parasitic and can only survive inside their hosts, P. aeruginosa adapts to and grows in various environments, those in water and soil, and those inside plants and animal tissues, and so on. Bacteria of the species P. aeruginosa communicate with each other by transmitting signals and their characteristics change largely once the number of the bacteria reaches a certain threshold. This mechanism called “quorum sensing” is rarely found with other bacterial species and in some sense comparable with intercellular signaling inside multicellular organisms such as humans. It is likely that this remarkable environmental adaptability of the organism, together with its opportunistic infectivity (i.e. its ability to change depending on host susceptibly) and its multi-drug resistancy (i.e. its ability to change by responding to drugs), all are of the same type of abilities, and underneath all these lying is an efficient mechanism of regulating transcription of a number of genes in various ways.

The present inventors have identified eight FFRPs (Nonpatent Document 3), by using the genomic sequence of P. aeruginosa publicly available. No FFRP has been found coded in many parasitic pathogens, and this can be a reflection of the fact that these pathogens are dependent on nutrition present inside their hosts and do not regulate their metabolisms. In contrast, the number of FFRPs of P. aeruginosa, i.e., eight, is the largest among eubacteria so far examined, strongly suggesting involvement of these proteins in the efficient transcription regulatory mechanism of P. aeruginosa. So far, no evidence has been found to prove direct involvement of FFRPs in multi-drug resistance or opportunistic infectivity of the organism. Even so, FFRPs will regulate the metabolism of P. aeruginosa as does that of E. coli, and thus, regardless of the detailed function of FFRPs, FFRPs are potential targets for designing drugs that can terminate growth of P. aeruginosa or eradicate this organism.

[Nonpatent Document 1]
Calvo, J. M., and Matthews, R. G., 1994, Microb. Rev., 58, pp. 466-490

[Nonpatent Document 2]
Stover, C. K. et al., 2000, Nature, 401, pp. 959-964

[Nonpatent Document 3]
Koike, H. et al., 2003, Proc. Japan. Acad., 79B, pp. 63-69

[Problems to be Solved by the Invention]

An object of the present invention is to identify the FFRPs which are potential targets while screening for antimicrobial agents and to provide a novel system for developing agents by using the identified target FFRPs, in particular, a novel method for developing agents acting against P. aeruginosa.

[Means for Solving the Problems]

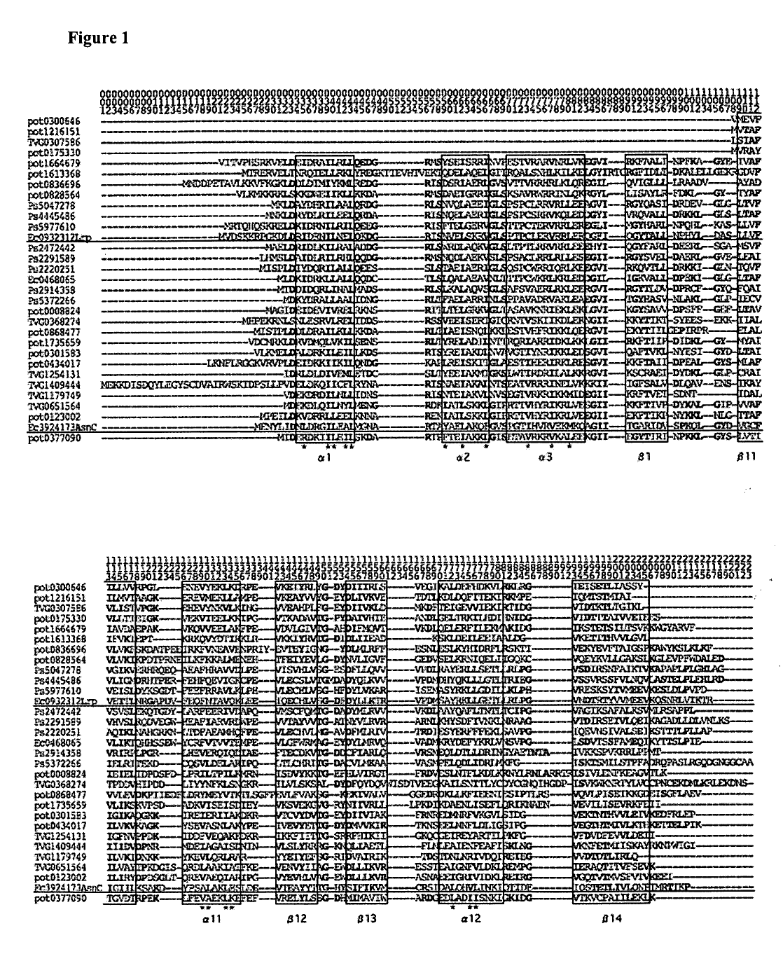

The present inventors have identified eight FFRPs of P. aeruginosa and have completed a multiple alignment (FIG. 1) by comparing the amino acid sequences of the P. aeruginosa FFRPs with those of FFRPs from other bacteria, in particular by analyzing possibilities of these sequences to form a particular set of secondary structural elements. In addition, the present inventors have crystallized an archaeal FFRP (pot1216151) in complex with a ligand, and, by using the 3D coordinates determined thereof, have identified the geometries of the amino acid side chains which will interact with pharmaceutical agents. Based on these findings, the inventors have identified, in the eight FFRPs of P. aeruginosa, the positions and types of amino acid residues with which agents to be developed to act against P. aeruginosa will interact. Furthermore, in order to minimize potential interaction of agents to be developed with E. coli in human body, the phylogenetic relationship of the P. aeruginosa FFRPs with FFRPs of other bacteria has been analyzed thereby identifying FFRPs of P. aeruginosa closely related with E. coli Lrp.

The present invention provides a method of identifying a target protein for its use in screening for an antimicrobial agent, which comprises:
1) from the amino acid sequences of all open reading frames identified using the genomic sequence of a target bacterium, selecting amino acid sequences having homologies of 20% or higher to one of feast/famine regulatory proteins (FFRPs) having amino acid sequences set forth in SEQ ID NOS. 9 to 31;
2) from the amino acid sequences selected in step 1), excluding any amino acid sequence that does not match with a multiple alignment by forming the same secondary structural elements, the multiple alignment being shown in FIG. 1; and
3) identifying a protein, which has an amino acid sequence obtained through steps 1) and 2), as the target protein for its use in the screening for the antimicrobial agent.

The above-described method may further comprise the step of excluding any amino acid sequence phylogenetically related with one of the E. coli FFRPs having amino acid sequences set forth in SEQ ID NOS. 9 to 11, by a bootstrap value of 900/1,000 or higher.

In step 1), amino acid sequences having, for example, FASTA Z scores of 180 or higher to one of FFRPs having amino acid sequences set forth in SEQ ID NOS. 9 to 31 may be selected.

In step 2), for example, any amino acid sequence that is unlikely to form α helices in five regions 34 to 43, 59 to 66, 70 to 82, 126 to 135, and 170 to 180 or β strands in five regions 90 to 96, 109 to 117, 142 to 148, 154 to 160, and 193 to 205 is excluded from the amino acid sequences selected by step 1), wherein these positions are identified using the numbering scheme shown in the multiple alignment in FIG. 1.

The present invention provides also a method of screening for an antimicrobial agent, which comprises the step of selecting a chemical compound as a candidate of the agent, wherein the chemical compound is able to bind specifically to a protein identified by the method described above or to its assembly, thereby altering the three-dimensional structure or the assembly form of the protein.

In an embodiment, the above-described method becomes a method of screening for an antimicrobial agent acting against Pseudomonas aeruginosa, which comprises the step of selecting a chemical compound as a candidate of the antimicrobial agent, wherein said chemical compound is able to bind specifically to a target protein or its assembly, thereby altering the three-dimensional structure or the assembly form of the protein, wherein the target protein is either
i) a protein having an amino acid sequence set forth in one of SEQ ID NOS. 1 to 8; or
ii) a protein which is derived from Pseudomonas aeruginosa, related to an amino acid sequence set forth in one of SEQ ID NOS. 1 to 8 by deletion, substitution, or insertion of up to several amino acid residues, and is able to act as an FFRP.

In another embodiment, the above-described method becomes a method of screening for an antimicrobial agent acting against Pseudomonas aeruginosa, which comprises the step of selecting a chemical compound as a candidate of the antimicrobial agent, said chemical compound being able to bind specifically to a target protein having an amino acid sequence set forth in one of SEQ ID NOS. 1 to 8 or its assembly, thereby altering the three-dimensional structure or the assembly form of the target protein.

In yet another embodiment, the method may be a method of screening for an antimicrobial agent acting against Pseudomonas aeruginosa, which comprises the step of selecting a chemical compound as a candidate of the antimicrobial agent, said chemical compound being able to bind specifically to a target protein having an amino acid sequence set forth in one of SEQ ID NOS. 1 to 7 or its assembly, thereby altering the three-dimensional structure or the assembly form of the target protein.

In the screening method of the present invention, for example, the chemical compound may be selected on the basis of interaction between the chemical compound and at least one of thirty-two amino acid residues in the target protein, said thirty-two amino acid residues being found at positions 113, 116, 119, 120, 126, 144, 145, 147, 148, 149, 150, 152, 153, 154, 155, 166, 169, 173, 176, 177, 181, 183, 185, 193, 195, 196, 197, 198, 199, 200, 201, and 202, respectively, wherein these positions are identified using FIG. 1.

Alternatively, the chemical compound is selected on the basis of interaction between the chemical compound and at least one of eighteen amino acid residues in the target protein, said eighteen amino acid residues being found at positions 113, 126, 147, 148, 149, 150, 152, 153, 154, 155, 176, 177, 181, 196, 197, 198, 199, and 202, respectively, wherein these positions are identified using FIG. 1.

Alternatively, the chemical compound is selected on the basis of interaction between the chemical compound and at least one of seven amino acid residues in the target protein, said seven amino acid residues being found at positions 147, 154, 169, 181, 199, 200, and 201, respectively, wherein these positions are identified using FIG. 1.

Alternatively, the chemical compound is selected on the basis of interaction between the chemical compound and at least one of seven amino acid residues in the target protein, said seven amino acid residues being found at positions 147, 149, 154, 169, 173, 200, and 202, respectively, wherein these positions are identified using FIG. 1.

Alternatively, the chemical compound is selected on the basis of interaction between the chemical compound and at least one of twenty-eight amino acid residues in the target protein, said twenty-eight amino acid residues being found at positions 116, 119, 120, 126, 144, 145, 147, 148, 149, 150, 152, 153, 154, 166, 169, 173, 177, 181, 183, 185, 193, 195, 196, 197, 198, 199, 200, and 201, respectively, wherein these positions are identified using FIG. 1.

### DESCRIPTION OF THE EMBODIMENTS

The present invention will now be described in detail.

1. Feast/Famine Regulatory Proteins (FFRPs)

In the present invention, the term “FFRPs” refers to proteins that regulate metabolism, growth, and infectivity of bacteria in response to changes in nutritional conditions outside. This term was derived from an expression, “feast/famine regulation”, used to summarize function of E. coli leucine-responsive regulatory protein (Lrp), a protein in this group. It has been identified that E. coli has three FFRPs: Lrp, AsnC, and a third FFRP of unknown function. FFRPs have been found in many other bacteria, such as those belonging to the genus Pyrococcus and the genus Sulfolobus.

FFRPs function by binding to promoter DNA regions positioned upstream of genes regulated by FFRPs, thereby activating or inactivating transcription of these genes. It is believed that association to or dissociation from promoter regions by FFRPs is regulated by the small molecules that reflect the nutritional conditions outside and that these small molecules introduce structural changes to the FFRPs, thereby affecting their abilities to bind to the promoter DNAs. The present invention relates to a method of screening for pharmaceutical agents that bind specifically to FFRPs thereby introducing structural changes, and thus able to regulate metabolism, growth, or infectivity of bacteria. In the present invention the term “antimicrobial agent” refers to “an agent interacting with a bacterium thereby regulating one of metabolism, growth, and infectivity of the bacterium and terminating its growth or eradicating the bacterium”. In particular, the present invention provides a method of screening for agents that can act against P. aeruginosa by targeting the P. aeruginosa FFRPs identified in this application.

2. Method of Identifying Target Proteins (FFRPs)

The present invention provides a method of identifying a target protein for its use in screening for an antimicrobial agent, which comprises the steps of:
1) from the amino acid sequences of all open reading frames identified using the genomic sequence of a target bacterium, selecting amino acid sequences having homologies of 20% or higher to one of feast/famine regulatory proteins (FFRPs) having amino acid sequences set forth in SEQ ID NOS. 9 to 31;
2) from the amino acid sequences selected in step 1), excluding any amino acid sequence that does not match with a multiple alignment by forming a particular set of secondary structural elements, the multiple alignment being shown in FIG. 1; and
3) identifying a protein, which has an amino acid sequence obtained through steps 1) and 2), as the target protein for use in the screening for the antimicrobial agent.
   2.1. Homology Search

Step 1 is a step of selecting amino acid sequences homologous to those of known FFRPs by using information obtained from a genomic sequence. This step may be performed using a homology search program, such as BLAST, FASTA, PSI-BLAST, or SSEARCH. The threshold is preferably set to a relatively low level so that no FFRP is overlooked. For example, if FASTA program is used, a low Z score, such as approximately 180, can be used as a threshold.

2.2. Multiple Alignment

Step 2) is a step of excluding amino acid sequences that do not form the same set of secondary structural elements as those formed by known FFRPs by using the multiple alignment (FIG. 1) made by the present inventors.

The multiple alignment shown in FIG. 1 is an improvement of an initial version made using a program such as CLUSTALW, PAM, or MultAlin. In each set of positions equivalent between proteins, the number of positions occupied by the same type of amino acid residues has been maximized. In order to confirm that regions equivalent between different proteins will form the same secondary structural elements and the same 3D structures, the following requirements have been satisfied:
1) inside regions predicted to form a-helices, hydrophobic residues should be best positioned in the same phases;
2) inside regions predicted to form secondary structural elements, amino acid residues that might prevent formation of these elements should best be avoided;
3) neither deletion nor insertion of amino acid residues will be found at the midst of regions predicted to form α helices;
4) proline, glycine, and tryptophan should best occupy the same positions among the FFRPs.

Theoretical identification of proteins is a process of finding, in a genomic sequence, candidate blocks whose sequences resemble a referential amino acid sequence. If a threshold used for judging resemblance is fixed too high, no candidate will be qualified. If the threshold is fixed too low, all the candidates will be qualified. Selecting an appropriate threshold is thus important and this process of fixing the threshold largely depends on experience. In addition, it is also difficult to align more than two amino acid sequences by matching sets of residues with each other. Furthermore, resembling or not resembling between twenty types of amino acid residues depends on the context, i.e., the function expected for the position. For example, arginine and glutamic acid have opposite electron charges, and thus they are different. However, both are hydrophilic and are often positioned on the surface of a protein. Once the arginine side chain is neutralized by electrostatic interactions with another amino acid side chain, the “stem” composed of hydrocarbons will behave in ways similar to the side chains of hydrophobic amino acid residues such as leucine. The multiple alignment such as the one shown in FIG. 1 can be produced only on the basis of an accumulation of experiences and by applying high analytic skills.

Amino acid sequences that do not form the same set of secondary structures shown along the multiple alignment in FIG. 1 are thus excluded. Namely, amino acid sequences that are not expected to form α helices in five regions 34-43, 59-66, 70-82, 126-135, and 170-180, or β strands in five regions 90-96, 109-117, 142-148, 154-160, and 193-205 are excluded.

2.3. Phylogenetic Relation

Various types of similarities are found between proteins. For example, human hemoglobin and porcine hemoglobin have essentially the same function but have differences originated in speciation between humans and swine. Proteins related by such similarity are defined as orthologous to each other. Proteins orthologous to each other are essentially interchangeable. Until recently, porcine insulin was used to treat patients replacing human insulin. During the Second World War, even tuna insulin was used. On the other hand, the similarity found between human hemoglobin and human myoglobin is of a different type. Functions of these proteins have deviated from each other and the proteins are not interchangeable. Proteins related by such similarity are called to be paralogous to each other. The group of FFRPs is a mixture of proteins orthologous or paralogous to each other.

By comparing FFRPs of two or more bacterial species and by screening for chemical compounds acting against FFRPs shared by these species, the method of the present invention can be used in order to develop agents able to act against a desired wide variety of bacteria. Alternatively, by screening for an FFRP present in a particular bacterial species only, pharmaceutical agents specifically acting against the species can be developed. For example, in order to develop an agent not interactive with the human symbiont E. coli, the method of the present invention may include the step of excluding the amino acid sequences that closely resemble the amino acid sequences (SEQ ID NOS. 9 to 11) of E. coli FFRPs.

“Orthologous/paralogous” relationships between FFRPs can be identified by analyzing their phylogeny. Phylogeny can be analyzed by using commercially available software, such as that in the PHYLIP package, while keeping the alignment shown in FIG. 1 unchanged. Preferably, a threshold usable for excluding remote proteins is a bootstrap value of approximately 900/1,000 or higher.

A bootstrap value is a measure for evaluating homogeneity of data from which a conclusion is deduced. Here, this value defines the number of trials in which the same diversification at a node is concluded out of 1,000 trials, while effectively changing a weight given to each amino acid position used for determining similarity. A pair of proteins related by a bootstrap value close to 1,000 are concluded to be phylogenically close.

3. FFRPs from P. aeruginosa

By using the method of the present invention, eight FFRPs were identified as coded in the genome of the standard strain PAO1 of P. aeruginosa (Stover, C. K. et al., 2000, Nature 406, 959-964):
Ps5047278 (SEQ ID NO. 1), Ps4445486 (SEQ ID NO. 2),
Ps2472442 (SEQ ID NO. 3), Ps2291589 (SEQ ID NO. 4),
Ps2220251 (SEQ ID NO. 5), Ps2914358 (SEQ ID NO. 6),
Ps5372266 (SEQ ID NO. 7), and Ps5977610 (SEQ ID NO. 8).

As has been discussed earlier in this specification, P. aeruginosa easily mutates, thereby acquiring resistance to drugs. Therefore, these FFRPs determined using the genomic sequence of the standard strain PAO1 of P. aeruginosa may also be mutated. The present invention also concerns modified proteins having amino acid sequences mutated from those described above by deletion, substitution, or insertion of up to several amino acid residues, when the modified proteins retain their function as FFRPs.

In order to maintain the original 3D structure, the above-described deletion, substitution, or insertion should not affect formation of the five a helices or five β strands at the regions indicated in FIG. 1. It is preferable that no deletion, substitution, or insertion should occur at amino acid positions that will interact with the pharmaceutical agents, i.e., positions 113, 116, 119, 120, 126, 144, 145, 147, 148, 149, 150, 152, 153, 154, 155, 166, 169, 173, 176, 177, 181, 183, 185, 193, 195, 196, 197, 198, 199, 200, 201, and 202 shown in FIG. 1.

By analyzing phylogeny between FFRPs of P. aeruginosa and E. coli, for reasons it has been confirmed that Ps5977610 is an ortholog of E. coli Lrp, with the bootstrap value being higher than 900. Thus, Ps5977610 (SEQ ID NO. 8) should better not be used as the target for developing pharmaceutical agents to be administered to humans in order to minimize possible effects of the agents on the human symbiont E. coli.

4. Method of Screening for Antimicrobial Agents

A general expectation is that upon transcription regulation by FFRPs small molecules (i.e., ligands) signaling the nutritional conditions outside bind to FFRPS, and change the 3D structure of FFRPs thereby changing the abilities of the FFRPs to bind promoter DNA regions. In other words, a chemical compound able to alter the 3D structure or the assembly form of an FFRP can be used as an antimicrobial agent, which will affect, via modification of transcription regulation by the FFRP, metabolism, growth, or infectivity of the bacterium.

The present invention provides a method of screening for antimicrobial agents, which will bind to a target protein identified by the present invention or its assembly, thereby changing the 3D structure or the assembly form of the target protein, where the target protein and its assembly are the target FFRP and its assembly.

The screening method of the present invention may include either any known ligand-screening system for characterizing changes in the 3D structure or the assembly form of an FFRP upon adding a ligand candidate (e.g., gel filtration, sedimentation coefficient measurement, or the like), or a virtual screening method using a computer.

4.1 3D Structural Information

The structure of an Lrp-like protein (i.e. an FFRP) derived from Pyrococcus furiosus and crystallized in the absence of any ligand and the secondary structural composition of the protein have been reported (Philip M. Leonard, et al., 2001, The EMBO Journal, vol. 20, No. 5, pp. 990-997). With the quality of these data being poor (i.e. an R-factor of approximately 30% or higher at a resolution of approximately 3 Å), the conformations of amino acid side chains, which are important for screening for ligands have not been specified.

The present inventors have succeeded in crystallizing another FFRP (pot1216151) in complex with a ligand, the FFRP being one of the fourteen FFRPs derived from Pyrococcus sp. OT3 (a.k.a. Pyrococcus horikoshii or Pyrococcus shinkaii, Japan Collection of Microorganisms, JCM Registration No. 9974). The 3D structure of the FFRP-ligand complex has been determined at a high resolution using an X-ray diffraction method. To date, this is the single 3D structure of any FFRP determined in complex with a ligand. With the high quality, i.e., R-factor of 21% at a resolution of 1.8 Å, this is the single structure of any FFRP where the accurate positions of amino acid side chains interacting with a ligand are determined.

Table 1 shows the 3D coordinates of pot1216151 determined by the present inventors. These 3D coordinates, or a graphical representation or numerical information derived from the 3D coordinates can provide the 3D structural information necessary for searching for a ligand able to interact with an FFRP. Such 3D structural information includes information concerning both the monomer and its assembly, and also information concerning both the protein alone and the protein in complex with the ligand.

In the present invention, the information, in particular, concerning “the gaps” or “the hole” formed upon assembling of FFRP molecules is useful. Here, “gap” refers to an empty space created between a pair of FFRP monomers, between their atoms, or between their amino acid residues, and “hole” refers to a larger empty space formed at around the center of the assembly. Such information is important for characterizing binding to FFRPs of ligands to be screened.

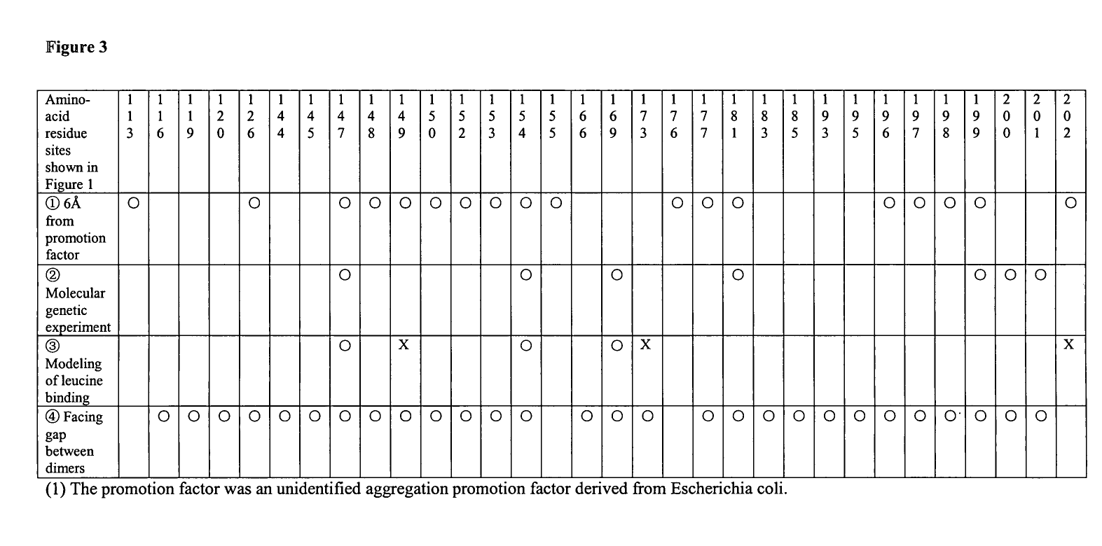

By using the sequence alignment shown in FIG. 1, the 3D information obtained from Table 1 becomes applicable to other FFRPs. In particular, information concerning amino acid residues facing the “hole” or the “gaps” in the assembly, which are ligand-binding positions, is provided. In this way, the present inventors have specified thirty-two positions of bacterial FFRPs, these positions being potential targets of pharmaceutical agents: 113, 116, 119, 120, 126, 144, 145, 147, 148, 149, 150, 152, 153, 154, 155, 166, 169, 173, 176, 177, 181, 183, 185, 193, 195, 196, 197, 198, 199, 200, 201 and 202 (FIG. 3), wherein these positions are identified using FIG. 1.

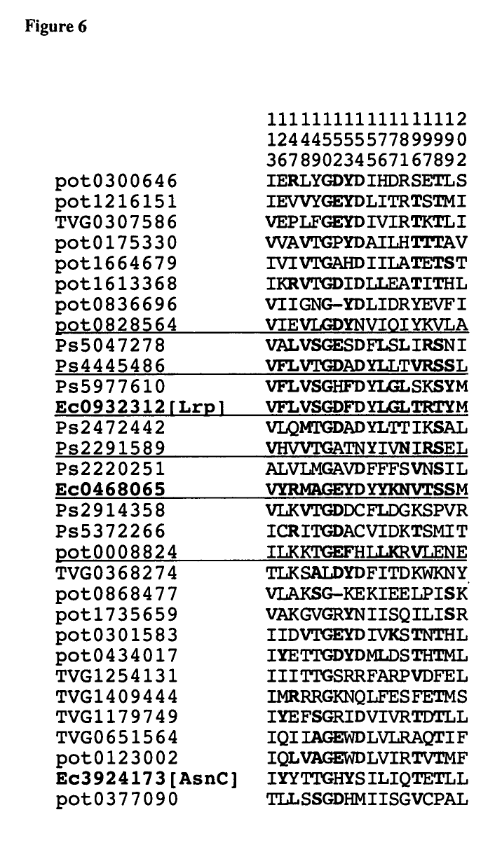

Of the above-described positions, the following eighteen positions (FIG. 6) are the positions, in pot1216151, identified as interacting with two molecules of an unidentified assembly promotion factor: 113, 126, 147, 148, 149, 150, 152, 153, 154, 155, 176, 177, 181, 196, 197, 198, 199, and 202 shown in FIG. 1.

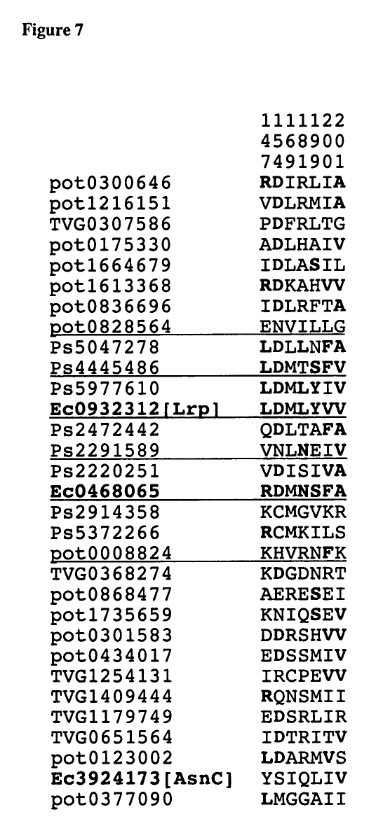

The following seven positions (FIG. 7) are the positions, in E. coli Lrp, identified to potentially interact with leucine: 147, 154, 169, 181, 199, 200, and 201 of FIG. 1.

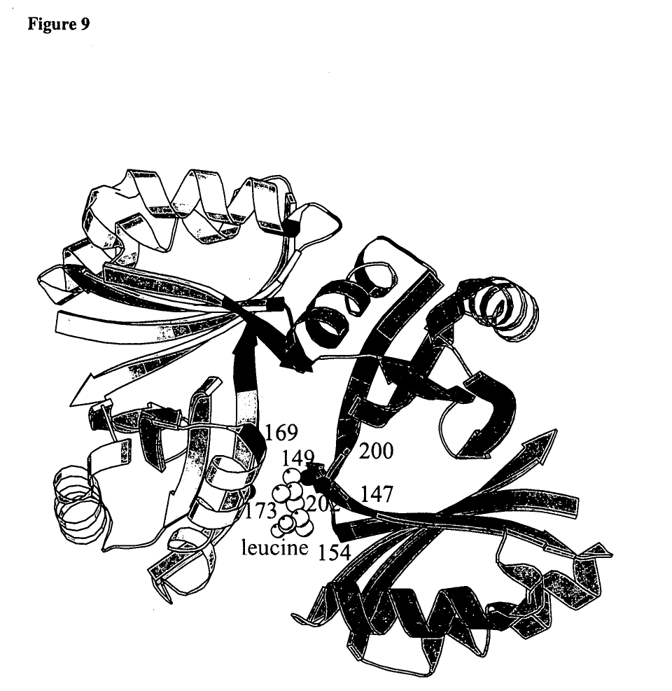

The following seven positions (FIG. 9) are the positions, in pot1216151, identified to potentially interact with leucine: 147, 149, 154, 169, 173, 200, and 202 of FIG. 1.

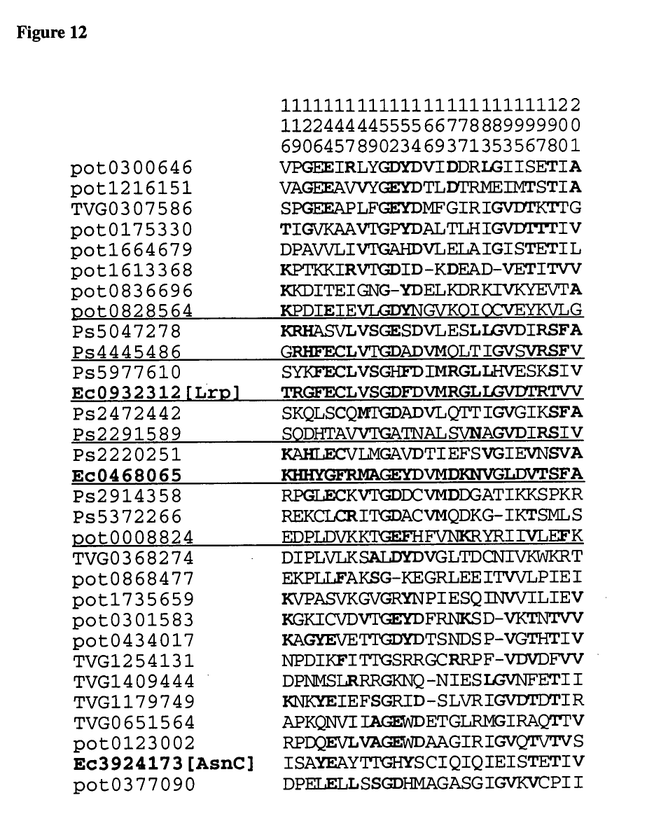

The following twenty-eight positions (FIG. 12) are the positions, in pot1216151, identified as facing the “gaps”: 116, 119, 120, 126, 144, 145, 147, 148, 149, 150, 152, 153, 154, 166, 169, 173, 177, 181, 183, 185, 193, 195, 196, 197, 198, 199, 200, and 201 of FIG. 1.

4.2. Virtual Screening and Drug Designing

In the present invention, agents can be virtually screened by using the original 3D atomic coordinates determined as above or coordinates derived therefrom.

Upon the virtual screening, the 3D atomic coordinates shown in Table 1 are input to a computer thereby obtaining graphic representation or any type of numerical information. All the coordinates shown in Table 1 or any necessary part may be input, such as the part defining the “hole” or the “gaps”, or the part defining the five α helices and the five β helices. Examples of a program usable for analyzing the 3D structure include those collected in CCP4 package, and program O, X-plor, MolScript, Insight II, and Grasp. Examples of “graphic representation” include any forms to produce visual information such as ribbon diagrams, 3D models, or types of computer graphics. Examples of “numerical information” include any information composed of numbers, such as diameters of crystals and widths and depths of the hole and the gaps.

The information obtained as above is input to a virtual library of chemical compounds in order to search for compounds that are candidates of the pharmaceutical agents. Examples of the virtual library include, but are not limited to, those commercially available. For example, screening software such as DOCK-4 (Kunts), and a 3D structural database such as MDDR (Prous Science) can be used.

The graphic representation or the numerical information described above can be used not only for screening for candidates of pharmaceutical agents but also for modeling or designing of agents using computers. Modeling may be performed using software such as FRODO or O, designed for analyzing crystal structure, and designing may be performed using QUANTA, InsightII, or the like. For, example, a ligand able to fit into the hole or the gap can be designed by using software for molecular designing, such as QUANTA, by consulting with a computer graphics made using the atomic coordinates of the FFRP.

### EXAMPLES

Although the present invention will now be described in detail by way of EXAMPLES and REFERENCE EXAMPLES, the present invention is not limited to these examples.

### Example 1

### Identification of P. aeruginosa FFRPs

All open reading frames (ORFs, each formally codes for 50 or more amino acid residues between the start and stop codons) found in the complete genomic sequence (Stover, C. K. et al., 2000, Nature 406, 959-964) of the standard strain (PAO1) of P. aeruginosa were identified and translated to amino acid sequences. ORFs of P. aeruginosa resembling the FFRPs of E. coli (genomic sequence of E. coli K strain: Blattner, F. R. et al., 1977, Science, 277, 1453-1474), or FFRPs of archaea, Pyrococcus sp. OT3 (JCM 9974) or Thermoplasma volcanium (these FFRPs listed in Suzuki, M et al., 2003, Proc. Japan Acad. 79B, pp. 92-98), were identified using the FASTA program (Peason, W. R., and Lipman, D. L., 1988, Proc. Natl. Acad. Sci. USA, 85, 2444-2448). A low Z score of approximately 180 was used as the threshold so that no FFRP was overlooked.

The amino acid sequences of the candidates of P. aeruginosa FFRPs identified as above and the amino acid sequences of FFRPs of the other bacteria were analyzed using the CLUSTALW program collected in the PHYLIP package (Thompson J. D. et al., 1994, Nucl. Acids Res., 22, 4673-4680), and a multiple alignment was made by correlating residues between different sequences with each other.

Two types of major improvements were made to this alignment. First, in each set of positions identified to be identical as many as possible should be occupied by the single type of amino acid residue (highlighted in bold at positions outside the α helices in FIG. 1). Second, identical regions in different FFRPs should form the same types of secondary structural elements (e.g., an α helix), so that these secondary structural elements formed in the same order will form essentially the same 3D structure. A program such as CLUSTALW is not usable for the latter type of improvement.

Importantly, it has been confirmed that inside the regions predicted to fold into α-helices, hydrophobic residues should be positioned by forming particular phasings: in an α helix, every 3.6 amino acid residues will face the same side, and thus hydrophobic residues arranged with this periodicity will stabilize a protein domain by interacting with other secondary structural elements. Also, amino acid residues that would prevent formation of particular types of second structural elements were best avoided from regions identified to form such elements. Deletion or insertion of amino acid residues was best avoided at the midst of the regions predicted to form secondary structural elements. The alignment was improved so that residues stereochemically atypical, such as proline, glycine, and tryptophan, were best occupying the same positions among the FFRPs.

Consequently, eight FFRPs of P. aeruginosa were identified, and a multiple alignment correlating their amino acid residues to those of FFRPs of other bacteria was finalized (FIG. 1).

### Example 2

### Identification of the Phylogenetic Relation Between FFRPs

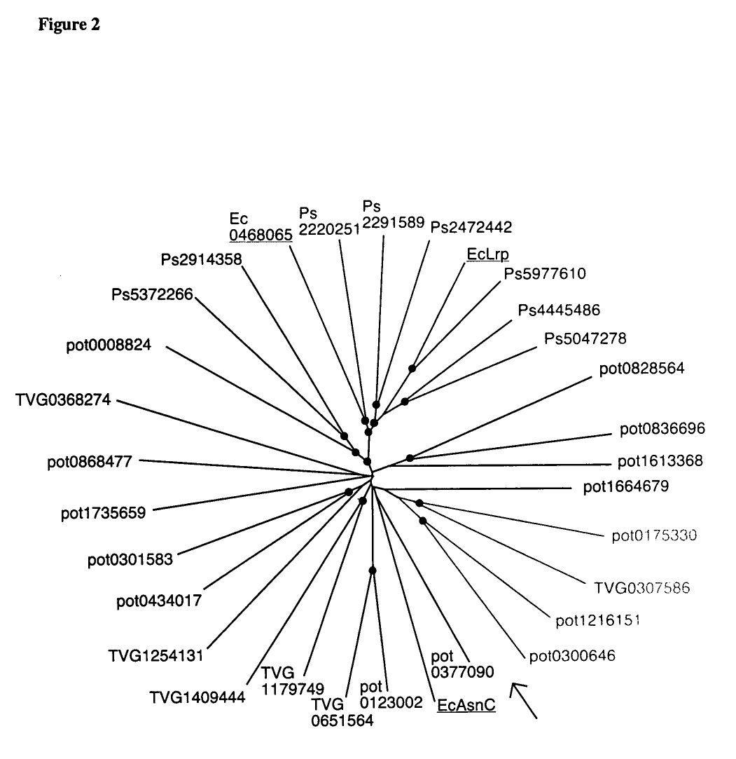

Without changing the correlation between residues in the multiple alignment in FIG. 1 and by using the PHYLIP package, FIG. 2 was made showing similarities between FFRPs. In FIG. 2, two FFRPs related more closely are separated by a shorter distance, this distance being defined as the sum of partial distances measured from the node separating the two FFRPs to the respective FFRPS. For example, the protein related closest to E. coli Lrp is Ps5977610 from P. aeruginosa.

A bootstrap value was calculated for each node in FIG. 2. A bootstrap value is a measure for evaluating degree of homogeneity of data used to deduce a conclusion: a conclusion needs to be unbiased, supported by essentially the whole of the data set. This value defines the number of times the same diversification at a node is concluded out of, for example, 1,000 trials, while effectively changing the weight given to each of the amino acid positions used for the analysis. In extreme cases, some positions might be excluded from the analysis or only several positions might be selected. Phylogenetic relation indicated by a node in a model is more accurate when the bootstrap value approaches 1,000.

Bootstrap values calculated to nodes inside regions including the eight FFRPs of P. aeruginosa (highlighted red or blue in FIG. 2) were generally high, indicating higher reliability of the phylogenetic relations determined between these proteins. In particular, the bootstrap value calculated to the node separating E. coli Lrp and Ps5977610 was higher than 900/1,000, and the orthologous relationship between the two proteins was unambiguous. The closest to Ec0468065 of E. coli was Ps2220251 of P. aeruginosa, but the bootstrap value calculated to the node relating the two was not very high (FIG. 2). No P. aeruginosa FFRP was found resembling E. coli AsnC.

### Example 3

### Identification of Ligand-Binding Site

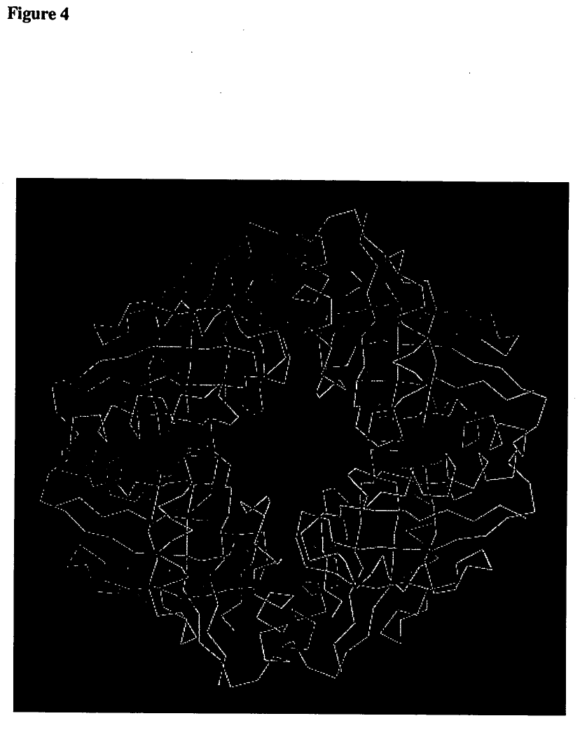

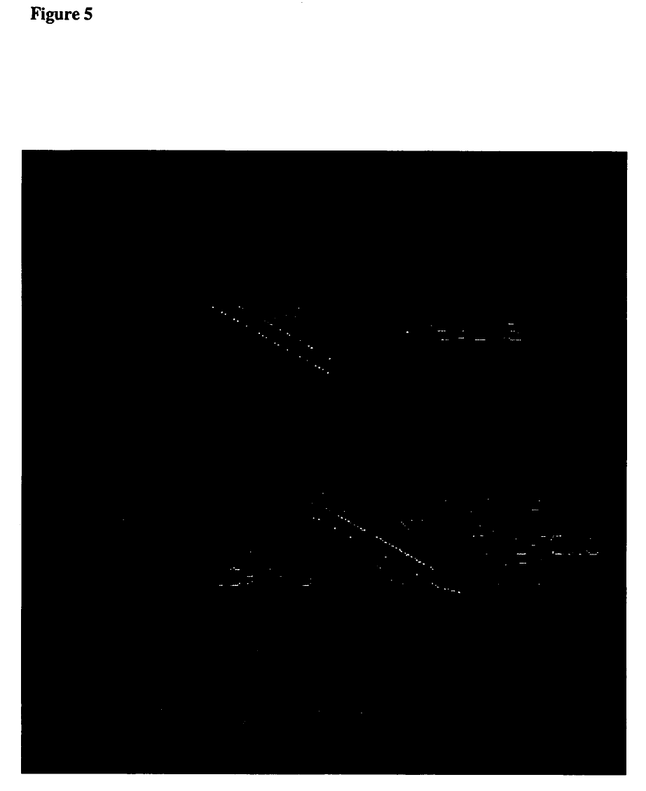

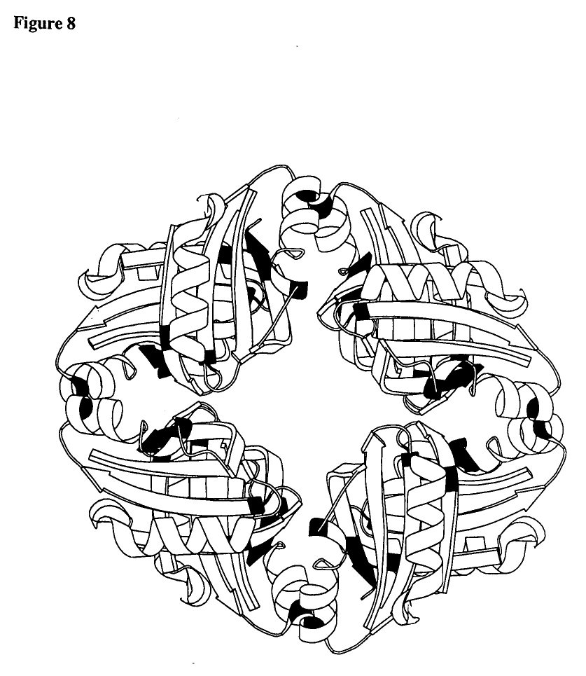

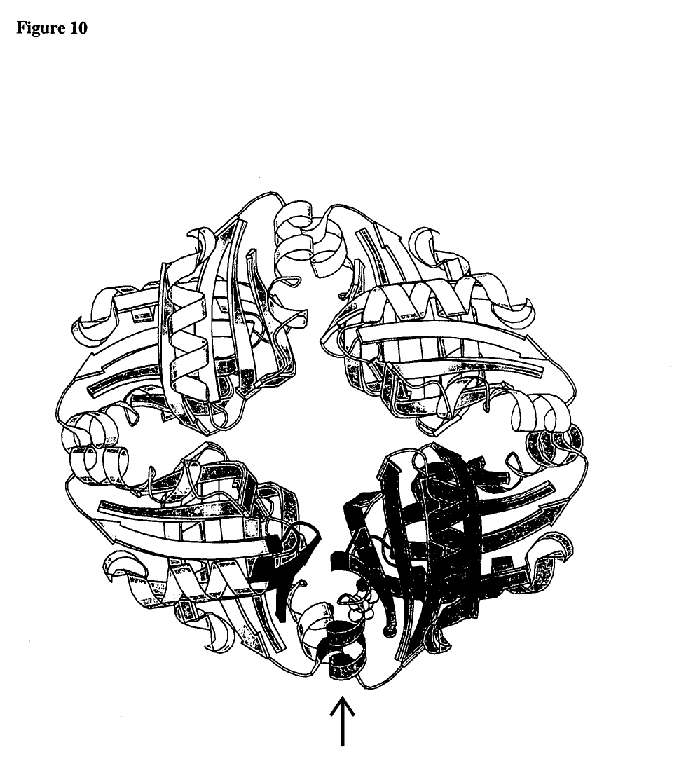

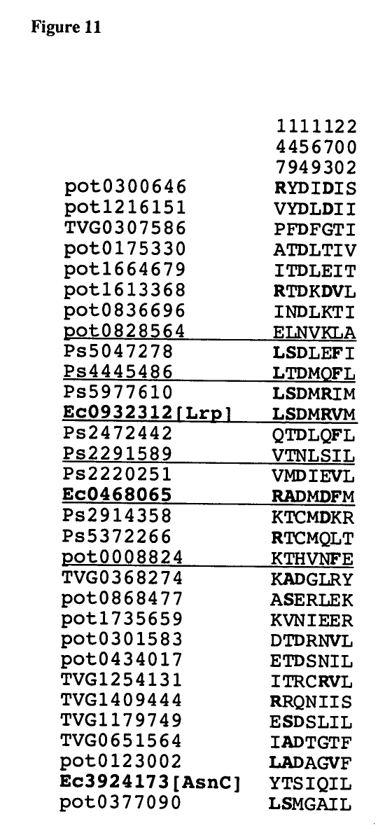

By using correlation of amino acid sequences between P. aeruginosa FFRPs and pot1216151 shown in FIG. 1 and by using findings obtained by analyzing the crystal coordinates of the archaeal FFRP, pot1216151 (see REFERENCE EXAMPLES and Patent Application No. 2001-384683), in total thirty-two amino acid positions were identified for each of the FFRPs from bacterial species as potential targets while developing a pharmaceutical agent (FIG. 3). The crystal structure of pot1216151 used for this identification is the only 3D structure of any FFRP determined to date in complex with a ligand. It is the single FFRP structure where the positions of the side chains of amino acid residues are determined precisely. The identification of these positions was carried out by four different approaches described below.
i) In the crystal of pot1216151, each octamer was found in complex with two molecules of an unidentified assembly promotion factor (i.e., a ligand), this ligand having being absorbed from E. coli cells upon expression of the protein (see REFERENCE EXAMPLES and Patent Application No. 2001-384683). Related by a crystallographic symmetry, the two ligand molecules are binding to identical sites in the pot1216151 assembly (FIG. 4). The size of the ligand was similar to that of a medium amino acid, e.g., valine: a valine molecule was modeled by filling the electron density of each ligand molecule (FIG. 5). Eighteen amino acid residues whose at least one non-hydrogen atom was positioned within 6 Å from the modeled valine, when measured from carbon, oxygen, or nitrogen atoms in the valine (FIG. 6). It has been concluded that in general in FFRPs, the eighteen positions, i.e., 113, 126, 147, 148, 149, 150, 152, 153, 154, 155, 176, 177, 181, 196, 197, 198, 199, and 202, have potentials to interact with ligands, wherein these positions are identified using FIG. 1.
   ii) Molecular genetic experiments using E. coli Lrp have shown that leucine-dependence of transcription regulation by Lrp was affected by mutation at each of seven positions (Platko, J. V., and Calvo, J. M., 1993, J. Bacteriol., 175, 1110-1117). These seven positions, in E. coli Lrp, are candidates which possibly interact with leucine. With the 3D structure of E. coli Lrp being unknown, residues of pot1216151, identified as occupying the equivalent positions (147, 154, 169, 181, 199, 200, and 201 in FIG. 1) were found surrounding the gap formed in the FFRP assembly (FIGS. 7 and 8). Among the seven residues of pot1216151, the side chain of Met (199) only was found facing away from the gap, facing into the protein domain, although its position was close to the gap. Of the remaining six residues, the main chain carboxyl group of Ala (201) and the side chains of Val (147), Asp (154), Leu (169), Arg (181), and Ile (200) were found facing the gap. It was concluded that with these seven positions generally in FFRPs, ligands interact potentially.
iii) Leucine interacts not only with E. coli Lrp (Marasco, R. et al., 1994, J. Bacteriol., 176, 5197-5201) but also with pot1216151 (REFERENCE EXAMPLES and Patent Application No. 2001-384683), thereby disassembling these FFRPs. Up to two molecules of leucine binds to E. coli Lrp per gap (Marasco, R. et al., 1994, J. Bacteriol., 176, 5197-5201) formed between a pair of dimers. In order to analyze possible stereochemical changes induced upon interaction between leucine and pot1216151, two leucine molecules were modeled using a computer and added to the crystal structure of pot1216151 by fitting into the gaps formed between a pair of dimers. Each leucine molecule was modeled near the seven residues described above. As shown in FIG. 9, the two gaps are related by a pseudo two-fold symmetry, each gap potentially interacting with one molecule of leucine. Here, only one leucine molecule is shown in FIG. 9, by fitting into the lower gap. FIG. 9 is a view of the complex when looked through the direction indicated by the arrow in FIG. 10, showing only the two dimers closest to the arrow. The side chain of the leucine molecule was modeled extending from the vicinity of Val (147) through Leu (169) towards Ile (200), so that hydrophobic interactions will be formed. The amido group of leucine was modeled approaching Asp (154) of pot1216151, while the amido group of leucine approached Val (147), both forming chemical bonds. The overall complementality between the leucine molecule and the gap was found ideal; however the leucine molecule was positioned too close to Tyr (149), Asp (173), and Ile (202) of pot1216151, creating van der Waals conflicts. Thus, this complex will not be stable, but might dynamically change. At the five positions from which interactions were made without a conflict, E. coli Lrp and pot1216151 share the same or similar amino acid residues (FIG. 11). It was thus concluded that ligands potentially bind to the seven positions of FFRPs in general.
iv) Using the 3D structure of pot1216151, twenty-eight amino acid positions were identified as forming the gaps between dimers. Equivalent positions in FFRPs in general are candidates potentially interacting with ligands. Namely, amino acid residues were identified as forming gaps when they were inaccessible by a probe of the radius 5.0 Å moving by contacting the surface of the assembly, but at least partially accessible by another probe of the radius 1.4 Å, when this accessible area was 5% or larger of the area exposed when the same type of amino acid residue was fully stretched between a pair of glycine residues. In the crystal structure, four gaps were formed unrelated by any crystallographic symmetry, and twenty-eight residues were identified as forming two or more of the four gaps (FIG. 12). These residues are found at 116, 119, 120, 126, 144, 145, 147, 148, 149, 150, 152, 153, 154, 166, 169, 173, 177, 181, 183, 185, 193, 195, 196, 197, 198, 199, 200, and 201 shown where these positions are identified using FIG. 1.

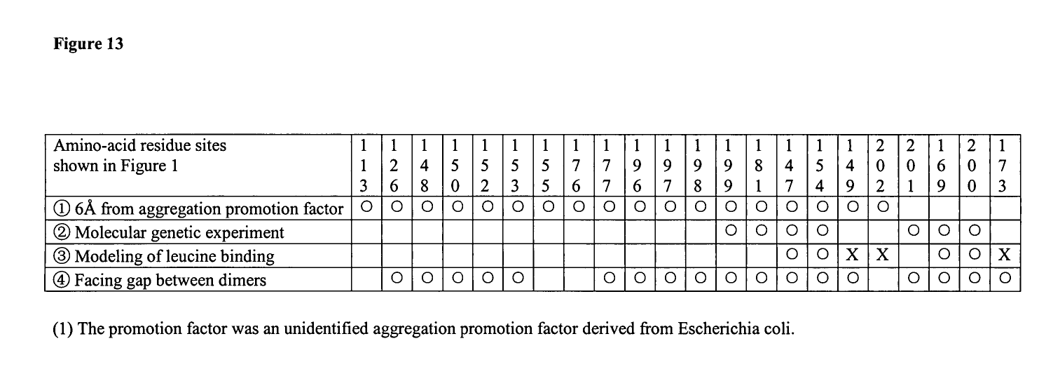

As the sum of identifications i) to iv), thirty-two positions are listed in FIG. 3. These positions are interpretable as forming two ligand-binding sites partially overlapping onto each other (*Typographical error existed here, noted by the translator). Of the positions listed in FIG. 3, when ten positions, 116, 119, 120, 144, 145, 166, 183, 185, 193, and 195, identified only in iv) but not in i)-iii) were removed, twenty-two positions shown in FIG. 13 remained. Of these, the E. coli unidentified assembly promotion factor binds around positions identified in i) only, or in both i) and iv), i.e., twelve positions on the left in FIG. 13. While, the disassembling factor, leucine, binds around four positions on the right in FIG. 13, identified in iv) and one or both of ii) and iii). At around the six positions in between, the two types of ligands might compete with each other for binding.

### Example 4

### Further Specifying Target P. aeruginosa FFRPs in Order to Prevent Possible Interaction of Pharmaceutical Agents to be Developed with E. coli

E. coli Lrp and P. aeruginosa Ps5977610 have very similar amino acid residues at positions important for interaction with ligands. The two amino acid sequences shown in FIG. 1 are similar to each other as a whole. These facts suggest that the two proteins are regulated in the same way by the same ligands including leucine. None of the P. aeruginosa FFRPs was found to be resembling E. coli AsnC. The protein most resembling E. coli Ec0468065 was Ps2225251, but the similarity between the two was not high. Of the other six P. aeruginosa FFRPs, Ps2914358 and Ps5372266 were found to be differentiated from all the three FFRPs of E. coli.

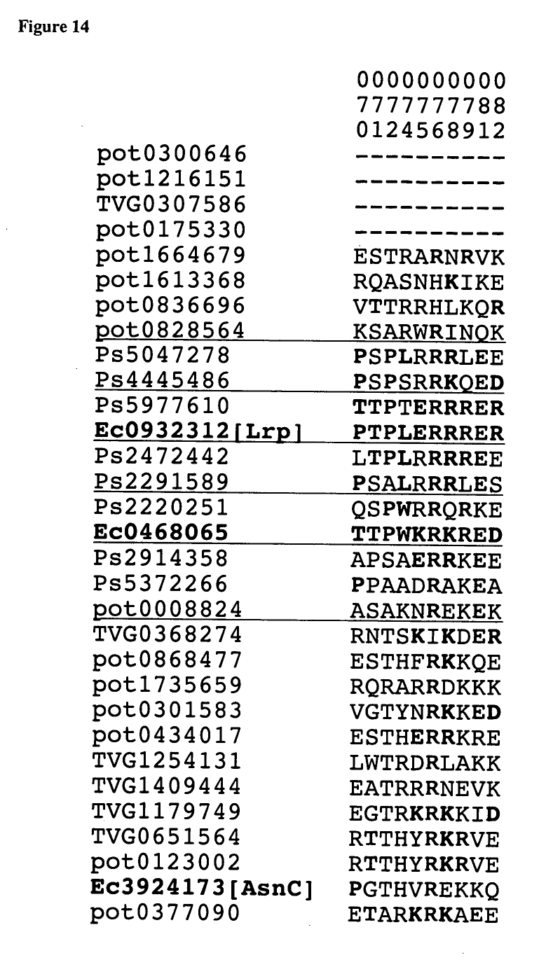

It has been discussed that, of the three a helices formed inside the N-terminal domain of an FFRP, the third helix is important for recognizing DNA bases, but that three positions of the third helix are unable to recognize DNA, since they are often occupied by hydrophobic residues, facing into the protein domain (Suzuki, M., et al., 2003, Proc. Japan Acad. 79B, 92-98). Based on the multiple alignment shown in FIG. 1, amino acid residues forming the third α helix of each P. aeruginosa FFRP were identified, and twelve positions potentially recognizing DNA bases were identified (FIG. 14). At these positions E. coli Lrp and P. aeruginosa Ps5977610 had very similar amino acid residues, suggesting that both will recognize the same DNA sequence. Ec0468065 and Ps2225251 were found sharing the same residues at four positions out of the twelve positions. Among the FFRPs of P. aeruginosa, Ps2914358 and Ps5372266 have differentiated farthest from the three E. coli FFRPs. No P. aeruginosa FFRP was found sharing three or more residues with E. coli AsnC at the same positions. These findings are consistent with the conclusion obtained by analyzing positions possibly interacting with ligands, and also with those drawn by comparing the whole amino acid sequences.

### Reference Example 1

### Crystallization of an FFRP

(1) Construction of a Vector for Expressing an FFRP

The following primers were synthesized in order to amplify a DNA fragment coding an FFRP (pot1216151) gene by PCR using the genomic DNA of Pyrococcus sp. OT3 (JCM 9974) as the template. PCR was carried out using the reaction solution, 20 μl, LA Tag (Takara Shuzo Co., Ltd.) and GeneAmp PCR system 9600 (Perkin Elmer): each cycle consisting of denaturation at 94° C., annealing at 55° C., and elongation at 72° C., and repeating this cycle 30 times.

The amplified DNA fragment was cleaved using restriction enzymes NdeI (Takara Shuzo Co., Ltd.) and BamHI (Takara Shuzo Co., Ltd.), and the fragment coding the gene was separated from the rest by electrophoresis using an agarose gel. The DNA fragment coding the FFRP (potl216151) gene was ligated with an expression vector, pET3 (Novagen), cleaved using restriction enzymes, NdeI and BamHI, thereby constructing an expression vector (pET3-LRPS01): 10 μl of the reaction solution, 660 mM Tris-HCl buffer (pH 7.6) containing 66 mM MgCl₂, 100 mM DTT, and 1 mM ATP, was kept at 16° C. overnight, in the presence of T4 DNA ligase (Takara Shuzo Co., Ltd.).

Primers:

(SEQ ID NO: 32)

Forward Strand:
5′-TGGTGATGACATATGGTGACGGCCTTTATCCTG-3′

(SEQ ID NO: 33)

Reverse Strand:
5′-GAACGGATCCATCAAATTGCTATCATAGTCGAGGTC-3′

(2) Expression of an FFRP using E. coli

The vector designed for expressing the FFRP (pot1216151) gene, pET3-LRPS01, was introduced into E. coli cells strain BL21 (DE3) (Novagen). The E. coli cells were cultured in 8 ml of the 2×YT medium containing 50 mg/ml ampicillin at 37° C. overnight, inoculated into 8 l of the same medium, and allowed to grow until the absorbance of the medium reached approximately 0.75 at 600 nm. Subsequently, 2 mM isopropylthiogalactoside (IPTG) was added to induce expression of the FFRP. After additional culture for 4 hours, cells were collected by centrifugation at 7,500 rpm for 5 minutes using a centrifuge (Beckman Avanti J-25).

(3) Purification of an FFRP (pot1216151)

The cells expressing the FFRP (pot1216151) were suspended into 250 ml of a 100 mM Tris-HCl buffer (pH 7.0) containing 1 mM EDTA, and treated using a French press (SLM). After centrifugation for 30 minutes at 4° C. at 25,000 rpm using a centrifuge (Beckman Avanti J-25) and a rotor (JA-25.5), the supernatant was collected and heated at 75° C. for 1 hour. After another centrifugation at 4° C. at 25,000 rpm for 30 minutes, the supernatant was dialyzed against a 30 mM Tris-HCl buffer (pH 7.0), and subjected to further purification: by twice repeating the following process, i.e., anion exchange column chromatography followed by gel filtration.

The supernatant was applied to an anion exchanger (1.6×18 cm, Resource Q, Pharmacia), in a column, equilibrated with 30 mM Tris-HCl buffer (pH 7.0), and the column was washed with the same buffer. Subsequently, the protein was eluted with a linear gradient, 0-2 M of NaCl in the buffer, with a flow rate of 2 ml/min, using FPLC column (Pharmacia). The solution eluted from the column was fractionated into fractions of 4 ml. By SDS electrophoresis, fractions containing the FFRP were identified and stored.

The stored protein solution was dialyzed against 100 mM Tris-HCl (pH 7.0) and subjected to gel filtration. The protein solution was applied to a gel column (2.6×60 cm, Superdex 75, Pharmacia) equilibrated with a 100 mM Tris-HCl buffer (pH 7.0), and eluted with the same buffer at a flow rate of 1 ml/min using the FPLC system (Pharmacia). The fractions obtained (2 ml each) were subjected to SDS electrophoresis, and those containing the FFRP were identified and stored. The above processes of anion exchange chromatography and gel filtration were alternately carried out two times to purify the FFRP until a single band was observed in an electrophoretogram.

(4) Crystallization of the FFRP (pot1216151)

The purified FFRP, 20 mg/ml, was dissolved in a 10 mM Tris-HCl buffer (pH 7.0) to prepare a sample for crystallization. The sample was crystallized on a 24-well crystallization plate (Hampton Research) by a vapor-diffusion method using a sitting-drop technique. Specifically, using a mixture of 4 μl of the sample and 4 μl of a reservoir solution (100 mM citrate buffer (pH 4.0) containing 10% by weight of PEG 6000 and 1.0 M lithium chloride) as the mother solution, the sample was allowed to be equilibrated with 0.8 ml of the reservoir solution by vapor diffusion at a constant temperature of 5° C. In approximately a week, six-sided pyramid crystals of about 0.2 mm were obtained.

### Reference Example 2

### Analysis of the 3D Structure of the FFRP

(1) X-Ray Diffraction Analysis

In order to determine the 3D structure by a heavy atom isomorphous replacement method, the FFRP (pot1216151) crystal obtained in REFERENCE EXAMPLE 1 was soaked in a preservative solution containing 10 mM platinocyanide (K₂[Pt(CN)₆]) or 0.1 mM gold chloride (K[AuCl₄]) at 5° C. for about 2 days.

Four sets i.e., two sets of FFRP original crystals and two sets of derivatives respectively derived from two heavy atoms (platinum and gold), were measured using two X-ray source: 1) a laboratory rotating anode X-ray source, and 2) a synchrotron radiation X-ray source.

1) Analysis Using a Laboratory Rotating Anode X-Ray Source

A set of original crystals of the FFRP (pot1216151) and two sets of derivatives from two heavy atoms (platinum and gold) were analyzed at room temperature by using the laboratory rotating anode X-ray source. Each set was placed in a quartz capillary (1.5 mm in diameter, produced by TOHO Co., Ltd.), and an excessive solution was removed. After both ends of the capillary were sealed with wax, the capillary was set in an X-ray diffractometer. The X-ray source (UltraX 18 produced by Rigaku Industrial Corporation) was operated with 50 kV and 100 mA, and diffraction data was recorded with a diffraction device (R-AXIS IV produced by Rigaku Industrial Corporation).

2) Analysis Using A Synchrotron Radiation X-Ray Source

In order to obtain data at a higher resolution, the original FFRP (pot1216151) crystal was measured in a frozen state by using the synchrotron radiation X-ray source. The synchrotron radiation X-ray source used was Hyogo-ken Beam Line BL24XU of photon factory SPring8. The crystal was immersed in a cryopreservation solution [20% (by weight) glycerol, 14% (by weight) PEG 6000, 1.0 M lithium chloride, and a 100 mM citrate buffer (pH 4.0)] for several minutes, mounted on a 0.2-mm mount loop (Cryoloop, produced by Hampton Research Corporation) for cryopreservation, and was frozen in liquid nitrogen (−196° C.). During the analysis, the temperature of the crystal was kept at −173° C. using a device designed to spray cold air to crystals (produced by Rigaku Industrial Co., Ltd.). Diffraction was recorded by a diffractometer (R-AXIS IV produced by Rigaku Industrial Co., Ltd.) using a synchrotron radiation X-ray having a wavelength of 0.834 Å.

(2) Data Analysis

The recorded diffraction was processed using a program designed for the diffractometer and the programs in the CCP4 package (Collaborative Computational Project, Number 4, Acta Crystallographica D50, 760-763, 1994). The space group of the crystals was identified as belonging to a space group P3₂21 or P3₁21, and the unit cell lengths were identified: a=b=96.3 Å, and c=97.1 Å. Based on these lengths, it was concluded that each asymmetric unit of these crystals contained three to six molecules of the FFRP.

(3) Building an Initial Model

The diffractions from the FFRP pot1216151 and the two heavy atom (platinum and gold) derivatives were integrated using programs in the CCP4 package, and used for the subsequent computational analysis. Positions of the heavy atoms in the derivatives were determined using difference Patterson maps. The gold and platinum derivatives, respectively, had two and three heavy metal atoms per asymmetric units. Numbers, positions, and occupancies of the heavy atoms were refined using the MLPHARE program in the CCP4 package. The overall figure of merit at this stage was calculated as 0.43 at 3.0 Å.

By using the phase obtained as above and by using the isomorphous replacement method, an electron density map was calculated. By examining the map, the phase was improved by the methods of solvent flattening and histogram matching, using the DM program in the CCP4 package. In the revised electron density map calculated using the improved phase, electron densities corresponding to right-handed α-helices were identified appropriately only when four molecules of the protein were assumed in each asymmetric unit and when these units were related by the space group P3₂21. A model was made by assembling four monomers (each consisting of 72 alanine residues), so that the model would best fit to the electron density map using program O (Jones T. A., Zou J. Y., Cowan S. W., and Kjeldgaard M., Improved methods for binding protein models in electron density maps and the location of errors in these models, Acta Crystallographica A47, 110-119, 1991).

(4) Refinement of the Model

The initial model was refined using the X-PLORE program (Brünger, A. X-PLORE v3.1 Manual (Yale University, New Haven, 1992)), so that the model would best satisfy both the diffraction data experimentally obtained and the standard geometric parameters (Engh and Huber) that were expected for protein 3D structures in general. The model was further refined using program O to correct the part of the model where large deviations from the electron density map were observed. Until this stage, the four monomers in the asymmetric unit were kept identical to each other. The R factor indicating the difference between the experimental data and the model was 39.1% at this stage.

In order to further refine the model, the high-resolution data obtained at SPring8 BL24XU were used. The aforementioned alanine model was refined using the X-PLORE program using data up to 3.0 Å, and other data up to 2.0 Å were incorporated while improving the phase using the wARP program (Perrakis, A., Sixma, T. K., Wilson, K. S., and Lamzin, V. S., wARP: Improvement and extension of crystallographic phases by weighted averaging multiple refined dummy atomic models, Acta Crystallographica D53, 448-455, 1997). Using the improved phase, a new electron density map was calculated, where electron densities of most of the atoms in the protein, including the atoms of side chains of the amino acid residues, were clearly identified. Based on this electron density map, another 3D structural model was made by including the amino acid side chains of the FFRP (pot1216151).

Further refinements were carried out at a resolution of 1.8 Å, by modeling solvent molecules so that there would match with electron densities that were clearly identified but not interpretable as any atoms in the protein, which will be discussed further in the following paragraph. At the final stage of refinement, the four monomers in the asymmetric unit were allowed to adopt non-identical 3D structures.

(5) Determination of the Final Model

The final model (FIG. 8) was assembled by four independent monomers of the FFRP (pot1216151). Each monomer comprises all the residues except for the first amino acid residue, i.e., methionine, at the N-terminal end. The R factor, evaluating the consistency between the experimental data (20-1.8 Å) and the final model, was calculated as 21.2%, indicating a high accuracy of the 3D model. The PROCHECK program in the CCP4 package was used to calculate a Ramachandran plot. In the plot, 98.5% of all the residues were found in energetically most stable regions. The average temperature factor (B-factor) was as small as 17.3, indicating that all the atoms were determined unambiguously, except for some side chains, which were positioned on the surface, and thus expected as possessing real flexibilities for movements.

### Reference Example 3

### Analysis of the FFRP by Gel Filtration

It has been reported that many FFRPs derived from microorganisms, including E. coli, form dimers or tetramers in solution. The FFRP expressed in E. coli was analyzed by gel filtration in order to estimate sizes of its assemblies in solution.

Specifically, the FFRP pot1216151 purified for the crystallization was diluted with a 50 mM Tris-HCl buffer (pH 7.0) containing 300 mM sodium chloride, yielding a concentration of approximately 100 μM. Gel filtration was carried out at a flow rate 1 ml/min (LC Module I plus System, Waters) using a matrix (Protein Pak 125, 7.8×300 mm, Waters) and the same buffer. Retention of the protein was recorded by measuring the UV absorption at 220 nm.

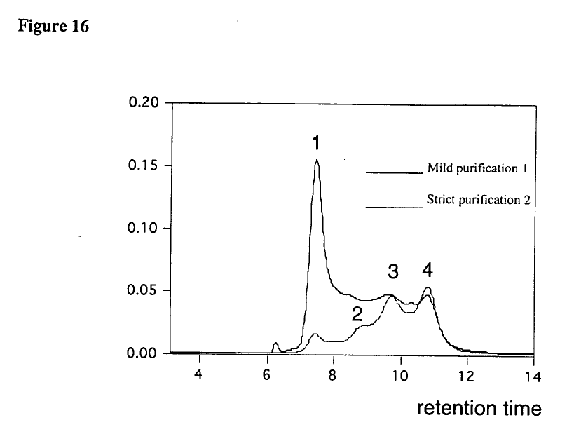

A multiple number of peaks were observed (FIG. 16, purification 1). In general upon gel filtration, assemblies of larger sizes will be retained for a shorter time. Accordingly, the first peak (peak 1 in FIG. 16) might correspond to an octamer, and other peaks retained longer should correspond to smaller assemblies. This protein forms a variety of assemblies up to an octamer depending on the condition. The function of the octamer may be different from those of smaller assemblies.

### Example 4

### Analysis of the 3D Coordinates of Atoms in the FFRP pot1216151

(1) The 3D Coordinates of Atoms in the FFRP

The atomic coordinates of the 3D structure of the FFRP pot1216151 determined are shown using the protein data bank (PDB) format (Table 1), describing the four independent monomers of the protein (A to D) in the asymmetric unit and 196 water molecules. Lines 1 to 7 of Table 1 specify the crystal symmetry to repeat the asymmetric unit, as is generally the case of any crystals. From line 8, eleven parameters (i)-(xi) described from left to right are: (i) serial numbers of the atoms (1 to 2,556); (ii) types of atoms, e.g., C, the carbon, further differentiated as, for example, CA, CB, indicating the positions inside the amino acid residues, also including information concerning chemical bonding; (iii) assignments of the atoms to 20 types of amino acid residues, using the 3 letter code, e.g., VAL for valine and HOH for the atoms belonging not to amino acid residues but to water molecules, and, in addition, their assignments to the four monomers (A to D) or to water molecule (W); (iv) amino acid residue numbers counted from the N terminus in each monomer, or numbers 1 to 196 for the water molecules; (v) the X coordinates in angstrom; (vi) the Y coordinates in angstrom; (vii) the Z coordinates in angstrom; (viii) occupancies (i.e., probabilities of the atoms occupying the given coordinates); (ix) isotropic temperature factors (i.e., measures of the flexibilities of atoms for thermal movements); (x) the atomic numbers defined by the periodic table (e.g., 6 for C, 7 for N, and 8 for 0); and (xi) types of the atoms, the same as in (ii) (e.g., C, O, and N).

The atoms are named according to the IUPAC-IUB nomenclature; however, in (ii), A, B, C, D, E, Z, and H were used (e.g., CA or CB) instead of α, β, γ, δ, ε, ζ, and η. The additional oxygen atoms present at the C termini were labeled as OXT (OT).

(2) Analysis of the 3D Coordinates

1) Formation of Assemblies of the FFRP pot1216151

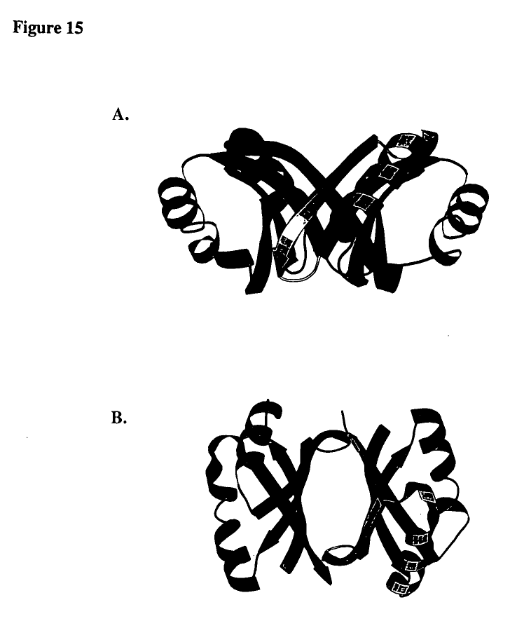

By analyzing the data shown in Table 1, it was found that each monomer of the FFRP is composed of a four-stranded β sheet and two α-helices (FIG. 15). Each pair of monomers assembled into a dimer, by forming a single β-strand composed of eight strands, i.e., four strands from each monomer (FIG. 15). In the crystal, four such dimers further assembled to form a disk-like octamer (FIG. 3). The diameter of the octamer disk was approximately 60 Å and the thickness thereof was approximately 40 Å.

2) Characteristics of the 3D Structure of the FFRP pot1216151

In the central region of the disk-like octamer of the FFRP pot1216151, there exists a hole, the overall shape resembling a Japanese 50-yen coin. Four gaps, each formed between a pair of dimers, extend from this hole, thereby forming a space having a cross-like shape (FIG. 8). The shape of this space, and types and coordinates of amino acid side chains surrounding this space are the 3D structural information important for screening for ligands interacting with the FFPR.

The hole in the central region resembled a cylinder having a diameter of approximately 12 Å and a height of approximately 30 Å. The hole was large enough to accommodate two amino acid molecules. Eight sets of amino acid side chains respectively from eight FFRPs in the assembly, valine 9, glutamic acid 37, tyrosine 38, methionine 68, and serine 70, face this hole (here numbers, such as 9, 37, or 38, are those of amino acid residues, counted from the N terminus in each FFRP monomer (see Table 1). Also, residues 37, 38, and 68 are positioned on the border between the hole and the gaps.

Each of the four gaps formed between the pairs of the dimers is large enough to accommodate two amino acids, or able to do so by being expanded by the two amino acids. Each gap was surrounded by two sets of side chains of the two dimers, glutamic acid 15, valine 33, tyrosine 35, aspartic acid 39, leucine 49, aspartic acid 53, isoleucine 56, threonine 57, arginine 61, threonines 69 and 71, and isoleucines 73 and 75.

3) Application of the 3D Information to Screening for Ligands

If a ligand binds to the space extending from the hole to the gaps and if the ligand is completely complementary to part of the space and thus able to fit into, the ligand will stabilize the octamer thereby activating the functions of the FFRP as an octamer. However, if the ligand is larger than the space and binds there with a high constant, it will enlarge the space, thereby dissociating the octamer, terminating the functions of the FFRP as an octamer, and activating functions as smaller assemblies. Most likely, this space is where interaction with natural ligands will occur.

As has been described, graphical and/or numerical information of the FFRP obtained from the 3D coordinates shown in Table 1 defines the space extending from the hole to the gaps and specify the positions of atomic groups, hydrophobic or hydrophilic, facing the space. Thus, this information provides a useful platform for screening for ligands interacting with the FFRP.

### Reference Example 5

### The Position of a Natural Ligand Present in the Crystal of FFRP

(1) The Presence of a Natural Ligand in the Crystal of the FFRP pot1216151

The presence of a natural ligand in the crystal obtained in REFERENCE EXAMPLE 1 was confirmed as described below.

1) Purification of the FFRP (pot1216151) using a Method Different from what was Described in REFERENCE EXAMPLE 1

The protein was purified by a more rigorous method with additional uses of ammonium sulfate fractionation and hydrophobic column chromatography. Ammonium sulfate fractionation was carried out after the heat treatment and centrifugation. Namely, ammonium sulfate was added to the supernatant, yielding 40% saturation. The solution was centrifuged at 4° C. at 18,000 rpm for 15 minutes. Ammonium sulfate was further added to the supernatant, yielding 80% saturation. After the centrifugation at 4° C. at 18,000 rpm for 15 minutes, the sediment was dissolved in 30 ml of a 30 mM Tris-HCl buffer (pH 7.0) and dialyzed against a 30 mM Tris-HCl buffer (pH 7.0). Hydrophobic column chromatography was carried out after the anion exchange column chromatography and the gel filtration described in REFERENCE EXAMPLE 1. Namely, after the gel filtration, ammonium sulfate was added to the protein solution, yielding a concentration of 1.6 M, and applied to a column (1.6×18 cm), containing Butyl-Toyopearl 650 M (Tosoh) pre-equilibrated with 50 mM Tris-HCl buffer (pH 7.0) containing 1.5 M ammonium sulfate. The column was washed with the same buffer. Subsequently, the protein was eluted using a line, 1.5-0 M, gradient of ammonium sulfate in the buffer with a flow rate 2 ml/min: the FPLC column (Pharmacia) was used. Fractions, 4 ml each, were subjected to SDS electrophoresis and those identified as containing the FFRP were stored.

2) Changes Between Assembly Forms

Changes in apparent molecular weight of the FFRP (pot1216151) in the solution, i.e., changes in the assembly form, were confirmed (FIG. 16, purification 2) by electrophoresis. Compared with the method employed in REFERENCE EXAMPLE 1, in this method, the protein is exposed to the environments accelerating, in general, denaturation of proteins. This suggests that the ligand, which has been derived from E. coli and stabilized the assembly of the FFRP, dissociated from the FFRP after the rigorous purification, thereby failing to form an octamer. The analysis thus suggests that a natural ligand that stabilizes the assembly of the FFRP exists in the cells of E. coli and that the crystal obtained in REFERENCE EXAMPLE 1 was in complex with this natural ligand.

(2) The Position of the Natural Ligand in the Crystal of FFRP (pot1216151)

The final model showing the 3D structure of the FFRP (pot1216151) was re-examined to analyze non-protein regions (the regions interpreted to correspond with 196 water molecules) in further detail. Electron densities which likely possibly correspond to two molecules of the ligand were identified at two positions inside the gaps formed between the dimers (FIG. 4).

As has been discussed, what can be determined experimentally by X-ray diffraction analysis is an electron density map, and the 3D coordinates obtained best fitting the map are still a model. While a large number of water molecules are generally contained in crystals, it is extremely difficult to identify molecules as small as water in an electron density map. Of the atoms constituting a water molecule, the two hydrogen atoms diffract X-rays very poorly, and thus, in short, they are invisible by X-ray analysis. Consequently, in order to identity a water molecule, a point-like density of the oxygen atom isolated from any other density needs to be identified in the electron density map. In the present model, densities identified as corresponding to molecules other than FFRP molecules are first assumed as derived from 196 water molecules, i.e., 196 oxygen atoms in particular. If a natural ligand originated in E. coli cells is present in the crystal, one of such electron densities should correspond to that of the natural ligand.

In order to identify the electron density of the ligand, water molecules modeled to relatively low electron densities were removed. Of these, 66 were found inside the hole at the center of the octamer. This was necessary to remove distortion of the map caused by possible misidentification of these atoms. Starting with this model, the process of 3D structure refinement was repeated several times, and the difference between the model and the electron density map (hereafter referred to as the difference Fourier map) was computed, expecting that a density should emerge, corresponding to the ligand. At each cycle, high densities newly found in the difference Fourier map (at counter levels of three or higher) were examined thoroughly. To these densities, which were isolated or which were close enough to hydrophilic groups in the protein so that ionic/hydrogen bonding was possible, new water molecules were modeled. Finally, an electron density larger than that expected for a water molecule, was identified in each asymmetric unit. Since an octamer is composed of two such asymmetric units two such densities were found in the octamer (FIG. 4). These two densities were interpreted as those of two molecules of the ligand, each positioned inside the gap formed between a pair of dimers. Each electron density has a size equivalent with four or more water molecules, or equivalent with a single molecule of a medium sized amino acid such as valine or isoleucine.

### Example 6

### Interaction Between the FFRP and Natural Ligands in Solution

It has been confirmed that various amino acids and some other biomolecules (e.g., metabolic intermediates) can act as natural ligands on assemblies of the FFRP (pot1216151).

(1) Changes in Crystal Structure by Amino Acids

It has been known that the regularity inside a crystal might be affected, if the structure of the protein changes upon interaction with a ligand which is incorporated from outside the crystal, thereby depolarizing the crystal. Crystals of the FFRP (pot1216151) were soaked into solutions, each containing one of twenty amino acids at the same concentration 10 mM. With isoleucine, valine, methionine, and leucine, the crystals depolarized after several hours. With the other amino acids, no change was observed. It is important to note that the changes caused by the four amino acids to the 3D structure of the protein are not necessarily the same, and that it is possible that the other amino acids, although they showed no effect, had no ability to diffuse into the crystal.

(2) Analysis Using Gel Filtration

Changes in assemblies of the FFRP pot1216151 induced by interaction with amino acids and metabolic intermediates were analyzed in solution using gel filtration.

1) Experimental Procedure

Gel filtration was carried out at a flow rate 1 ml/min, using a matrix (Protein Pak 125, 7.8×300 mm, Waters) and a 50 mM Tris-HCl buffer (pH 7.0) containing 300 mM sodium chloride. Flow of the protein was monitored by measuring the UV absorbance at 220 nm (LC Module I plus system, Waters). The FFRP pot1216151 purified by method 2, i.e., the rigorous method, was dissolved in 50 mM Tris-HCl buffer (pH 7.0) containing 200 mM sodium chloride, and kept at −80° C. until it was diluted by 10-fold with a 50 mM Tris-HCl buffer (pH 7.0) containing 300 mM sodium chloride, and mixed with each potential ligand for at least 30 minutes at room temperature (about 25° C.). In the mixture, the monomer concentration of the FFRP was approximately 100 μM. While, the concentration of each-amino acid was 1 mM and that of 5 metabolic intermediates, i.e., malic acid, 2-oxoglutaric acid, oxaloacetic acid, pyruvic acid, and 3-phosphoglyceric acid, was 10 mM.

2) Results

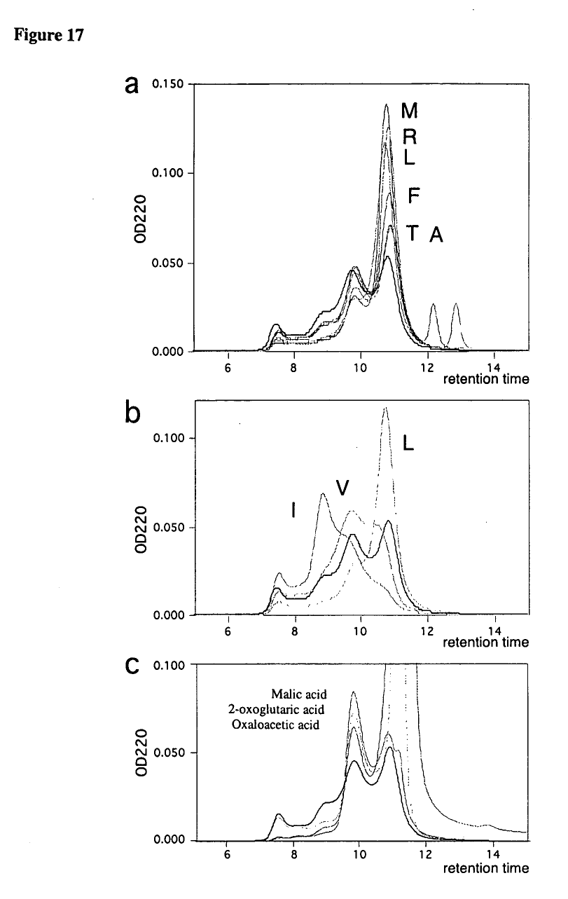

In the sample containing isoleucine, the second peak increased. In the sample containing valine, the third peak increased (FIG. 17a). In samples containing other six amino acids, i.e., methionine, arginine, leucine, phenylalanine, alanine, and threonine, the fourth peak increased (FIG. 17b). None of the other twelve amino acids showed any significant effect. When one of the three metabolic intermediates, each containing four or more carbon atoms (i.e., malic acid, 2-oxoglutaric acid, and oxaloacetic acid) was mixed, the third peak increased (FIG. 17c). In contrast, no significant effect was observed with two other metabolic intermediates (i.e., pyruvic acid and 3-phosphoglyceric acid), each containing three carbon atoms.

Peaks associated with smaller retention time should correspond to assemblies of larger molecular weights. Thus it is concluded that isoleucine accelerates assembling of the protein, six other amino acids, i.e., methionine, arginine, leucine, phenylalanine, alanine, and threonine, accelerate disassembling of the protein, and valine and three metabolic intermediates (malic acid, 2-oxoglutaric acid, and oxaloacetic acid) have effects of stabilizing intermediate assemblies.

TABLE 1
3D atomic coordinates of the 3D structure of the FFRP
(pot1216151)

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |

| | | | | | | |
|---|---|---|---|---|---|---|
| CRYST1 | 96.345 | 96.345 | 97.056 | 90.00 | 90.00 | 120.00 |

| | | | | |
|---|---|---|---|---|
| SCALE1 | 0.010379 | 0.005993 | 0.000000 | 0.00000 |
| SCALE2 | 0.000000 | 0.011985 | 0.000000 | 0.00000 |
| SCALE3 | 0.000000 | 0.000000 | 0.010303 | 0.00000 |

| | | | | | | | | | | | | |
|---|---|---|---|---|---|---|---|---|---|---|---|---|
| ATOM | 1 | CB | VAL | A | 2 | −2.073 | 48.359 | 7.218 | 1.00 | 7.25 | 6 | C |
| ATOM | 2 | CG1 | VAL | A | 2 | −0.814 | 47.539 | 6.836 | 1.00 | 8.49 | 6 | C |
| ATOM | 3 | CG2 | VAL | A | 2 | −3.080 | 48.341 | 6.088 | 1.00 | 7.16 | 6 | C |
| ATOM | 4 | C | VAL | A | 2 | −1.718 | 47.700 | 9.595 | 1.00 | 7.20 | 6 | C |
| ATOM | 5 | O | VAL | A | 2 | −1.191 | 48.725 | 10.010 | 1.00 | 8.49 | 8 | O |
| ATOM | 6 | N | VAL | A | 2 | −4.005 | 48.669 | 8.832 | 1.00 | 7.12 | 7 | N |
| ATOM | 7 | CA | VAL | A | 2 | −2.752 | 47.840 | 8.490 | 1.00 | 7.39 | 6 | C |
| ATOM | 8 | N | THR | A | 3 | −1.376 | 46.456 | 9.992 | 1.00 | 7.51 | 7 | N |
| ATOM | 9 | CA | THR | A | 3 | −0.236 | 46.219 | 10.863 | 1.00 | 8.84 | 6 | C |
| ATOM | 10 | CB | THR | A | 3 | −0.541 | 45.367 | 12.111 | 1.00 | 9.16 | 6 | C |
| ATOM | 11 | OG1 | THR | A | 3 | −1.620 | 46.010 | 12.820 | 1.00 | 9.25 | 8 | O |
| ATOM | 12 | CG2 | THR | A | 3 | 0.616 | 45.208 | 13.110 | 1.00 | 8.76 | 6 | C |
| ATOM | 13 | C | THR | A | 3 | 0.836 | 45.449 | 10.073 | 1.00 | 6.50 | 6 | C |
| ATOM | 14 | O | THR | A | 3 | 0.454 | 44.528 | 9.380 | 1.00 | 4.22 | 8 | O |
| ATOM | 15 | N | ALA | A | 4 | 2.098 | 45.907 | 10.132 | 1.00 | 6.94 | 7 | N |
| ATOM | 16 | CA | ALA | A | 4 | 3.101 | 45.068 | 9.420 | 1.00 | 6.58 | 6 | C |
| ATOM | 17 | CB | ALA | A | 4 | 3.286 | 45.669 | 8.021 | 1.00 | 6.97 | 6 | C |
| ATOM | 18 | C | ALA | A | 4 | 4.402 | 45.022 | 10.211 | 1.00 | 7.80 | 6 | C |
| ATOM | 19 | O | ALA | A | 4 | 4.635 | 45.828 | 11.124 | 1.00 | 7.68 | 8 | O |
| ATOM | 20 | N | PHE | A | 5 | 5.272 | 44.058 | 9.877 | 1.00 | 5.61 | 7 | N |
| ATOM | 21 | CA | PHE | A | 5 | 6.561 | 44.001 | 10.554 | 1.00 | 7.24 | 6 | C |
| ATOM | 22 | CB | PHE | A | 5 | 6.806 | 42.707 | 11.355 | 1.00 | 6.59 | 6 | C |
| ATOM | 23 | CG | PHE | A | 5 | 5.969 | 42.618 | 12.605 | 1.00 | 7.48 | 6 | C |
| ATOM | 24 | CD1 | PHE | A | 5 | 4.628 | 42.271 | 12.533 | 1.00 | 7.25 | 6 | C |
| ATOM | 25 | CD2 | PHE | A | 5 | 6.511 | 42.995 | 13.830 | 1.00 | 8.05 | 6 | C |
| ATOM | 26 | CE1 | PHE | A | 5 | 3.833 | 42.218 | 13.670 | 1.00 | 8.84 | 6 | C |
| ATOM | 27 | CE2 | PHE | A | 5 | 5.734 | 42.907 | 14.983 | 1.00 | 6.40 | 6 | C |
| ATOM | 28 | CZ | PHE | A | 5 | 4.430 | 42.493 | 14.877 | 1.00 | 7.39 | 6 | C |
| ATOM | 29 | C | PHE | A | 5 | 7.648 | 44.035 | 9.432 | 1.00 | 7.62 | 6 | C |
| ATOM | 30 | O | PHE | A | 5 | 7.556 | 43.301 | 8.456 | 1.00 | 4.61 | 8 | O |
| ATOM | 31 | N | ILE | A | 6 | 8.535 | 45.005 | 9.654 | 1.00 | 7.29 | 7 | N |
| ATOM | 32 | CA | ILE | A | 6 | 9.625 | 45.211 | 8.729 | 1.00 | 7.19 | 6 | C |
| ATOM | 33 | CB | ILE | A | 6 | 9.692 | 46.692 | 8.314 | 1.00 | 5.96 | 6 | C |
| ATOM | 34 | CG2 | ILE | A | 6 | 10.728 | 46.809 | 7.191 | 1.00 | 5.34 | 6 | C |
| ATOM | 35 | CG1 | ILE | A | 6 | 8.337 | 47.188 | 7.774 | 1.00 | 7.13 | 6 | C |
| ATOM | 36 | CD1 | ILE | A | 6 | 8.404 | 48.686 | 7.432 | 1.00 | 9.39 | 6 | C |
| ATOM | 37 | C | ILE | A | 6 | 10.957 | 44.758 | 9.348 | 1.00 | 6.37 | 6 | C |
| ATOM | 38 | O | ILE | A | 6 | 11.373 | 45.243 | 10.412 | 1.00 | 6.22 | 8 | O |
| ATOM | 39 | N | LEU | A | 7 | 11.568 | 43.783 | 8.725 | 1.00 | 7.34 | 7 | N |
| ATOM | 40 | CA | LEU | A | 7 | 12.832 | 43.204 | 9.137 | 1.00 | 9.69 | 6 | C |
| ATOM | 41 | CB | LEU | A | 7 | 12.897 | 41.701 | 8.927 | 1.00 | 12.76 | 6 | C |
| ATOM | 42 | CG | LEU | A | 7 | 11.781 | 40.816 | 9.451 | 1.00 | 17.25 | 6 | C |
| ATOM | 43 | CD1 | LEU | A | 7 | 12.038 | 39.325 | 9.258 | 1.00 | 18.40 | 6 | C |
| ATOM | 44 | CD2 | LEU | A | 7 | 11.550 | 41.078 | 10.922 | 1.00 | 17.00 | 6 | C |
| ATOM | 45 | C | LEU | A | 7 | 13.973 | 43.797 | 8.292 | 1.00 | 9.33 | 6 | C |
| ATOM | 46 | O | LEU | A | 7 | 13.711 | 44.012 | 7.118 | 1.00 | 7.50 | 8 | O |
| ATOM | 47 | N | MET | A | 8 | 15.012 | 44.351 | 8.930 | 1.00 | 9.25 | 7 | N |
| ATOM | 48 | CA | MET | A | 8 | 16.013 | 45.098 | 8.227 | 1.00 | 11.96 | 6 | C |
| ATOM | 49 | CB | MET | A | 8 | 15.954 | 46.584 | 8.663 | 1.00 | 13.86 | 6 | C |
| ATOM | 50 | CG | MET | A | 8 | 14.610 | 47.251 | 8.492 | 1.00 | 18.88 | 6 | C |
| ATOM | 51 | SD | MET | A | 8 | 14.490 | 48.890 | 9.189 | 1.00 | 16.93 | 16 | S |
| ATOM | 52 | CE | MET | A | 8 | 13.643 | 48.403 | 10.698 | 1.00 | 23.90 | 6 | C |
| ATOM | 53 | C | MET | A | 8 | 17.458 | 44.653 | 8.580 | 1.00 | 14.57 | 6 | C |
| ATOM | 54 | O | MET | A | 8 | 17.730 | 44.249 | 9.707 | 1.00 | 12.69 | 8 | O |
| ATOM | 55 | N | VAL | A | 9 | 18.357 | 44.743 | 7.615 | 1.00 | 13.97 | 7 | N |
| ATOM | 56 | CA | VAL | A | 9 | 19.787 | 44.503 | 7.800 | 1.00 | 14.26 | 6 | C |
| ATOM | 57 | CB | VAL | A | 9 | 20.417 | 43.674 | 6.668 | 1.00 | 16.26 | 6 | C |
| ATOM | 58 | CG1 | VAL | A | 9 | 21.972 | 43.738 | 6.697 | 1.00 | 16.52 | 6 | C |
| ATOM | 59 | CG2 | VAL | A | 9 | 19.994 | 42.242 | 6.760 | 1.00 | 14.67 | 6 | C |
| ATOM | 60 | C | VAL | A | 9 | 20.346 | 45.902 | 7.807 | 1.00 | 15.95 | 6 | C |
| ATOM | 61 | O | VAL | A | 9 | 19.933 | 46.661 | 6.900 | 1.00 | 20.59 | 8 | O |
| ATOM | 62 | N | THR | A | 10 | 21.152 | 46.424 | 8.712 | 1.00 | 15.01 | 7 | N |
| ATOM | 63 | CA | THR | A | 10 | 21.660 | 47.783 | 8.609 | 1.00 | 13.75 | 6 | C |
| ATOM | 64 | CB | THR | A | 10 | 21.540 | 48.675 | 9.859 | 1.00 | 13.36 | 6 | C |
| ATOM | 65 | OG1 | THR | A | 10 | 22.194 | 48.066 | 11.001 | 1.00 | 11.93 | 8 | O |
| ATOM | 66 | CG2 | THR | A | 10 | 20.070 | 48.913 | 10.248 | 1.00 | 13.19 | 6 | C |
| ATOM | 67 | C | THR | A | 10 | 23.190 | 47.710 | 8.376 | 1.00 | 15.09 | 6 | C |
| ATOM | 68 | O | THR | A | 10 | 23.728 | 46.650 | 8.648 | 1.00 | 15.54 | 8 | O |
| ATOM | 69 | N | ALA | A | 11 | 23.809 | 48.827 | 8.039 | 1.00 | 16.40 | 7 | N |
| ATOM | 70 | CA | ALA | A | 11 | 25.306 | 48.728 | 7.997 | 1.00 | 17.23 | 6 | C |
| ATOM | 71 | CB | ALA | A | 11 | 25.835 | 50.065 | 7.564 | 1.00 | 16.26 | 6 | C |
| ATOM | 72 | C | ALA | A | 11 | 25.722 | 48.352 | 9.413 | 1.00 | 17.14 | 6 | C |
| ATOM | 73 | O | ALA | A | 11 | 25.090 | 48.782 | 10.388 | 1.00 | 16.25 | 8 | O |
| ATOM | 74 | N | ALA | A | 12 | 26.758 | 47.550 | 9.599 | 1.00 | 17.62 | 7 | N |
| ATOM | 75 | CA | ALA | A | 12 | 27.170 | 47.140 | 10.952 | 1.00 | 17.18 | 6 | C |
| ATOM | 76 | CB | ALA | A | 12 | 28.389 | 46.228 | 10.790 | 1.00 | 18.07 | 6 | C |
| ATOM | 77 | C | ALA | A | 12 | 27.431 | 48.312 | 11.870 | 1.00 | 16.00 | 6 | C |
| ATOM | 78 | O | ALA | A | 12 | 28.132 | 49.260 | 11.528 | 1.00 | 14.87 | 8 | O |
| ATOM | 79 | N | GLY | A | 13 | 26.855 | 48.330 | 13.093 | 1.00 | 14.71 | 7 | N |
| ATOM | 80 | CA | GLY | A | 13 | 27.171 | 49.433 | 14.022 | 1.00 | 14.32 | 6 | C |
| ATOM | 81 | C | GLY | A | 13 | 26.236 | 50.639 | 13.929 | 1.00 | 15.21 | 6 | C |
| ATOM | 82 | O | GLY | A | 13 | 26.360 | 51.575 | 14.690 | 1.00 | 15.60 | 8 | O |
| ATOM | 83 | N | LYS | A | 14 | 25.288 | 50.635 | 13.004 | 1.00 | 15.71 | 7 | N |
| ATOM | 84 | CA | LYS | A | 14 | 24.320 | 51.724 | 12.873 | 1.00 | 16.25 | 6 | C |
| ATOM | 85 | CB | LYS | A | 14 | 24.207 | 52.109 | 11.375 | 1.00 | 17.80 | 6 | C |
| ATOM | 86 | CG | LYS | A | 14 | 25.566 | 52.622 | 10.845 | 1.00 | 21.86 | 6 | C |
| ATOM | 87 | CD | LYS | A | 14 | 25.885 | 53.964 | 11.484 | 1.00 | 21.55 | 6 | C |
| ATOM | 88 | CE | LYS | A | 14 | 27.300 | 54.385 | 11.090 | 1.00 | 24.44 | 6 | C |
| ATOM | 89 | NZ | LYS | A | 14 | 27.432 | 55.819 | 11.485 | 1.00 | 25.93 | 7 | N |
| ATOM | 90 | C | LYS | A | 14 | 22.920 | 51.288 | 13.302 | 1.00 | 14.39 | 6 | C |
| ATOM | 91 | O | LYS | A | 14 | 22.013 | 52.111 | 13.269 | 1.00 | 15.68 | 8 | O |
| ATOM | 92 | N | GLU | A | 15 | 22.738 | 50.034 | 13.653 | 1.00 | 13.01 | 7 | N |
| ATOM | 93 | CA | GLU | A | 15 | 21.393 | 49.571 | 14.001 | 1.00 | 15.99 | 6 | C |
| ATOM | 94 | CB | GLU | A | 15 | 21.452 | 48.127 | 14.521 | 1.00 | 17.36 | 6 | C |
| ATOM | 95 | CG | GLU | A | 15 | 22.248 | 48.147 | 15.825 | 1.00 | 25.39 | 6 | C |
| ATOM | 96 | CD | GLU | A | 15 | 22.807 | 46.808 | 16.246 | 1.00 | 31.23 | 6 | C |
| ATOM | 97 | OE1 | GLU | A | 15 | 22.135 | 45.923 | 15.626 | 1.00 | 35.29 | 8 | O |
| ATOM | 98 | OE2 | GLU | A | 15 | 23.723 | 46.692 | 17.119 | 1.00 | 30.70 | 8 | O |
| ATOM | 99 | C | GLU | A | 15 | 20.705 | 50.444 | 15.061 | 1.00 | 15.48 | 6 | C |
| ATOM | 100 | O | GLU | A | 15 | 19.530 | 50.761 | 14.944 | 1.00 | 12.54 | 8 | O |
| ATOM | 101 | N | ARG | A | 16 | 21.415 | 50.780 | 16.143 | 1.00 | 16.35 | 7 | N |
| ATOM | 102 | CA | ARG | A | 16 | 20.787 | 51.555 | 17.210 | 1.00 | 19.56 | 6 | C |
| ATOM | 103 | CB | ARG | A | 16 | 21.667 | 51.542 | 18.462 | 1.00 | 23.74 | 6 | C |
| ATOM | 104 | CG | ARG | A | 16 | 21.326 | 52.436 | 19.616 | 1.00 | 29.54 | 6 | C |
| ATOM | 105 | CD | ARG | A | 16 | 21.727 | 53.910 | 19.477 | 1.00 | 35.88 | 6 | C |
| ATOM | 106 | NE | ARG | A | 16 | 21.060 | 54.702 | 20.540 | 1.00 | 41.43 | 7 | N |
| ATOM | 107 | CZ | ARG | A | 16 | 21.427 | 54.741 | 21.819 | 1.00 | 43.05 | 6 | C |
| ATOM | 108 | NH1 | ARG | A | 16 | 22.488 | 54.052 | 22.253 | 1.00 | 45.29 | 7 | N |
| ATOM | 109 | NH2 | ARG | A | 16 | 20.720 | 55.492 | 22.656 | 1.00 | 44.30 | 7 | N |
| ATOM | 110 | C | ARG | A | 16 | 20.424 | 52.947 | 16.752 | 1.00 | 18.28 | 6 | C |
| ATOM | 111 | O | ARG | A | 16 | 19.346 | 53.412 | 17.143 | 1.00 | 16.68 | 8 | O |
| ATOM | 112 | N | GLU | A | 17 | 21.313 | 53.637 | 16.018 | 1.00 | 16.79 | 7 | N |
| ATOM | 113 | CA | GLU | A | 17 | 20.956 | 54.980 | 15.563 | 1.00 | 15.83 | 6 | C |
| ATOM | 114 | CB | GLU | A | 17 | 22.123 | 55.767 | 15.012 | 1.00 | 18.26 | 6 | C |
| ATOM | 115 | CG | GLU | A | 17 | 22.870 | 55.079 | 13.891 | 1.00 | 21.97 | 6 | C |
| ATOM | 116 | CD | GLU | A | 17 | 22.345 | 55.501 | 12.538 | 1.00 | 25.19 | 6 | C |
| ATOM | 117 | OE1 | GLU | A | 17 | 21.453 | 56.393 | 12.521 | 1.00 | 26.12 | 8 | O |
| ATOM | 118 | OE2 | GLU | A | 17 | 22.822 | 54.970 | 11.512 | 1.00 | 27.57 | 8 | O |
| ATOM | 119 | C | GLU | A | 17 | 19.819 | 54.887 | 14.538 | 1.00 | 14.37 | 6 | C |
| ATOM | 120 | O | GLU | A | 17 | 18.952 | 55.773 | 14.540 | 1.00 | 12.63 | 8 | O |
| ATOM | 121 | N | VAL | A | 18 | 19.744 | 53.871 | 13.711 | 1.00 | 11.94 | 7 | N |
| ATOM | 122 | CA | VAL | A | 18 | 18.618 | 53.738 | 12.799 | 1.00 | 12.82 | 6 | C |
| ATOM | 123 | CB | VAL | A | 18 | 18.806 | 52.622 | 11.773 | 1.00 | 13.64 | 6 | C |
| ATOM | 124 | CG1 | VAL | A | 18 | 17.503 | 52.290 | 11.014 | 1.00 | 10.60 | 6 | C |
| ATOM | 125 | CG2 | VAL | A | 18 | 19.882 | 53.077 | 10.757 | 1.00 | 13.42 | 6 | C |
| ATOM | 126 | C | VAL | A | 18 | 17.359 | 53.493 | 13.631 | 1.00 | 14.02 | 6 | C |
| ATOM | 127 | O | VAL | A | 18 | 16.319 | 54.112 | 13.357 | 1.00 | 14.65 | 8 | O |
| ATOM | 128 | N | MET | A | 19 | 17.442 | 52.601 | 14.594 | 1.00 | 13.17 | 7 | N |
| ATOM | 129 | CA | MET | A | 19 | 16.271 | 52.267 | 15.425 | 1.00 | 13.50 | 6 | C |
| ATOM | 130 | CB | MET | A | 19 | 16.681 | 51.279 | 16.512 | 1.00 | 14.15 | 6 | C |
| ATOM | 131 | CG | MET | A | 19 | 15.459 | 50.893 | 17.328 | 1.00 | 15.47 | 6 | C |
| ATOM | 132 | SD | MET | A | 19 | 15.745 | 49.773 | 18.610 | 1.00 | 16.28 | 16 | S |
| ATOM | 133 | CE | MET | A | 19 | 16.553 | 50.600 | 19.921 | 1.00 | 18.98 | 6 | C |
| ATOM | 134 | C | MET | A | 19 | 15.711 | 53.484 | 16.132 | 1.00 | 14.53 | 6 | C |
| ATOM | 135 | O | MET | A | 19 | 14.486 | 53.703 | 16.175 | 1.00 | 13.43 | 8 | O |
| ATOM | 136 | N | GLU | A | 20 | 16.611 | 54.320 | 16.649 | 1.00 | 14.59 | 7 | N |
| ATOM | 137 | CA | GLU | A | 20 | 16.163 | 55.531 | 17.322 | 1.00 | 18.00 | 6 | C |
| ATOM | 138 | CB | GLU | A | 20 | 17.306 | 56.289 | 18.025 | 1.00 | 20.38 | 6 | C |
| ATOM | 139 | CG | GLU | A | 20 | 17.844 | 55.506 | 19.220 | 1.00 | 23.46 | 6 | C |
| ATOM | 140 | CD | GLU | A | 20 | 16.821 | 55.001 | 20.202 | 1.00 | 27.70 | 6 | C |
| ATOM | 141 | OE1 | GLU | A | 20 | 15.771 | 55.673 | 20.405 | 1.00 | 31.74 | 8 | O |
| ATOM | 142 | OE2 | GLU | A | 20 | 17.017 | 53.914 | 20.797 | 1.00 | 29.38 | 8 | O |
| ATOM | 143 | C | GLU | A | 20 | 15.420 | 56.452 | 16.373 | 1.00 | 17.62 | 6 | C |
| ATOM | 144 | O | GLU | A | 20 | 14.387 | 56.983 | 16.753 | 1.00 | 17.09 | 8 | O |
| ATOM | 145 | N | LYS | A | 21 | 15.914 | 56.708 | 15.172 | 1.00 | 16.59 | 7 | N |
| ATOM | 146 | CA | LYS | A | 21 | 15.260 | 57.495 | 14.164 | 1.00 | 15.48 | 6 | C |
| ATOM | 147 | CB | LYS | A | 21 | 16.106 | 57.424 | 12.866 | 1.00 | 15.60 | 6 | C |
| ATOM | 148 | CG | LYS | A | 21 | 17.132 | 58.542 | 12.743 | 1.00 | 19.35 | 6 | C |
| ATOM | 149 | CD | LYS | A | 21 | 18.166 | 58.319 | 11.668 | 1.00 | 20.39 | 6 | C |
| ATOM | 150 | CE | LYS | A | 21 | 19.434 | 59.162 | 11.914 | 1.00 | 23.18 | 6 | C |
| ATOM | 151 | NZ | LYS | A | 21 | 20.429 | 58.883 | 10.813 | 1.00 | 21.66 | 7 | N |
| ATOM | 152 | C | LYS | A | 21 | 13.879 | 56.889 | 13.850 | 1.00 | 14.96 | 6 | C |
| ATOM | 153 | O | LYS | A | 21 | 12.905 | 57.633 | 13.658 | 1.00 | 15.15 | 8 | O |
| ATOM | 154 | N | LEU | A | 22 | 13.759 | 55.566 | 13.682 | 1.00 | 10.45 | 7 | N |
| ATOM | 155 | CA | LEU | A | 22 | 12.469 | 54.967 | 13.372 | 1.00 | 11.33 | 6 | C |
| ATOM | 156 | CB | LEU | A | 22 | 12.651 | 53.463 | 13.095 | 1.00 | 11.29 | 6 | C |
| ATOM | 157 | CG | LEU | A | 22 | 13.490 | 53.153 | 11.855 | 1.00 | 12.08 | 6 | C |
| ATOM | 158 | CD1 | LEU | A | 22 | 13.725 | 51.647 | 11.748 | 1.00 | 14.54 | 6 | C |
| ATOM | 159 | CD2 | LEU | A | 22 | 12.857 | 53.652 | 10.579 | 1.00 | 11.38 | 6 | C |
| ATOM | 160 | C | LEU | A | 22 | 11.460 | 55.109 | 14.514 | 1.00 | 11.56 | 6 | C |
| ATOM | 161 | O | LEU | A | 22 | 10.270 | 55.364 | 14.275 | 1.00 | 10.86 | 8 | O |
| ATOM | 162 | N | LEU | A | 23 | 11.918 | 54.921 | 15.746 | 1.00 | 13.00 | 7 | N |
| ATOM | 163 | CA | LEU | A | 23 | 11.054 | 55.014 | 16.917 | 1.00 | 16.61 | 6 | C |
| ATOM | 164 | CB | LEU | A | 23 | 11.801 | 54.718 | 18.209 | 1.00 | 16.55 | 6 | C |
| ATOM | 165 | CG | LEU | A | 23 | 11.858 | 53.264 | 18.610 | 1.00 | 18.39 | 6 | C |
| ATOM | 166 | CD1 | LEU | A | 23 | 12.928 | 53.050 | 19.675 | 1.00 | 16.32 | 6 | C |
| ATOM | 167 | CD2 | LEU | A | 23 | 10.501 | 52.804 | 19.142 | 1.00 | 17.70 | 6 | C |
| ATOM | 168 | C | LEU | A | 23 | 10.404 | 56.393 | 17.064 | 1.00 | 15.89 | 6 | C |
| ATOM | 169 | O | LEU | A | 23 | 9.282 | 56.440 | 17.556 | 1.00 | 17.60 | 8 | O |
| ATOM | 170 | N | ALA | A | 24 | 10.998 | 57.440 | 16.551 | 1.00 | 16.01 | 7 | N |
| ATOM | 171 | CA | ALA | A | 24 | 10.422 | 58.773 | 16.532 | 1.00 | 18.43 | 6 | C |
| ATOM | 172 | CB | ALA | A | 24 | 11.533 | 59.775 | 16.231 | 1.00 | 16.56 | 6 | C |
| ATOM | 173 | C | ALA | A | 24 | 9.330 | 58.951 | 15.481 | 1.00 | 18.75 | 6 | C |
| ATOM | 174 | O | ALA | A | 24 | 8.778 | 60.058 | 15.427 | 1.00 | 19.41 | 8 | O |
| ATOM | 175 | N | MET | A | 25 | 9.188 | 58.039 | 14.512 | 1.00 | 15.42 | 7 | N |
| ATOM | 176 | CA | MET | A | 25 | 8.189 | 58.202 | 13.466 | 1.00 | 15.12 | 6 | C |
| ATOM | 177 | CB | MET | A | 25 | 8.570 | 57.409 | 12.208 | 1.00 | 14.61 | 6 | C |
| ATOM | 178 | CG | MET | A | 25 | 9.787 | 57.989 | 11.504 | 1.00 | 16.53 | 6 | C |
| ATOM | 179 | SD | MET | A | 25 | 10.471 | 56.816 | 10.317 | 1.00 | 15.91 | 16 | S |
| ATOM | 180 | CE | MET | A | 25 | 12.118 | 57.576 | 10.131 | 1.00 | 18.16 | 6 | C |
| ATOM | 181 | C | MET | A | 25 | 6.845 | 57.715 | 13.992 | 1.00 | 15.09 | 6 | C |
| ATOM | 182 | O | MET | A | 25 | 6.801 | 56.666 | 14.654 | 1.00 | 15.91 | 8 | O |
| ATOM | 183 | N | PRO | A | 26 | 5.757 | 58.441 | 13.743 | 1.00 | 14.86 | 7 | N |
| ATOM | 184 | CD | PRO | A | 26 | 5.755 | 59.703 | 12.955 | 1.00 | 14.19 | 6 | C |
| ATOM | 185 | CA | PRO | A | 26 | 4.455 | 58.083 | 14.242 | 1.00 | 14.29 | 6 | C |
| ATOM | 186 | CB | PRO | A | 26 | 3.504 | 59.246 | 13.796 | 1.00 | 15.81 | 6 | C |
| ATOM | 187 | CG | PRO | A | 26 | 4.265 | 59.970 | 12.737 | 1.00 | 16.25 | 6 | C |
| ATOM | 188 | C | PRO | A | 26 | 3.900 | 56.773 | 13.810 | 1.00 | 13.72 | 6 | C |
| ATOM | 189 | O | PRO | A | 26 | 3.192 | 56.130 | 14.625 | 1.00 | 14.68 | 8 | O |
| ATOM | 190 | N | GLU | A | 27 | 4.291 | 56.227 | 12.645 | 1.00 | 13.34 | 7 | N |
| ATOM | 191 | CA | GLU | A | 27 | 3.806 | 54.923 | 12.235 | 1.00 | 13.47 | 6 | C |
| ATOM | 192 | CB | GLU | A | 27 | 4.091 | 54.625 | 10.760 | 1.00 | 17.16 | 6 | C |
| ATOM | 193 | CG | GLU | A | 27 | 3.995 | 55.541 | 9.606 | 1.00 | 21.17 | 6 | C |
| ATOM | 194 | CD | GLU | A | 27 | 4.560 | 56.955 | 9.586 | 1.00 | 23.93 | 6 | C |
| ATOM | 195 | OE1 | GLU | A | 27 | 5.418 | 57.453 | 10.335 | 1.00 | 20.18 | 8 | O |
| ATOM | 196 | OE2 | GLU | A | 27 | 4.072 | 57.616 | 8.610 | 1.00 | 25.99 | 8 | O |
| ATOM | 197 | C | GLU | A | 27 | 4.444 | 53.764 | 13.004 | 1.00 | 10.60 | 6 | C |
| ATOM | 198 | O | GLU | A | 27 | 3.954 | 52.644 | 13.011 | 1.00 | 7.90 | 8 | O |
| ATOM | 199 | N | VAL | A | 28 | 5.617 | 53.989 | 13.561 | 1.00 | 9.98 | 7 | N |
| ATOM | 200 | CA | VAL | A | 28 | 6.394 | 52.946 | 14.202 | 1.00 | 8.91 | 6 | C |
| ATOM | 201 | CB | VAL | A | 28 | 7.896 | 53.306 | 14.215 | 1.00 | 8.00 | 6 | C |
| ATOM | 202 | CG1 | VAL | A | 28 | 8.683 | 52.277 | 15.029 | 1.00 | 6.92 | 6 | C |
| ATOM | 203 | CG2 | VAL | A | 28 | 8.442 | 53.420 | 12.791 | 1.00 | 6.99 | 6 | C |
| ATOM | 204 | C | VAL | A | 28 | 5.878 | 52.729 | 15.620 | 1.00 | 10.15 | 6 | C |
| ATOM | 205 | O | VAL | A | 28 | 5.925 | 53.618 | 16.428 | 1.00 | 10.34 | 8 | O |
| ATOM | 206 | N | LYS | A | 29 | 5.411 | 51.543 | 15.933 | 1.00 | 10.04 | 7 | N |
| ATOM | 207 | CA | LYS | A | 29 | 4.865 | 51.238 | 17.225 | 1.00 | 12.44 | 6 | C |
| ATOM | 208 | CB | LYS | A | 29 | 3.643 | 50.303 | 17.106 | 1.00 | 13.57 | 6 | C |
| ATOM | 209 | CG | LYS | A | 29 | 2.432 | 50.906 | 16.421 | 1.00 | 14.50 | 6 | C |
| ATOM | 210 | CD | LYS | A | 29 | 2.302 | 52.410 | 16.610 | 1.00 | 15.93 | 6 | C |
| ATOM | 211 | CE | LYS | A | 29 | 1.142 | 52.994 | 15.832 | 1.00 | 20.88 | 6 | C |
| ATOM | 212 | NZ | LYS | A | 29 | 1.224 | 54.497 | 15.684 | 1.00 | 22.58 | 7 | N |
| ATOM | 213 | C | LYS | A | 29 | 5.891 | 50.550 | 18.129 | 1.00 | 12.91 | 6 | C |
| ATOM | 214 | O | LYS | A | 29 | 5.769 | 50.754 | 19.332 | 1.00 | 11.55 | 8 | O |
| ATOM | 215 | N | GLU | A | 30 | 6.810 | 49.746 | 17.592 | 1.00 | 10.53 | 7 | N |
| ATOM | 216 | CA | GLU | A | 30 | 7.785 | 49.053 | 18.424 | 1.00 | 11.26 | 6 | C |
| ATOM | 217 | CB | GLU | A | 30 | 7.350 | 47.633 | 18.831 | 1.00 | 13.54 | 6 | C |
| ATOM | 218 | CG | GLU | A | 30 | 6.072 | 47.456 | 19.598 | 1.00 | 16.71 | 6 | C |
| ATOM | 219 | CD | GLU | A | 30 | 5.654 | 46.018 | 19.905 | 1.00 | 18.00 | 6 | C |
| ATOM | 220 | OE1 | GLU | A | 30 | 4.403 | 45.814 | 19.971 | 1.00 | 17.65 | 8 | O |
| ATOM | 221 | OE2 | GLU | A | 30 | 6.554 | 45.183 | 20.077 | 1.00 | 15.08 | 8 | O |
| ATOM | 222 | C | GLU | A | 30 | 9.025 | 48.844 | 17.522 | 1.00 | 8.70 | 6 | C |
| ATOM | 223 | O | GLU | A | 30 | 8.821 | 48.760 | 16.305 | 1.00 | 6.20 | 8 | O |
| ATOM | 224 | N | ALA | A | 31 | 10.198 | 48.728 | 18.108 | 1.00 | 8.38 | 7 | N |
| ATOM | 225 | CA | ALA | A | 31 | 11.417 | 48.529 | 17.293 | 1.00 | 5.18 | 6 | C |
| ATOM | 226 | CB | ALA | A | 31 | 11.894 | 49.862 | 16.752 | 1.00 | 7.29 | 6 | C |
| ATOM | 227 | C | ALA | A | 31 | 12.462 | 47.902 | 18.163 | 1.00 | 9.36 | 6 | C |
| ATOM | 228 | O | ALA | A | 31 | 12.517 | 48.224 | 19.344 | 1.00 | 9.20 | 8 | O |
| ATOM | 229 | N | TYR | A | 32 | 13.162 | 46.872 | 17.670 | 1.00 | 6.49 | 7 | N |
| ATOM | 230 | CA | TYR | A | 32 | 14.166 | 46.191 | 18.428 | 1.00 | 8.44 | 6 | C |
| ATOM | 231 | CB | TYR | A | 32 | 13.698 | 44.827 | 18.938 | 1.00 | 6.51 | 6 | C |
| ATOM | 232 | CG | TYR | A | 32 | 12.632 | 44.881 | 20.030 | 1.00 | 7.98 | 6 | C |
| ATOM | 233 | CD1 | TYR | A | 32 | 12.981 | 44.818 | 21.371 | 1.00 | 9.50 | 6 | C |
| ATOM | 234 | CE1 | TYR | A | 32 | 12.017 | 44.881 | 22.369 | 1.00 | 12.01 | 6 | C |
| ATOM | 235 | CD2 | TYR | A | 32 | 11.293 | 45.016 | 19.698 | 1.00 | 8.48 | 6 | C |
| ATOM | 236 | CE2 | TYR | A | 32 | 10.324 | 45.079 | 20.666 | 1.00 | 9.99 | 6 | C |
| ATOM | 237 | CZ | TYR | A | 32 | 10.685 | 44.997 | 21.998 | 1.00 | 11.97 | 6 | C |
| ATOM | 238 | OH | TYR | A | 32 | 9.698 | 45.024 | 22.951 | 1.00 | 12.74 | 8 | O |
| ATOM | 239 | C | TYR | A | 32 | 15.396 | 45.801 | 17.567 | 1.00 | 8.57 | 6 | C |
| ATOM | 240 | O | TYR | A | 32 | 15.211 | 45.300 | 16.462 | 1.00 | 7.72 | 8 | O |
| ATOM | 241 | N | VAL | A | 33 | 16.568 | 45.985 | 18.158 | 1.00 | 7.80 | 7 | N |
| ATOM | 242 | CA | VAL | A | 33 | 17.785 | 45.424 | 17.605 | 1.00 | 6.39 | 6 | C |
| ATOM | 243 | CB | VAL | A | 33 | 19.048 | 46.137 | 18.150 | 1.00 | 8.75 | 6 | C |
| ATOM | 244 | CG1 | VAL | A | 33 | 20.273 | 45.355 | 17.658 | 1.00 | 8.40 | 6 | C |
| ATOM | 245 | CG2 | VAL | A | 33 | 19.021 | 47.589 | 17.720 | 1.00 | 7.33 | 6 | C |
| ATOM | 246 | C | VAL | A | 33 | 17.812 | 43.939 | 17.896 | 1.00 | 5.76 | 6 | C |
| ATOM | 247 | O | VAL | A | 33 | 17.615 | 43.509 | 19.054 | 1.00 | 7.13 | 8 | O |
| ATOM | 248 | N | VAL | A | 34 | 18.079 | 43.073 | 16.894 | 1.00 | 4.78 | 7 | N |
| ATOM | 249 | CA | VAL | A | 34 | 18.004 | 41.632 | 17.161 | 1.00 | 6.41 | 6 | C |
| ATOM | 250 | CB | VAL | A | 34 | 16.731 | 41.014 | 16.512 | 1.00 | 6.60 | 6 | C |
| ATOM | 251 | CG1 | VAL | A | 34 | 15.430 | 41.454 | 17.214 | 1.00 | 5.42 | 6 | C |
| ATOM | 252 | CG2 | VAL | A | 34 | 16.583 | 41.384 | 15.032 | 1.00 | 6.47 | 6 | C |
| ATOM | 253 | C | VAL | A | 34 | 19.219 | 40.873 | 16.622 | 1.00 | 8.92 | 6 | C |
| ATOM | 254 | O | VAL | A | 34 | 19.953 | 41.359 | 15.772 | 1.00 | 10.44 | 8 | O |
| ATOM | 255 | N | TYR | A | 35 | 19.405 | 39.621 | 16.980 | 1.00 | 10.34 | 7 | N |
| ATOM | 256 | CA | TYR | A | 35 | 20.429 | 38.735 | 16.482 | 1.00 | 10.98 | 6 | C |
| ATOM | 257 | CB | TYR | A | 35 | 20.884 | 37.774 | 17.543 | 1.00 | 11.65 | 6 | C |
| ATOM | 258 | CG | TYR | A | 35 | 21.645 | 38.403 | 18.665 | 1.00 | 14.04 | 6 | C |
| ATOM | 259 | CD1 | TYR | A | 35 | 22.970 | 38.793 | 18.465 | 1.00 | 17.64 | 6 | C |
| ATOM | 260 | CE1 | TYR | A | 35 | 23.712 | 39.294 | 19.521 | 1.00 | 18.93 | 6 | C |
| ATOM | 261 | CD2 | TYR | A | 35 | 21.070 | 38.581 | 19.905 | 1.00 | 15.53 | 6 | C |
| ATOM | 262 | CE2 | TYR | A | 35 | 21.807 | 39.100 | 20.951 | 1.00 | 16.48 | 6 | C |
| ATOM | 263 | CZ | TYR | A | 35 | 23.122 | 39.436 | 20.762 | 1.00 | 19.05 | 6 | C |
| ATOM | 264 | OH | TYR | A | 35 | 23.870 | 39.944 | 21.812 | 1.00 | 20.64 | 8 | O |
| ATOM | 265 | C | TYR | A | 35 | 19.815 | 37.881 | 15.371 | 1.00 | 11.75 | 6 | C |
| ATOM | 266 | O | TYR | A | 35 | 18.624 | 37.596 | 15.481 | 1.00 | 14.45 | 8 | O |
| ATOM | 267 | N | GLY | A | 36 | 20.585 | 37.514 | 14.415 | 1.00 | 12.14 | 7 | N |
| ATOM | 268 | CA | GLY | A | 36 | 20.085 | 36.727 | 13.250 | 1.00 | 15.97 | 6 | C |
| ATOM | 269 | C | GLY | A | 36 | 20.689 | 37.429 | 12.036 | 1.00 | 16.50 | 6 | C |
| ATOM | 270 | O | GLY | A | 36 | 21.418 | 38.441 | 12.153 | 1.00 | 21.32 | 8 | O |
| ATOM | 271 | N | GLU | A | 37 | 20.171 | 37.205 | 10.883 | 1.00 | 20.97 | 7 | N |
| ATOM | 272 | CA | GLU | A | 37 | 20.478 | 37.746 | 9.563 | 1.00 | 24.00 | 6 | C |
| ATOM | 273 | CB | GLU | A | 37 | 19.730 | 36.869 | 8.521 | 1.00 | 28.29 | 6 | C |
| ATOM | 274 | CG | GLU | A | 37 | 19.992 | 37.381 | 7.113 | 1.00 | 31.96 | 6 | C |
| ATOM | 275 | CD | GLU | A | 37 | 19.034 | 36.898 | 6.059 | 1.00 | 35.65 | 6 | C |
| ATOM | 276 | OE1 | GLU | A | 37 | 18.268 | 35.905 | 6.213 | 1.00 | 37.44 | 8 | O |
| ATOM | 277 | OE2 | GLU | A | 37 | 19.114 | 37.582 | 5.011 | 1.00 | 38.02 | 8 | O |
| ATOM | 278 | C | GLU | A | 37 | 19.912 | 39.147 | 9.418 | 1.00 | 23.50 | 6 | C |
| ATOM | 279 | O | GLU | A | 37 | 20.409 | 40.033 | 8.717 | 1.00 | 24.72 | 8 | O |
| ATOM | 280 | N | TYR | A | 38 | 18.815 | 39.382 | 10.151 | 1.00 | 21.31 | 7 | N |
| ATOM | 281 | CA | TYR | A | 38 | 18.292 | 40.761 | 10.215 | 1.00 | 18.05 | 6 | C |
| ATOM | 282 | CB | TYR | A | 38 | 16.758 | 40.730 | 10.202 | 1.00 | 18.36 | 6 | C |
| ATOM | 283 | CG | TYR | A | 38 | 16.285 | 40.221 | 8.856 | 1.00 | 21.89 | 6 | C |
| ATOM | 284 | CD1 | TYR | A | 38 | 16.438 | 41.026 | 7.736 | 1.00 | 23.00 | 6 | C |
| ATOM | 285 | CE1 | TYR | A | 38 | 15.971 | 40.590 | 6.514 | 1.00 | 25.96 | 6 | C |
| ATOM | 286 | CD2 | TYR | A | 38 | 15.624 | 39.002 | 8.750 | 1.00 | 24.51 | 6 | C |
| ATOM | 287 | CE2 | TYR | A | 38 | 15.160 | 38.565 | 7.520 | 1.00 | 26.07 | 6 | C |
| ATOM | 288 | CZ | TYR | A | 38 | 15.410 | 39.342 | 6.414 | 1.00 | 25.27 | 6 | C |
| ATOM | 289 | OH | TYR | A | 38 | 14.955 | 38.933 | 5.173 | 1.00 | 29.39 | 8 | O |
| ATOM | 290 | C | TYR | A | 38 | 18.810 | 41.336 | 11.545 | 1.00 | 15.22 | 6 | C |
| ATOM | 291 | O | TYR | A | 38 | 18.883 | 40.635 | 12.546 | 1.00 | 16.00 | 8 | O |
| ATOM | 292 | N | ASP | A | 39 | 18.955 | 42.613 | 11.599 | 1.00 | 14.06 | 7 | N |
| ATOM | 293 | CA | ASP | A | 39 | 19.565 | 43.412 | 12.643 | 1.00 | 13.19 | 6 | C |
| ATOM | 294 | CB | ASP | A | 39 | 20.378 | 44.510 | 11.873 | 1.00 | 11.78 | 6 | C |
| ATOM | 295 | CG | ASP | A | 39 | 21.527 | 43.891 | 11.130 | 1.00 | 13.09 | 6 | C |
| ATOM | 296 | OD1 | ASP | A | 39 | 21.795 | 42.747 | 11.415 | 1.00 | 12.32 | 8 | O |
| ATOM | 297 | OD2 | ASP | A | 39 | 22.173 | 44.518 | 10.302 | 1.00 | 16.95 | 8 | O |
| ATOM | 298 | C | ASP | A | 39 | 18.532 | 44.178 | 13.447 | 1.00 | 10.56 | 6 | C |
| ATOM | 299 | O | ASP | A | 39 | 18.763 | 44.513 | 14.618 | 1.00 | 8.22 | 8 | O |
| ATOM | 300 | N | LEU | A | 40 | 17.434 | 44.496 | 12.735 | 1.00 | 9.46 | 7 | N |
| ATOM | 301 | CA | LEU | A | 40 | 16.344 | 45.250 | 13.355 | 1.00 | 9.22 | 6 | C |
| ATOM | 302 | CB | LEU | A | 40 | 16.246 | 46.683 | 12.865 | 1.00 | 12.49 | 6 | C |
| ATOM | 303 | CG | LEU | A | 40 | 17.180 | 47.814 | 13.176 | 1.00 | 14.92 | 6 | C |
| ATOM | 304 | CD1 | LEU | A | 40 | 16.680 | 49.055 | 12.461 | 1.00 | 15.41 | 6 | C |
| ATOM | 305 | CD2 | LEU | A | 40 | 17.274 | 48.049 | 14.672 | 1.00 | 17.24 | 6 | C |
| ATOM | 306 | C | LEU | A | 40 | 14.966 | 44.704 | 12.880 | 1.00 | 8.69 | 6 | C |
| ATOM | 307 | O | LEU | A | 40 | 14.829 | 44.337 | 11.705 | 1.00 | 6.37 | 8 | O |
| ATOM | 308 | N | ILE | A | 41 | 14.001 | 44.818 | 13.778 | 1.00 | 7.55 | 7 | N |
| ATOM | 309 | CA | ILE | A | 41 | 12.598 | 44.417 | 13.498 | 1.00 | 8.63 | 6 | C |
| ATOM | 310 | CB | ILE | A | 41 | 12.087 | 43.119 | 14.076 | 1.00 | 9.76 | 6 | C |
| ATOM | 311 | CG2 | ILE | A | 41 | 12.326 | 42.903 | 15.566 | 1.00 | 11.62 | 6 | C |
| ATOM | 312 | CG1 | ILE | A | 41 | 10.575 | 42.937 | 13.807 | 1.00 | 10.77 | 6 | C |
| ATOM | 313 | CD1 | ILE | A | 41 | 10.193 | 41.468 | 13.867 | 1.00 | 13.08 | 6 | C |
| ATOM | 314 | C | ILE | A | 41 | 11.750 | 45.593 | 13.967 | 1.00 | 10.12 | 6 | C |
| ATOM | 315 | O | ILE | A | 41 | 12.005 | 46.153 | 15.053 | 1.00 | 7.54 | 8 | O |
| ATOM | 316 | N | VAL | A | 42 | 10.953 | 46.090 | 13.043 | 1.00 | 8.66 | 7 | N |
| ATOM | 317 | CA | VAL | A | 42 | 10.139 | 47.268 | 13.395 | 1.00 | 10.64 | 6 | C |
| ATOM | 318 | CB | VAL | A | 42 | 10.656 | 48.463 | 12.600 | 1.00 | 14.70 | 6 | C |
| ATOM | 319 | CG1 | VAL | A | 42 | 9.705 | 49.597 | 12.514 | 1.00 | 14.15 | 6 | C |
| ATOM | 320 | CG2 | VAL | A | 42 | 11.929 | 49.038 | 13.295 | 1.00 | 17.64 | 6 | C |
| ATOM | 321 | C | VAL | A | 42 | 8.676 | 46.957 | 13.114 | 1.00 | 9.69 | 6 | C |
| ATOM | 322 | O | VAL | A | 42 | 8.404 | 46.277 | 12.123 | 1.00 | 9.29 | 8 | O |
| ATOM | 323 | N | LYS | A | 43 | 7.785 | 47.249 | 14.067 | 1.00 | 9.12 | 7 | N |
| ATOM | 324 | CA | LYS | A | 43 | 6.352 | 47.043 | 13.919 | 1.00 | 7.52 | 6 | C |
| ATOM | 325 | CB | LYS | A | 43 | 5.757 | 46.570 | 15.274 | 1.00 | 8.78 | 6 | C |
| ATOM | 326 | CG | LYS | A | 43 | 4.246 | 46.290 | 15.145 | 1.00 | 12.03 | 6 | C |
| ATOM | 327 | CD | LYS | A | 43 | 3.692 | 45.912 | 16.541 | 1.00 | 11.71 | 6 | C |
| ATOM | 328 | CE | LYS | A | 43 | 2.200 | 45.790 | 16.524 | 1.00 | 13.09 | 6 | C |
| ATOM | 329 | NZ | LYS | A | 43 | 1.593 | 45.210 | 17.748 | 1.00 | 11.39 | 7 | N |
| ATOM | 330 | C | LYS | A | 43 | 5.697 | 48.367 | 13.564 | 1.00 | 7.31 | 6 | C |
| ATOM | 331 | O | LYS | A | 43 | 5.845 | 49.413 | 14.216 | 1.00 | 6.38 | 8 | O |
| ATOM | 332 | N | VAL | A | 44 | 4.971 | 48.402 | 12.451 | 1.00 | 6.74 | 7 | N |
| ATOM | 333 | CA | VAL | A | 44 | 4.336 | 49.585 | 11.930 | 1.00 | 7.54 | 6 | C |
| ATOM | 334 | CB | VAL | A | 44 | 4.898 | 50.065 | 10.573 | 1.00 | 11.01 | 6 | C |
| ATOM | 335 | CG1 | VAL | A | 44 | 6.434 | 50.232 | 10.650 | 1.00 | 12.68 | 6 | C |
| ATOM | 336 | CG2 | VAL | A | 44 | 4.541 | 49.098 | 9.469 | 1.00 | 10.39 | 6 | C |
| ATOM | 337 | C | VAL | A | 44 | 2.825 | 49.371 | 11.758 | 1.00 | 9.21 | 6 | C |
| ATOM | 338 | O | VAL | A | 44 | 2.292 | 48.264 | 11.561 | 1.00 | 8.93 | 8 | O |
| ATOM | 339 | N | GLU | A | 45 | 2.134 | 50.493 | 11.957 | 1.00 | 10.36 | 7 | N |
| ATOM | 340 | CA | GLU | A | 45 | 0.665 | 50.496 | 11.858 | 1.00 | 12.06 | 6 | C |
| ATOM | 341 | CB | GLU | A | 45 | 0.004 | 50.496 | 13.238 | 1.00 | 12.25 | 6 | C |
| ATOM | 342 | CG | GLU | A | 45 | 0.012 | 49.086 | 13.798 | 1.00 | 15.23 | 6 | C |
| ATOM | 343 | CD | GLU | A | 45 | −0.408 | 48.927 | 15.233 | 1.00 | 18.39 | 6 | C |
| ATOM | 344 | OE1 | GLU | A | 45 | −0.169 | 47.876 | 15.848 | 1.00 | 21.70 | 8 | O |
| ATOM | 345 | OE2 | GLU | A | 45 | −1.021 | 49.852 | 15.769 | 1.00 | 21.18 | 8 | O |
| ATOM | 346 | C | GLU | A | 45 | 0.236 | 51.698 | 11.047 | 1.00 | 10.21 | 6 | C |
| ATOM | 347 | O | GLU | A | 45 | 0.783 | 52.780 | 11.208 | 1.00 | 10.74 | 8 | O |
| ATOM | 348 | N | THR | A | 46 | −0.422 | 51.431 | 9.912 | 1.00 | 10.02 | 7 | N |
| ATOM | 349 | CA | THR | A | 46 | −0.794 | 52.546 | 9.056 | 1.00 | 10.52 | 6 | C |
| ATOM | 350 | CB | THR | A | 46 | −0.030 | 52.589 | 7.701 | 1.00 | 12.10 | 6 | C |
| ATOM | 351 | OG1 | THR | A | 46 | −0.280 | 51.395 | 6.931 | 1.00 | 9.65 | 8 | O |
| ATOM | 352 | CG2 | THR | A | 46 | 1.482 | 52.648 | 7.918 | 1.00 | 14.04 | 6 | C |
| ATOM | 353 | C | THR | A | 46 | −2.297 | 52.431 | 8.783 | 1.00 | 10.40 | 6 | C |
| ATOM | 354 | O | THR | A | 46 | −2.889 | 51.353 | 8.880 | 1.00 | 6.84 | 8 | O |
| ATOM | 355 | N | ASP | A | 47 | −2.838 | 53.524 | 8.240 | 1.00 | 10.42 | 7 | N |
| ATOM | 356 | CA | ASP | A | 47 | −4.298 | 53.427 | 7.930 | 1.00 | 13.28 | 6 | C |
| ATOM | 357 | CB | ASP | A | 47 | −4.956 | 54.763 | 7.696 | 1.00 | 17.79 | 6 | C |
| ATOM | 358 | CG | ASP | A | 47 | −4.912 | 55.703 | 8.873 | 1.00 | 22.85 | 6 | C |
| ATOM | 359 | OD1 | ASP | A | 47 | −5.187 | 55.282 | 10.024 | 1.00 | 24.70 | 8 | O |
| ATOM | 360 | OD2 | ASP | A | 47 | −4.546 | 56.871 | 8.607 | 1.00 | 26.41 | 8 | O |
| ATOM | 361 | C | ASP | A | 47 | −4.556 | 52.525 | 6.736 | 1.00 | 12.26 | 6 | C |
| ATOM | 362 | O | ASP | A | 47 | −5.638 | 51.891 | 6.708 | 1.00 | 9.75 | 8 | O |
| ATOM | 363 | N | THR | A | 48 | −3.726 | 52.630 | 5.681 | 1.00 | 10.33 | 7 | N |
| ATOM | 364 | CA | THR | A | 48 | −3.998 | 51.918 | 4.451 | 1.00 | 10.32 | 6 | C |
| ATOM | 365 | CB | THR | A | 48 | −4.553 | 52.795 | 3.306 | 1.00 | 12.96 | 6 | C |
| ATOM | 366 | OG1 | THR | A | 48 | −3.520 | 53.774 | 3.020 | 1.00 | 11.60 | 8 | O |
| ATOM | 367 | CG2 | THR | A | 48 | −5.883 | 53.503 | 3.608 | 1.00 | 15.12 | 6 | C |
| ATOM | 368 | C | THR | A | 48 | −2.701 | 51.285 | 3.893 | 1.00 | 11.08 | 6 | C |
| ATOM | 369 | O | THR | A | 48 | −1.596 | 51.629 | 4.290 | 1.00 | 10.17 | 8 | O |
| ATOM | 370 | N | LEU | A | 49 | −2.831 | 50.326 | 3.009 | 1.00 | 11.90 | 7 | N |
| ATOM | 371 | CA | LEU | A | 49 | −1.700 | 49.679 | 2.357 | 1.00 | 13.32 | 6 | C |
| ATOM | 372 | CB | LEU | A | 49 | −2.134 | 48.604 | 1.395 | 1.00 | 14.57 | 6 | C |
| ATOM | 373 | CG | LEU | A | 49 | −1.094 | 47.810 | 0.623 | 1.00 | 17.35 | 6 | C |
| ATOM | 374 | CD1 | LEU | A | 49 | −0.056 | 47.181 | 1.546 | 1.00 | 15.04 | 6 | C |
| ATOM | 375 | CD2 | LEU | A | 49 | −1.808 | 46.682 | −0.141 | 1.00 | 17.00 | 6 | C |
| ATOM | 376 | C | LEU | A | 49 | −0.852 | 50.701 | 1.596 | 1.00 | 14.01 | 6 | C |
| ATOM | 377 | O | LEU | A | 49 | 0.391 | 50.684 | 1.581 | 1.00 | 13.28 | 8 | O |
| ATOM | 378 | N | LYS | A | 50 | −1.512 | 51.662 | 0.995 | 1.00 | 14.48 | 7 | N |
| ATOM | 379 | CA | LYS | A | 50 | −0.901 | 52.771 | 0.305 | 1.00 | 17.54 | 6 | C |
| ATOM | 380 | CB | LYS | A | 50 | −2.009 | 53.660 | −0.266 | 1.00 | 21.43 | 6 | C |
| ATOM | 381 | CG | LYS | A | 50 | −1.525 | 54.829 | −1.120 | 1.00 | 27.31 | 6 | C |
| ATOM | 382 | CD | LYS | A | 50 | −2.665 | 55.246 | −2.068 | 1.00 | 30.92 | 6 | C |
| ATOM | 383 | CE | LYS | A | 50 | −2.165 | 55.936 | −3.327 | 1.00 | 35.13 | 6 | C |
| ATOM | 384 | NZ | LYS | A | 50 | −1.681 | 55.003 | −4.385 | 1.00 | 37.07 | 7 | N |
| ATOM | 385 | C | LYS | A | 50 | −0.042 | 53.546 | 1.285 | 1.00 | 15.32 | 6 | C |
| ATOM | 386 | O | LYS | A | 50 | 1.063 | 53.914 | 0.938 | 1.00 | 14.61 | 8 | O |
| ATOM | 387 | N | ASP | A | 51 | −0.491 | 53.838 | 2.496 | 1.00 | 14.52 | 7 | N |
| ATOM | 388 | CA | ASP | A | 51 | 0.314 | 54.507 | 3.504 | 1.00 | 13.63 | 6 | C |
| ATOM | 389 | CB | ASP | A | 51 | −0.439 | 54.839 | 4.775 | 1.00 | 16.05 | 6 | C |
| ATOM | 390 | CG | ASP | A | 51 | −1.564 | 55.875 | 4.586 | 1.00 | 19.66 | 6 | C |
| ATOM | 391 | OD1 | ASP | A | 51 | −1.389 | 56.778 | 3.763 | 1.00 | 18.33 | 8 | O |
| ATOM | 392 | OD2 | ASP | A | 51 | −2.609 | 55.712 | 5.280 | 1.00 | 20.80 | 8 | O |
| ATOM | 393 | C | ASP | A | 51 | 1.563 | 53.630 | 3.824 | 1.00 | 12.48 | 6 | C |
| ATOM | 394 | O | ASP | A | 51 | 2.638 | 54.196 | 4.026 | 1.00 | 10.69 | 8 | O |
| ATOM | 395 | N | LEU | A | 52 | 1.429 | 52.318 | 3.906 | 1.00 | 10.26 | 7 | N |
| ATOM | 396 | CA | LEU | A | 52 | 2.587 | 51.461 | 4.209 | 1.00 | 10.76 | 6 | C |
| ATOM | 397 | CB | LEU | A | 52 | 2.161 | 50.001 | 4.439 | 1.00 | 8.04 | 6 | C |
| ATOM | 398 | CG | LEU | A | 52 | 3.303 | 48.994 | 4.717 | 1.00 | 8.93 | 6 | C |
| ATOM | 399 | CD1 | LEU | A | 52 | 4.036 | 49.344 | 5.991 | 1.00 | 8.54 | 6 | C |
| ATOM | 400 | CD2 | LEU | A | 52 | 2.727 | 47.582 | 4.789 | 1.00 | 6.69 | 6 | C |
| ATOM | 401 | C | LEU | A | 52 | 3.609 | 51.536 | 3.086 | 1.00 | 10.23 | 6 | C |
| ATOM | 402 | O | LEU | A | 52 | 4.803 | 51.700 | 3.307 | 1.00 | 10.81 | 8 | O |
| ATOM | 403 | N | ASP | A | 53 | 3.139 | 51.505 | 1.847 | 1.00 | 12.35 | 7 | N |
| ATOM | 404 | CA | ASP | A | 53 | 3.945 | 51.675 | 0.658 | 1.00 | 14.81 | 6 | C |
| ATOM | 405 | CB | ASP | A | 53 | 3.000 | 51.762 | −0.539 | 1.00 | 16.01 | 6 | C |
| ATOM | 406 | CG | ASP | A | 53 | 2.380 | 50.470 | −0.981 | 1.00 | 16.50 | 6 | C |
| ATOM | 407 | OD1 | ASP | A | 53 | 2.487 | 49.314 | −0.542 | 1.00 | 16.66 | 8 | O |
| ATOM | 408 | OD2 | ASP | A | 53 | 1.600 | 50.652 | −1.934 | 1.00 | 19.89 | 8 | O |
| ATOM | 409 | C | ASP | A | 53 | 4.760 | 52.960 | 0.728 | 1.00 | 16.37 | 6 | C |
| ATOM | 410 | O | ASP | A | 53 | 5.984 | 52.976 | 0.478 | 1.00 | 13.89 | 8 | O |
| ATOM | 411 | N | GLN | A | 54 | 4.067 | 54.021 | 1.166 | 1.00 | 16.03 | 7 | N |
| ATOM | 412 | CA | GLN | A | 54 | 4.736 | 55.341 | 1.262 | 1.00 | 17.59 | 6 | C |
| ATOM | 413 | CB | GLN | A | 54 | 3.640 | 56.376 | 1.250 | 1.00 | 22.16 | 6 | C |
| ATOM | 414 | CG | GLN | A | 54 | 3.822 | 57.848 | 1.338 | 1.00 | 30.35 | 6 | C |
| ATOM | 415 | CD | GLN | A | 54 | 2.485 | 58.612 | 1.418 | 1.00 | 35.56 | 6 | C |
| ATOM | 416 | OE1 | GLN | A | 54 | 1.366 | 58.133 | 1.134 | 1.00 | 37.13 | 8 | O |
| ATOM | 417 | NE2 | GLN | A | 54 | 2.602 | 59.867 | 1.844 | 1.00 | 36.35 | 7 | N |
| ATOM | 418 | C | GLN | A | 54 | 5.722 | 55.402 | 2.394 | 1.00 | 15.60 | 6 | C |
| ATOM | 419 | O | GLN | A | 54 | 6.791 | 56.008 | 2.287 | 1.00 | 16.58 | 8 | O |
| ATOM | 420 | N | PHE | A | 55 | 5.539 | 54.709 | 3.507 | 1.00 | 14.54 | 7 | N |
| ATOM | 421 | CA | PHE | A | 55 | 6.508 | 54.584 | 4.590 | 1.00 | 12.24 | 6 | C |
| ATOM | 422 | CB | PHE | A | 55 | 5.901 | 53.747 | 5.733 | 1.00 | 12.77 | 6 | C |
| ATOM | 423 | CG | PHE | A | 55 | 6.785 | 53.705 | 6.955 | 1.00 | 11.74 | 6 | C |
| ATOM | 424 | CD1 | PHE | A | 55 | 6.962 | 54.849 | 7.699 | 1.00 | 13.70 | 6 | C |
| ATOM | 425 | CD2 | PHE | A | 55 | 7.483 | 52.560 | 7.281 | 1.00 | 11.58 | 6 | C |
| ATOM | 426 | CE1 | PHE | A | 55 | 7.786 | 54.861 | 8.806 | 1.00 | 11.72 | 6 | C |
| ATOM | 427 | CE2 | PHE | A | 55 | 8.345 | 52.564 | 8.372 | 1.00 | 13.77 | 6 | C |
| ATOM | 428 | CZ | PHE | A | 55 | 8.485 | 53.705 | 9.134 | 1.00 | 13.93 | 6 | C |
| ATOM | 429 | C | PHE | A | 55 | 7.793 | 53.901 | 4.107 | 1.00 | 11.18 | 6 | C |
| ATOM | 430 | O | PHE | A | 55 | 8.914 | 54.384 | 4.344 | 1.00 | 11.64 | 8 | O |
| ATOM | 431 | N | ILE | A | 56 | 7.694 | 52.778 | 3.413 | 1.00 | 9.30 | 7 | N |
| ATOM | 432 | CA | ILE | A | 56 | 8.772 | 52.040 | 2.820 | 1.00 | 10.44 | 6 | C |
| ATOM | 433 | CB | ILE | A | 56 | 8.350 | 50.766 | 2.043 | 1.00 | 12.03 | 6 | C |
| ATOM | 434 | CG2 | ILE | A | 56 | 9.595 | 50.037 | 1.526 | 1.00 | 11.62 | 6 | C |
| ATOM | 435 | CG1 | ILE | A | 56 | 7.507 | 49.761 | 2.823 | 1.00 | 13.57 | 6 | C |
| ATOM | 436 | CD1 | ILE | A | 56 | 8.161 | 49.397 | 4.141 | 1.00 | 16.82 | 6 | C |
| ATOM | 437 | C | ILE | A | 56 | 9.521 | 52.936 | 1.813 | 1.00 | 10.94 | 6 | C |
| ATOM | 438 | O | ILE | A | 56 | 10.737 | 53.141 | 2.008 | 1.00 | 10.83 | 8 | O |
| ATOM | 439 | N | THR | A | 57 | 8.820 | 53.526 | 0.843 | 1.00 | 10.32 | 7 | N |
| ATOM | 440 | CA | THR | A | 57 | 9.547 | 54.292 | −0.177 | 1.00 | 15.45 | 6 | C |
| ATOM | 441 | CB | THR | A | 57 | 8.786 | 54.444 | −1.513 | 1.00 | 15.05 | 6 | C |
| ATOM | 442 | OG1 | THR | A | 57 | 7.636 | 55.254 | −1.248 | 1.00 | 17.59 | 8 | O |
| ATOM | 443 | CG2 | THR | A | 57 | 8.341 | 53.042 | −1.938 | 1.00 | 15.90 | 6 | C |
| ATOM | 444 | C | THR | A | 57 | 10.046 | 55.637 | 0.282 | 1.00 | 18.09 | 6 | C |
| ATOM | 445 | O | THR | A | 57 | 11.145 | 56.074 | −0.104 | 1.00 | 17.72 | 8 | O |
| ATOM | 446 | N | GLU | A | 58 | 9.248 | 56.355 | 1.064 | 1.00 | 18.70 | 7 | N |
| ATOM | 447 | CA | GLU | A | 58 | 9.648 | 57.717 | 1.436 | 1.00 | 21.41 | 6 | C |
| ATOM | 448 | CB | GLU | A | 58 | 8.422 | 58.659 | 1.495 | 1.00 | 23.68 | 6 | C |
| ATOM | 449 | CG | GLU | A | 58 | 7.668 | 58.633 | 0.164 | 1.00 | 28.38 | 6 | C |
| ATOM | 450 | CD | GLU | A | 58 | 8.510 | 58.884 | −1.083 | 1.00 | 32.18 | 6 | C |
| ATOM | 451 | OE1 | GLU | A | 58 | 9.164 | 59.955 | −1.157 | 1.00 | 34.50 | 8 | O |
| ATOM | 452 | OE2 | GLU | A | 58 | 8.542 | 58.044 | −2.033 | 1.00 | 31.90 | 8 | O |
| ATOM | 453 | C | GLU | A | 58 | 10.458 | 57.767 | 2.715 | 1.00 | 20.49 | 6 | C |
| ATOM | 454 | O | GLU | A | 58 | 11.158 | 58.771 | 2.852 | 1.00 | 19.41 | 8 | O |
| ATOM | 455 | N | LYS | A | 59 | 10.333 | 56.793 | 3.610 | 1.00 | 17.03 | 7 | N |
| ATOM | 456 | CA | LYS | A | 59 | 11.082 | 56.882 | 4.860 | 1.00 | 18.11 | 6 | C |
| ATOM | 457 | CB | LYS | A | 59 | 10.157 | 56.735 | 6.085 | 1.00 | 20.27 | 6 | C |
| ATOM | 458 | CG | LYS | A | 59 | 9.242 | 57.974 | 6.156 | 1.00 | 25.90 | 6 | C |
| ATOM | 459 | CD | LYS | A | 59 | 8.467 | 58.103 | 7.454 | 1.00 | 31.51 | 6 | C |
| ATOM | 460 | CE | LYS | A | 59 | 7.799 | 59.461 | 7.620 | 1.00 | 33.05 | 6 | C |
| ATOM | 461 | NZ | LYS | A | 59 | 6.928 | 59.580 | 8.833 | 1.00 | 33.55 | 7 | N |
| ATOM | 462 | C | LYS | A | 59 | 12.227 | 55.877 | 4.872 | 1.00 | 16.26 | 6 | C |
| ATOM | 463 | O | LYS | A | 59 | 13.411 | 56.261 | 4.886 | 1.00 | 14.64 | 8 | O |
| ATOM | 464 | N | ILE | A | 60 | 11.861 | 54.602 | 4.787 | 1.00 | 13.46 | 7 | N |
| ATOM | 465 | CA | ILE | A | 60 | 12.891 | 53.543 | 4.870 | 1.00 | 12.61 | 6 | C |
| ATOM | 466 | CB | ILE | A | 60 | 12.205 | 52.176 | 4.758 | 1.00 | 12.63 | 6 | C |
| ATOM | 467 | CG2 | ILE | A | 60 | 13.152 | 50.999 | 4.855 | 1.00 | 14.47 | 6 | C |
| ATOM | 468 | CG1 | ILE | A | 60 | 11.078 | 52.045 | 5.811 | 1.00 | 13.49 | 6 | C |
| ATOM | 469 | CD1 | ILE | A | 60 | 11.557 | 52.160 | 7.226 | 1.00 | 15.68 | 6 | C |
| ATOM | 470 | C | ILE | A | 60 | 13.933 | 53.683 | 3.769 | 1.00 | 12.10 | 6 | C |
| ATOM | 471 | O | ILE | A | 60 | 15.130 | 53.686 | 4.030 | 1.00 | 11.15 | 8 | O |
| ATOM | 472 | N | ARG | A | 61 | 13.477 | 53.685 | 2.531 | 1.00 | 11.05 | 7 | N |
| ATOM | 473 | CA | ARG | A | 61 | 14.398 | 53.702 | 1.390 | 1.00 | 14.35 | 6 | C |
| ATOM | 474 | CB | ARG | A | 61 | 13.613 | 53.280 | 0.121 | 1.00 | 12.92 | 6 | C |
| ATOM | 475 | CG | ARG | A | 61 | 13.408 | 51.765 | 0.293 | 1.00 | 15.09 | 6 | C |
| ATOM | 476 | CD | ARG | A | 61 | 12.740 | 51.100 | −0.894 | 1.00 | 14.74 | 6 | C |
| ATOM | 477 | NE | ARG | A | 61 | 12.694 | 49.638 | −0.750 | 1.00 | 10.39 | 7 | N |
| ATOM | 478 | CZ | ARG | A | 61 | 12.155 | 48.865 | −1.683 | 1.00 | 10.84 | 6 | C |
| ATOM | 479 | NH1 | ARG | A | 61 | 11.705 | 49.379 | −2.843 | 1.00 | 8.51 | 7 | N |
| ATOM | 480 | NH2 | ARG | A | 61 | 12.149 | 47.541 | −1.520 | 1.00 | 7.77 | 7 | N |
| ATOM | 481 | C | ARG | A | 61 | 15.230 | 54.952 | 1.199 | 1.00 | 12.68 | 6 | C |
| ATOM | 482 | O | ARG | A | 61 | 16.126 | 54.985 | 0.379 | 1.00 | 12.57 | 8 | O |
| ATOM | 483 | N | LYS | A | 62 | 14.965 | 56.045 | 1.874 | 1.00 | 11.92 | 7 | N |
| ATOM | 484 | CA | LYS | A | 62 | 15.767 | 57.215 | 1.937 | 1.00 | 15.99 | 6 | C |
| ATOM | 485 | CB | LYS | A | 62 | 14.866 | 58.469 | 2.069 | 1.00 | 18.60 | 6 | C |
| ATOM | 486 | CG | LYS | A | 62 | 14.165 | 58.660 | 0.733 | 1.00 | 21.84 | 6 | C |
| ATOM | 487 | CD | LYS | A | 62 | 13.529 | 60.019 | 0.531 | 1.00 | 25.52 | 6 | C |
| ATOM | 488 | CE | LYS | A | 62 | 12.694 | 59.946 | −0.762 | 1.00 | 25.02 | 6 | C |
| ATOM | 489 | NZ | LYS | A | 62 | 12.156 | 61.305 | −1.045 | 1.00 | 27.48 | 7 | N |
| ATOM | 490 | C | LYS | A | 62 | 16.820 | 57.155 | 3.057 | 1.00 | 17.44 | 6 | C |
| ATOM | 491 | O | LYS | A | 62 | 17.554 | 58.146 | 3.135 | 1.00 | 17.35 | 8 | O |
| ATOM | 492 | N | MET | A | 63 | 16.892 | 56.117 | 3.867 | 1.00 | 14.00 | 7 | N |
| ATOM | 493 | CA | MET | A | 63 | 17.923 | 56.000 | 4.889 | 1.00 | 16.24 | 6 | C |
| ATOM | 494 | CB | MET | A | 63 | 17.312 | 55.433 | 6.183 | 1.00 | 17.88 | 6 | C |
| ATOM | 495 | CG | MET | A | 63 | 16.270 | 56.338 | 6.811 | 1.00 | 21.60 | 6 | C |
| ATOM | 496 | SD | MET | A | 63 | 15.470 | 55.691 | 8.306 | 1.00 | 20.40 | 16 | S |
| ATOM | 497 | CE | MET | A | 63 | 14.362 | 54.509 | 7.598 | 1.00 | 24.10 | 6 | C |
| ATOM | 498 | C | MET | A | 63 | 19.041 | 55.086 | 4.400 | 1.00 | 14.86 | 6 | C |
| ATOM | 499 | O | MET | A | 63 | 18.889 | 53.879 | 4.279 | 1.00 | 11.39 | 8 | O |
| ATOM | 500 | N | PRO | A | 64 | 20.235 | 55.621 | 4.123 | 1.00 | 16.04 | 7 | N |
| ATOM | 501 | CD | PRO | A | 64 | 20.544 | 57.076 | 4.204 | 1.00 | 16.05 | 6 | C |
| ATOM | 502 | CA | PRO | A | 64 | 21.320 | 54.831 | 3.549 | 1.00 | 16.25 | 6 | C |
| ATOM | 503 | CB | PRO | A | 64 | 22.349 | 55.886 | 3.119 | 1.00 | 17.65 | 6 | C |
| ATOM | 504 | CG | PRO | A | 64 | 22.041 | 57.059 | 4.001 | 1.00 | 18.37 | 6 | C |
| ATOM | 505 | CG | PRO | A | 64 | 21.892 | 53.725 | 4.391 | 1.00 | 16.20 | 6 | C |
| ATOM | 506 | O | PRO | A | 64 | 22.415 | 52.724 | 3.846 | 1.00 | 17.51 | 8 | O |
| ATOM | 507 | N | GLU | A | 65 | 21.774 | 53.746 | 5.713 | 1.00 | 16.16 | 7 | N |
| ATOM | 508 | CA | GLU | A | 65 | 22.214 | 52.697 | 6.620 | 1.00 | 19.24 | 6 | C |
| ATOM | 509 | CB | GLU | A | 65 | 22.275 | 53.230 | 8.060 | 1.00 | 21.22 | 6 | C |
| ATOM | 510 | CG | GLU | A | 65 | 22.899 | 54.616 | 8.078 | 1.00 | 25.48 | 6 | C |
| ATOM | 511 | CD | GLU | A | 65 | 21.970 | 55.758 | 7.731 | 1.00 | 30.11 | 6 | C |
| ATOM | 512 | OE1 | GLU | A | 65 | 20.719 | 55.794 | 7.576 | 1.00 | 26.69 | 8 | O |
| ATOM | 513 | OE2 | GLU | A | 65 | 22.562 | 56.925 | 7.697 | 1.00 | 35.08 | 8 | O |
| ATOM | 514 | C | GLU | A | 65 | 21.322 | 51.462 | 6.576 | 1.00 | 18.67 | 6 | C |
| ATOM | 515 | O | GLU | A | 65 | 21.812 | 50.337 | 6.859 | 1.00 | 17.95 | 8 | O |
| ATOM | 516 | N | ILE | A | 66 | 20.094 | 51.634 | 6.036 | 1.00 | 15.76 | 7 | N |
| ATOM | 517 | CA | ILE | A | 66 | 19.264 | 50.426 | 5.886 | 1.00 | 15.12 | 6 | C |
| ATOM | 518 | CB | ILE | A | 66 | 17.761 | 50.790 | 6.040 | 1.00 | 15.29 | 6 | C |
| ATOM | 519 | CG2 | ILE | A | 66 | 16.985 | 49.503 | 5.763 | 1.00 | 17.90 | 6 | C |
| ATOM | 520 | CG1 | ILE | A | 66 | 17.536 | 51.382 | 7.403 | 1.00 | 15.51 | 6 | C |
| ATOM | 521 | CD1 | ILE | A | 66 | 16.130 | 51.794 | 7.783 | 1.00 | 18.15 | 6 | C |
| ATOM | 522 | C | ILE | A | 66 | 19.547 | 49.790 | 4.557 | 1.00 | 15.35 | 6 | C |
| ATOM | 523 | O | ILE | A | 66 | 19.500 | 50.521 | 3.558 | 1.00 | 16.62 | 8 | O |
| ATOM | 524 | N | GLN | A | 67 | 19.964 | 48.553 | 4.453 | 1.00 | 17.21 | 7 | N |
| ATOM | 525 | CA | GLN | A | 67 | 20.439 | 47.860 | 3.282 | 1.00 | 19.06 | 6 | C |
| ATOM | 526 | CB | GLN | A | 67 | 21.783 | 47.155 | 3.681 | 1.00 | 22.67 | 6 | C |
| ATOM | 527 | CG | GLN | A | 67 | 22.821 | 48.236 | 4.024 | 1.00 | 27.17 | 6 | C |
| ATOM | 528 | CD | GLN | A | 67 | 24.172 | 47.665 | 4.393 | 1.00 | 29.47 | 6 | C |
| ATOM | 529 | OE1 | GLN | A | 67 | 25.008 | 48.411 | 4.916 | 1.00 | 30.95 | 8 | O |
| ATOM | 530 | NE2 | GLN | A | 67 | 24.408 | 46.398 | 4.137 | 1.00 | 28.71 | 7 | N |
| ATOM | 531 | C | GLN | A | 67 | 19.599 | 45.747 | 2.673 | 1.00 | 20.41 | 6 | C |
| ATOM | 532 | O | GLN | A | 67 | 19.769 | 46.389 | 1.495 | 1.00 | 21.52 | 8 | O |
| ATOM | 533 | N | MET | A | 68 | 18.877 | 45.948 | 3.425 | 1.00 | 18.04 | 7 | N |
| ATOM | 534 | CA | MET | A | 68 | 17.961 | 44.941 | 2.966 | 1.00 | 16.99 | 6 | C |
| ATOM | 535 | CB | MET | A | 68 | 18.584 | 43.560 | 2.923 | 1.00 | 19.00 | 6 | C |
| ATOM | 536 | CG | MET | A | 68 | 18.006 | 42.708 | 1.809 | 1.00 | 25.20 | 6 | C |
| ATOM | 537 | SD | MET | A | 68 | 18.507 | 40.980 | 1.906 | 1.00 | 32.29 | 16 | S |
| ATOM | 538 | CE | MET | A | 68 | 20.191 | 41.033 | 1.269 | 1.00 | 31.84 | 6 | C |
| ATOM | 539 | C | MET | A | 68 | 16.753 | 44.876 | 3.930 | 1.00 | 14.82 | 6 | C |
| ATOM | 540 | O | MET | A | 68 | 16.955 | 44.821 | 5.156 | 1.00 | 9.54 | 8 | O |
| ATOM | 541 | N | THR | A | 69 | 15.567 | 44.888 | 3.325 | 1.00 | 10.34 | 7 | N |
| ATOM | 542 | CA | THR | A | 69 | 14.343 | 44.824 | 4.099 | 1.00 | 11.13 | 6 | C |
| ATOM | 543 | CB | THR | A | 69 | 13.513 | 46.151 | 4.085 | 1.00 | 11.22 | 6 | C |
| ATOM | 544 | OG1 | THR | A | 69 | 13.145 | 46.401 | 2.740 | 1.00 | 11.52 | 8 | O |
| ATOM | 545 | CG2 | THR | A | 69 | 14.291 | 47.328 | 4.617 | 1.00 | 10.08 | 6 | C |
| ATOM | 546 | C | THR | A | 69 | 13.428 | 43.733 | 3.590 | 1.00 | 12.72 | 6 | C |
| ATOM | 547 | O | THR | A | 69 | 13.574 | 43.310 | 2.431 | 1.00 | 12.83 | 8 | O |
| ATOM | 548 | N | SER | A | 70 | 12.650 | 43.124 | 4.475 | 1.00 | 10.14 | 7 | N |
| ATOM | 549 | CA | SER | A | 70 | 11.693 | 42.093 | 4.182 | 1.00 | 8.64 | 6 | C |
| ATOM | 550 | CB | SER | A | 70 | 12.094 | 40.688 | 4.647 | 1.00 | 8.17 | 6 | C |
| ATOM | 551 | OG | SER | A | 70 | 11.039 | 39.830 | 4.251 | 1.00 | 11.27 | 8 | O |
| ATOM | 552 | C | SER | A | 70 | 10.464 | 42.513 | 5.055 | 1.00 | 12.16 | 6 | C |
| ATOM | 553 | O | SER | A | 70 | 10.659 | 42.602 | 6.288 | 1.00 | 9.70 | 8 | O |
| ATOM | 554 | N | THR | A | 71 | 9.352 | 42.753 | 4.380 | 1.00 | 10.49 | 7 | N |
| ATOM | 555 | CA | THR | A | 71 | 8.153 | 43.198 | 5.110 | 1.00 | 11.09 | 6 | C |
| ATOM | 556 | CB | THR | A | 71 | 7.587 | 44.481 | 4.499 | 1.00 | 11.77 | 6 | C |
| ATOM | 557 | OG1 | THR | A | 71 | 8.593 | 45.476 | 4.690 | 1.00 | 10.42 | 8 | O |
| ATOM | 558 | CG2 | THR | A | 71 | 6.295 | 45.014 | 5.189 | 1.00 | 13.37 | 6 | C |
| ATOM | 559 | C | THR | A | 71 | 7.092 | 42.123 | 5.214 | 1.00 | 10.51 | 6 | C |
| ATOM | 560 | O | THR | A | 71 | 6.771 | 41.472 | 4.217 | 1.00 | 10.60 | 8 | O |
| ATOM | 561 | N | MET | A | 72 | 6.591 | 41.918 | 6.423 | 1.00 | 8.76 | 7 | N |
| ATOM | 562 | CA | MET | A | 72 | 5.484 | 40.937 | 6.575 | 1.00 | 11.11 | 6 | C |
| ATOM | 563 | CB | MET | A | 72 | 5.816 | 39.916 | 7.651 | 1.00 | 13.22 | 6 | C |
| ATOM | 564 | CG | MET | A | 72 | 6.324 | 38.626 | 6.987 | 1.00 | 21.65 | 6 | C |
| ATOM | 565 | SD | MET | A | 72 | 7.682 | 37.932 | 7.913 | 1.00 | 27.13 | 16 | S |
| ATOM | 566 | CE | MET | A | 72 | 9.041 | 38.835 | 7.191 | 1.00 | 25.46 | 6 | C |
| ATOM | 567 | C | MET | A | 72 | 4.216 | 41.662 | 7.019 | 1.00 | 10.65 | 6 | C |
| ATOM | 568 | O | MET | A | 72 | 4.247 | 42.322 | 8.052 | 1.00 | 8.92 | 8 | O |
| ATOM | 569 | N | ILE | A | 73 | 3.140 | 41.593 | 6.255 | 1.00 | 10.57 | 7 | N |
| ATOM | 570 | CA | ILE | A | 73 | 1.905 | 42.309 | 6.581 | 1.00 | 8.83 | 6 | C |
| ATOM | 571 | CB | ILE | A | 73 | 1.359 | 42.913 | 5.279 | 1.00 | 8.76 | 6 | C |
| ATOM | 572 | CG2 | ILE | A | 73 | 0.061 | 43.700 | 5.504 | 1.00 | 7.15 | 6 | C |
| ATOM | 573 | CG1 | ILE | A | 73 | 2.472 | 43.768 | 4.604 | 1.00 | 8.63 | 6 | C |
| ATOM | 574 | CD1 | ILE | A | 73 | 2.028 | 44.206 | 3.186 | 1.00 | 8.44 | 6 | C |
| ATOM | 575 | C | ILE | A | 73 | 0.877 | 41.401 | 7.223 | 1.00 | 10.71 | 6 | C |
| ATOM | 576 | O | ILE | A | 73 | 0.582 | 40.281 | 6.799 | 1.00 | 12.18 | 8 | O |
| ATOM | 577 | N | ALA | A | 74 | 0.305 | 41.845 | 8.320 | 1.00 | 11.15 | 7 | N |
| ATOM | 578 | CA | ALA | A | 74 | −0.650 | 41.052 | 9.086 | 1.00 | 11.38 | 6 | C |
| ATOM | 579 | CB | ALA | A | 74 | −0.976 | 41.637 | 10.453 | 1.00 | 8.81 | 6 | C |
| ATOM | 580 | C | ALA | A | 74 | −1.900 | 40.883 | 8.226 | 1.00 | 13.75 | 6 | C |
| ATOM | 581 | O | ALA | A | 74 | −2.334 | 41.884 | 7.674 | 1.00 | 9.08 | 8 | O |
| ATOM | 582 | N | ILE | A | 75 | −2.464 | 39.673 | 8.299 | 1.00 | 14.79 | 7 | N |
| ATOM | 583 | CA | ILE | A | 75 | −3.640 | 39.439 | 7.473 | 1.00 | 20.01 | 6 | C |
| ATOM | 584 | CB | ILE | A | 75 | −3.364 | 38.627 | 6.190 | 1.00 | 20.01 | 6 | C |
| ATOM | 585 | CG2 | ILE | A | 75 | −2.744 | 39.438 | 5.070 | 1.00 | 20.74 | 6 | C |
| ATOM | 586 | CG1 | ILE | A | 75 | −2.502 | 37.432 | 6.567 | 1.00 | 21.65 | 6 | C |
| ATOM | 587 | CD1 | ILE | A | 75 | −2.352 | 36.330 | 5.553 | 1.00 | 23.37 | 6 | C |
| ATOM | 588 | C | ILE | A | 75 | −4.656 | 38.685 | 8.333 | 1.00 | 21.16 | 6 | C |
| ATOM | 589 | O | ILE | A | 75 | −4.293 | 38.301 | 9.452 | 1.00 | 19.96 | 8 | O |
| ATOM | 590 | OT | ILE | A | 75 | −5.709 | 38.325 | 7.766 | 1.00 | 24.56 | 8 | O |
| ATOM | 591 | CB | VAL | B | 2 | 15.203 | 36.576 | 27.110 | 1.00 | 10.81 | 6 | C |
| ATOM | 592 | CG1 | VAL | B | 2 | 15.489 | 36.311 | 25.630 | 1.00 | 10.60 | 6 | C |
| ATOM | 593 | CG2 | VAL | B | 2 | 16.487 | 36.270 | 27.887 | 1.00 | 9.64 | 6 | C |
| ATOM | 594 | C | VAL | B | 2 | 13.491 | 38.209 | 26.316 | 1.00 | 9.81 | 6 | C |
| ATOM | 595 | O | VAL | B | 2 | 12.467 | 37.543 | 26.535 | 1.00 | 8.96 | 8 | O |
| ATOM | 596 | N | VAL | B | 2 | 14.395 | 38.441 | 28.691 | 1.00 | 10.90 | 7 | N |
| ATOM | 597 | CA | VAL | B | 2 | 14.702 | 38.012 | 27.247 | 1.00 | 9.65 | 6 | C |
| ATOM | 598 | N | THR | B | 3 | 13.641 | 39.006 | 25.278 | 1.00 | 8.35 | 7 | N |
| ATOM | 599 | CA | THR | B | 3 | 12.571 | 39.256 | 24.318 | 1.00 | 7.94 | 6 | C |
| ATOM | 600 | CB | THR | B | 3 | 12.241 | 40.729 | 24.045 | 1.00 | 8.58 | 6 | C |
| ATOM | 601 | OG1 | THR | B | 3 | 11.836 | 41.393 | 25.244 | 1.00 | 8.95 | 8 | O |
| ATOM | 602 | CG2 | THR | B | 3 | 11.075 | 40.881 | 23.044 | 1.00 | 6.74 | 6 | C |
| ATOM | 603 | C | THR | B | 3 | 13.026 | 38.662 | 22.981 | 1.00 | 10.25 | 6 | C |
| ATOM | 604 | O | THR | B | 3 | 14.248 | 38.762 | 22.725 | 1.00 | 6.93 | 8 | O |
| ATOM | 605 | N | ALA | B | 4 | 12.186 | 37.851 | 22.328 | 1.00 | 7.68 | 7 | N |
| ATOM | 606 | CA | ALA | B | 4 | 12.535 | 37.308 | 21.033 | 1.00 | 8.07 | 6 | C |
| ATOM | 607 | CB | ALA | B | 4 | 13.073 | 35.859 | 21.152 | 1.00 | 5.87 | 6 | C |
| ATOM | 608 | C | ALA | B | 4 | 11.315 | 37.351 | 20.096 | 1.00 | 9.20 | 6 | C |
| ATOM | 609 | O | ALA | B | 4 | 10.154 | 37.299 | 20.559 | 1.00 | 9.29 | 8 | O |
| ATOM | 610 | N | PHE | B | 5 | 11.596 | 37.401 | 18.802 | 1.00 | 6.28 | 7 | N |
| ATOM | 611 | CA | PHE | B | 5 | 10.592 | 37.272 | 17.760 | 1.00 | 8.35 | 6 | C |
| ATOM | 612 | CB | PHE | B | 5 | 10.612 | 38.420 | 16.752 | 1.00 | 6.55 | 6 | C |
| ATOM | 613 | CG | PHE | B | 5 | 10.185 | 39.729 | 17.409 | 1.00 | 7.88 | 6 | C |
| ATOM | 614 | CD1 | PHE | B | 5 | 11.087 | 40.520 | 18.080 | 1.00 | 6.54 | 6 | C |
| ATOM | 615 | CD2 | PHE | B | 5 | 8.853 | 40.140 | 17.310 | 1.00 | 8.11 | 6 | C |
| ATOM | 616 | CE1 | PHE | B | 5 | 10.653 | 41.713 | 18.654 | 1.00 | 8.22 | 6 | C |
| ATOM | 617 | CE2 | PHE | B | 5 | 8.436 | 41.346 | 17.873 | 1.00 | 7.71 | 6 | C |
| ATOM | 618 | CZ | PHE | B | 5 | 9.335 | 42.140 | 18.531 | 1.00 | 7.27 | 6 | C |
| ATOM | 619 | C | PHE | B | 5 | 10.793 | 35.963 | 16.987 | 1.00 | 7.00 | 6 | C |
| ATOM | 620 | O | PHE | B | 5 | 11.904 | 35.640 | 16.514 | 1.00 | 6.91 | 8 | O |
| ATOM | 621 | N | ILE | B | 6 | 9.771 | 35.121 | 16.971 | 1.00 | 7.10 | 7 | N |
| ATOM | 622 | CA | ILE | B | 6 | 9.898 | 33.824 | 16.270 | 1.00 | 7.30 | 6 | C |
| ATOM | 623 | CB | ILE | B | 6 | 9.190 | 32.706 | 17.057 | 1.00 | 7.52 | 6 | C |
| ATOM | 624 | CG2 | ILE | B | 6 | 9.353 | 31.396 | 16.284 | 1.00 | 6.04 | 6 | C |
| ATOM | 625 | CG1 | ILE | B | 6 | 9.708 | 32.559 | 18.504 | 1.00 | 6.96 | 6 | C |
| ATOM | 626 | CD1 | ILE | B | 6 | 8.811 | 31.481 | 19.176 | 1.00 | 12.29 | 6 | C |
| ATOM | 627 | C | ILE | B | 6 | 9.210 | 33.940 | 14.917 | 1.00 | 10.08 | 6 | C |
| ATOM | 628 | O | ILE | B | 6 | 7.991 | 34.222 | 14.923 | 1.00 | 10.99 | 8 | O |
| ATOM | 629 | N | LEU | B | 7 | 9.882 | 33.668 | 13.819 | 1.00 | 11.51 | 7 | N |
| ATOM | 630 | CA | LEU | B | 7 | 9.237 | 33.678 | 12.492 | 1.00 | 12.43 | 6 | C |
| ATOM | 631 | CB | LEU | B | 7 | 10.173 | 34.267 | 11.447 | 1.00 | 12.72 | 6 | C |
| ATOM | 632 | CG | LEU | B | 7 | 10.630 | 35.703 | 11.722 | 1.00 | 17.00 | 6 | C |
| ATOM | 633 | CD1 | LEU | B | 7 | 11.870 | 36.078 | 10.883 | 1.00 | 17.22 | 6 | C |
| ATOM | 634 | CD2 | LEU | B | 7 | 9.485 | 36.665 | 11.508 | 1.00 | 18.19 | 6 | C |
| ATOM | 635 | C | LEU | B | 7 | 8.919 | 32.224 | 12.235 | 1.00 | 14.19 | 6 | C |
| ATOM | 636 | O | LEU | B | 7 | 9.746 | 31.328 | 12.500 | 1.00 | 13.47 | 8 | O |
| ATOM | 637 | N | MET | B | 8 | 7.688 | 31.875 | 11.846 | 1.00 | 13.99 | 7 | N |
| ATOM | 638 | CA | MET | B | 8 | 7.306 | 30.486 | 11.708 | 1.00 | 13.46 | 6 | C |
| ATOM | 639 | CB | MET | B | 8 | 6.368 | 30.028 | 12.835 | 1.00 | 16.11 | 6 | C |
| ATOM | 640 | CG | MET | B | 8 | 6.691 | 30.264 | 14.271 | 1.00 | 18.68 | 6 | C |
| ATOM | 641 | SD | MET | B | 8 | 5.428 | 29.803 | 15.535 | 1.00 | 20.37 | 16 | S |
| ATOM | 642 | CE | MET | B | 8 | 4.118 | 29.468 | 14.437 | 1.00 | 17.84 | 6 | C |
| ATOM | 643 | C | MET | B | 8 | 6.609 | 30.158 | 10.390 | 1.00 | 15.90 | 6 | C |
| ATOM | 644 | O | MET | B | 8 | 5.832 | 30.941 | 9.852 | 1.00 | 14.92 | 8 | O |
| ATOM | 645 | N | VAL | B | 9 | 6.857 | 28.937 | 9.914 | 1.00 | 16.65 | 7 | N |
| ATOM | 646 | CA | VAL | B | 9 | 6.126 | 28.443 | 8.730 | 1.00 | 16.44 | 6 | C |
| ATOM | 647 | CB | VAL | B | 9 | 7.072 | 27.868 | 7.674 | 1.00 | 17.96 | 6 | C |
| ATOM | 648 | CG1 | VAL | B | 9 | 6.361 | 27.078 | 6.558 | 1.00 | 16.89 | 6 | C |
| ATOM | 649 | CG2 | VAL | B | 9 | 7.856 | 29.021 | 7.048 | 1.00 | 17.27 | 6 | C |
| ATOM | 650 | C | VAL | B | 9 | 5.258 | 27.333 | 9.285 | 1.00 | 17.36 | 6 | C |
| ATOM | 651 | O | VAL | B | 9 | 5.868 | 26.512 | 9.992 | 1.00 | 18.71 | 8 | O |
| ATOM | 652 | N | THR | B | 10 | 3.947 | 27.306 | 9.086 | 1.00 | 16.83 | 7 | N |
| ATOM | 653 | CA | THR | B | 10 | 3.149 | 26.199 | 9.609 | 1.00 | 16.97 | 6 | C |
| ATOM | 654 | CB | THR | B | 10 | 1.860 | 26.674 | 10.313 | 1.00 | 17.72 | 6 | C |
| ATOM | 655 | OG1 | THR | B | 10 | 1.014 | 27.388 | 9.376 | 1.00 | 16.78 | 8 | O |
| ATOM | 656 | CG2 | THR | B | 10 | 2.219 | 27.659 | 11.422 | 1.00 | 15.85 | 6 | C |
| ATOM | 657 | C | THR | B | 10 | 2.690 | 25.289 | 8.460 | 1.00 | 18.72 | 6 | C |
| ATOM | 658 | O | THR | B | 10 | 2.941 | 25.560 | 7.279 | 1.00 | 19.05 | 8 | O |
| ATOM | 659 | N | ALA | B | 11 | 2.101 | 24.137 | 8.755 | 1.00 | 20.00 | 7 | N |
| ATOM | 660 | CA | ALA | B | 11 | 1.480 | 23.354 | 7.657 | 1.00 | 20.04 | 6 | C |
| ATOM | 661 | CB | ALA | B | 11 | 0.916 | 22.098 | 8.243 | 1.00 | 20.18 | 6 | C |
| ATOM | 662 | C | ALA | B | 11 | 0.384 | 24.233 | 7.072 | 1.00 | 22.64 | 6 | C |
| ATOM | 663 | O | ALA | B | 11 | −0.259 | 24.987 | 7.818 | 1.00 | 22.86 | 8 | O |
| ATOM | 664 | N | ALA | B | 12 | 0.184 | 24.225 | 5.759 | 1.00 | 24.51 | 7 | N |
| ATOM | 665 | CA | ALA | B | 12 | −0.856 | 25.096 | 5.158 | 1.00 | 24.49 | 6 | C |
| ATOM | 666 | CB | ALA | B | 12 | −0.865 | 24.883 | 3.649 | 1.00 | 23.68 | 6 | C |
| ATOM | 667 | C | ALA | B | 12 | −2.233 | 24.886 | 5.770 | 1.00 | 22.73 | 6 | C |
| ATOM | 668 | O | ALA | B | 12 | −2.587 | 23.826 | 6.290 | 1.00 | 22.75 | 8 | O |
| ATOM | 669 | N | GLY | B | 13 | −2.983 | 25.998 | 5.943 | 1.00 | 23.23 | 7 | N |
| ATOM | 670 | CA | GLY | B | 13 | −4.307 | 25.962 | 6.557 | 1.00 | 20.92 | 6 | C |
| ATOM | 671 | C | GLY | B | 13 | −4.310 | 25.750 | 8.059 | 1.00 | 22.76 | 6 | C |
| ATOM | 672 | O | GLY | B | 13 | −5.358 | 25.433 | 8.643 | 1.00 | 23.11 | 8 | O |
| ATOM | 673 | N | LYS | B | 14 | −3.158 | 25.825 | 8.750 | 1.00 | 20.91 | 7 | N |
| ATOM | 674 | CA | LYS | B | 14 | −3.162 | 25.571 | 10.173 | 1.00 | 20.15 | 6 | C |
| ATOM | 675 | CB | LYS | B | 14 | −2.198 | 24.425 | 10.520 | 1.00 | 21.56 | 6 | C |
| ATOM | 676 | CG | LYS | B | 14 | −2.514 | 23.101 | 9.799 | 1.00 | 23.27 | 6 | C |
| ATOM | 677 | CD | LYS | B | 14 | −3.830 | 22.532 | 10.369 | 1.00 | 25.36 | 6 | C |
| ATOM | 678 | CE | LYS | B | 14 | −4.059 | 21.130 | 9.715 | 1.00 | 27.62 | 6 | C |
| ATOM | 679 | NZ | LYS | B | 14 | −5.327 | 20.576 | 10.272 | 1.00 | 29.04 | 7 | N |
| ATOM | 680 | C | LYS | B | 14 | −2.790 | 26.865 | 10.903 | 1.00 | 18.58 | 6 | C |
| ATOM | 681 | O | LYS | B | 14 | −2.848 | 26.865 | 12.120 | 1.00 | 18.56 | 8 | O |
| ATOM | 682 | N | GLU | B | 15 | −2.503 | 27.914 | 10.171 | 1.00 | 19.78 | 7 | N |
| ATOM | 683 | CA | GLU | B | 15 | −1.978 | 29.156 | 10.731 | 1.00 | 21.26 | 6 | C |
| ATOM | 684 | CB | GLU | B | 15 | −1.998 | 30.269 | 9.681 | 1.00 | 22.71 | 6 | C |
| ATOM | 685 | CG | GLU | B | 15 | −0.900 | 30.330 | 8.665 | 1.00 | 25.31 | 6 | C |
| ATOM | 686 | CD | GLU | B | 15 | −1.205 | 29.466 | 7.441 | 1.00 | 28.76 | 6 | C |
| ATOM | 687 | OE1 | GLU | B | 15 | −2.177 | 28.683 | 7.478 | 1.00 | 29.58 | 8 | O |
| ATOM | 688 | OE2 | GLU | B | 15 | −0.430 | 29.519 | 6.444 | 1.00 | 29.72 | 8 | O |
| ATOM | 689 | C | GLU | B | 15 | −2.786 | 29.696 | 11.894 | 1.00 | 21.72 | 6 | C |
| ATOM | 690 | O | GLU | B | 15 | −2.302 | 29.933 | 13.001 | 1.00 | 21.00 | 8 | O |
| ATOM | 691 | N | ARG | B | 16 | −4.093 | 29.876 | 11.651 | 1.00 | 22.71 | 7 | N |
| ATOM | 692 | CA | ARG | B | 16 | −5.022 | 30.366 | 12.661 | 1.00 | 22.83 | 6 | C |
| ATOM | 693 | CB | ARG | B | 16 | −6.422 | 30.552 | 12.073 | 1.00 | 26.64 | 6 | C |
| ATOM | 694 | CG | ARG | B | 16 | −6.635 | 31.630 | 11.048 | 1.00 | 30.52 | 6 | C |
| ATOM | 695 | CD | ARG | B | 16 | −6.555 | 33.048 | 11.602 | 1.00 | 35.34 | 6 | C |
| ATOM | 696 | NE | ARG | B | 16 | −6.384 | 33.959 | 10.475 | 1.00 | 39.66 | 7 | N |
| ATOM | 697 | CZ | ARG | B | 16 | −6.162 | 35.258 | 10.382 | 1.00 | 41.31 | 6 | C |
| ATOM | 698 | NH1 | ARG | B | 16 | −6.070 | 36.144 | 11.364 | 1.00 | 42.11 | 7 | N |
| ATOM | 699 | NH2 | ARG | B | 16 | −6.050 | 35.658 | 9.104 | 1.00 | 41.95 | 7 | N |
| ATOM | 700 | C | ARG | B | 16 | −5.125 | 29.436 | 13.851 | 1.00 | 21.36 | 6 | C |
| ATOM | 701 | O | ARG | B | 16 | −5.111 | 29.904 | 15.001 | 1.00 | 21.13 | 8 | O |
| ATOM | 702 | N | GLU | B | 17 | −5.120 | 28.107 | 13.675 | 1.00 | 19.62 | 7 | N |
| ATOM | 703 | CA | GLU | B | 17 | −5.244 | 27.313 | 14.924 | 1.00 | 19.14 | 6 | C |
| ATOM | 704 | CB | GLU | B | 17 | −5.774 | 25.898 | 14.657 | 1.00 | 22.22 | 6 | C |
| ATOM | 705 | CG | GLU | B | 17 | −4.832 | 25.235 | 13.662 | 1.00 | 26.79 | 6 | C |
| ATOM | 706 | CD | GLU | B | 17 | −4.940 | 23.734 | 13.633 | 1.00 | 31.06 | 6 | C |
| ATOM | 707 | OE1 | GLU | B | 17 | −6.094 | 23.198 | 13.654 | 1.00 | 33.97 | 8 | O |
| ATOM | 708 | OE2 | GLU | B | 17 | −3.794 | 23.223 | 13.550 | 1.00 | 32.92 | 8 | O |
| ATOM | 709 | C | GLU | B | 17 | −3.922 | 27.312 | 15.672 | 1.00 | 16.82 | 6 | C |
| ATOM | 710 | O | GLU | B | 17 | −3.902 | 27.254 | 16.914 | 1.00 | 16.12 | 8 | O |
| ATOM | 711 | N | VAL | B | 18 | −2.801 | 27.360 | 14.949 | 1.00 | 16.92 | 7 | N |
| ATOM | 712 | CA | VAL | B | 18 | −1.508 | 27.472 | 15.634 | 1.00 | 16.56 | 6 | C |
| ATOM | 713 | CB | VAL | B | 18 | −0.319 | 27.395 | 14.663 | 1.00 | 16.85 | 6 | C |
| ATOM | 714 | CG1 | VAL | B | 18 | 1.050 | 27.672 | 15.311 | 1.00 | 16.29 | 6 | C |
| ATOM | 715 | CG2 | VAL | B | 18 | −0.228 | 25.929 | 14.201 | 1.00 | 16.98 | 6 | C |
| ATOM | 716 | C | VAL | B | 18 | −1.548 | 28.791 | 16.416 | 1.00 | 14.71 | 6 | C |
| ATOM | 717 | O | VAL | B | 18 | −1.156 | 28.849 | 17.560 | 1.00 | 13.81 | 8 | O |
| ATOM | 718 | N | MET | B | 19 | −1.977 | 29.869 | 15.764 | 1.00 | 15.18 | 7 | N |
| ATOM | 719 | CA | MET | B | 19 | −2.023 | 31.157 | 16.469 | 1.00 | 16.64 | 6 | C |
| ATOM | 720 | CB | MET | B | 19 | −2.587 | 32.281 | 15.598 | 1.00 | 17.04 | 6 | C |
| ATOM | 721 | CG | MET | B | 19 | −2.598 | 33.599 | 16.417 | 1.00 | 20.44 | 6 | C |
| ATOM | 722 | SD | MET | B | 19 | −2.923 | 34.981 | 15.256 | 1.00 | 23.31 | 16 | S |
| ATOM | 723 | CE | MET | B | 19 | −1.290 | 34.646 | 14.586 | 1.00 | 24.64 | 6 | C |
| ATOM | 724 | C | MET | B | 19 | −2.802 | 31.103 | 17.757 | 1.00 | 16.32 | 6 | C |
| ATOM | 725 | O | MET | B | 19 | −2.336 | 31.571 | 18.803 | 1.00 | 13.62 | 8 | O |
| ATOM | 726 | N | GLU | B | 20 | −3.983 | 30.445 | 17.736 | 1.00 | 18.19 | 7 | N |
| ATOM | 727 | CA | GLU | B | 20 | −4.773 | 30.394 | 18.973 | 1.00 | 17.75 | 6 | C |
| ATOM | 728 | CB | GLU | B | 20 | −6.220 | 29.959 | 18.669 | 1.00 | 20.39 | 6 | C |
| ATOM | 729 | CG | GLU | B | 20 | −6.902 | 30.808 | 17.607 | 1.00 | 22.11 | 6 | C |
| ATOM | 730 | CD | GLU | B | 20 | −6.977 | 32.322 | 17.735 | 1.00 | 25.01 | 6 | C |
| ATOM | 731 | OE1 | GLU | B | 20 | −7.101 | 32.971 | 18.786 | 1.00 | 22.59 | 8 | O |
| ATOM | 732 | OE2 | GLU | B | 20 | −6.869 | 33.012 | 16.673 | 1.00 | 27.61 | 8 | O |
| ATOM | 733 | C | GLU | B | 20 | −4.070 | 29.571 | 20.027 | 1.00 | 17.39 | 6 | C |
| ATOM | 734 | O | GLU | B | 20 | −4.083 | 30.001 | 21.202 | 1.00 | 15.65 | 8 | O |
| ATOM | 735 | N | LYS | B | 21 | −3.400 | 28.447 | 19.654 | 1.00 | 15.60 | 7 | N |
| ATOM | 736 | CA | LYS | B | 21 | −2.647 | 27.767 | 20.701 | 1.00 | 14.17 | 6 | C |
| ATOM | 737 | CB | LYS | B | 21 | −1.968 | 26.465 | 20.239 | 1.00 | 17.22 | 6 | C |
| ATOM | 738 | CG | LYS | B | 21 | −3.009 | 25.422 | 19.806 | 1.00 | 20.24 | 6 | C |
| ATOM | 739 | CD | LYS | B | 21 | −2.479 | 24.019 | 19.721 | 1.00 | 20.81 | 6 | C |
| ATOM | 740 | CE | LYS | B | 21 | −3.531 | 23.001 | 19.287 | 1.00 | 23.49 | 6 | C |
| ATOM | 741 | NZ | LYS | B | 21 | −3.546 | 22.690 | 17.821 | 1.00 | 24.31 | 7 | N |
| ATOM | 742 | C | LYS | B | 21 | −1.559 | 28.704 | 21.255 | 1.00 | 12.69 | 6 | C |
| ATOM | 743 | O | LYS | B | 21 | −1.277 | 28.673 | 22.450 | 1.00 | 9.62 | 8 | O |
| ATOM | 744 | N | LEU | B | 22 | −0.810 | 29.387 | 20.376 | 1.00 | 12.07 | 7 | N |
| ATOM | 745 | CA | LEU | B | 22 | 0.286 | 30.235 | 20.919 | 1.00 | 10.01 | 6 | C |
| ATOM | 746 | CB | LEU | B | 22 | 0.994 | 30.891 | 19.717 | 1.00 | 10.07 | 6 | C |
| ATOM | 747 | CG | LEU | B | 22 | 1.765 | 29.929 | 18.772 | 1.00 | 11.87 | 6 | C |
| ATOM | 748 | CD1 | LEU | B | 22 | 2.156 | 30.691 | 17.484 | 1.00 | 10.37 | 6 | C |
| ATOM | 749 | CD2 | LEU | B | 22 | 2.966 | 29.369 | 19.452 | 1.00 | 9.92 | 6 | C |
| ATOM | 750 | C | LEU | B | 22 | −0.229 | 31.325 | 21.840 | 1.00 | 10.76 | 6 | C |
| ATOM | 751 | O | LEU | B | 22 | 0.328 | 31.647 | 22.890 | 1.00 | 11.14 | 8 | O |
| ATOM | 752 | N | LEU | B | 23 | −1.358 | 31.960 | 21.525 | 1.00 | 9.40 | 7 | N |
| ATOM | 753 | CA | LEU | B | 23 | −1.921 | 33.024 | 22.345 | 1.00 | 11.73 | 6 | C |
| ATOM | 754 | CB | LEU | B | 23 | −3.079 | 33.727 | 21.621 | 1.00 | 11.69 | 6 | C |
| ATOM | 755 | CG | LEU | B | 23 | −2.745 | 34.512 | 20.356 | 1.00 | 11.81 | 6 | C |
| ATOM | 756 | CD1 | LEU | B | 23 | −4.016 | 34.922 | 19.616 | 1.00 | 11.79 | 6 | C |
| ATOM | 757 | CD2 | LEU | B | 23 | −1.938 | 35.790 | 20.695 | 1.00 | 12.26 | 6 | C |
| ATOM | 758 | C | LEU | B | 23 | −2.357 | 32.553 | 23.727 | 1.00 | 13.43 | 6 | C |
| ATOM | 759 | O | LEU | B | 23 | −2.425 | 33.433 | 24.579 | 1.00 | 13.81 | 8 | O |
| ATOM | 760 | N | ALA | B | 24 | −2.580 | 31.252 | 23.978 | 1.00 | 14.24 | 7 | N |
| ATOM | 761 | CA | ALA | B | 24 | −2.824 | 30.782 | 25.339 | 1.00 | 15.64 | 6 | C |
| ATOM | 762 | CB | ALA | B | 24 | −3.502 | 29.408 | 25.345 | 1.00 | 15.75 | 6 | C |
| ATOM | 763 | C | ALA | B | 24 | −1.514 | 30.588 | 26.104 | 1.00 | 16.64 | 6 | C |
| ATOM | 764 | O | ALA | B | 24 | −1.600 | 30.289 | 27.290 | 1.00 | 17.40 | 8 | O |
| ATOM | 765 | N | MET | B | 25 | −0.349 | 30.681 | 25.462 | 1.00 | 14.70 | 7 | N |
| ATOM | 766 | CA | MET | B | 25 | 0.901 | 30.516 | 26.249 | 1.00 | 15.45 | 6 | C |
| ATOM | 767 | CB | MET | B | 25 | 2.001 | 29.980 | 25.331 | 1.00 | 14.21 | 6 | C |
| ATOM | 768 | CG | MET | B | 25 | 1.578 | 28.734 | 24.564 | 1.00 | 15.72 | 6 | C |
| ATOM | 769 | SD | MET | B | 25 | 2.874 | 28.259 | 23.361 | 1.00 | 15.01 | 16 | S |
| ATOM | 770 | CE | MET | B | 25 | 2.028 | 26.880 | 22.566 | 1.00 | 14.16 | 6 | C |
| ATOM | 771 | C | MET | B | 25 | 1.267 | 31.832 | 26.875 | 1.00 | 13.99 | 6 | C |
| ATOM | 772 | O | MET | B | 25 | 1.300 | 32.872 | 26.197 | 1.00 | 13.82 | 8 | O |
| ATOM | 773 | N | PRO | B | 26 | 1.573 | 31.882 | 28.180 | 1.00 | 15.20 | 7 | N |
| ATOM | 774 | CD | PRO | B | 26 | 1.680 | 30.670 | 29.044 | 1.00 | 15.96 | 6 | C |
| ATOM | 775 | CA | PRO | B | 26 | 1.948 | 33.094 | 28.857 | 1.00 | 14.72 | 6 | C |
| ATOM | 776 | CB | PRO | B | 26 | 2.160 | 32.681 | 30.320 | 1.00 | 15.60 | 6 | C |
| ATOM | 777 | CG | PRO | B | 26 | 2.374 | 31.223 | 30.279 | 1.00 | 15.14 | 6 | C |
| ATOM | 778 | C | PRO | B | 26 | 3.255 | 33.744 | 28.338 | 1.00 | 14.07 | 6 | C |
| ATOM | 779 | O | PRO | B | 26 | 3.350 | 34.960 | 28.438 | 1.00 | 13.92 | 8 | O |
| ATOM | 780 | N | GLU | B | 27 | 4.170 | 33.002 | 27.747 | 1.00 | 11.08 | 7 | N |
| ATOM | 781 | CA | GLU | B | 27 | 5.391 | 33.603 | 27.183 | 1.00 | 13.20 | 6 | C |
| ATOM | 782 | CB | GLU | B | 27 | 6.371 | 32.491 | 26.747 | 1.00 | 13.28 | 6 | C |
| ATOM | 783 | CG | GLU | B | 27 | 6.822 | 31.666 | 27.944 | 1.00 | 16.85 | 6 | C |
| ATOM | 784 | CD | GLU | B | 27 | 6.036 | 30.398 | 28.231 | 1.00 | 18.72 | 6 | C |
| ATOM | 785 | OE1 | GLU | B | 27 | 4.890 | 30.149 | 27.819 | 1.00 | 18.84 | 8 | O |
| ATOM | 786 | OE2 | GLU | B | 27 | 6.595 | 29.540 | 28.940 | 1.00 | 19.28 | 8 | O |
| ATOM | 787 | C | GLU | B | 27 | 5.057 | 34.427 | 25.939 | 1.00 | 11.33 | 6 | C |
| ATOM | 788 | O | GLU | B | 27 | 5.821 | 35.259 | 25.512 | 1.00 | 11.54 | 8 | O |
| ATOM | 789 | N | VAL | B | 28 | 3.990 | 34.102 | 25.231 | 1.00 | 10.97 | 7 | N |
| ATOM | 790 | CA | VAL | B | 28 | 3.634 | 34.767 | 23.972 | 1.00 | 10.37 | 6 | C |
| ATOM | 791 | CB | VAL | B | 28 | 2.695 | 33.897 | 23.121 | 1.00 | 10.11 | 6 | C |
| ATOM | 792 | CG1 | VAL | B | 28 | 2.226 | 34.564 | 21.814 | 1.00 | 7.99 | 6 | C |
| ATOM | 793 | CG2 | VAL | B | 28 | 3.397 | 32.551 | 22.912 | 1.00 | 7.50 | 6 | C |
| ATOM | 794 | C | VAL | B | 28 | 2.948 | 36.083 | 24.254 | 1.00 | 11.91 | 6 | C |
| ATOM | 795 | O | VAL | B | 28 | 1.843 | 36.130 | 24.822 | 1.00 | 13.08 | 8 | O |
| ATOM | 796 | N | LYS | B | 29 | 3.565 | 37.163 | 23.832 | 1.00 | 10.47 | 7 | N |
| ATOM | 797 | CA | LYS | B | 29 | 3.011 | 38.482 | 24.072 | 1.00 | 10.88 | 6 | C |
| ATOM | 798 | CB | LYS | B | 29 | 4.110 | 39.487 | 24.379 | 1.00 | 11.16 | 5 | C |
| ATOM | 799 | CG | LYS | B | 29 | 5.048 | 38.925 | 25.483 | 1.00 | 12.65 | 6 | C |
| ATOM | 800 | CD | LYS | B | 29 | 4.298 | 38.989 | 26.830 | 1.00 | 16.71 | 6 | C |
| ATOM | 801 | CE | LYS | B | 29 | 4.738 | 37.821 | 27.706 | 1.00 | 19.48 | 6 | C |
| ATOM | 802 | NZ | LYS | B | 29 | 4.388 | 38.062 | 29.136 | 1.00 | 23.26 | 7 | N |
| ATOM | 803 | C | LYS | B | 29 | 2.264 | 38.986 | 22.859 | 1.00 | 11.85 | 6 | C |
| ATOM | 804 | O | LYS | B | 29 | 1.369 | 39.856 | 23.035 | 1.00 | 11.08 | 8 | O |
| ATOM | 805 | N | GLU | B | 30 | 2.695 | 38.590 | 21.651 | 1.00 | 9.93 | 7 | N |
| ATOM | 806 | CA | GLU | B | 30 | 1.998 | 39.086 | 20.459 | 1.00 | 9.60 | 6 | C |
| ATOM | 807 | CB | GLU | B | 30 | 2.546 | 40.384 | 19.899 | 1.00 | 13.47 | 6 | C |
| ATOM | 808 | CG | GLU | B | 30 | 2.577 | 41.628 | 20.771 | 1.00 | 11.99 | 6 | C |
| ATOM | 809 | CD | GLU | B | 30 | 3.193 | 42.838 | 20.116 | 1.00 | 13.20 | 6 | C |
| ATOM | 810 | OE1 | GLU | B | 30 | 3.579 | 43.769 | 20.856 | 1.00 | 11.60 | 8 | O |
| ATOM | 811 | OE2 | GLU | B | 30 | 3.107 | 42.989 | 18.886 | 1.00 | 13.89 | 8 | O |
| ATOM | 812 | C | GLU | B | 30 | 2.104 | 37.967 | 19.437 | 1.00 | 11.24 | 6 | C |
| ATOM | 813 | O | GLU | B | 30 | 3.137 | 37.255 | 19.467 | 1.00 | 9.55 | 8 | O |
| ATOM | 814 | N | ALA | B | 31 | 1.076 | 37.818 | 18.606 | 1.00 | 9.40 | 7 | N |
| ATOM | 815 | CA | ALA | B | 31 | 1.198 | 36.803 | 17.543 | 1.00 | 8.70 | 6 | C |
| ATOM | 816 | CB | ALA | B | 31 | 0.698 | 35.429 | 17.973 | 1.00 | 7.99 | 6 | C |
| ATOM | 817 | C | ALA | B | 31 | 0.357 | 37.314 | 16.390 | 1.00 | 9.99 | 6 | C |
| ATOM | 818 | O | ALA | B | 31 | −0.761 | 37.841 | 16.609 | 1.00 | 10.67 | 8 | O |
| ATOM | 819 | N | TYR | B | 32 | 0.854 | 37.192 | 15.160 | 1.00 | 7.33 | 7 | N |
| ATOM | 820 | CA | TYR | B | 32 | 0.119 | 37.639 | 14.013 | 1.00 | 7.98 | 6 | C |
| ATOM | 821 | CB | TYR | B | 32 | 0.604 | 38.961 | 13.443 | 1.00 | 6.68 | 6 | C |
| ATOM | 822 | CG | TYR | B | 32 | 0.423 | 40.157 | 14.312 | 1.00 | 8.26 | 6 | C |
| ATOM | 823 | CD1 | TYR | B | 32 | −0.728 | 40.934 | 14.064 | 1.00 | 8.39 | 6 | C |
| ATOM | 824 | CE1 | TYR | B | 32 | −0.914 | 42.086 | 14.840 | 1.00 | 9.14 | 6 | C |
| ATOM | 825 | CD2 | TYR | B | 32 | 1.304 | 40.489 | 15.344 | 1.00 | 6.70 | 6 | C |
| ATOM | 826 | CE2 | TYR | B | 32 | 1.089 | 41.623 | 16.105 | 1.00 | 7.28 | 6 | C |
| ATOM | 827 | CZ | TYR | B | 32 | −0.020 | 42.387 | 15.849 | 1.00 | 8.06 | 6 | C |
| ATOM | 828 | OH | TYR | B | 32 | −0.272 | 43.545 | 16.575 | 1.00 | 11.13 | 8 | O |
| ATOM | 829 | C | TYR | B | 32 | 0.359 | 36.711 | 12.791 | 1.00 | 9.26 | 6 | C |
| ATOM | 830 | O | TYR | B | 32 | 1.455 | 36.211 | 12.595 | 1.00 | 7.38 | 8 | O |
| ATOM | 831 | N | VAL | B | 33 | −0.774 | 36.442 | 12.119 | 1.00 | 7.76 | 7 | N |
| ATOM | 832 | CA | VAL | B | 33 | −0.738 | 35.690 | 10.887 | 1.00 | 8.25 | 6 | C |
| ATOM | 833 | CB | VAL | B | 33 | −2.103 | 35.103 | 10.485 | 1.00 | 9.77 | 6 | C |
| ATOM | 834 | CG1 | VAL | B | 33 | −1.928 | 34.416 | 9.141 | 1.00 | 9.20 | 6 | C |
| ATOM | 835 | CG2 | VAL | B | 33 | −2.588 | 34.125 | 11.538 | 1.00 | 10.44 | 6 | C |
| ATOM | 836 | C | VAL | B | 33 | −0.313 | 36.695 | 9.830 | 1.00 | 6.92 | 6 | C |
| ATOM | 837 | O | VAL | B | 33 | −0.847 | 37.804 | 9.767 | 1.00 | 8.47 | 8 | O |
| ATOM | 838 | N | VAL | B | 34 | 0.698 | 36.343 | 9.020 | 1.00 | 8.02 | 7 | N |
| ATOM | 839 | CA | VAL | B | 34 | 1.176 | 37.342 | 8.101 | 1.00 | 8.86 | 6 | C |
| ATOM | 840 | CB | VAL | B | 34 | 2.565 | 37.938 | 8.440 | 1.00 | 8.81 | 6 | C |
| ATOM | 841 | CG1 | VAL | B | 34 | 2.493 | 38.825 | 9.666 | 1.00 | 8.55 | 6 | C |
| ATOM | 842 | CG2 | VAL | B | 34 | 3.567 | 36.787 | 8.612 | 1.00 | 8.06 | 6 | C |
| ATOM | 843 | C | VAL | B | 34 | 1.265 | 36.792 | 6.688 | 1.00 | 11.20 | 6 | C |
| ATOM | 844 | O | VAL | B | 34 | 1.427 | 35.613 | 6.470 | 1.00 | 13.52 | 8 | O |
| ATOM | 845 | N | TYR | B | 35 | 1.323 | 37.793 | 5.807 | 1.00 | 13.07 | 7 | N |
| ATOM | 846 | CA | TYR | B | 35 | 1.667 | 37.464 | 4.421 | 1.00 | 15.59 | 6 | C |
| ATOM | 847 | CB | TYR | B | 35 | 0.814 | 38.417 | 3.594 | 1.00 | 17.52 | 6 | C |
| ATOM | 848 | CG | TYR | B | 35 | 1.217 | 38.209 | 2.156 | 1.00 | 21.88 | 6 | C |
| ATOM | 849 | CD1 | TYR | B | 35 | 0.716 | 37.086 | 1.532 | 1.00 | 25.26 | 6 | C |
| ATOM | 850 | CE1 | TYR | B | 35 | 1.050 | 36.814 | 0.212 | 1.00 | 28.20 | 6 | C |
| ATOM | 851 | CD2 | TYR | B | 35 | 2.102 | 39.034 | 1.521 | 1.00 | 24.78 | 6 | C |
| ATOM | 852 | CE2 | TYR | B | 35 | 2.530 | 38.731 | 0.240 | 1.00 | 26.74 | 6 | C |
| ATOM | 853 | CZ | TYR | B | 35 | 1.938 | 37.685 | −0.398 | 1.00 | 28.56 | 6 | C |
| ATOM | 854 | OH | TYR | B | 35 | 2.277 | 37.427 | −1.709 | 1.00 | 33.78 | 8 | O |
| ATOM | 855 | C | TYR | B | 35 | 3.152 | 37.824 | 4.207 | 1.00 | 16.52 | 6 | C |
| ATOM | 856 | O | TYR | B | 35 | 3.517 | 38.932 | 4.657 | 1.00 | 18.14 | 8 | O |
| ATOM | 857 | N | GLY | B | 36 | 3.904 | 37.025 | 3.511 | 1.00 | 15.07 | 7 | N |
| ATOM | 858 | CA | GLY | B | 36 | 5.297 | 37.441 | 3.214 | 1.00 | 18.36 | 6 | C |
| ATOM | 859 | C | GLY | B | 36 | 6.143 | 36.151 | 3.231 | 1.00 | 19.05 | 6 | C |
| ATOM | 860 | O | GLY | B | 36 | 5.557 | 35.061 | 3.058 | 1.00 | 16.65 | 8 | O |
| ATOM | 861 | N | GLU | B | 37 | 7.434 | 36.257 | 3.609 | 1.00 | 21.01 | 7 | N |
| ATOM | 862 | CA | GLU | B | 37 | 8.152 | 34.953 | 3.595 | 1.00 | 23.51 | 6 | C |
| ATOM | 863 | CB | GLU | B | 37 | 9.663 | 35.177 | 3.504 | 1.00 | 28.70 | 6 | C |
| ATOM | 864 | CG | GLU | B | 37 | 10.269 | 35.983 | 4.630 | 1.00 | 32.35 | 6 | C |
| ATOM | 865 | CD | GLU | B | 37 | 11.798 | 35.903 | 4.612 | 1.00 | 36.24 | 6 | C |
| ATOM | 866 | OE1 | GLU | B | 37 | 12.437 | 36.685 | 5.372 | 1.00 | 35.75 | 8 | O |
| ATOM | 867 | OE2 | GLU | B | 37 | 12.317 | 35.044 | 3.847 | 1.00 | 37.65 | 8 | O |
| ATOM | 868 | C | GLU | B | 37 | 7.845 | 33.981 | 4.714 | 1.00 | 23.07 | 6 | C |
| ATOM | 869 | O | GLU | B | 37 | 8.259 | 32.834 | 4.540 | 1.00 | 23.02 | 8 | O |
| ATOM | 870 | N | TYR | B | 38 | 7.232 | 34.337 | 5.840 | 1.00 | 21.40 | 7 | N |
| ATOM | 871 | CA | TYR | B | 38 | 6.879 | 33.361 | 6.889 | 1.00 | 19.67 | 6 | C |
| ATOM | 872 | CB | TYR | B | 38 | 7.610 | 33.823 | 8.162 | 1.00 | 20.72 | 6 | C |
| ATOM | 873 | CG | TYR | B | 38 | 9.064 | 33.414 | 8.146 | 1.00 | 23.64 | 6 | C |
| ATOM | 874 | CD1 | TYR | B | 38 | 9.401 | 32.089 | 8.412 | 1.00 | 25.30 | 6 | C |
| ATOM | 875 | CE1 | TYR | B | 38 | 10.714 | 31.666 | 8.416 | 1.00 | 26.52 | 6 | C |
| ATOM | 876 | CD2 | TYR | B | 38 | 10.090 | 34.313 | 7.871 | 1.00 | 25.13 | 6 | C |
| ATOM | 877 | CE2 | TYR | B | 38 | 11.410 | 33.895 | 7.884 | 1.00 | 25.61 | 6 | C |
| ATOM | 878 | CZ | TYR | B | 38 | 11.710 | 32.591 | 8.145 | 1.00 | 27.23 | 6 | C |
| ATOM | 879 | OH | TYR | B | 38 | 13.035 | 32.149 | 8.152 | 1.00 | 29.72 | 8 | O |
| ATOM | 880 | C | TYR | B | 38 | 5.376 | 33.329 | 7.149 | 1.00 | 17.30 | 6 | C |
| ATOM | 881 | O | TYR | B | 38 | 4.744 | 34.238 | 6.603 | 1.00 | 16.96 | 8 | O |
| ATOM | 882 | N | ASP | B | 39 | 4.742 | 32.399 | 7.856 | 1.00 | 16.10 | 7 | N |
| ATOM | 883 | CA | ASP | B | 39 | 3.318 | 32.359 | 8.062 | 1.00 | 15.22 | 6 | C |
| ATOM | 884 | CB | ASP | B | 39 | 2.879 | 30.893 | 8.284 | 1.00 | 17.21 | 6 | C |
| ATOM | 885 | CG | ASP | B | 39 | 2.939 | 30.170 | 6.935 | 1.00 | 19.01 | 6 | C |
| ATOM | 886 | OD1 | ASP | B | 39 | 2.735 | 30.787 | 5.869 | 1.00 | 19.17 | 8 | O |
| ATOM | 887 | OD2 | ASP | B | 39 | 3.263 | 28.976 | 7.028 | 1.00 | 20.31 | 8 | O |
| ATOM | 888 | C | ASP | B | 39 | 2.815 | 33.108 | 9.297 | 1.00 | 15.48 | 6 | C |
| ATOM | 889 | O | ASP | B | 39 | 1.726 | 33.694 | 9.290 | 1.00 | 13.65 | 8 | O |
| ATOM | 890 | N | LEU | B | 40 | 3.590 | 33.064 | 10.383 | 1.00 | 13.35 | 7 | N |
| ATOM | 891 | CA | LEU | B | 40 | 3.266 | 33.717 | 11.639 | 1.00 | 12.10 | 6 | C |
| ATOM | 892 | CB | LEU | B | 40 | 2.872 | 32.708 | 12.724 | 1.00 | 13.67 | 6 | C |
| ATOM | 893 | CG | LEU | B | 40 | 1.601 | 31.893 | 12.552 | 1.00 | 17.69 | 6 | C |
| ATOM | 894 | CD1 | LEU | B | 40 | 1.329 | 30.939 | 13.715 | 1.00 | 20.32 | 6 | C |
| ATOM | 895 | CD2 | LEU | B | 40 | 0.394 | 32.825 | 12.479 | 1.00 | 20.29 | 6 | C |
| ATOM | 896 | C | LEU | B | 40 | 4.471 | 34.470 | 12.223 | 1.00 | 11.17 | 6 | C |
| ATOM | 897 | O | LEU | B | 40 | 5.646 | 34.118 | 11.961 | 1.00 | 10.75 | 8 | O |
| ATOM | 898 | N | ILE | B | 41 | 4.237 | 35.543 | 12.943 | 1.00 | 8.24 | 7 | N |
| ATOM | 899 | CA | ILE | B | 41 | 5.339 | 36.230 | 13.626 | 1.00 | 9.66 | 6 | C |
| ATOM | 900 | CB | ILE | B | 41 | 5.712 | 37.587 | 13.108 | 1.00 | 12.11 | 6 | C |
| ATOM | 901 | CG2 | ILE | B | 41 | 4.557 | 38.563 | 13.016 | 1.00 | 15.03 | 6 | C |
| ATOM | 902 | CG1 | ILE | B | 41 | 6.735 | 38.248 | 14.096 | 1.00 | 13.84 | 6 | C |
| ATOM | 903 | CD1 | ILE | B | 41 | 7.691 | 39.105 | 13.319 | 1.00 | 15.84 | 6 | C |
| ATOM | 904 | C | ILE | B | 41 | 4.851 | 36.269 | 15.074 | 1.00 | 10.80 | 6 | C |
| ATOM | 905 | O | ILE | B | 41 | 3.678 | 36.570 | 15.329 | 1.00 | 9.87 | 8 | O |
| ATOM | 906 | N | VAL | B | 42 | 5.666 | 35.853 | 16.019 | 1.00 | 9.90 | 7 | N |
| ATOM | 907 | CA | VAL | B | 42 | 5.269 | 35.779 | 17.415 | 1.00 | 8.61 | 6 | C |
| ATOM | 908 | CB | VAL | B | 42 | 5.231 | 34.269 | 17.758 | 1.00 | 11.63 | 6 | C |
| ATOM | 909 | CG1 | VAL | B | 42 | 4.728 | 33.994 | 19.179 | 1.00 | 11.77 | 6 | C |
| ATOM | 910 | CG2 | VAL | B | 42 | 4.389 | 33.446 | 16.776 | 1.00 | 11.85 | 6 | C |
| ATOM | 911 | C | VAL | B | 42 | 6.265 | 36.480 | 18.307 | 1.00 | 9.85 | 6 | C |
| ATOM | 912 | O | VAL | B | 42 | 7.476 | 36.173 | 18.143 | 1.00 | 9.17 | 8 | O |
| ATOM | 913 | N | LYS | B | 43 | 5.811 | 37.419 | 19.136 | 1.00 | 7.78 | 7 | N |
| ATOM | 914 | CA | LYS | B | 43 | 6.740 | 38.088 | 20.055 | 1.00 | 7.65 | 6 | C |
| ATOM | 915 | CB | LYS | B | 43 | 6.282 | 39.519 | 20.345 | 1.00 | 9.86 | 6 | C |
| ATOM | 916 | CG | LYS | B | 43 | 7.306 | 40.251 | 21.245 | 1.00 | 10.71 | 6 | C |
| ATOM | 917 | CD | LYS | B | 43 | 6.614 | 41.604 | 21.531 | 1.00 | 9.74 | 6 | C |
| ATOM | 918 | CE | LYS | B | 43 | 7.585 | 42.476 | 22.315 | 1.00 | 10.47 | 6 | C |
| ATOM | 919 | NZ | LYS | B | 43 | 6.920 | 43.783 | 22.618 | 1.00 | 6.81 | 7 | N |
| ATOM | 920 | C | LYS | B | 43 | 6.694 | 37.349 | 21.396 | 1.00 | 9.77 | 6 | C |
| ATOM | 921 | O | LYS | B | 43 | 5.585 | 37.200 | 21.979 | 1.00 | 8.09 | 8 | O |
| ATOM | 922 | N | VAL | B | 44 | 7.840 | 36.891 | 21.897 | 1.00 | 8.63 | 7 | N |
| ATOM | 923 | CA | VAL | B | 44 | 7.818 | 36.118 | 23.143 | 1.00 | 8.80 | 6 | C |
| ATOM | 924 | CB | VAL | B | 44 | 8.104 | 34.612 | 22.973 | 1.00 | 10.38 | 6 | C |
| ATOM | 925 | CG1 | VAL | B | 44 | 7.208 | 33.936 | 21.922 | 1.00 | 8.21 | 6 | C |
| ATOM | 926 | CG2 | VAL | B | 44 | 9.563 | 34.281 | 22.597 | 1.00 | 9.95 | 6 | C |
| ATOM | 927 | C | VAL | B | 44 | 8.782 | 36.742 | 24.138 | 1.00 | 9.71 | 6 | C |
| ATOM | 928 | O | VAL | B | 44 | 9.699 | 37.510 | 23.750 | 1.00 | 5.89 | 8 | O |
| ATOM | 929 | N | GLU | B | 45 | 8.479 | 36.528 | 25.418 | 1.00 | 10.09 | 7 | N |
| ATOM | 930 | CA | GLU | B | 45 | 9.296 | 37.043 | 26.518 | 1.00 | 13.68 | 6 | C |
| ATOM | 931 | CB | GLU | B | 45 | 8.689 | 38.315 | 27.137 | 1.00 | 14.51 | 6 | C |
| ATOM | 932 | CG | GLU | B | 45 | 8.798 | 39.552 | 26.269 | 1.00 | 18.31 | 6 | C |
| ATOM | 933 | CD | GLU | B | 45 | 8.037 | 40.752 | 26.792 | 1.00 | 21.50 | 6 | C |
| ATOM | 934 | OE1 | GLU | B | 45 | 7.964 | 41.733 | 26.010 | 1.00 | 22.57 | 8 | O |
| ATOM | 935 | OE2 | GLU | B | 45 | 7.590 | 40.680 | 27.967 | 1.00 | 20.85 | 8 | O |
| ATOM | 936 | C | GLU | B | 45 | 9.424 | 35.985 | 27.622 | 1.00 | 15.23 | 6 | C |
| ATOM | 937 | O | GLU | B | 45 | 8.491 | 35.243 | 27.941 | 1.00 | 12.79 | 8 | O |
| ATOM | 938 | N | THR | B | 46 | 10.617 | 35.776 | 28.144 | 1.00 | 15.49 | 7 | N |
| ATOM | 939 | CA | THR | B | 46 | 10.988 | 34.815 | 29.157 | 1.00 | 15.12 | 6 | C |
| ATOM | 940 | CB | THR | B | 46 | 11.682 | 33.534 | 28.689 | 1.00 | 16.31 | 6 | C |
| ATOM | 941 | OG1 | THR | B | 46 | 12.959 | 33.763 | 28.010 | 1.00 | 15.78 | 8 | O |
| ATOM | 942 | CG2 | THR | B | 46 | 10.833 | 32.730 | 27.693 | 1.00 | 17.84 | 6 | C |
| ATOM | 943 | C | THR | B | 46 | 11.930 | 35.522 | 30.142 | 1.00 | 17.32 | 6 | C |
| ATOM | 944 | O | THR | B | 46 | 12.524 | 36.584 | 29.858 | 1.00 | 16.70 | 8 | O |
| ATOM | 945 | N | ASP | B | 47 | 12.010 | 34.957 | 31.363 | 1.00 | 17.97 | 7 | N |
| ATOM | 946 | CA | ASP | B | 47 | 12.845 | 35.635 | 32.353 | 1.00 | 20.36 | 6 | C |
| ATOM | 947 | CB | ASP | B | 47 | 12.598 | 35.223 | 33.813 | 1.00 | 25.58 | 6 | C |
| ATOM | 948 | CG | ASP | B | 47 | 12.664 | 33.736 | 34.035 | 1.00 | 29.81 | 6 | C |
| ATOM | 949 | OD1 | ASP | B | 47 | 12.448 | 33.010 | 33.027 | 1.00 | 31.62 | 8 | O |
| ATOM | 950 | OD2 | ASP | B | 47 | 12.956 | 33.250 | 35.162 | 1.00 | 32.24 | 8 | O |
| ATOM | 951 | C | ASP | B | 47 | 14.301 | 35.373 | 31.978 | 1.00 | 16.72 | 6 | C |
| ATOM | 952 | O | ASP | B | 47 | 15.112 | 36.307 | 32.039 | 1.00 | 17.65 | 8 | O |
| ATOM | 953 | N | THR | B | 48 | 14.620 | 34.173 | 31.551 | 1.00 | 15.74 | 7 | N |
| ATOM | 954 | CA | THR | B | 48 | 16.043 | 33.987 | 31.213 | 1.00 | 16.40 | 6 | C |
| ATOM | 955 | CB | THR | B | 48 | 16.770 | 33.069 | 32.215 | 1.00 | 15.67 | 6 | C |
| ATOM | 956 | OG1 | THR | B | 48 | 16.288 | 31.739 | 31.991 | 1.00 | 16.49 | 8 | O |
| ATOM | 957 | CG2 | THR | B | 48 | 16.605 | 33.444 | 33.691 | 1.00 | 17.54 | 6 | C |
| ATOM | 958 | C | THR | B | 48 | 16.231 | 33.405 | 29.817 | 1.00 | 15.74 | 6 | C |
| ATOM | 959 | O | THR | B | 48 | 15.286 | 32.952 | 29.210 | 1.00 | 16.51 | 8 | O |
| ATOM | 960 | N | LEU | B | 49 | 17.488 | 33.424 | 29.331 | 1.00 | 14.22 | 7 | N |
| ATOM | 961 | CA | LEU | B | 49 | 17.828 | 32.808 | 28.078 | 1.00 | 12.99 | 6 | C |
| ATOM | 962 | CB | LEU | B | 49 | 19.309 | 33.047 | 27.731 | 1.00 | 13.45 | 6 | C |
| ATOM | 963 | CG | LEU | B | 49 | 19.772 | 32.442 | 26.399 | 1.00 | 12.86 | 6 | C |
| ATOM | 964 | CD1 | LEU | B | 49 | 18.885 | 32.972 | 25.269 | 1.00 | 12.52 | 6 | C |
| ATOM | 965 | CD2 | LEU | B | 49 | 21.248 | 32.799 | 26.176 | 1.00 | 12.69 | 6 | C |
| ATOM | 966 | C | LEU | B | 49 | 17.641 | 31.314 | 28.138 | 1.00 | 14.59 | 6 | C |
| ATOM | 967 | O | LEU | B | 49 | 17.102 | 30.681 | 27.223 | 1.00 | 15.53 | 8 | O |
| ATOM | 968 | N | LYS | B | 50 | 17.970 | 30.682 | 29.262 | 1.00 | 16.17 | 7 | N |
| ATOM | 969 | CA | LYS | B | 50 | 17.720 | 29.246 | 29.381 | 1.00 | 19.48 | 6 | C |
| ATOM | 970 | CB | LYS | B | 50 | 18.193 | 28.727 | 30.758 | 1.00 | 22.06 | 6 | C |
| ATOM | 971 | CG | LYS | B | 50 | 19.650 | 28.768 | 31.070 | 1.00 | 27.90 | 6 | C |
| ATOM | 972 | CD | LYS | B | 50 | 20.542 | 27.872 | 30.226 | 1.00 | 30.91 | 6 | C |
| ATOM | 973 | CE | LYS | B | 50 | 21.605 | 27.205 | 31.107 | 1.00 | 34.16 | 6 | C |
| ATOM | 974 | NZ | LYS | B | 50 | 22.915 | 27.924 | 31.080 | 1.00 | 35.27 | 7 | N |
| ATOM | 975 | C | LYS | B | 50 | 16.214 | 28.924 | 29.306 | 1.00 | 14.90 | 6 | C |
| ATOM | 976 | O | LYS | B | 50 | 15.819 | 27.823 | 28.917 | 1.00 | 14.68 | 8 | O |
| ATOM | 977 | N | ASP | B | 51 | 15.380 | 29.793 | 29.876 | 1.00 | 17.81 | 7 | N |
| ATOM | 978 | CA | ASP | B | 51 | 13.908 | 29.507 | 29.814 | 1.00 | 17.69 | 6 | C |
| ATOM | 979 | CB | ASP | B | 51 | 13.219 | 30.482 | 30.729 | 1.00 | 19.51 | 6 | C |
| ATOM | 980 | CG | ASP | B | 51 | 13.360 | 30.134 | 32.206 | 1.00 | 20.72 | 6 | C |
| ATOM | 981 | OD1 | ASP | B | 51 | 13.708 | 29.003 | 32.574 | 1.00 | 21.31 | 8 | O |
| ATOM | 982 | OD2 | ASP | B | 51 | 13.092 | 31.053 | 32.984 | 1.00 | 20.55 | 8 | O |
| ATOM | 983 | C | ASP | B | 51 | 13.371 | 29.632 | 28.398 | 1.00 | 16.44 | 6 | C |
| ATOM | 984 | O | ASP | B | 51 | 12.715 | 28.703 | 27.890 | 1.00 | 17.42 | 8 | O |
| ATOM | 985 | N | LEU | B | 52 | 13.941 | 30.582 | 27.633 | 1.00 | 15.23 | 7 | N |
| ATOM | 986 | CA | LEU | B | 52 | 13.645 | 30.699 | 26.207 | 1.00 | 13.82 | 6 | C |
| ATOM | 987 | CB | LEU | B | 52 | 14.396 | 31.920 | 25.587 | 1.00 | 14.63 | 6 | C |
| ATOM | 988 | CG | LEU | B | 52 | 14.076 | 32.194 | 24.117 | 1.00 | 15.53 | 6 | C |
| ATOM | 989 | CD1 | LEU | B | 52 | 12.710 | 32.945 | 24.006 | 1.00 | 14.67 | 6 | C |
| ATOM | 990 | CD2 | LEU | B | 52 | 15.158 | 32.974 | 23.399 | 1.00 | 13.85 | 6 | C |
| ATOM | 991 | C | LEU | B | 52 | 14.079 | 29.449 | 25.476 | 1.00 | 14.79 | 6 | C |
| ATOM | 992 | O | LEU | B | 52 | 13.369 | 28.870 | 24.658 | 1.00 | 14.97 | 8 | O |
| ATOM | 993 | N | ASP | B | 53 | 15.306 | 28.967 | 25.785 | 1.00 | 14.38 | 7 | N |
| ATOM | 994 | CA | ASP | B | 53 | 15.821 | 27.759 | 25.148 | 1.00 | 16.61 | 6 | C |
| ATOM | 995 | CB | ASP | B | 53 | 17.158 | 27.294 | 25.758 | 1.00 | 16.33 | 6 | C |
| ATOM | 996 | CG | ASP | B | 53 | 18.329 | 28.180 | 25.461 | 1.00 | 17.82 | 6 | C |
| ATOM | 997 | OD1 | ASP | B | 53 | 18.339 | 29.043 | 24.561 | 1.00 | 16.77 | 8 | O |
| ATOM | 998 | OD2 | ASP | B | 53 | 19.340 | 28.168 | 26.224 | 1.00 | 21.96 | 8 | O |
| ATOM | 999 | C | ASP | B | 53 | 14.800 | 26.626 | 25.231 | 1.00 | 16.88 | 6 | C |
| ATOM | 1000 | O | ASP | B | 53 | 14.575 | 25.898 | 24.270 | 1.00 | 16.79 | 8 | O |
| ATOM | 1001 | N | GLN | B | 54 | 14.351 | 26.352 | 26.437 | 1.00 | 19.65 | 7 | N |
| ATOM | 1002 | CA | GLN | B | 54 | 13.406 | 25.269 | 26.733 | 1.00 | 23.28 | 6 | C |
| ATOM | 1003 | CB | GLN | B | 54 | 13.240 | 25.185 | 28.249 | 1.00 | 26.46 | 6 | C |
| ATOM | 1004 | CG | GLN | B | 54 | 12.272 | 24.200 | 28.879 | 1.00 | 32.94 | 6 | C |
| ATOM | 1005 | CD | GLN | B | 54 | 12.330 | 24.433 | 30.398 | 1.00 | 37.85 | 6 | C |
| ATOM | 1006 | OE1 | GLN | B | 54 | 11.846 | 25.474 | 30.887 | 1.00 | 39.47 | 8 | O |
| ATOM | 1007 | NE2 | GLN | B | 54 | 12.945 | 23.515 | 31.160 | 1.00 | 38.13 | 7 | N |
| ATOM | 1008 | C | GLN | B | 54 | 12.051 | 25.545 | 26.083 | 1.00 | 21.13 | 6 | C |
| ATOM | 1009 | O | GLN | B | 54 | 11.499 | 24.633 | 25.489 | 1.00 | 23.10 | 8 | O |
| ATOM | 1010 | N | PHE | B | 55 | 11.558 | 26.769 | 26.135 | 1.00 | 20.48 | 7 | N |
| ATOM | 1011 | CA | PHE | B | 55 | 10.290 | 27.164 | 25.514 | 1.00 | 17.72 | 6 | C |
| ATOM | 1012 | CB | PHE | B | 55 | 9.979 | 28.639 | 25.834 | 1.00 | 17.24 | 6 | C |
| ATOM | 1013 | CG | PHE | B | 55 | 8.715 | 29.086 | 25.132 | 1.00 | 17.59 | 6 | C |
| ATOM | 1014 | CD1 | PHE | B | 55 | 7.479 | 28.564 | 25.536 | 1.00 | 16.74 | 6 | C |
| ATOM | 1015 | CD2 | PHE | B | 55 | 8.753 | 29.980 | 24.075 | 1.00 | 16.08 | 6 | C |
| ATOM | 1016 | CE1 | PHE | B | 55 | 6.317 | 28.943 | 24.873 | 1.00 | 15.53 | 6 | C |
| ATOM | 1017 | CE2 | PHE | B | 55 | 7.594 | 30.392 | 23.472 | 1.00 | 15.86 | 6 | C |
| ATOM | 1018 | CZ | PHE | B | 55 | 6.369 | 29.852 | 23.853 | 1.00 | 16.04 | 6 | C |
| ATOM | 1019 | C | PHE | B | 55 | 10.271 | 26.871 | 24.028 | 1.00 | 17.99 | 6 | C |
| ATOM | 1020 | O | PHE | B | 55 | 9.434 | 26.131 | 23.471 | 1.00 | 14.83 | 8 | O |
| ATOM | 1021 | N | ILE | B | 56 | 11.365 | 27.316 | 23.361 | 1.00 | 16.17 | 7 | N |
| ATOM | 1022 | CA | ILE | B | 56 | 11.462 | 27.092 | 21.932 | 1.00 | 17.08 | 6 | C |
| ATOM | 1023 | CB | ILE | B | 56 | 12.504 | 28.050 | 21.294 | 1.00 | 17.13 | 6 | C |
| ATOM | 1024 | CG2 | ILE | B | 56 | 12.639 | 27.710 | 19.833 | 1.00 | 17.70 | 6 | C |
| ATOM | 1025 | CG1 | ILE | B | 56 | 11.991 | 29.470 | 21.490 | 1.00 | 18.12 | 6 | C |
| ATOM | 1026 | CD1 | ILE | B | 56 | 12.819 | 30.561 | 20.895 | 1.00 | 22.50 | 6 | C |
| ATOM | 1027 | C | ILE | B | 56 | 11.776 | 25.645 | 21.585 | 1.00 | 17.95 | 6 | C |
| ATOM | 1028 | O | ILE | B | 56 | 11.122 | 25.106 | 20.683 | 1.00 | 17.47 | 8 | O |
| ATOM | 1029 | N | THR | B | 57 | 12.732 | 24.990 | 22.239 | 1.00 | 18.22 | 7 | N |
| ATOM | 1030 | CA | THR | B | 57 | 13.053 | 23.632 | 21.790 | 1.00 | 20.69 | 6 | C |
| ATOM | 1031 | CB | THR | B | 57 | 14.411 | 23.126 | 22.315 | 1.00 | 20.99 | 6 | C |
| ATOM | 1032 | OG1 | THR | B | 57 | 14.470 | 23.336 | 23.722 | 1.00 | 23.13 | 8 | O |
| ATOM | 1033 | CG2 | THR | B | 57 | 15.565 | 23.877 | 21.657 | 1.00 | 21.52 | 6 | C |
| ATOM | 1034 | C | THR | B | 57 | 11.986 | 22.615 | 22.194 | 1.00 | 22.22 | 6 | C |
| ATOM | 1035 | O | THR | B | 57 | 11.712 | 21.730 | 21.385 | 1.00 | 21.32 | 8 | O |
| ATOM | 1036 | N | GLU | B | 58 | 11.456 | 22.829 | 23.392 | 1.00 | 23.89 | 7 | N |
| ATOM | 1037 | CA | GLU | B | 58 | 10.513 | 21.872 | 23.941 | 1.00 | 28.94 | 6 | C |
| ATOM | 1038 | CB | GLU | B | 58 | 10.777 | 21.648 | 25.449 | 1.00 | 30.54 | 6 | C |
| ATOM | 1039 | CG | GLU | B | 58 | 12.155 | 21.058 | 25.706 | 1.00 | 33.21 | 6 | C |
| ATOM | 1040 | CD | GLU | B | 58 | 12.442 | 19.835 | 24.844 | 1.00 | 36.23 | 6 | C |
| ATOM | 1041 | OE1 | GLU | B | 58 | 11.537 | 18.989 | 24.608 | 1.00 | 36.52 | 8 | O |
| ATOM | 1042 | OE2 | GLU | B | 58 | 13.596 | 19.742 | 24.361 | 1.00 | 36.20 | 8 | O |
| ATOM | 1043 | C | GLU | B | 58 | 9.052 | 22.194 | 23.689 | 1.00 | 27.95 | 6 | C |
| ATOM | 1044 | O | GLU | B | 58 | 8.347 | 21.187 | 23.496 | 1.00 | 30.27 | 8 | O |
| ATOM | 1045 | N | LYS | B | 59 | 8.594 | 23.432 | 23.616 | 1.00 | 20.94 | 7 | N |
| ATOM | 1046 | CA | LYS | B | 59 | 7.178 | 23.648 | 23.327 | 1.00 | 25.11 | 6 | C |
| ATOM | 1047 | CB | LYS | B | 59 | 6.578 | 24.692 | 24.255 | 1.00 | 27.02 | 6 | C |
| ATOM | 1048 | CG | LYS | B | 59 | 6.244 | 24.161 | 25.639 | 1.00 | 30.90 | 6 | C |
| ATOM | 1049 | CD | LYS | B | 59 | 5.624 | 25.236 | 26.549 | 1.00 | 34.58 | 6 | C |
| ATOM | 1050 | CE | LYS | B | 59 | 5.699 | 24.798 | 28.020 | 1.00 | 34.94 | 6 | C |
| ATOM | 1051 | NZ | LYS | B | 59 | 5.725 | 25.944 | 28.972 | 1.00 | 36.11 | 7 | N |
| ATOM | 1052 | C | LYS | B | 59 | 6.911 | 24.004 | 21.857 | 1.00 | 23.87 | 6 | C |
| ATOM | 1053 | O | LYS | B | 59 | 6.423 | 23.147 | 21.109 | 1.00 | 23.36 | 8 | O |
| ATOM | 1054 | N | ILE | B | 60 | 7.548 | 25.056 | 21.351 | 1.00 | 19.85 | 7 | N |
| ATOM | 1055 | CA | ILE | B | 60 | 7.274 | 25.554 | 20.003 | 1.00 | 17.05 | 6 | C |
| ATOM | 1056 | CB | ILE | B | 60 | 8.007 | 26.905 | 19.765 | 1.00 | 15.53 | 6 | C |
| ATOM | 1057 | CG2 | ILE | B | 60 | 7.825 | 27.435 | 18.363 | 1.00 | 13.69 | 6 | C |
| ATOM | 1058 | CG1 | ILE | B | 60 | 7.481 | 27.986 | 20.736 | 1.00 | 16.51 | 6 | C |
| ATOM | 1059 | CD1 | ILE | B | 60 | 5.991 | 28.251 | 20.476 | 1.00 | 15.98 | 6 | C |
| ATOM | 1060 | C | ILE | B | 60 | 7.609 | 24.563 | 18.917 | 1.00 | 17.34 | 6 | C |
| ATOM | 1061 | O | ILE | B | 60 | 6.772 | 24.162 | 18.130 | 1.00 | 14.52 | 8 | O |
| ATOM | 1062 | N | ARG | B | 61 | 8.875 | 24.103 | 18.890 | 1.00 | 18.70 | 7 | N |
| ATOM | 1063 | CA | ARG | B | 61 | 9.371 | 23.212 | 17.852 | 1.00 | 20.52 | 6 | C |
| ATOM | 1064 | CB | ARG | B | 61 | 10.920 | 23.301 | 17.802 | 1.00 | 20.89 | 6 | C |
| ATOM | 1065 | CG | ARG | B | 61 | 11.306 | 24.662 | 17.216 | 1.00 | 21.37 | 6 | C |
| ATOM | 1066 | CD | ARG | B | 61 | 12.788 | 24.911 | 17.048 | 1.00 | 21.93 | 6 | C |
| ATOM | 1067 | NE | ARG | B | 61 | 13.411 | 24.028 | 16.080 | 1.00 | 21.23 | 7 | N |
| ATOM | 1068 | CZ | ARG | B | 61 | 13.511 | 24.024 | 14.776 | 1.00 | 18.33 | 6 | C |
| ATOM | 1069 | NH1 | ARG | B | 61 | 12.968 | 24.992 | 14.081 | 1.00 | 18.18 | 7 | N |
| ATOM | 1070 | NH2 | ARG | B | 61 | 14.210 | 23.051 | 14.197 | 1.00 | 22.37 | 7 | N |
| ATOM | 1071 | C | ARG | B | 61 | 8.849 | 21.789 | 17.877 | 1.00 | 20.48 | 6 | C |
| ATOM | 1072 | O | ARG | B | 61 | 8.872 | 21.139 | 16.823 | 1.00 | 19.46 | 8 | O |
| ATOM | 1073 | N | LYS | B | 62 | 8.189 | 21.397 | 18.948 | 1.00 | 19.89 | 7 | N |
| ATOM | 1074 | CA | LYS | B | 62 | 7.486 | 20.126 | 19.010 | 1.00 | 22.12 | 6 | C |
| ATOM | 1075 | CB | LYS | B | 62 | 7.564 | 19.510 | 20.415 | 1.00 | 22.87 | 6 | C |
| ATOM | 1076 | CG | LYS | B | 62 | 8.998 | 19.480 | 20.908 | 1.00 | 25.05 | 6 | C |
| ATOM | 1077 | CD | LYS | B | 62 | 9.842 | 18.467 | 20.132 | 1.00 | 26.73 | 6 | C |
| ATOM | 1078 | CE | LYS | B | 62 | 11.164 | 18.267 | 20.879 | 1.00 | 28.67 | 6 | C |
| ATOM | 1079 | NZ | LYS | B | 62 | 11.865 | 17.052 | 20.376 | 1.00 | 29.88 | 7 | N |
| ATOM | 1080 | C | LYS | B | 62 | 6.018 | 20.266 | 18.653 | 1.00 | 22.89 | 6 | C |
| ATOM | 1081 | O | LYS | B | 62 | 5.371 | 19.221 | 18.738 | 1.00 | 24.68 | 8 | O |
| ATOM | 1082 | N | MET | B | 63 | 5.493 | 21.455 | 18.346 | 1.00 | 22.05 | 7 | N |
| ATOM | 1083 | CA | MET | B | 63 | 4.085 | 21.535 | 17.935 | 1.00 | 21.24 | 6 | C |
| ATOM | 1084 | CB | MET | B | 63 | 3.596 | 22.986 | 17.770 | 1.00 | 21.67 | 6 | C |
| ATOM | 1085 | CG | MET | B | 63 | 3.515 | 23.697 | 19.108 | 1.00 | 24.24 | 6 | C |
| ATOM | 1086 | SD | MET | B | 63 | 2.958 | 25.403 | 19.105 | 1.00 | 25.86 | 16 | S |
| ATOM | 1087 | CE | MET | B | 63 | 1.266 | 25.330 | 18.546 | 1.00 | 22.54 | 6 | C |
| ATOM | 1088 | C | MET | B | 63 | 3.994 | 20.877 | 16.578 | 1.00 | 20.21 | 6 | C |
| ATOM | 1089 | O | MET | B | 63 | 4.679 | 21.365 | 15.693 | 1.00 | 19.21 | 8 | O |
| ATOM | 1090 | N | PRO | B | 64 | 3.013 | 20.020 | 16.328 | 1.00 | 20.66 | 7 | N |
| ATOM | 1091 | CD | PRO | B | 64 | 2.171 | 19.423 | 17.400 | 1.00 | 21.16 | 6 | C |
| ATOM | 1092 | CA | PRO | B | 64 | 3.009 | 19.225 | 15.113 | 1.00 | 20.71 | 6 | C |
| ATOM | 1093 | CB | PRO | B | 64 | 1.914 | 18.185 | 15.357 | 1.00 | 22.37 | 6 | C |
| ATOM | 1094 | CG | PRO | B | 64 | 1.645 | 18.137 | 16.824 | 1.00 | 20.69 | 6 | C |
| ATOM | 1095 | C | PRO | B | 64 | 2.817 | 20.011 | 13.840 | 1.00 | 20.17 | 6 | C |
| ATOM | 1096 | O | PRO | B | 64 | 3.344 | 19.668 | 12.765 | 1.00 | 19.17 | 8 | O |
| ATOM | 1097 | N | GLU | B | 65 | 2.077 | 21.113 | 13.928 | 1.00 | 18.92 | 7 | N |
| ATOM | 1098 | CA | GLU | B | 65 | 1.808 | 21.925 | 12.750 | 1.00 | 20.82 | 6 | C |
| ATOM | 1099 | CB | GLU | B | 65 | 0.451 | 22.625 | 12.962 | 1.00 | 23.06 | 6 | C |
| ATOM | 1100 | CG | GLU | B | 65 | −0.570 | 21.732 | 13.649 | 1.00 | 24.65 | 6 | C |
| ATOM | 1101 | CD | GLU | B | 65 | −0.701 | 22.037 | 15.135 | 1.00 | 28.89 | 6 | C |
| ATOM | 1102 | OE1 | GLU | B | 65 | 0.281 | 21.999 | 15.947 | 1.00 | 26.57 | 8 | O |
| ATOM | 1103 | OE2 | GLU | B | 65 | −1.900 | 22.338 | 15.495 | 1.00 | 30.49 | 8 | O |
| ATOM | 1104 | C | GLU | B | 65 | 2.866 | 22.965 | 12.446 | 1.00 | 20.76 | 6 | C |
| ATOM | 1105 | O | GLU | B | 65 | 2.822 | 23.524 | 11.345 | 1.00 | 23.34 | 8 | O |
| ATOM | 1106 | N | ILE | B | 66 | 3.880 | 23.130 | 13.292 | 1.00 | 19.85 | 7 | N |
| ATOM | 1107 | CA | ILE | B | 66 | 4.988 | 24.066 | 12.991 | 1.00 | 18.23 | 6 | C |
| ATOM | 1108 | CB | ILE | B | 66 | 5.574 | 24.580 | 14.324 | 1.00 | 18.27 | 6 | C |
| ATOM | 1109 | CG2 | ILE | B | 66 | 6.951 | 25.202 | 14.145 | 1.00 | 18.20 | 6 | C |
| ATOM | 1110 | CG1 | ILE | B | 66 | 4.502 | 25.528 | 14.888 | 1.00 | 18.67 | 6 | C |
| ATOM | 1111 | CD1 | ILE | B | 66 | 4.911 | 26.360 | 16.092 | 1.00 | 19.78 | 6 | C |
| ATOM | 1112 | C | ILE | B | 66 | 6.023 | 23.331 | 12.155 | 1.00 | 19.56 | 6 | C |
| ATOM | 1113 | O | ILE | B | 66 | 6.493 | 22.275 | 12.616 | 1.00 | 18.62 | 8 | O |
| ATOM | 1114 | N | GLN | B | 67 | 6.373 | 23.821 | 10.957 | 1.00 | 18.63 | 7 | N |
| ATOM | 1115 | CA | GLN | B | 67 | 7.296 | 23.106 | 10.117 | 1.00 | 19.98 | 6 | C |
| ATOM | 1116 | CB | GLN | B | 67 | 6.901 | 23.271 | 8.625 | 1.00 | 21.88 | 6 | C |
| ATOM | 1117 | CG | GLN | B | 67 | 5.496 | 22.762 | 8.421 | 1.00 | 26.02 | 6 | C |
| ATOM | 1118 | CD | GLN | B | 67 | 5.042 | 22.459 | 7.026 | 1.00 | 28.55 | 6 | C |
| ATOM | 1119 | OE1 | GLN | B | 67 | 4.361 | 21.434 | 6.858 | 1.00 | 31.69 | 8 | O |
| ATOM | 1120 | NE2 | GLN | B | 67 | 5.284 | 23.306 | 6.034 | 1.00 | 29.80 | 7 | N |
| ATOM | 1121 | C | GLN | B | 67 | 8.706 | 23.681 | 10.243 | 1.00 | 21.32 | 6 | C |
| ATOM | 1122 | O | GLN | B | 67 | 9.715 | 23.099 | 9.843 | 1.00 | 20.33 | 8 | O |
| ATOM | 1123 | N | MET | B | 68 | 8.749 | 24.966 | 10.583 | 1.00 | 21.16 | 7 | N |
| ATOM | 1124 | CA | MET | B | 68 | 10.009 | 25.693 | 10.650 | 1.00 | 20.94 | 6 | C |
| ATOM | 1125 | CB | MET | B | 68 | 10.295 | 26.285 | 9.262 | 1.00 | 24.01 | 6 | C |
| ATOM | 1126 | CG | MET | B | 68 | 10.902 | 25.394 | 8.237 | 1.00 | 30.45 | 6 | C |
| ATOM | 1127 | SD | MET | B | 68 | 11.853 | 26.281 | 7.020 | 1.00 | 38.87 | 16 | S |
| ATOM | 1128 | CE | MET | B | 68 | 10.602 | 26.712 | 5.822 | 1.00 | 35.81 | 6 | C |
| ATOM | 1129 | C | MET | B | 68 | 9.911 | 26.899 | 11.558 | 1.00 | 17.78 | 6 | C |
| ATOM | 1130 | O | MET | B | 68 | 8.870 | 27.576 | 11.556 | 1.00 | 16.20 | 8 | O |
| ATOM | 1131 | N | THR | B | 69 | 10.977 | 27.268 | 12.235 | 1.00 | 15.58 | 7 | N |
| ATOM | 1132 | CA | THR | B | 69 | 11.017 | 28.506 | 13.025 | 1.00 | 12.38 | 6 | C |
| ATOM | 1133 | CB | THR | B | 69 | 10.793 | 28.278 | 14.527 | 1.00 | 13.97 | 6 | C |
| ATOM | 1134 | OG1 | THR | B | 69 | 11.853 | 27.497 | 15.116 | 1.00 | 14.07 | 8 | O |
| ATOM | 1135 | CG2 | THR | B | 69 | 9.458 | 27.596 | 14.822 | 1.00 | 11.50 | 6 | C |
| ATOM | 1136 | C | THR | B | 69 | 12.360 | 29.171 | 12.790 | 1.00 | 14.55 | 6 | C |
| ATOM | 1137 | O | THR | B | 69 | 13.364 | 28.457 | 12.587 | 1.00 | 13.00 | 8 | O |
| ATOM | 1138 | N | SER | B | 70 | 12.431 | 30.484 | 12.926 | 1.00 | 11.76 | 7 | N |
| ATOM | 1139 | CA | SER | B | 70 | 13.653 | 31.254 | 12.816 | 1.00 | 11.23 | 6 | C |
| ATOM | 1140 | CB | SER | B | 70 | 13.770 | 31.802 | 11.403 | 1.00 | 10.94 | 6 | C |
| ATOM | 1141 | OG | SER | B | 70 | 14.807 | 32.763 | 11.395 | 1.00 | 13.40 | 8 | O |
| ATOM | 1142 | C | SER | B | 70 | 13.467 | 32.327 | 13.909 | 1.00 | 13.08 | 6 | C |
| ATOM | 1143 | O | SER | B | 70 | 12.493 | 33.086 | 13.794 | 1.00 | 11.11 | 8 | O |
| ATOM | 1144 | N | THR | B | 71 | 14.234 | 32.225 | 15.001 | 1.00 | 9.49 | 7 | N |
| ATOM | 1145 | CA | THR | B | 71 | 14.004 | 33.081 | 16.152 | 1.00 | 9.27 | 6 | C |
| ATOM | 1146 | CB | THR | B | 71 | 14.023 | 32.166 | 17.406 | 1.00 | 11.05 | 6 | C |
| ATOM | 1147 | OG1 | THR | B | 71 | 12.964 | 31.212 | 17.347 | 1.00 | 10.77 | 8 | O |
| ATOM | 1148 | CG2 | THR | B | 71 | 13.880 | 33.018 | 18.664 | 1.00 | 12.37 | 6 | C |
| ATOM | 1149 | C | THR | B | 71 | 15.047 | 34.188 | 16.282 | 1.00 | 8.22 | 6 | C |
| ATOM | 1150 | O | THR | B | 71 | 16.249 | 33.931 | 16.119 | 1.00 | 10.11 | 8 | O |
| ATOM | 1151 | N | MET | B | 72 | 14.635 | 35.405 | 16.449 | 1.00 | 6.69 | 7 | N |
| ATOM | 1152 | CA | MET | B | 72 | 15.517 | 36.547 | 16.585 | 1.00 | 7.80 | 6 | C |
| ATOM | 1153 | CB | MET | B | 72 | 15.111 | 37.662 | 15.627 | 1.00 | 10.55 | 6 | C |
| ATOM | 1154 | CG | MET | B | 72 | 15.251 | 37.183 | 14.165 | 1.00 | 16.67 | 6 | C |
| ATOM | 1155 | SD | MET | B | 72 | 14.478 | 38.386 | 13.048 | 1.00 | 20.04 | 16 | S |
| ATOM | 1156 | CE | MET | B | 72 | 14.819 | 37.455 | 11.545 | 1.00 | 23.19 | 6 | C |
| ATOM | 1157 | C | MET | B | 72 | 15.467 | 37.079 | 18.005 | 1.00 | 7.77 | 6 | C |
| ATOM | 1158 | O | MET | B | 72 | 14.442 | 37.688 | 18.349 | 1.00 | 7.18 | 8 | O |
| ATOM | 1159 | N | ILE | B | 73 | 16.559 | 36.867 | 18.744 | 1.00 | 6.41 | 7 | N |
| ATOM | 1160 | CA | ILE | B | 73 | 16.580 | 37.347 | 20.117 | 1.00 | 8.84 | 6 | C |
| ATOM | 1161 | CB | ILE | B | 73 | 17.656 | 36.575 | 20.926 | 1.00 | 7.95 | 6 | C |
| ATOM | 1162 | CG2 | ILE | B | 73 | 17.878 | 37.273 | 22.301 | 1.00 | 9.41 | 6 | C |
| ATOM | 1163 | CG1 | ILE | B | 73 | 17.217 | 35.102 | 21.017 | 1.00 | 8.05 | 6 | C |
| ATOM | 1164 | CD1 | ILE | B | 73 | 18.284 | 34.103 | 21.467 | 1.00 | 11.03 | 6 | C |
| ATOM | 1165 | C | ILE | B | 73 | 16.774 | 38.842 | 20.097 | 1.00 | 11.08 | 6 | C |
| ATOM | 1166 | O | ILE | B | 73 | 17.663 | 39.307 | 19.357 | 1.00 | 12.77 | 8 | O |
| ATOM | 1167 | N | ALA | B | 74 | 16.112 | 39.633 | 20.955 | 1.00 | 11.74 | 7 | N |
| ATOM | 1168 | CA | ALA | B | 74 | 16.349 | 41.049 | 21.012 | 1.00 | 11.91 | 6 | C |
| ATOM | 1169 | CB | ALA | B | 74 | 15.190 | 41.968 | 21.481 | 1.00 | 11.66 | 6 | C |
| ATOM | 1170 | C | ALA | B | 74 | 17.560 | 41.357 | 21.904 | 1.00 | 14.51 | 6 | C |
| ATOM | 1171 | O | ALA | B | 74 | 17.794 | 40.714 | 22.942 | 1.00 | 11.20 | 8 | O |
| ATOM | 1172 | N | ILE | B | 75 | 18.109 | 42.519 | 21.630 | 1.00 | 17.53 | 7 | N |
| ATOM | 1173 | CA | ILE | B | 75 | 19.305 | 43.071 | 22.262 | 1.00 | 24.22 | 6 | C |
| ATOM | 1174 | CB | ILE | B | 75 | 20.071 | 43.938 | 21.234 | 1.00 | 25.52 | 6 | C |
| ATOM | 1175 | CG2 | ILE | B | 75 | 20.752 | 45.181 | 21.763 | 1.00 | 27.56 | 6 | C |
| ATOM | 1176 | CG1 | ILE | B | 75 | 21.106 | 43.078 | 20.489 | 1.00 | 25.82 | 6 | C |
| ATOM | 1177 | CD1 | ILE | B | 75 | 22.210 | 42.628 | 21.402 | 1.00 | 28.79 | 6 | C |
| ATOM | 1178 | C | ILE | B | 75 | 18.913 | 43.902 | 23.470 | 1.00 | 27.45 | 6 | C |
| ATOM | 1179 | O | ILE | B | 75 | 17.779 | 44.405 | 23.469 | 1.00 | 29.37 | 8 | O |
| ATOM | 1180 | OT | ILE | B | 75 | 19.762 | 44.028 | 24.395 | 1.00 | 31.78 | 8 | O |
| ATOM | 1181 | CB | VAL | C | 2 | 39.332 | 19.443 | 4.171 | 1.00 | 13.51 | 6 | C |
| ATOM | 1182 | CG1 | VAL | C | 2 | 38.145 | 20.355 | 3.825 | 1.00 | 13.69 | 6 | C |
| ATOM | 1183 | CG2 | VAL | C | 2 | 40.295 | 19.357 | 2.968 | 1.00 | 11.61 | 6 | C |
| ATOM | 1184 | C | VAL | C | 2 | 39.163 | 20.233 | 6.562 | 1.00 | 13.74 | 6 | C |
| ATOM | 1185 | O | VAL | C | 2 | 38.668 | 19.280 | 7.202 | 1.00 | 13.05 | 8 | O |
| ATOM | 1186 | N | VAL | C | 2 | 41.199 | 18.998 | 5.711 | 1.00 | 13.50 | 7 | N |
| ATOM | 1187 | CA | VAL | C | 2 | 40.102 | 19.970 | 5.380 | 1.00 | 13.12 | 6 | C |
| ATOM | 1188 | N | THR | C | 3 | 39.031 | 21.486 | 6.942 | 1.00 | 13.22 | 7 | N |
| ATOM | 1189 | CA | THR | C | 3 | 38.168 | 21.900 | 8.039 | 1.00 | 14.11 | 6 | C |
| ATOM | 1190 | CB | THR | C | 3 | 38.833 | 22.798 | 9.084 | 1.00 | 12.14 | 6 | C |
| ATOM | 1191 | OG1 | THR | C | 3 | 39.931 | 22.193 | 9.744 | 1.00 | 12.53 | 8 | O |
| ATOM | 1192 | CG2 | THR | C | 3 | 37.829 | 23.094 | 10.246 | 1.00 | 10.76 | 6 | C |
| ATOM | 1193 | C | THR | C | 3 | 36.960 | 22.646 | 7.426 | 1.00 | 13.61 | 6 | C |
| ATOM | 1194 | O | THR | C | 3 | 37.202 | 23.489 | 6.563 | 1.00 | 12.66 | 8 | O |
| ATOM | 1195 | N | ALA | C | 4 | 35.746 | 22.325 | 7.836 | 1.00 | 11.41 | 7 | N |
| ATOM | 1196 | CA | ALA | C | 4 | 34.586 | 22.976 | 7.285 | 1.00 | 11.20 | 6 | C |
| ATOM | 1197 | CB | ALA | C | 4 | 33.979 | 22.223 | 6.107 | 1.00 | 8.89 | 6 | C |
| ATOM | 1198 | C | ALA | C | 4 | 33.477 | 23.136 | 8.345 | 1.00 | 11.12 | 6 | C |
| ATOM | 1199 | O | ALA | C | 4 | 33.347 | 22.322 | 9.266 | 1.00 | 10.90 | 8 | O |
| ATOM | 1200 | N | PHE | C | 5 | 32.693 | 24.214 | 8.185 | 1.00 | 9.67 | 7 | N |
| ATOM | 1201 | CA | PHE | C | 5 | 31.550 | 24.453 | 9.036 | 1.00 | 9.21 | 6 | C |
| ATOM | 1202 | CB | PHE | C | 5 | 31.422 | 25.860 | 9.633 | 1.00 | 9.50 | 6 | C |
| ATOM | 1203 | CG | PHE | C | 5 | 32.397 | 26.100 | 10.752 | 1.00 | 10.21 | 6 | C |
| ATOM | 1204 | CD1 | PHE | C | 5 | 33.702 | 26.499 | 10.428 | 1.00 | 8.38 | 6 | C |
| ATOM | 1205 | CD2 | PHE | C | 5 | 32.076 | 25.900 | 12.080 | 1.00 | 9.76 | 6 | C |
| ATOM | 1206 | CE1 | PHE | C | 5 | 34.665 | 26.663 | 11.392 | 1.00 | 10.44 | 6 | C |
| ATOM | 1207 | CE2 | PHE | C | 5 | 33.051 | 26.086 | 13.050 | 1.00 | 8.18 | 6 | C |
| ATOM | 1208 | CZ | PHE | C | 5 | 34.318 | 26.446 | 12.727 | 1.00 | 9.99 | 6 | C |
| ATOM | 1209 | C | PHE | C | 5 | 30.312 | 24.105 | 8.196 | 1.00 | 11.44 | 6 | C |
| ATOM | 1210 | O | PHE | C | 5 | 30.128 | 24.588 | 7.060 | 1.00 | 13.44 | 8 | O |
| ATOM | 1211 | N | ILE | C | 6 | 29.458 | 23.243 | 8.739 | 1.00 | 9.23 | 7 | N |
| ATOM | 1212 | CA | ILE | C | 6 | 28.227 | 22.977 | 7.980 | 1.00 | 10.67 | 6 | C |
| ATOM | 1213 | CB | ILE | C | 6 | 28.075 | 21.453 | 7.793 | 1.00 | 12.19 | 6 | C |
| ATOM | 1214 | CG2 | ILE | C | 6 | 26.744 | 21.189 | 7.118 | 1.00 | 11.24 | 6 | C |
| ATOM | 1215 | CG1 | ILE | C | 6 | 29.268 | 20.837 | 7.020 | 1.00 | 12.66 | 6 | C |
| ATOM | 1216 | CD1 | ILE | C | 6 | 29.037 | 19.318 | 6.949 | 1.00 | 12.74 | 6 | C |
| ATOM | 1217 | C | ILE | C | 6 | 27.055 | 23.571 | 8.739 | 1.00 | 9.95 | 6 | C |
| ATOM | 1218 | O | ILE | C | 6 | 26.932 | 23.245 | 9.922 | 1.00 | 7.94 | 8 | O |
| ATOM | 1219 | N | LEU | C | 7 | 26.275 | 24.443 | 8.129 | 1.00 | 10.46 | 7 | N |
| ATOM | 1220 | CA | LEU | C | 7 | 25.123 | 25.054 | 8.768 | 1.00 | 11.52 | 6 | C |
| ATOM | 1221 | CB | LEU | C | 7 | 24.963 | 26.522 | 8.306 | 1.00 | 14.75 | 6 | C |
| ATOM | 1222 | CG | LEU | C | 7 | 26.163 | 27.363 | 8.764 | 1.00 | 16.86 | 6 | C |
| ATOM | 1223 | CD1 | LEU | C | 7 | 25.971 | 28.807 | 8.310 | 1.00 | 19.73 | 6 | C |
| ATOM | 1224 | CD2 | LEU | C | 7 | 26.301 | 27.276 | 10.262 | 1.00 | 17.56 | 6 | C |
| ATOM | 1225 | C | LEU | C | 7 | 23.887 | 24.324 | 8.262 | 1.00 | 13.74 | 6 | C |
| ATOM | 1226 | O | LEU | C | 7 | 23.864 | 24.231 | 7.024 | 1.00 | 15.75 | 8 | O |
| ATOM | 1227 | N | MET | C | 8 | 22.967 | 23.861 | 9.073 | 1.00 | 12.28 | 7 | N |
| ATOM | 1228 | CA | MET | C | 8 | 21.871 | 23.050 | 8.619 | 1.00 | 15.30 | 6 | C |
| ATOM | 1229 | CB | MET | C | 8 | 22.064 | 21.613 | 9.116 | 1.00 | 16.92 | 6 | C |
| ATOM | 1230 | CG | MET | C | 8 | 23.275 | 20.809 | 8.666 | 1.00 | 18.76 | 6 | C |
| ATOM | 1231 | SD | MET | C | 8 | 23.600 | 19.314 | 9.582 | 1.00 | 20.83 | 16 | S |
| ATOM | 1232 | CE | MET | C | 8 | 24.791 | 19.921 | 10.776 | 1.00 | 24.28 | 6 | C |
| ATOM | 1233 | C | MET | C | 8 | 20.510 | 23.492 | 9.187 | 1.00 | 17.65 | 6 | C |
| ATOM | 1234 | O | MET | C | 8 | 20.409 | 23.926 | 10.346 | 1.00 | 16.91 | 8 | O |
| ATOM | 1235 | N | VAL | C | 9 | 19.496 | 23.240 | 8.362 | 1.00 | 19.14 | 7 | N |
| ATOM | 1236 | CA | VAL | C | 9 | 18.083 | 23.374 | 8.761 | 1.00 | 19.28 | 6 | C |
| ATOM | 1237 | CB | VAL | C | 9 | 17.168 | 23.967 | 7.718 | 1.00 | 19.18 | 6 | C |
| ATOM | 1238 | CG1 | VAL | C | 9 | 15.697 | 23.905 | 8.170 | 1.00 | 19.22 | 6 | C |
| ATOM | 1239 | CG2 | VAL | C | 9 | 17.579 | 25.418 | 7.540 | 1.00 | 19.16 | 6 | C |
| ATOM | 1240 | C | VAL | C | 9 | 17.598 | 21.966 | 9.060 | 1.00 | 19.88 | 6 | C |
| ATOM | 1241 | O | VAL | C | 9 | 18.106 | 21.035 | 8.414 | 1.00 | 23.28 | 8 | O |
| ATOM | 1242 | N | THR | C | 10 | 16.890 | 21.717 | 10.155 | 1.00 | 19.30 | 7 | N |
| ATOM | 1243 | CA | THR | C | 10 | 16.411 | 20.364 | 10.393 | 1.00 | 17.92 | 6 | C |
| ATOM | 1244 | CB | THR | C | 10 | 16.843 | 19.661 | 11.672 | 1.00 | 16.38 | 6 | C |
| ATOM | 1245 | OG1 | THR | C | 10 | 16.133 | 20.315 | 12.741 | 1.00 | 18.11 | 8 | O |
| ATOM | 1246 | CG2 | THR | C | 10 | 18.340 | 19.783 | 11.879 | 1.00 | 18.62 | 6 | C |
| ATOM | 1247 | C | THR | C | 10 | 14.873 | 20.411 | 10.478 | 1.00 | 19.30 | 6 | C |
| ATOM | 1248 | O | THR | C | 10 | 14.321 | 21.487 | 10.541 | 1.00 | 18.20 | 8 | O |
| ATOM | 1249 | N | ALA | C | 11 | 14.222 | 19.244 | 10.430 | 1.00 | 19.87 | 7 | N |
| ATOM | 1250 | CA | ALA | C | 11 | 12.729 | 19.379 | 10.557 | 1.00 | 21.46 | 6 | C |
| ATOM | 1251 | CB | ALA | C | 11 | 12.177 | 17.984 | 10.450 | 1.00 | 20.40 | 6 | C |
| ATOM | 1252 | C | ALA | C | 11 | 12.554 | 20.024 | 11.947 | 1.00 | 22.53 | 6 | C |
| ATOM | 1253 | O | ALA | C | 11 | 13.387 | 19.737 | 12.828 | 1.00 | 20.28 | 8 | O |
| ATOM | 1254 | N | ALA | C | 12 | 11.570 | 20.893 | 12.172 | 1.00 | 22.83 | 7 | N |
| ATOM | 1255 | CA | ALA | C | 12 | 11.388 | 21.485 | 13.491 | 1.00 | 21.67 | 6 | C |
| ATOM | 1256 | CB | ALA | C | 12 | 10.050 | 22.226 | 13.511 | 1.00 | 21.82 | 6 | C |
| ATOM | 1257 | C | ALA | C | 12 | 11.279 | 20.386 | 14.546 | 1.00 | 22.13 | 6 | C |
| ATOM | 1258 | O | ALA | C | 12 | 10.431 | 19.494 | 14.387 | 1.00 | 21.92 | 8 | O |
| ATOM | 1259 | N | GLY | C | 13 | 12.059 | 20.441 | 15.634 | 1.00 | 21.75 | 7 | N |
| ATOM | 1260 | CA | GLY | C | 13 | 11.867 | 19.456 | 16.708 | 1.00 | 20.46 | 6 | C |
| ATOM | 1261 | C | GLY | C | 13 | 12.824 | 18.291 | 16.688 | 1.00 | 20.35 | 6 | C |
| ATOM | 1262 | O | GLY | C | 13 | 12.994 | 17.498 | 17.614 | 1.00 | 21.82 | 8 | O |
| ATOM | 1263 | N | LYS | C | 14 | 13.508 | 18.073 | 15.581 | 1.00 | 21.21 | 7 | N |
| ATOM | 1264 | CA | LYS | C | 14 | 14.496 | 17.032 | 15.406 | 1.00 | 20.28 | 6 | C |
| ATOM | 1265 | CB | LYS | C | 14 | 14.312 | 16.435 | 13.999 | 1.00 | 22.02 | 6 | C |
| ATOM | 1266 | CG | LYS | C | 14 | 12.877 | 15.929 | 13.752 | 1.00 | 23.94 | 6 | C |
| ATOM | 1267 | CD | LYS | C | 14 | 12.495 | 14.817 | 14.711 | 1.00 | 24.03 | 6 | C |
| ATOM | 1268 | CE | LYS | C | 14 | 11.142 | 14.233 | 14.252 | 1.00 | 27.62 | 6 | C |
| ATOM | 1269 | NZ | LYS | C | 14 | 10.815 | 13.033 | 15.090 | 1.00 | 29.36 | 7 | N |
| ATOM | 1270 | C | LYS | C | 14 | 15.931 | 17.544 | 15.561 | 1.00 | 20.24 | 6 | C |
| ATOM | 1271 | O | LYS | C | 14 | 16.810 | 16.665 | 15.603 | 1.00 | 20.05 | 8 | O |
| ATOM | 1272 | N | GLU | C | 15 | 16.201 | 18.853 | 15.711 | 1.00 | 17.96 | 7 | N |
| ATOM | 1273 | CA | GLU | C | 15 | 17.600 | 19.258 | 15.842 | 1.00 | 17.39 | 6 | C |
| ATOM | 1274 | CB | GLU | C | 15 | 17.723 | 20.771 | 16.040 | 1.00 | 18.15 | 6 | C |
| ATOM | 1275 | CG | GLU | C | 15 | 16.929 | 21.378 | 17.155 | 1.00 | 21.05 | 6 | C |
| ATOM | 1276 | CD | GLU | C | 15 | 15.446 | 21.691 | 16.877 | 1.00 | 23.06 | 6 | C |
| ATOM | 1277 | OE1 | GLU | C | 15 | 14.902 | 22.458 | 17.720 | 1.00 | 22.27 | 8 | O |
| ATOM | 1278 | OE2 | GLU | C | 15 | 14.811 | 21.237 | 15.899 | 1.00 | 19.49 | 8 | O |
| ATOM | 1279 | C | GLU | C | 15 | 18.439 | 18.541 | 16.875 | 1.00 | 17.98 | 6 | C |
| ATOM | 1280 | O | GLU | C | 15 | 19.633 | 18.251 | 16.612 | 1.00 | 17.01 | 8 | O |
| ATOM | 1281 | N | ARG | C | 16 | 17.951 | 18.262 | 18.084 | 1.00 | 19.93 | 7 | N |
| ATOM | 1282 | CA | ARG | C | 16 | 18.758 | 17.602 | 19.095 | 1.00 | 23.34 | 6 | C |
| ATOM | 1283 | CB | ARG | C | 16 | 18.142 | 17.638 | 20.485 | 1.00 | 26.49 | 6 | C |
| ATOM | 1284 | CG | ARG | C | 16 | 18.577 | 18.836 | 21.313 | 1.00 | 32.57 | 6 | C |
| ATOM | 1285 | CD | ARG | C | 16 | 17.739 | 18.911 | 22.588 | 1.00 | 36.19 | 6 | C |
| ATOM | 1286 | NE | ARG | C | 16 | 17.646 | 20.313 | 23.027 | 1.00 | 40.59 | 7 | N |
| ATOM | 1287 | CZ | ARG | C | 16 | 16.894 | 20.698 | 24.064 | 1.00 | 43.15 | 6 | C |
| ATOM | 1288 | NH1 | ARG | C | 16 | 16.218 | 19.820 | 24.813 | 1.00 | 45.02 | 7 | N |
| ATOM | 1289 | NH2 | ARG | C | 16 | 16.882 | 21.992 | 24.344 | 1.00 | 43.41 | 7 | N |
| ATOM | 1290 | C | ARG | C | 16 | 19.121 | 16.155 | 18.749 | 1.00 | 23.41 | 6 | C |
| ATOM | 1291 | O | ARG | C | 16 | 20.244 | 15.713 | 19.020 | 1.00 | 24.50 | 8 | O |
| ATOM | 1292 | N | GLU | C | 17 | 18.196 | 15.378 | 18.198 | 1.00 | 22.54 | 7 | N |
| ATOM | 1293 | CA | GLU | C | 17 | 18.534 | 13.994 | 17.878 | 1.00 | 22.99 | 6 | C |
| ATOM | 1294 | CB | GLU | C | 17 | 17.267 | 13.167 | 17.843 | 1.00 | 23.42 | 6 | C |
| ATOM | 1295 | CG | GLU | C | 17 | 16.247 | 13.469 | 16.765 | 1.00 | 26.37 | 6 | C |
| ATOM | 1296 | CD | GLU | C | 17 | 16.705 | 12.793 | 15.478 | 1.00 | 29.84 | 6 | C |
| ATOM | 1297 | OE1 | GLU | C | 17 | 17.540 | 11.856 | 15.651 | 1.00 | 30.85 | 8 | O |
| ATOM | 1298 | OE2 | GLU | C | 17 | 16.264 | 13.213 | 14.403 | 1.00 | 30.82 | 8 | O |
| ATOM | 1299 | C | GLU | C | 17 | 19.392 | 13.983 | 16.617 | 1.00 | 21.91 | 6 | C |
| ATOM | 1300 | O | GLU | C | 17 | 20.201 | 13.066 | 16.406 | 1.00 | 23.15 | 8 | O |
| ATOM | 1301 | N | VAL | C | 18 | 19.222 | 14.931 | 15.686 | 1.00 | 20.55 | 7 | N |
| ATOM | 1302 | CA | VAL | C | 18 | 20.142 | 14.929 | 14.546 | 1.00 | 18.68 | 6 | C |
| ATOM | 1303 | CB | VAL | C | 18 | 19.763 | 16.002 | 13.524 | 1.00 | 18.86 | 6 | C |
| ATOM | 1304 | CG1 | VAL | C | 18 | 20.916 | 16.280 | 12.556 | 1.00 | 17.78 | 6 | C |
| ATOM | 1305 | CG2 | VAL | C | 18 | 18.534 | 15.567 | 12.751 | 1.00 | 17.95 | 6 | C |
| ATOM | 1306 | C | VAL | C | 18 | 21.529 | 15.200 | 15.104 | 1.00 | 18.89 | 6 | C |
| ATOM | 1307 | O | VAL | C | 18 | 22.532 | 14.523 | 14.868 | 1.00 | 20.52 | 8 | O |
| ATOM | 1308 | N | MET | C | 19 | 21.637 | 16.181 | 15.993 | 1.00 | 18.99 | 7 | N |
| ATOM | 1309 | CA | MET | C | 19 | 22.925 | 16.541 | 16.559 | 1.00 | 19.72 | 6 | C |
| ATOM | 1310 | CB | MET | C | 19 | 22.769 | 17.748 | 17.479 | 1.00 | 19.34 | 6 | C |
| ATOM | 1311 | CG | MET | C | 19 | 24.112 | 18.134 | 18.123 | 1.00 | 19.03 | 6 | C |
| ATOM | 1312 | SD | MET | C | 19 | 23.905 | 19.552 | 19.172 | 1.00 | 19.97 | 16 | S |
| ATOM | 1313 | CE | MET | C | 19 | 22.982 | 18.859 | 20.519 | 1.00 | 21.11 | 6 | C |
| ATOM | 1314 | C | MET | C | 19 | 23.606 | 15.412 | 17.319 | 1.00 | 20.76 | 6 | C |
| ATOM | 1315 | O | MET | C | 19 | 24.841 | 15.407 | 17.408 | 1.00 | 20.12 | 8 | O |
| ATOM | 1316 | N | GLU | C | 20 | 22.856 | 14.570 | 18.009 | 1.00 | 21.72 | 7 | N |
| ATOM | 1317 | CA | GLU | C | 20 | 23.500 | 13.493 | 18.805 | 1.00 | 22.57 | 6 | C |
| ATOM | 1318 | CB | GLU | C | 20 | 22.492 | 12.946 | 19.823 | 1.00 | 23.97 | 6 | C |
| ATOM | 1319 | CG | GLU | C | 20 | 22.242 | 13.984 | 20.941 | 1.00 | 26.93 | 6 | C |
| ATOM | 1320 | CD | GLU | C | 20 | 23.506 | 14.388 | 21.687 | 1.00 | 28.95 | 6 | C |
| ATOM | 1321 | OE1 | GLU | C | 20 | 24.338 | 13.471 | 21.927 | 1.00 | 29.90 | 8 | O |
| ATOM | 1322 | OE2 | GLU | C | 20 | 23.742 | 15.589 | 22.041 | 1.00 | 29.95 | 8 | O |
| ATOM | 1323 | C | GLU | C | 20 | 24.055 | 12.461 | 17.840 | 1.00 | 21.95 | 6 | C |
| ATOM | 1324 | O | GLU | C | 20 | 25.107 | 11.816 | 18.043 | 1.00 | 23.26 | 8 | O |
| ATOM | 1325 | N | LYS | C | 21 | 23.317 | 12.251 | 16.749 | 1.00 | 19.49 | 7 | N |
| ATOM | 1326 | CA | LYS | C | 21 | 23.826 | 11.337 | 15.739 | 1.00 | 19.77 | 6 | C |
| ATOM | 1327 | CB | LYS | C | 21 | 22.813 | 11.186 | 14.594 | 1.00 | 19.76 | 6 | C |
| ATOM | 1328 | CG | LYS | C | 21 | 21.640 | 10.325 | 15.093 | 1.00 | 21.20 | 6 | C |
| ATOM | 1329 | CD | LYS | C | 21 | 20.679 | 10.149 | 13.933 | 1.00 | 22.36 | 6 | C |
| ATOM | 1330 | CE | LYS | C | 21 | 19.385 | 9.482 | 14.443 | 1.00 | 24.72 | 6 | C |
| ATOM | 1331 | NZ | LYS | C | 21 | 18.396 | 9.485 | 13.330 | 1.00 | 25.52 | 7 | N |
| ATOM | 1332 | C | LYS | C | 21 | 25.117 | 11.882 | 15.155 | 1.00 | 20.89 | 6 | C |
| ATOM | 1333 | O | LYS | C | 21 | 26.020 | 11.102 | 14.816 | 1.00 | 22.01 | 8 | O |
| ATOM | 1334 | N | LEU | C | 22 | 25.168 | 13.196 | 14.848 | 1.00 | 20.08 | 7 | N |
| ATOM | 1335 | CA | LEU | C | 22 | 26.349 | 13.828 | 14.265 | 1.00 | 17.68 | 6 | C |
| ATOM | 1336 | CB | LEU | C | 22 | 26.026 | 15.282 | 13.809 | 1.00 | 17.13 | 6 | C |
| ATOM | 1337 | CG | LEU | C | 22 | 25.157 | 15.440 | 12.582 | 1.00 | 17.15 | 6 | C |
| ATOM | 1338 | CD1 | LEU | C | 22 | 24.528 | 16.851 | 12.515 | 1.00 | 19.16 | 6 | C |
| ATOM | 1339 | CD2 | LEU | C | 22 | 25.864 | 15.262 | 11.272 | 1.00 | 18.59 | 6 | C |
| ATOM | 1340 | C | LEU | C | 22 | 27.497 | 13.777 | 15.240 | 1.00 | 18.53 | 6 | C |
| ATOM | 1341 | O | LEU | C | 22 | 28.660 | 13.456 | 14.925 | 1.00 | 18.17 | 8 | O |
| ATOM | 1342 | N | LEU | C | 23 | 27.270 | 14.064 | 16.528 | 1.00 | 19.42 | 7 | N |
| ATOM | 1343 | CA | LEU | C | 23 | 28.369 | 13.962 | 17.481 | 1.00 | 19.76 | 6 | C |
| ATOM | 1344 | CB | LEU | C | 23 | 27.928 | 14.386 | 18.870 | 1.00 | 19.18 | 6 | C |
| ATOM | 1345 | CG | LEU | C | 23 | 27.619 | 15.872 | 18.970 | 1.00 | 19.72 | 6 | C |
| ATOM | 1346 | CD1 | LEU | C | 23 | 26.733 | 16.088 | 20.201 | 1.00 | 19.30 | 6 | C |
| ATOM | 1347 | CD2 | LEU | C | 23 | 28.940 | 16.627 | 19.057 | 1.00 | 20.29 | 6 | C |
| ATOM | 1348 | C | LEU | C | 23 | 28.921 | 12.547 | 17.596 | 1.00 | 20.32 | 6 | C |
| ATOM | 1349 | O | LEU | C | 23 | 29.996 | 12.403 | 18.217 | 1.00 | 24.12 | 8 | O |
| ATOM | 1350 | N | ALA | C | 24 | 28.288 | 11.498 | 17.091 | 1.00 | 21.26 | 7 | N |
| ATOM | 1351 | CA | ALA | C | 24 | 28.945 | 10.187 | 17.254 | 1.00 | 23.37 | 6 | C |
| ATOM | 1352 | CB | ALA | C | 24 | 27.913 | 9.105 | 17.455 | 1.00 | 22.08 | 6 | C |
| ATOM | 1353 | C | ALA | C | 24 | 29.909 | 9.985 | 16.096 | 1.00 | 23.91 | 6 | C |
| ATOM | 1354 | O | ALA | C | 24 | 30.794 | 9.135 | 16.181 | 1.00 | 26.64 | 8 | O |
| ATOM | 1355 | N | MET | C | 25 | 29.831 | 10.763 | 15.018 | 1.00 | 22.85 | 7 | N |
| ATOM | 1356 | CA | MET | C | 25 | 30.719 | 10.545 | 13.862 | 1.00 | 19.77 | 6 | C |
| ATOM | 1357 | CB | MET | C | 25 | 30.066 | 11.120 | 12.615 | 1.00 | 20.26 | 6 | C |
| ATOM | 1358 | CG | MET | C | 25 | 28.610 | 10.697 | 12.416 | 1.00 | 18.66 | 6 | C |
| ATOM | 1359 | SD | MET | C | 25 | 27.853 | 11.632 | 11.049 | 1.00 | 20.09 | 16 | S |
| ATOM | 1360 | CE | MET | C | 25 | 26.186 | 10.959 | 11.067 | 1.00 | 18.76 | 6 | C |
| ATOM | 1361 | C | MET | C | 25 | 32.084 | 11.124 | 14.126 | 1.00 | 20.66 | 6 | C |
| ATOM | 1362 | O | MET | C | 25 | 32.191 | 12.215 | 14.649 | 1.00 | 20.74 | 8 | O |
| ATOM | 1363 | N | PRO | C | 26 | 33.161 | 10.396 | 13.787 | 1.00 | 19.82 | 7 | N |
| ATOM | 1364 | CD | PRO | C | 26 | 33.098 | 9.044 | 13.168 | 1.00 | 20.83 | 6 | C |
| ATOM | 1365 | CA | PRO | C | 26 | 34.498 | 10.816 | 14.024 | 1.00 | 19.76 | 6 | C |
| ATOM | 1366 | CB | PRO | C | 26 | 35.376 | 9.630 | 13.588 | 1.00 | 19.77 | 6 | C |
| ATOM | 1367 | CG | PRO | C | 26 | 34.515 | 8.779 | 12.738 | 1.00 | 20.30 | 6 | C |
| ATOM | 1368 | C | PRO | C | 26 | 34.873 | 12.109 | 13.315 | 1.00 | 17.68 | 6 | C |
| ATOM | 1369 | O | PRO | C | 26 | 35.713 | 12.838 | 13.852 | 1.00 | 17.43 | 8 | O |
| ATOM | 1370 | N | GLU | C | 27 | 34.287 | 12.423 | 12.176 | 1.00 | 17.43 | 7 | N |
| ATOM | 1371 | CA | GLU | C | 27 | 34.610 | 13.635 | 11.418 | 1.00 | 16.36 | 6 | C |
| ATOM | 1372 | CB | GLU | C | 27 | 33.964 | 13.666 | 10.045 | 1.00 | 18.01 | 6 | C |
| ATOM | 1373 | CG | GLU | C | 27 | 33.994 | 12.573 | 9.040 | 1.00 | 21.06 | 6 | C |
| ATOM | 1374 | CD | GLU | C | 27 | 33.460 | 11.209 | 9.439 | 1.00 | 24.48 | 6 | C |
| ATOM | 1375 | OE1 | GLU | C | 27 | 32.905 | 10.975 | 10.544 | 1.00 | 22.13 | 8 | O |
| ATOM | 1376 | OE2 | GLU | C | 27 | 33.612 | 10.328 | 8.548 | 1.00 | 26.95 | 8 | O |
| ATOM | 1377 | C | GLU | C | 27 | 34.072 | 14.894 | 12.132 | 1.00 | 15.77 | 6 | C |
| ATOM | 1378 | O | GLU | C | 27 | 34.608 | 16.018 | 12.001 | 1.00 | 13.07 | 8 | O |
| ATOM | 1379 | N | VAL | C | 28 | 32.985 | 14.693 | 12.889 | 1.00 | 14.39 | 7 | N |
| ATOM | 1380 | CA | VAL | C | 28 | 32.385 | 15.833 | 13.582 | 1.00 | 14.23 | 6 | C |
| ATOM | 1381 | CB | VAL | C | 28 | 30.911 | 15.621 | 13.946 | 1.00 | 13.36 | 6 | C |
| ATOM | 1382 | CG1 | VAL | C | 28 | 30.347 | 16.905 | 14.607 | 1.00 | 13.52 | 6 | C |
| ATOM | 1383 | CG2 | VAL | C | 28 | 30.089 | 15.300 | 12.718 | 1.00 | 11.93 | 6 | C |
| ATOM | 1384 | C | VAL | C | 28 | 33.224 | 16.259 | 14.766 | 1.00 | 14.33 | 6 | C |
| ATOM | 1385 | O | VAL | C | 28 | 33.347 | 15.476 | 15.722 | 1.00 | 15.29 | 8 | O |
| ATOM | 1386 | N | LYS | C | 29 | 33.694 | 17.504 | 14.806 | 1.00 | 13.37 | 7 | N |
| ATOM | 1387 | CA | LYS | C | 29 | 34.438 | 17.954 | 15.988 | 1.00 | 13.98 | 6 | C |
| ATOM | 1388 | CB | LYS | C | 29 | 35.625 | 18.884 | 15.662 | 1.00 | 13.92 | 6 | C |
| ATOM | 1389 | CG | LYS | C | 29 | 36.534 | 18.189 | 14.630 | 1.00 | 16.47 | 6 | C |
| ATOM | 1390 | CD | LYS | C | 29 | 36.875 | 16.761 | 15.038 | 1.00 | 16.96 | 6 | C |
| ATOM | 1391 | CE | LYS | C | 29 | 38.221 | 16.374 | 14.398 | 1.00 | 22.04 | 6 | C |
| ATOM | 1392 | NZ | LYS | C | 29 | 38.262 | 14.911 | 14.030 | 1.00 | 24.30 | 7 | N |
| ATOM | 1393 | C | LYS | C | 29 | 33.640 | 18.713 | 17.033 | 1.00 | 14.44 | 6 | C |
| ATOM | 1394 | O | LYS | C | 29 | 34.018 | 18.680 | 18.202 | 1.00 | 12.53 | 8 | O |
| ATOM | 1395 | N | GLU | C | 30 | 32.583 | 19.419 | 16.575 | 1.00 | 13.94 | 7 | N |
| ATOM | 1396 | CA | GLU | C | 30 | 31.766 | 20.156 | 17.551 | 1.00 | 13.46 | 6 | C |
| ATOM | 1397 | CB | GLU | C | 30 | 32.435 | 21.389 | 18.153 | 1.00 | 14.91 | 6 | C |
| ATOM | 1398 | CG | GLU | C | 30 | 32.868 | 22.376 | 17.121 | 1.00 | 17.59 | 6 | C |
| ATOM | 1399 | CD | GLU | C | 30 | 33.308 | 23.755 | 17.492 | 1.00 | 18.48 | 6 | C |
| ATOM | 1400 | OE1 | GLU | C | 30 | 33.818 | 24.439 | 16.555 | 1.00 | 19.49 | 8 | O |
| ATOM | 1401 | OE2 | GLU | C | 30 | 33.093 | 24.296 | 18.587 | 1.00 | 18.98 | 8 | O |
| ATOM | 1402 | C | GLU | C | 30 | 30.423 | 20.378 | 16.853 | 1.00 | 12.44 | 6 | C |
| ATOM | 1403 | O | GLU | C | 30 | 30.303 | 20.285 | 15.629 | 1.00 | 10.96 | 8 | O |
| ATOM | 1404 | N | ALA | C | 31 | 29.366 | 20.505 | 17.626 | 1.00 | 11.16 | 7 | N |
| ATOM | 1405 | CA | ALA | C | 31 | 28.021 | 20.599 | 17.048 | 1.00 | 10.93 | 6 | C |
| ATOM | 1406 | CB | ALA | C | 31 | 27.428 | 19.201 | 16.822 | 1.00 | 10.63 | 6 | C |
| ATOM | 1407 | C | ALA | C | 31 | 27.190 | 21.315 | 18.113 | 1.00 | 10.97 | 6 | C |
| ATOM | 1408 | O | ALA | C | 31 | 27.360 | 21.054 | 19.316 | 1.00 | 9.38 | 8 | O |
| ATOM | 1409 | N | TYR | C | 32 | 26.391 | 22.275 | 17.649 | 1.00 | 9.53 | 7 | N |
| ATOM | 1410 | CA | TYR | C | 32 | 25.530 | 23.025 | 18.552 | 1.00 | 8.67 | 6 | C |
| ATOM | 1411 | CB | TYR | C | 32 | 26.086 | 24.395 | 18.891 | 1.00 | 9.25 | 6 | C |
| ATOM | 1412 | CG | TYR | C | 32 | 27.408 | 24.481 | 19.615 | 1.00 | 10.08 | 6 | C |
| ATOM | 1413 | CD1 | TYR | C | 32 | 27.357 | 24.671 | 20.988 | 1.00 | 11.88 | 6 | C |
| ATOM | 1414 | CE1 | TYR | C | 32 | 28.503 | 24.740 | 21.794 | 1.00 | 12.28 | 6 | C |
| ATOM | 1415 | CD2 | TYR | C | 32 | 28.646 | 24.324 | 18.992 | 1.00 | 10.95 | 6 | C |
| ATOM | 1416 | CE2 | TYR | C | 32 | 29.799 | 24.403 | 19.764 | 1.00 | 11.49 | 6 | C |
| ATOM | 1417 | CZ | TYR | C | 32 | 29.709 | 24.623 | 21.121 | 1.00 | 12.94 | 6 | C |
| ATOM | 1418 | OH | TYR | C | 32 | 30.827 | 24.777 | 21.921 | 1.00 | 15.26 | 8 | O |
| ATOM | 1419 | C | TYR | C | 32 | 24.204 | 23.305 | 17.843 | 1.00 | 10.59 | 6 | C |
| ATOM | 1420 | O | TYR | C | 32 | 24.121 | 23.444 | 16.603 | 1.00 | 10.55 | 8 | O |
| ATOM | 1421 | N | VAL | C | 33 | 23.162 | 23.336 | 18.661 | 1.00 | 9.72 | 7 | N |
| ATOM | 1422 | CA | VAL | C | 33 | 21.851 | 23.736 | 18.161 | 1.00 | 9.11 | 6 | C |
| ATOM | 1423 | CB | VAL | C | 33 | 20.683 | 23.161 | 18.968 | 1.00 | 9.44 | 6 | C |
| ATOM | 1424 | CG1 | VAL | C | 33 | 19.379 | 23.787 | 18.505 | 1.00 | 11.05 | 6 | C |
| ATOM | 1425 | CG2 | VAL | C | 33 | 20.646 | 21.639 | 18.742 | 1.00 | 9.65 | 6 | C |
| ATOM | 1426 | C | VAL | C | 33 | 21.845 | 25.259 | 18.352 | 1.00 | 8.78 | 6 | C |
| ATOM | 1427 | O | VAL | C | 33 | 22.349 | 25.739 | 19.368 | 1.00 | 11.19 | 8 | O |
| ATOM | 1428 | N | VAL | C | 34 | 21.478 | 26.012 | 17.342 | 1.00 | 8.87 | 7 | N |
| ATOM | 1429 | CA | VAL | C | 34 | 21.543 | 27.472 | 17.359 | 1.00 | 7.42 | 6 | C |
| ATOM | 1430 | CB | VAL | C | 34 | 22.674 | 27.896 | 16.409 | 1.00 | 7.82 | 6 | C |
| ATOM | 1431 | CG1 | VAL | C | 34 | 24.065 | 27.329 | 16.849 | 1.00 | 6.17 | 6 | C |
| ATOM | 1432 | CG2 | VAL | C | 34 | 22.466 | 27.457 | 14.933 | 1.00 | 5.87 | 6 | C |
| ATOM | 1433 | C | VAL | C | 34 | 20.225 | 28.140 | 16.954 | 1.00 | 9.54 | 6 | C |
| ATOM | 1434 | O | VAL | C | 34 | 19.395 | 27.580 | 16.223 | 1.00 | 8.27 | 8 | O |
| ATOM | 1435 | N | TYR | C | 35 | 20.027 | 29.392 | 17.383 | 1.00 | 10.13 | 7 | N |
| ATOM | 1436 | CA | TYR | C | 35 | 18.976 | 30.284 | 16.947 | 1.00 | 11.95 | 6 | C |
| ATOM | 1437 | CB | TYR | C | 35 | 18.775 | 31.483 | 17.876 | 1.00 | 12.68 | 6 | C |
| ATOM | 1438 | CG | TYR | C | 35 | 18.185 | 31.039 | 19.195 | 1.00 | 17.52 | 6 | C |
| ATOM | 1439 | CD1 | TYR | C | 35 | 16.816 | 31.015 | 19.360 | 1.00 | 18.77 | 6 | C |
| ATOM | 1440 | CE1 | TYR | C | 35 | 16.271 | 30.631 | 20.564 | 1.00 | 18.30 | 6 | C |
| ATOM | 1441 | CD2 | TYR | C | 35 | 18.994 | 30.662 | 20.262 | 1.00 | 17.66 | 6 | C |
| ATOM | 1442 | CE2 | TYR | C | 35 | 18.444 | 30.266 | 21.460 | 1.00 | 18.18 | 6 | C |
| ATOM | 1443 | CZ | TYR | C | 35 | 17.067 | 30.261 | 21.601 | 1.00 | 18.43 | 6 | C |
| ATOM | 1444 | OH | TYR | C | 35 | 16.493 | 29.868 | 22.789 | 1.00 | 18.93 | 8 | O |
| ATOM | 1445 | C | TYR | C | 35 | 19.300 | 30.955 | 15.604 | 1.00 | 12.91 | 6 | C |
| ATOM | 1446 | O | TYR | C | 35 | 20.482 | 31.218 | 15.319 | 1.00 | 12.84 | 8 | O |
| ATOM | 1447 | N | GLY | C | 36 | 18.299 | 31.196 | 14.782 | 1.00 | 14.24 | 7 | N |
| ATOM | 1448 | CA | GLY | C | 36 | 18.520 | 31.859 | 13.478 | 1.00 | 12.60 | 6 | C |
| ATOM | 1449 | C | GLY | C | 36 | 17.864 | 31.056 | 12.389 | 1.00 | 14.26 | 6 | C |
| ATOM | 1450 | O | GLY | C | 36 | 17.106 | 30.079 | 12.649 | 1.00 | 18.80 | 8 | O |
| ATOM | 1451 | N | GLU | C | 37 | 18.194 | 31.299 | 11.145 | 1.00 | 18.17 | 7 | N |
| ATOM | 1452 | CA | GLU | C | 37 | 17.633 | 30.564 | 10.002 | 1.00 | 23.09 | 6 | C |
| ATOM | 1453 | CB | GLU | C | 37 | 18.169 | 31.203 | 8.714 | 1.00 | 27.51 | 6 | C |
| ATOM | 1454 | CG | GLU | C | 37 | 17.650 | 30.595 | 7.407 | 1.00 | 32.04 | 6 | C |
| ATOM | 1455 | CD | GLU | C | 37 | 18.546 | 31.061 | 6.256 | 1.00 | 35.48 | 6 | C |
| ATOM | 1456 | OE1 | GLU | C | 37 | 19.323 | 30.257 | 5.686 | 1.00 | 37.55 | 8 | O |
| ATOM | 1457 | OE2 | GLU | C | 37 | 18.521 | 32.254 | 5.909 | 1.00 | 36.51 | 8 | O |
| ATOM | 1458 | C | GLU | C | 37 | 18.066 | 29.104 | 10.001 | 1.00 | 23.63 | 6 | C |
| ATOM | 1459 | O | GLU | C | 37 | 17.322 | 28.198 | 9.618 | 1.00 | 23.98 | 8 | O |
| ATOM | 1460 | N | TYR | C | 38 | 19.316 | 28.847 | 10.403 | 1.00 | 21.56 | 7 | N |
| ATOM | 1461 | CA | TYR | C | 38 | 19.836 | 27.509 | 10.574 | 1.00 | 19.52 | 6 | C |
| ATOM | 1462 | CB | TYR | C | 38 | 21.368 | 27.543 | 10.441 | 1.00 | 22.39 | 6 | C |
| ATOM | 1463 | CG | TYR | C | 38 | 21.703 | 28.026 | 9.039 | 1.00 | 24.37 | 6 | C |
| ATOM | 1464 | CD1 | TYR | C | 38 | 21.292 | 27.258 | 7.969 | 1.00 | 25.06 | 6 | C |
| ATOM | 1465 | CE1 | TYR | C | 38 | 21.584 | 27.690 | 6.693 | 1.00 | 28.43 | 6 | C |
| ATOM | 1466 | CD2 | TYR | C | 38 | 22.404 | 29.209 | 8.861 | 1.00 | 25.04 | 6 | C |
| ATOM | 1467 | CE2 | TYR | C | 38 | 22.726 | 29.599 | 7.572 | 1.00 | 27.79 | 6 | C |
| ATOM | 1468 | CZ | TYR | C | 38 | 22.292 | 28.843 | 6.501 | 1.00 | 27.61 | 6 | C |
| ATOM | 1469 | OH | THR | C | 38 | 22.596 | 29.243 | 5.230 | 1.00 | 31.14 | 8 | O |
| ATOM | 1470 | C | THR | C | 38 | 19.568 | 27.040 | 12.010 | 1.00 | 17.33 | 6 | C |
| ATOM | 1471 | O | THR | C | 38 | 19.308 | 27.808 | 12.915 | 1.00 | 18.03 | 8 | O |
| ATOM | 1472 | N | ASP | C | 39 | 19.479 | 25.752 | 12.164 | 1.00 | 15.50 | 7 | N |
| ATOM | 1473 | CA | ASP | C | 39 | 19.069 | 25.122 | 13.403 | 1.00 | 16.04 | 6 | C |
| ATOM | 1474 | CB | ASP | C | 39 | 18.092 | 24.006 | 13.020 | 1.00 | 14.43 | 6 | C |
| ATOM | 1475 | CG | ASP | C | 39 | 16.840 | 24.566 | 12.407 | 1.00 | 16.60 | 6 | C |
| ATOM | 1476 | OD1 | ASP | C | 39 | 16.453 | 25.687 | 12.752 | 1.00 | 15.98 | 8 | O |
| ATOM | 1477 | OD2 | ASP | C | 39 | 16.250 | 23.864 | 11.558 | 1.00 | 20.27 | 8 | O |
| ATOM | 1478 | C | ASP | C | 39 | 20.282 | 24.495 | 14.086 | 1.00 | 13.53 | 6 | C |
| ATOM | 1479 | O | ASP | C | 39 | 20.326 | 24.344 | 15.307 | 1.00 | 14.94 | 8 | O |
| ATOM | 1480 | N | LEU | C | 40 | 21.232 | 24.154 | 13.194 | 1.00 | 12.61 | 7 | N |
| ATOM | 1481 | CA | LEU | C | 40 | 22.439 | 23.489 | 13.689 | 1.00 | 13.53 | 6 | C |
| ATOM | 1482 | CB | LEU | C | 40 | 22.190 | 22.022 | 13.285 | 1.00 | 15.55 | 6 | C |
| ATOM | 1483 | CG | LEU | C | 40 | 22.710 | 20.831 | 14.001 | 1.00 | 19.18 | 6 | C |
| ATOM | 1484 | CD1 | LEU | C | 40 | 22.457 | 20.879 | 15.503 | 1.00 | 19.65 | 6 | C |
| ATOM | 1485 | CD2 | LEU | C | 40 | 21.942 | 19.557 | 13.514 | 1.00 | 18.15 | 6 | C |
| ATOM | 1486 | C | LEU | C | 40 | 23.716 | 23.928 | 12.995 | 1.00 | 11.96 | 6 | C |
| ATOM | 1487 | O | LEU | C | 40 | 23.749 | 24.157 | 11.769 | 1.00 | 10.54 | 8 | O |
| ATOM | 1488 | N | ILE | C | 41 | 24.785 | 23.932 | 13.784 | 1.00 | 11.74 | 7 | N |
| ATOM | 1489 | CA | ILE | C | 41 | 26.123 | 24.205 | 13.183 | 1.00 | 11.84 | 6 | C |
| ATOM | 1490 | CB | ILE | C | 41 | 26.679 | 25.588 | 13.482 | 1.00 | 12.75 | 6 | C |
| ATOM | 1491 | CG2 | ILE | C | 41 | 26.699 | 25.790 | 14.971 | 1.00 | 13.88 | 6 | C |
| ATOM | 1492 | CG1 | ILE | C | 41 | 28.120 | 25.681 | 12.875 | 1.00 | 13.00 | 6 | C |
| ATOM | 1493 | CD1 | ILE | C | 41 | 28.527 | 27.134 | 12.744 | 1.00 | 14.55 | 6 | C |
| ATOM | 1494 | C | ILE | C | 41 | 27.072 | 23.118 | 13.664 | 1.00 | 10.85 | 6 | C |
| ATOM | 1495 | O | ILE | C | 41 | 27.076 | 22.742 | 14.846 | 1.00 | 10.67 | 8 | O |
| ATOM | 1496 | N | VAL | C | 42 | 27.801 | 22.553 | 12.699 | 1.00 | 10.11 | 7 | N |
| ATOM | 1497 | CA | VAL | C | 42 | 28.738 | 21.454 | 12.964 | 1.00 | 11.25 | 6 | C |
| ATOM | 1498 | CB | VAL | C | 42 | 28.087 | 20.228 | 12.232 | 1.00 | 14.86 | 6 | C |
| ATOM | 1499 | CG1 | VAL | C | 42 | 28.985 | 19.118 | 11.842 | 1.00 | 13.65 | 6 | C |
| ATOM | 1500 | CG2 | VAL | C | 42 | 26.998 | 19.628 | 13.169 | 1.00 | 15.56 | 6 | C |
| ATOM | 1501 | C | VAL | C | 42 | 30.084 | 21.759 | 12.362 | 1.00 | 11.05 | 6 | C |
| ATOM | 1502 | O | VAL | C | 42 | 30.168 | 22.412 | 11.300 | 1.00 | 11.42 | 8 | O |
| ATOM | 1503 | N | LYS | C | 43 | 31.164 | 21.525 | 13.084 | 1.00 | 11.17 | 7 | N |
| ATOM | 1504 | CA | LYS | C | 43 | 32.510 | 21.655 | 12.560 | 1.00 | 10.83 | 6 | C |
| ATOM | 1505 | CB | LYS | C | 43 | 33.423 | 22.361 | 13.542 | 1.00 | 10.74 | 6 | C |
| ATOM | 1506 | CG | LYS | C | 43 | 34.834 | 22.632 | 12.996 | 1.00 | 12.24 | 6 | C |
| ATOM | 1507 | CD | LYS | C | 43 | 35.626 | 22.992 | 14.268 | 1.00 | 15.84 | 6 | C |
| ATOM | 1508 | CE | LYS | C | 43 | 37.101 | 23.230 | 14.123 | 1.00 | 15.74 | 6 | C |
| ATOM | 1509 | NZ | LYS | C | 43 | 37.562 | 23.826 | 15.456 | 1.00 | 14.56 | 7 | N |
| ATOM | 1510 | C | LYS | C | 43 | 33.051 | 20.251 | 12.241 | 1.00 | 11.80 | 6 | C |
| ATOM | 1511 | O | LYS | C | 43 | 33.135 | 19.345 | 13.083 | 1.00 | 10.83 | 8 | O |
| ATOM | 1512 | N | VAL | C | 44 | 33.453 | 20.042 | 10.989 | 1.00 | 11.36 | 7 | N |
| ATOM | 1513 | CA | VAL | C | 44 | 33.934 | 18.777 | 10.453 | 1.00 | 11.94 | 6 | C |
| ATOM | 1514 | CB | VAL | C | 44 | 32.975 | 18.306 | 9.341 | 1.00 | 13.11 | 6 | C |
| ATOM | 1515 | CG1 | VAL | C | 44 | 33.430 | 17.009 | 8.657 | 1.00 | 16.19 | 6 | C |
| ATOM | 1516 | CG2 | VAL | C | 44 | 31.583 | 17.978 | 9.900 | 1.00 | 13.88 | 6 | C |
| ATOM | 1517 | C | VAL | C | 44 | 35.387 | 18.934 | 10.004 | 1.00 | 13.35 | 6 | C |
| ATOM | 1518 | O | VAL | C | 44 | 35.899 | 20.015 | 9.598 | 1.00 | 13.28 | 8 | O |
| ATOM | 1519 | N | GLU | C | 45 | 36.207 | 17.906 | 10.191 | 1.00 | 14.07 | 7 | N |
| ATOM | 1520 | CA | GLU | C | 45 | 37.622 | 17.831 | 9.808 | 1.00 | 14.76 | 6 | C |
| ATOM | 1521 | CB | GLU | C | 45 | 38.680 | 18.063 | 10.878 | 1.00 | 16.30 | 6 | C |
| ATOM | 1522 | CG | GLU | C | 45 | 38.631 | 19.447 | 11.511 | 1.00 | 19.96 | 6 | C |
| ATOM | 1523 | CD | GLU | C | 45 | 39.528 | 19.670 | 12.698 | 1.00 | 22.48 | 6 | C |
| ATOM | 1524 | OE1 | GLU | C | 45 | 39.541 | 20.843 | 13.173 | 1.00 | 25.62 | 8 | O |
| ATOM | 1525 | OE2 | GLU | C | 45 | 40.204 | 18.732 | 13.168 | 1.00 | 22.11 | 8 | O |
| ATOM | 1526 | C | GLU | C | 45 | 37.840 | 16.458 | 9.161 | 1.00 | 16.36 | 6 | C |
| ATOM | 1527 | O | GLU | C | 45 | 37.106 | 15.509 | 9.540 | 1.00 | 15.71 | 8 | O |
| ATOM | 1528 | N | THR | C | 46 | 38.215 | 16.526 | 7.857 | 1.00 | 16.74 | 7 | N |
| ATOM | 1529 | CA | THR | C | 46 | 38.348 | 15.349 | 7.020 | 1.00 | 16.32 | 6 | C |
| ATOM | 1530 | CB | THR | C | 46 | 37.338 | 15.094 | 5.916 | 1.00 | 17.33 | 6 | C |
| ATOM | 1531 | CG1 | THR | C | 46 | 37.480 | 16.090 | 4.875 | 1.00 | 17.34 | 8 | O |
| ATOM | 1532 | CG2 | THR | C | 46 | 35.887 | 15.114 | 6.379 | 1.00 | 18.83 | 6 | C |
| ATOM | 1533 | C | THR | C | 46 | 39.742 | 15.413 | 6.391 | 1.00 | 16.88 | 6 | C |
| ATOM | 1534 | O | THR | C | 46 | 40.446 | 16.425 | 6.502 | 1.00 | 16.93 | 8 | O |
| ATOM | 1535 | N | ASP | C | 47 | 40.290 | 14.231 | 6.023 | 1.00 | 19.87 | 7 | N |
| ATOM | 1536 | CA | ASP | C | 47 | 41.700 | 14.317 | 5.576 | 1.00 | 21.73 | 6 | C |
| ATOM | 1537 | CB | ASP | C | 47 | 42.426 | 12.954 | 5.586 | 1.00 | 26.37 | 6 | C |
| ATOM | 1538 | CG | ASP | C | 47 | 41.716 | 12.020 | 4.630 | 1.00 | 29.72 | 6 | C |
| ATOM | 1539 | OD1 | ASP | C | 47 | 40.591 | 12.393 | 4.229 | 1.00 | 31.09 | 8 | O |
| ATOM | 1540 | OD2 | ASP | C | 47 | 42.171 | 10.932 | 4.213 | 1.00 | 33.95 | 8 | O |
| ATOM | 1541 | C | ASP | C | 47 | 41.738 | 14.992 | 4.194 | 1.00 | 19.55 | 6 | C |
| ATOM | 1542 | O | ASP | C | 47 | 42.634 | 15.814 | 4.039 | 1.00 | 20.55 | 8 | O |
| ATOM | 1543 | N | THR | C | 48 | 40.768 | 14.797 | 3.313 | 1.00 | 17.36 | 7 | N |
| ATOM | 1544 | CA | THR | C | 48 | 40.777 | 15.364 | 1.972 | 1.00 | 16.80 | 6 | C |
| ATOM | 1545 | CB | THR | C | 48 | 40.998 | 14.279 | 0.889 | 1.00 | 17.66 | 6 | C |
| ATOM | 1546 | OG1 | THR | C | 48 | 39.843 | 13.418 | 0.841 | 1.00 | 19.76 | 8 | O |
| ATOM | 1547 | CG2 | THR | C | 48 | 42.226 | 13.399 | 1.066 | 1.00 | 18.46 | 6 | C |
| ATOM | 1548 | C | THR | C | 48 | 39.390 | 15.962 | 1.645 | 1.00 | 17.80 | 6 | C |
| ATOM | 1549 | O | THR | C | 48 | 38.410 | 15.767 | 2.371 | 1.00 | 17.96 | 8 | O |
| ATOM | 1550 | N | LEU | C | 49 | 39.276 | 16.693 | 0.539 | 1.00 | 17.38 | 7 | N |
| ATOM | 1551 | CA | LEU | C | 49 | 38.041 | 17.313 | 0.104 | 1.00 | 17.64 | 6 | C |
| ATOM | 1552 | CB | LEU | C | 49 | 38.373 | 18.361 | −0.982 | 1.00 | 18.40 | 6 | C |
| ATOM | 1553 | CG | LEU | C | 49 | 37.081 | 19.124 | −1.386 | 1.00 | 19.66 | 6 | C |
| ATOM | 1554 | CD1 | LEU | C | 49 | 36.587 | 19.885 | −0.153 | 1.00 | 18.61 | 6 | C |
| ATOM | 1555 | CD2 | LEU | C | 49 | 37.331 | 20.095 | −2.523 | 1.00 | 20.55 | 6 | C |
| ATOM | 1556 | C | LEU | C | 49 | 37.073 | 16.254 | −0.415 | 1.00 | 19.38 | 6 | C |
| ATOM | 1557 | O | LEU | C | 49 | 35.854 | 16.268 | −0.227 | 1.00 | 17.37 | 8 | O |
| ATOM | 1558 | N | LYS | C | 50 | 37.667 | 15.245 | −1.085 | 1.00 | 19.62 | 7 | N |
| ATOM | 1559 | CA | LYS | C | 50 | 36.914 | 14.069 | −1.516 | 1.00 | 22.17 | 6 | C |
| ATOM | 1560 | CB | LYS | C | 50 | 37.782 | 13.055 | −2.251 | 1.00 | 25.37 | 6 | C |
| ATOM | 1561 | CG | LYS | C | 50 | 37.245 | 11.638 | −2.416 | 1.00 | 29.73 | 6 | C |
| ATOM | 1562 | CD | LYS | C | 50 | 38.410 | 10.633 | −2.435 | 1.00 | 32.69 | 6 | C |
| ATOM | 1563 | CE | LYS | C | 50 | 38.020 | 9.201 | −2.769 | 1.00 | 34.00 | 6 | C |
| ATOM | 1564 | NZ | LYS | C | 50 | 38.205 | 8.880 | −4.230 | 1.00 | 36.40 | 7 | N |
| ATOM | 1565 | C | LYS | C | 50 | 36.286 | 13.404 | −0.280 | 1.00 | 19.70 | 6 | C |
| ATOM | 1566 | O | LYS | C | 50 | 35.097 | 13.147 | −0.311 | 1.00 | 18.54 | 8 | O |
| ATOM | 1567 | N | ASP | C | 51 | 37.028 | 13.195 | 0.810 | 1.00 | 20.24 | 7 | N |
| ATOM | 1568 | CA | ASP | C | 51 | 36.392 | 12.664 | 2.031 | 1.00 | 19.47 | 6 | C |
| ATOM | 1569 | CB | ASP | C | 51 | 37.475 | 12.383 | 3.087 | 1.00 | 20.84 | 6 | C |
| ATOM | 1570 | CG | ASP | C | 51 | 38.327 | 11.194 | 2.601 | 1.00 | 22.11 | 6 | C |
| ATOM | 1571 | OD1 | ASP | C | 51 | 37.801 | 10.426 | 1.779 | 1.00 | 22.72 | 8 | O |
| ATOM | 1572 | OD2 | ASP | C | 51 | 39.478 | 11.066 | 3.025 | 1.00 | 20.72 | 8 | O |
| ATOM | 1573 | C | ASP | C | 51 | 35.320 | 13.573 | 2.580 | 1.00 | 19.16 | 6 | C |
| ATOM | 1574 | O | ASP | C | 51 | 34.292 | 13.068 | 3.019 | 1.00 | 21.04 | 8 | O |
| ATOM | 1575 | N | LEU | C | 52 | 35.448 | 14.912 | 2.479 | 1.00 | 18.94 | 7 | N |
| ATOM | 1576 | CA | LEU | C | 52 | 34.381 | 15.799 | 2.945 | 1.00 | 15.62 | 6 | C |
| ATOM | 1577 | CB | LEU | C | 52 | 34.813 | 17.271 | 2.792 | 1.00 | 15.17 | 6 | C |
| ATOM | 1578 | CG | LEU | C | 52 | 33.760 | 18.354 | 3.078 | 1.00 | 12.83 | 6 | C |
| ATOM | 1579 | CD1 | LEU | C | 52 | 33.238 | 18.194 | 4.507 | 1.00 | 14.01 | 6 | C |
| ATOM | 1580 | CD2 | LEU | C | 52 | 34.453 | 19.713 | 2.883 | 1.00 | 13.45 | 6 | C |
| ATOM | 1581 | C | LEU | C | 52 | 33.132 | 15.606 | 2.142 | 1.00 | 16.81 | 6 | C |
| ATOM | 1582 | O | LEU | C | 52 | 32.026 | 15.503 | 2.644 | 1.00 | 16.79 | 8 | O |
| ATOM | 1583 | N | ASP | C | 53 | 33.296 | 15.652 | 0.811 | 1.00 | 20.54 | 7 | N |
| ATOM | 1584 | CA | ASP | C | 53 | 32.156 | 15.528 | −0.100 | 1.00 | 23.66 | 6 | C |
| ATOM | 1585 | CB | ASP | C | 53 | 32.600 | 15.643 | −1.558 | 1.00 | 24.36 | 6 | C |
| ATOM | 1586 | CG | ASP | C | 53 | 31.433 | 15.675 | −2.527 | 1.00 | 27.18 | 6 | C |
| ATOM | 1587 | OD1 | ASP | C | 53 | 30.847 | 14.603 | −2.831 | 1.00 | 28.04 | 8 | O |
| ATOM | 1588 | OD2 | ASP | C | 53 | 31.048 | 16.750 | −3.064 | 1.00 | 28.27 | 8 | O |
| ATOM | 1589 | C | ASP | C | 53 | 31.465 | 14.198 | 0.183 | 1.00 | 24.54 | 6 | C |
| ATOM | 1590 | O | ASP | C | 53 | 30.237 | 14.171 | 0.266 | 1.00 | 27.34 | 8 | O |
| ATOM | 1591 | N | GLN | C | 54 | 32.212 | 13.126 | 0.480 | 1.00 | 25.41 | 7 | N |
| ATOM | 1592 | CA | GLN | C | 54 | 31.528 | 11.856 | 0.704 | 1.00 | 27.72 | 6 | C |
| ATOM | 1593 | CB | GLN | C | 54 | 32.540 | 10.726 | 0.543 | 1.00 | 29.95 | 6 | C |
| ATOM | 1594 | CG | GLN | C | 54 | 31.771 | 9.421 | 0.229 | 1.00 | 35.88 | 6 | C |
| ATOM | 1595 | CD | GLN | C | 54 | 32.776 | 8.260 | 0.234 | 1.00 | 38.59 | 6 | C |
| ATOM | 1596 | OE1 | GLN | C | 54 | 33.997 | 8.535 | 0.208 | 1.00 | 40.18 | 8 | O |
| ATOM | 1597 | NE2 | GLN | C | 54 | 32.222 | 7.057 | 0.258 | 1.00 | 38.68 | 7 | N |
| ATOM | 1598 | C | GLN | C | 54 | 30.861 | 11.758 | 2.073 | 1.00 | 26.83 | 6 | C |
| ATOM | 1599 | O | GLN | C | 54 | 29.910 | 10.973 | 2.244 | 1.00 | 27.40 | 8 | O |
| ATOM | 1600 | N | PHE | C | 55 | 31.350 | 12.565 | 3.019 | 1.00 | 23.70 | 7 | N |
| ATOM | 1601 | CA | PHE | C | 55 | 30.710 | 12.620 | 4.348 | 1.00 | 21.86 | 6 | C |
| ATOM | 1602 | CB | PHE | C | 55 | 31.702 | 13.193 | 5.357 | 1.00 | 19.93 | 6 | C |
| ATOM | 1603 | CG | PHE | C | 55 | 30.987 | 13.609 | 6.614 | 1.00 | 20.88 | 6 | C |
| ATOM | 1604 | CD1 | PHE | C | 55 | 30.884 | 12.742 | 7.683 | 1.00 | 19.06 | 6 | C |
| ATOM | 1605 | CD2 | PHE | C | 55 | 30.394 | 14.873 | 6.690 | 1.00 | 19.80 | 6 | C |
| ATOM | 1606 | CE1 | PHE | C | 55 | 30.209 | 13.115 | 8.823 | 1.00 | 17.49 | 6 | C |
| ATOM | 1607 | CE2 | PHE | C | 55 | 29.715 | 15.249 | 7.830 | 1.00 | 20.56 | 6 | C |
| ATOM | 1608 | CZ | PHE | C | 55 | 29.643 | 14.352 | 8.895 | 1.00 | 19.05 | 6 | C |
| ATOM | 1609 | C | PHE | C | 55 | 29.387 | 13.339 | 4.235 | 1.00 | 21.61 | 6 | C |
| ATOM | 1610 | O | PHE | C | 55 | 28.353 | 12.900 | 4.766 | 1.00 | 22.20 | 8 | O |
| ATOM | 1611 | N | ILE | C | 56 | 29.352 | 14.429 | 3.454 | 1.00 | 21.99 | 7 | N |
| ATOM | 1612 | CA | ILE | C | 56 | 28.092 | 15.134 | 3.220 | 1.00 | 22.47 | 6 | C |
| ATOM | 1613 | CB | ILE | C | 56 | 28.328 | 16.386 | 2.337 | 1.00 | 22.35 | 6 | C |
| ATOM | 1614 | CG2 | ILE | C | 56 | 27.039 | 16.904 | 1.709 | 1.00 | 21.09 | 6 | C |
| ATOM | 1615 | CG1 | ILE | C | 56 | 29.004 | 17.487 | 3.171 | 1.00 | 21.32 | 6 | C |
| ATOM | 1616 | CD1 | ILE | C | 56 | 29.601 | 18.543 | 2.264 | 1.00 | 24.85 | 6 | C |
| ATOM | 1617 | C | ILE | C | 56 | 27.044 | 14.280 | 2.523 | 1.00 | 24.70 | 6 | C |
| ATOM | 1618 | O | ILE | C | 56 | 25.851 | 14.393 | 2.839 | 1.00 | 26.85 | 8 | O |
| ATOM | 1619 | N | THR | C | 57 | 27.403 | 13.577 | 1.446 | 1.00 | 26.48 | 7 | N |
| ATOM | 1620 | CA | THR | C | 57 | 26.407 | 12.847 | 0.667 | 1.00 | 27.16 | 6 | C |
| ATOM | 1621 | CB | THR | C | 57 | 27.007 | 12.425 | −0.687 | 1.00 | 27.37 | 6 | C |
| ATOM | 1622 | OG1 | THR | C | 57 | 28.236 | 11.757 | −0.326 | 1.00 | 30.53 | 8 | O |
| ATOM | 1623 | CG2 | THR | C | 57 | 27.367 | 13.571 | −1.616 | 1.00 | 25.93 | 6 | C |
| ATOM | 1624 | C | THR | C | 57 | 25.977 | 11.580 | 1.383 | 1.00 | 29.79 | 6 | C |
| ATOM | 1625 | O | THR | C | 57 | 24.792 | 11.181 | 1.373 | 1.00 | 32.61 | 8 | O |
| ATOM | 1626 | N | GLU | C | 58 | 26.951 | 10.884 | 1.965 | 1.00 | 30.00 | 7 | N |
| ATOM | 1627 | CA | GLU | C | 58 | 26.591 | 9.627 | 2.622 | 1.00 | 31.50 | 6 | C |
| ATOM | 1628 | CB | GLU | C | 58 | 27.826 | 8.747 | 2.774 | 1.00 | 33.53 | 6 | C |
| ATOM | 1629 | CG | GLU | C | 58 | 28.512 | 8.540 | 1.428 | 1.00 | 36.71 | 6 | C |
| ATOM | 1630 | CD | GLU | C | 58 | 27.648 | 8.445 | 0.182 | 1.00 | 39.49 | 6 | C |
| ATOM | 1631 | OE1 | GLU | C | 58 | 26.556 | 7.813 | 0.197 | 1.00 | 39.86 | 8 | O |
| ATOM | 1632 | OE2 | GLU | C | 58 | 28.074 | 8.990 | −0.885 | 1.00 | 40.22 | 8 | O |
| ATOM | 1633 | C | GLU | C | 58 | 25.848 | 9.921 | 3.911 | 1.00 | 31.88 | 6 | C |
| ATOM | 1634 | O | GLU | C | 58 | 24.713 | 9.432 | 4.067 | 1.00 | 32.24 | 8 | O |
| ATOM | 1635 | N | LYS | C | 59 | 26.476 | 10.754 | 4.754 | 1.00 | 31.30 | 7 | N |
| ATOM | 1636 | CA | LYS | C | 59 | 25.833 | 11.012 | 6.046 | 1.00 | 29.92 | 6 | C |
| ATOM | 1637 | CB | LYS | C | 59 | 26.896 | 11.140 | 7.160 | 1.00 | 30.17 | 6 | C |
| ATOM | 1638 | CG | LYS | C | 59 | 28.102 | 10.256 | 6.978 | 1.00 | 31.15 | 6 | C |
| ATOM | 1639 | CD | LYS | C | 59 | 28.432 | 9.440 | 8.232 | 1.00 | 34.64 | 6 | C |
| ATOM | 1640 | CE | LYS | C | 59 | 29.922 | 9.072 | 8.262 | 1.00 | 35.28 | 6 | C |
| ATOM | 1641 | NZ | LYS | C | 59 | 30.375 | 8.487 | 9.562 | 1.00 | 35.48 | 7 | N |
| ATOM | 1642 | C | LYS | C | 59 | 24.914 | 12.203 | 6.056 | 1.00 | 28.84 | 6 | C |
| ATOM | 1643 | O | LYS | C | 59 | 23.891 | 12.075 | 6.757 | 1.00 | 32.65 | 8 | O |
| ATOM | 1644 | N | ILE | C | 60 | 25.105 | 13.337 | 5.401 | 1.00 | 27.08 | 7 | N |
| ATOM | 1645 | CA | ILE | C | 60 | 24.205 | 14.461 | 5.598 | 1.00 | 24.04 | 6 | C |
| ATOM | 1646 | CB | ILE | C | 60 | 24.917 | 15.824 | 5.491 | 1.00 | 22.38 | 6 | C |
| ATOM | 1647 | CG2 | ILE | C | 60 | 23.980 | 17.028 | 5.513 | 1.00 | 22.26 | 6 | C |
| ATOM | 1648 | CG1 | ILE | C | 60 | 25.945 | 15.987 | 6.614 | 1.00 | 22.82 | 6 | C |
| ATOM | 1649 | CD1 | ILE | C | 60 | 25.479 | 15.800 | 8.043 | 1.00 | 22.99 | 6 | C |
| ATOM | 1650 | C | ILE | C | 60 | 22.982 | 14.420 | 4.717 | 1.00 | 26.78 | 6 | C |
| ATOM | 1651 | O | ILE | C | 60 | 21.885 | 14.876 | 5.126 | 1.00 | 27.54 | 8 | O |
| ATOM | 1652 | N | ARG | C | 61 | 23.097 | 13.874 | 3.518 | 1.00 | 27.90 | 7 | N |
| ATOM | 1653 | CA | ARG | C | 61 | 21.922 | 13.822 | 2.639 | 1.00 | 28.63 | 6 | C |
| ATOM | 1654 | CB | ARG | C | 61 | 22.337 | 14.115 | 1.194 | 1.00 | 28.99 | 6 | C |
| ATOM | 1655 | CG | ARG | C | 61 | 22.193 | 15.571 | 0.776 | 1.00 | 29.41 | 6 | C |
| ATOM | 1656 | CD | ARG | C | 61 | 23.149 | 15.907 | −0.362 | 1.00 | 30.77 | 6 | C |
| ATOM | 1657 | NE | ARG | C | 61 | 23.058 | 17.293 | −0.839 | 1.00 | 30.00 | 7 | N |
| ATOM | 1658 | CZ | ARG | C | 61 | 23.823 | 17.717 | −1.861 | 1.00 | 31.73 | 6 | C |
| ATOM | 1659 | NH1 | ARG | C | 61 | 24.650 | 16.865 | −2.519 | 1.00 | 30.90 | 7 | N |
| ATOM | 1660 | NH2 | ARG | C | 61 | 23.747 | 18.977 | −2.334 | 1.00 | 31.15 | 7 | N |
| ATOM | 1661 | C | ARG | C | 61 | 21.144 | 12.525 | 2.785 | 1.00 | 29.56 | 6 | C |
| ATOM | 1662 | O | ARG | C | 61 | 20.054 | 12.488 | 2.195 | 1.00 | 32.69 | 8 | O |
| ATOM | 1663 | N | LYS | C | 62 | 21.507 | 11.519 | 3.565 | 1.00 | 28.81 | 7 | N |
| ATOM | 1664 | CA | LYS | C | 62 | 20.703 | 10.354 | 3.850 | 1.00 | 28.73 | 6 | C |
| ATOM | 1665 | CB | LYS | C | 62 | 21.495 | 9.038 | 3.876 | 1.00 | 29.41 | 6 | C |
| ATOM | 1666 | CG | LYS | C | 62 | 21.713 | 8.469 | 2.472 | 1.00 | 30.20 | 6 | C |
| ATOM | 1667 | CD | LYS | C | 62 | 22.852 | 7.434 | 2.430 | 1.00 | 32.21 | 6 | C |
| ATOM | 1668 | CE | LYS | C | 62 | 23.356 | 7.366 | 0.974 | 1.00 | 32.81 | 6 | C |
| ATOM | 1669 | NZ | LYS | C | 62 | 24.510 | 6.429 | 0.843 | 1.00 | 33.64 | 7 | N |
| ATOM | 1670 | C | LYS | C | 62 | 20.038 | 10.515 | 5.230 | 1.00 | 30.02 | 6 | C |
| ATOM | 1671 | O | LYS | C | 62 | 19.651 | 9.527 | 5.885 | 1.00 | 32.12 | 8 | O |
| ATOM | 1672 | N | MET | C | 63 | 20.177 | 11.713 | 5.823 | 1.00 | 28.76 | 7 | N |
| ATOM | 1673 | CA | MET | C | 63 | 19.493 | 11.980 | 7.098 | 1.00 | 26.81 | 6 | C |
| ATOM | 1674 | CB | MET | C | 63 | 20.435 | 12.737 | 8.034 | 1.00 | 27.75 | 6 | C |
| ATOM | 1675 | CG | MET | C | 63 | 20.688 | 11.900 | 9.255 | 1.00 | 28.92 | 6 | C |
| ATOM | 1676 | SD | MET | C | 63 | 21.342 | 12.751 | 10.713 | 1.00 | 34.47 | 16 | S |
| ATOM | 1677 | CE | MET | C | 63 | 23.079 | 12.594 | 10.294 | 1.00 | 29.00 | 6 | C |
| ATOM | 1678 | C | MET | C | 63 | 18.231 | 12.718 | 6.738 | 1.00 | 25.17 | 6 | C |
| ATOM | 1679 | O | MET | C | 63 | 18.220 | 13.864 | 6.271 | 1.00 | 27.77 | 8 | O |
| ATOM | 1680 | N | PRO | C | 64 | 17.097 | 12.006 | 6.766 | 1.00 | 23.96 | 7 | N |
| ATOM | 1681 | CD | PRO | C | 64 | 16.986 | 10.619 | 7.289 | 1.00 | 24.17 | 6 | C |
| ATOM | 1682 | CA | PRO | C | 64 | 15.808 | 12.538 | 6.355 | 1.00 | 24.05 | 6 | C |
| ATOM | 1683 | CB | PRO | C | 64 | 14.878 | 11.345 | 6.357 | 1.00 | 24.26 | 6 | C |
| ATOM | 1684 | CG | PRO | C | 64 | 15.491 | 10.380 | 7.310 | 1.00 | 24.02 | 6 | C |
| ATOM | 1685 | C | PRO | C | 64 | 15.351 | 13.693 | 7.238 | 1.00 | 23.26 | 6 | C |
| ATOM | 1686 | O | PRO | C | 64 | 14.634 | 14.599 | 6.786 | 1.00 | 23.48 | 8 | O |
| ATOM | 1687 | N | GLU | C | 65 | 15.894 | 13.773 | 8.458 | 1.00 | 23.82 | 7 | N |
| ATOM | 1688 | CA | GLU | C | 65 | 15.503 | 14.946 | 9.287 | 1.00 | 25.38 | 6 | C |
| ATOM | 1689 | CB | GLU | C | 65 | 15.737 | 14.529 | 10.742 | 1.00 | 26.46 | 6 | C |
| ATOM | 1690 | CG | GLU | C | 65 | 14.861 | 13.328 | 11.046 | 1.00 | 29.88 | 6 | C |
| ATOM | 1691 | CD | GLU | C | 65 | 15.581 | 12.403 | 11.996 | 1.00 | 33.43 | 6 | C |
| ATOM | 1692 | OE1 | GLU | C | 65 | 14.905 | 11.607 | 12.707 | 1.00 | 36.06 | 8 | O |
| ATOM | 1693 | OE2 | GLU | C | 65 | 16.826 | 12.495 | 12.168 | 1.00 | 34.58 | 8 | O |
| ATOM | 1694 | C | GLU | C | 65 | 16.224 | 16.228 | 8.933 | 1.00 | 25.09 | 6 | C |
| ATOM | 1695 | O | GLU | C | 65 | 15.716 | 17.320 | 9.257 | 1.00 | 25.89 | 8 | O |
| ATOM | 1696 | N | ILE | C | 66 | 17.369 | 16.098 | 8.233 | 1.00 | 23.79 | 7 | N |
| ATOM | 1697 | CA | ILE | C | 66 | 18.156 | 17.256 | 7.814 | 1.00 | 22.74 | 6 | C |
| ATOM | 1698 | CB | ILE | C | 66 | 19.635 | 16.857 | 7.670 | 1.00 | 22.13 | 6 | C |
| ATOM | 1699 | CG2 | ILE | C | 66 | 20.468 | 17.984 | 7.086 | 1.00 | 22.33 | 6 | C |
| ATOM | 1700 | CG1 | ILE | C | 66 | 20.165 | 16.412 | 9.012 | 1.00 | 20.58 | 6 | C |
| ATOM | 1701 | CD1 | ILE | C | 66 | 21.611 | 15.995 | 9.067 | 1.00 | 23.30 | 6 | C |
| ATOM | 1702 | C | ILE | C | 66 | 17.578 | 17.729 | 6.511 | 1.00 | 24.00 | 6 | C |
| ATOM | 1703 | O | ILE | C | 66 | 17.301 | 16.854 | 5.665 | 1.00 | 26.96 | 8 | O |
| ATOM | 1704 | N | GLN | C | 67 | 17.361 | 19.007 | 6.283 | 1.00 | 25.25 | 7 | N |
| ATOM | 1705 | CA | GLN | C | 67 | 16.593 | 19.488 | 5.154 | 1.00 | 25.62 | 6 | C |
| ATOM | 1706 | CB | GLN | C | 67 | 15.371 | 20.251 | 5.750 | 1.00 | 27.64 | 6 | C |
| ATOM | 1707 | CG | GLN | C | 67 | 14.314 | 19.213 | 6.197 | 1.00 | 30.98 | 6 | C |
| ATOM | 1708 | CD | GLN | C | 67 | 13.082 | 19.895 | 6.738 | 1.00 | 32.31 | 6 | C |
| ATOM | 1709 | OE1 | GLN | C | 67 | 12.934 | 21.113 | 6.514 | 1.00 | 35.41 | 8 | O |
| ATOM | 1710 | NE2 | GLN | C | 67 | 12.264 | 19.145 | 7.471 | 1.00 | 33.33 | 7 | N |
| ATOM | 1711 | C | GLN | C | 67 | 17.276 | 20.434 | 4.200 | 1.00 | 28.28 | 6 | C |
| ATOM | 1712 | O | GLN | C | 67 | 16.880 | 20.489 | 3.003 | 1.00 | 30.74 | 8 | O |
| ATOM | 1713 | N | MET | C | 68 | 18.156 | 21.310 | 4.658 | 1.00 | 27.47 | 7 | N |
| ATOM | 1714 | CA | MET | C | 68 | 18.862 | 22.263 | 3.802 | 1.00 | 24.68 | 6 | C |
| ATOM | 1715 | CB | MET | C | 68 | 18.008 | 23.515 | 3.700 | 1.00 | 26.70 | 6 | C |
| ATOM | 1716 | CG | MET | C | 68 | 18.697 | 24.745 | 3.118 | 1.00 | 31.25 | 6 | C |
| ATOM | 1717 | SD | MET | C | 68 | 17.815 | 26.277 | 3.584 | 1.00 | 38.51 | 16 | S |
| ATOM | 1718 | CE | MET | C | 68 | 19.212 | 27.404 | 3.757 | 1.00 | 35.34 | 6 | C |
| ATOM | 1719 | C | MET | C | 68 | 20.189 | 22.645 | 4.477 | 1.00 | 23.75 | 6 | C |
| ATOM | 1720 | O | MET | C | 68 | 20.099 | 23.063 | 5.644 | 1.00 | 19.66 | 8 | O |
| ATOM | 1721 | N | THR | C | 69 | 21.313 | 22.411 | 3.789 | 1.00 | 21.36 | 7 | N |
| ATOM | 1722 | CA | THR | C | 69 | 22.618 | 22.639 | 4.357 | 1.00 | 19.81 | 6 | C |
| ATOM | 1723 | CB | THR | C | 69 | 23.404 | 21.315 | 4.525 | 1.00 | 20.43 | 6 | C |
| ATOM | 1724 | OG1 | THR | C | 69 | 24.167 | 21.019 | 3.362 | 1.00 | 22.61 | 8 | O |
| ATOM | 1725 | CG2 | THR | C | 69 | 22.506 | 20.131 | 4.834 | 1.00 | 17.73 | 6 | C |
| ATOM | 1726 | C | THR | C | 69 | 23.490 | 23.637 | 3.618 | 1.00 | 18.75 | 6 | C |
| ATOM | 1727 | O | THR | C | 69 | 23.440 | 23.819 | 2.409 | 1.00 | 20.19 | 8 | O |
| ATOM | 1728 | N | SER | C | 70 | 24.295 | 24.391 | 4.359 | 1.00 | 16.80 | 7 | N |
| ATOM | 1729 | CA | SER | C | 70 | 25.258 | 25.283 | 3.740 | 1.00 | 17.93 | 6 | C |
| ATOM | 1730 | CB | SER | C | 70 | 24.713 | 26.687 | 3.927 | 1.00 | 17.55 | 6 | C |
| ATOM | 1731 | OG | SER | C | 70 | 25.650 | 27.578 | 3.348 | 1.00 | 22.26 | 8 | O |
| ATOM | 1732 | C | SER | C | 70 | 26.657 | 24.999 | 4.302 | 1.00 | 17.09 | 6 | C |
| ATOM | 1733 | O | SER | C | 70 | 26.847 | 25.050 | 5.520 | 1.00 | 17.01 | 8 | O |
| ATOM | 1734 | N | THR | C | 71 | 27.600 | 24.667 | 3.423 | 1.00 | 14.94 | 7 | N |
| ATOM | 1735 | CA | THR | C | 71 | 28.959 | 24.328 | 3.889 | 1.00 | 15.34 | 6 | C |
| ATOM | 1736 | CB | THR | C | 71 | 29.491 | 23.036 | 3.200 | 1.00 | 15.88 | 6 | C |
| ATOM | 1737 | OG1 | THR | C | 71 | 28.576 | 21.980 | 3.539 | 1.00 | 15.96 | 8 | O |
| ATOM | 1738 | CG2 | THR | C | 71 | 30.856 | 22.573 | 3.655 | 1.00 | 16.01 | 6 | C |
| ATOM | 1739 | C | THR | C | 71 | 29.906 | 25.472 | 3.588 | 1.00 | 14.07 | 6 | C |
| ATOM | 1740 | O | THR | C | 71 | 29.908 | 26.005 | 2.467 | 1.00 | 13.13 | 8 | O |
| ATOM | 1741 | N | MET | C | 72 | 30.733 | 25.786 | 4.576 | 1.00 | 12.04 | 7 | N |
| ATOM | 1742 | CA | MET | C | 72 | 31.806 | 26.752 | 4.438 | 1.00 | 12.02 | 6 | C |
| ATOM | 1743 | CB | MET | C | 72 | 31.612 | 27.963 | 5.351 | 1.00 | 11.60 | 6 | C |
| ATOM | 1744 | CG | MET | C | 72 | 30.249 | 28.638 | 5.153 | 1.00 | 17.54 | 6 | C |
| ATOM | 1745 | SD | MET | C | 72 | 29.862 | 29.742 | 6.517 | 1.00 | 18.74 | 16 | S |
| ATOM | 1746 | CE | MET | C | 72 | 28.292 | 30.398 | 6.028 | 1.00 | 21.26 | 6 | C |
| ATOM | 1747 | C | MET | C | 72 | 33.168 | 26.143 | 4.823 | 1.00 | 11.23 | 6 | C |
| ATOM | 1748 | O | MET | C | 72 | 33.471 | 25.902 | 6.015 | 1.00 | 8.34 | 8 | O |
| ATOM | 1749 | N | ILE | C | 73 | 33.996 | 25.961 | 3.813 | 1.00 | 10.73 | 7 | N |
| ATOM | 1750 | CA | ILE | C | 73 | 35.331 | 25.404 | 4.056 | 1.00 | 10.84 | 6 | C |
| ATOM | 1751 | CB | ILE | C | 73 | 35.953 | 24.995 | 2.723 | 1.00 | 13.41 | 6 | C |
| ATOM | 1752 | CG2 | ILE | C | 73 | 37.443 | 24.649 | 2.885 | 1.00 | 12.03 | 6 | C |
| ATOM | 1753 | CG1 | ILE | C | 73 | 35.105 | 23.853 | 2.157 | 1.00 | 14.14 | 6 | C |
| ATOM | 1754 | CD1 | ILE | C | 73 | 35.758 | 23.179 | 0.954 | 1.00 | 16.09 | 6 | C |
| ATOM | 1755 | C | ILE | C | 73 | 36.203 | 26.456 | 4.725 | 1.00 | 13.62 | 6 | C |
| ATOM | 1756 | O | ILE | C | 73 | 36.216 | 27.633 | 4.359 | 1.00 | 13.19 | 8 | O |
| ATOM | 1757 | N | ALA | C | 74 | 36.934 | 26.033 | 5.735 | 1.00 | 14.74 | 7 | N |
| ATOM | 1758 | CA | ALA | C | 74 | 37.896 | 26.893 | 6.398 | 1.00 | 18.25 | 6 | C |
| ATOM | 1759 | CB | ALA | C | 74 | 37.993 | 26.485 | 7.858 | 1.00 | 18.16 | 6 | C |
| ATOM | 1760 | C | ALA | C | 74 | 39.226 | 26.939 | 5.669 | 1.00 | 22.37 | 6 | C |
| ATOM | 1761 | O | ALA | C | 74 | 39.598 | 26.057 | 4.867 | 1.00 | 24.32 | 8 | O |
| ATOM | 1762 | N | ILE | C | 75 | 39.770 | 28.140 | 5.533 | 1.00 | 24.13 | 7 | N |
| ATOM | 1763 | CA | ILE | C | 75 | 41.072 | 28.446 | 4.996 | 1.00 | 26.43 | 6 | C |
| ATOM | 1764 | CB | ILE | C | 75 | 41.185 | 29.580 | 3.977 | 1.00 | 25.92 | 6 | C |
| ATOM | 1765 | CG2 | ILE | C | 75 | 42.611 | 29.746 | 3.459 | 1.00 | 25.06 | 6 | C |
| ATOM | 1766 | CG1 | ILE | C | 75 | 40.232 | 29.433 | 2.812 | 1.00 | 25.87 | 6 | C |
| ATOM | 1767 | CD1 | ILE | C | 75 | 39.600 | 30.788 | 2.473 | 1.00 | 25.99 | 6 | C |
| ATOM | 1768 | C | ILE | C | 75 | 41.832 | 28.964 | 6.239 | 1.00 | 29.95 | 6 | C |
| ATOM | 1769 | O | ILE | C | 75 | 41.645 | 28.455 | 7.393 | 1.00 | 29.32 | 8 | O |
| ATOM | 1770 | OT | ILE | C | 75 | 42.313 | 30.128 | 6.106 | 1.00 | 33.07 | 8 | O |
| ATOM | 1771 | CB | VAL | D | 2 | 25.991 | 33.212 | 26.292 | 1.00 | 10.82 | 6 | C |
| ATOM | 1772 | CG1 | VAL | D | 2 | 25.191 | 33.260 | 24.963 | 1.00 | 10.54 | 6 | C |
| ATOM | 1773 | CG2 | VAL | D | 2 | 24.998 | 33.476 | 27.435 | 1.00 | 11.04 | 6 | C |
| ATOM | 1774 | C | VAL | D | 2 | 27.631 | 31.627 | 25.373 | 1.00 | 9.57 | 6 | C |
| ATOM | 1775 | O | VAL | D | 2 | 28.621 | 32.340 | 25.380 | 1.00 | 12.23 | 8 | O |
| ATOM | 1776 | N | VAL | D | 2 | 27.227 | 31.645 | 27.864 | 1.00 | 11.94 | 7 | N |
| ATOM | 1777 | CA | VAL | D | 2 | 26.629 | 31.841 | 26.486 | 1.00 | 10.20 | 6 | C |
| ATOM | 1778 | N | THR | D | 3 | 27.387 | 30.772 | 24.418 | 1.00 | 10.51 | 7 | N |
| ATOM | 1779 | CA | THR | D | 3 | 28.201 | 30.539 | 23.233 | 1.00 | 12.03 | 6 | C |
| ATOM | 1780 | CB | THR | D | 3 | 28.465 | 29.034 | 23.075 | 1.00 | 12.43 | 6 | C |
| ATOM | 1781 | OG1 | THR | D | 3 | 29.142 | 28.582 | 24.249 | 1.00 | 13.41 | 8 | O |
| ATOM | 1782 | CG2 | THR | D | 3 | 29.325 | 28.600 | 21.892 | 1.00 | 9.36 | 6 | C |
| ATOM | 1783 | C | THR | D | 3 | 27.453 | 31.041 | 21.984 | 1.00 | 10.73 | 6 | C |
| ATOM | 1784 | O | THR | D | 3 | 26.224 | 30.920 | 21.844 | 1.00 | 8.60 | 8 | O |
| ATOM | 1785 | N | ALA | D | 4 | 28.180 | 31.725 | 21.110 | 1.00 | 8.65 | 7 | N |
| ATOM | 1786 | CA | ALA | D | 4 | 27.522 | 32.150 | 19.858 | 1.00 | 7.98 | 6 | C |
| ATOM | 1787 | CB | ALA | D | 4 | 27.122 | 33.625 | 20.025 | 1.00 | 6.69 | 6 | C |
| ATOM | 1788 | C | ALA | D | 4 | 28.519 | 31.981 | 18.712 | 1.00 | 8.86 | 6 | C |
| ATOM | 1789 | O | ALA | D | 4 | 29.734 | 32.022 | 18.997 | 1.00 | 6.46 | 8 | O |
| ATOM | 1790 | N | PHE | D | 5 | 28.015 | 31.833 | 17.488 | 1.00 | 6.65 | 7 | N |
| ATOM | 1791 | CA | PHE | D | 5 | 28.883 | 31.817 | 16.321 | 1.00 | 6.21 | 6 | C |
| ATOM | 1792 | CB | PHE | D | 5 | 28.691 | 30.573 | 15.466 | 1.00 | 6.58 | 6 | C |
| ATOM | 1793 | CG | PHE | D | 5 | 29.300 | 29.326 | 16.045 | 1.00 | 6.64 | 6 | C |
| ATOM | 1794 | CD1 | PHE | D | 5 | 28.596 | 28.550 | 16.929 | 1.00 | 8.03 | 6 | C |
| ATOM | 1795 | CD2 | PHE | D | 5 | 30.594 | 28.964 | 15.709 | 1.00 | 8.47 | 6 | C |
| ATOM | 1796 | CE1 | PHE | D | 5 | 29.137 | 27.383 | 17.485 | 1.00 | 8.72 | 6 | C |
| ATOM | 1797 | CE2 | PHE | D | 5 | 31.157 | 27.771 | 16.245 | 1.00 | 9.33 | 6 | C |
| ATOM | 1798 | CZ | PHE | D | 5 | 30.417 | 27.024 | 17.108 | 1.00 | 9.51 | 6 | C |
| ATOM | 1799 | C | PHE | D | 5 | 28.529 | 33.046 | 15.478 | 1.00 | 8.92 | 6 | C |
| ATOM | 1800 | O | PHE | D | 5 | 27.347 | 33.369 | 15.226 | 1.00 | 7.43 | 8 | O |
| ATOM | 1801 | N | ILE | D | 6 | 29.571 | 33.778 | 15.043 | 1.00 | 6.33 | 7 | N |
| ATOM | 1802 | CA | ILE | D | 6 | 29.321 | 35.020 | 14.346 | 1.00 | 5.35 | 6 | C |
| ATOM | 1803 | CB | ILE | D | 6 | 29.968 | 36.242 | 14.991 | 1.00 | 7.05 | 6 | C |
| ATOM | 1804 | CG2 | ILE | D | 6 | 29.512 | 37.544 | 14.306 | 1.00 | 6.83 | 6 | C |
| ATOM | 1805 | CG1 | ILE | D | 6 | 29.682 | 36.312 | 16.498 | 1.00 | 9.71 | 6 | C |
| ATOM | 1806 | CD1 | ILE | D | 6 | 30.583 | 37.312 | 17.230 | 1.00 | 10.38 | 6 | C |
| ATOM | 1807 | C | ILE | D | 6 | 29.965 | 34.952 | 12.940 | 1.00 | 7.08 | 6 | C |
| ATOM | 1808 | O | ILE | D | 6 | 31.167 | 34.685 | 12.840 | 1.00 | 6.63 | 8 | O |
| ATOM | 1809 | N | LEU | D | 7 | 29.158 | 35.223 | 11.940 | 1.00 | 6.66 | 7 | N |
| ATOM | 1810 | CA | LEU | D | 7 | 29.569 | 35.267 | 10.534 | 1.00 | 8.26 | 6 | C |
| ATOM | 1811 | CB | LEU | D | 7 | 28.490 | 34.694 | 9.625 | 1.00 | 9.39 | 6 | C |
| ATOM | 1812 | CG | LEU | D | 7 | 28.278 | 33.182 | 9.719 | 1.00 | 12.12 | 6 | C |
| ATOM | 1813 | CD1 | LEU | D | 7 | 26.964 | 32.819 | 9.001 | 1.00 | 12.30 | 6 | C |
| ATOM | 1814 | CD2 | LEU | D | 7 | 29.452 | 32.442 | 9.113 | 1.00 | 12.62 | 6 | C |
| ATOM | 1815 | C | LEU | D | 7 | 29.753 | 36.727 | 10.173 | 1.00 | 9.44 | 6 | C |
| ATOM | 1816 | O | LEU | D | 7 | 28.969 | 37.612 | 10.588 | 1.00 | 8.72 | 8 | O |
| ATOM | 1817 | N | MET | D | 8 | 30.883 | 37.069 | 9.557 | 1.00 | 7.27 | 7 | N |
| ATOM | 1818 | CA | MET | D | 8 | 31.248 | 38.430 | 9.267 | 1.00 | 7.95 | 6 | C |
| ATOM | 1819 | CB | MET | D | 8 | 32.497 | 38.770 | 10.154 | 1.00 | 9.16 | 6 | C |
| ATOM | 1820 | CG | MET | D | 8 | 32.240 | 38.575 | 11.637 | 1.00 | 12.23 | 6 | C |
| ATOM | 1821 | SD | MET | D | 8 | 33.642 | 39.025 | 12.668 | 1.00 | 9.99 | 16 | S |
| ATOM | 1822 | CE | MET | D | 8 | 33.323 | 38.190 | 14.198 | 1.00 | 15.38 | 6 | C |
| ATOM | 1823 | C | MET | D | 8 | 31.695 | 38.640 | 7.819 | 1.00 | 9.51 | 6 | C |
| ATOM | 1824 | O | MET | D | 8 | 32.466 | 37.818 | 7.278 | 1.00 | 7.50 | 8 | O |
| ATOM | 1825 | N | VAL | D | 9 | 31.387 | 39.829 | 7.340 | 1.00 | 8.02 | 7 | N |
| ATOM | 1826 | CA | VAL | D | 9 | 31.905 | 40.303 | 6.047 | 1.00 | 10.82 | 6 | C |
| ATOM | 1827 | CB | VAL | D | 9 | 30.854 | 40.707 | 5.000 | 1.00 | 11.20 | 6 | C |
| ATOM | 1828 | CG1 | VAL | D | 9 | 31.500 | 41.169 | 3.666 | 1.00 | 11.05 | 6 | C |
| ATOM | 1829 | CG2 | VAL | D | 9 | 29.940 | 39.525 | 4.650 | 1.00 | 10.58 | 6 | C |
| ATOM | 1830 | C | VAL | D | 9 | 32.812 | 41.482 | 6.401 | 1.00 | 10.74 | 6 | C |
| ATOM | 1831 | O | VAL | D | 9 | 32.330 | 42.332 | 7.162 | 1.00 | 11.16 | 8 | O |
| ATOM | 1832 | N | THR | D | 10 | 34.049 | 41.503 | 5.908 | 1.00 | 8.55 | 7 | N |
| ATOM | 1833 | CA | THR | D | 10 | 34.950 | 42.605 | 6.235 | 1.00 | 9.63 | 6 | C |
| ATOM | 1834 | CB | THR | D | 10 | 36.316 | 42.145 | 6.788 | 1.00 | 7.41 | 6 | C |
| ATOM | 1835 | OG1 | THR | D | 10 | 37.064 | 41.489 | 5.731 | 1.00 | 7.90 | 8 | O |
| ATOM | 1836 | CG2 | THR | D | 10 | 36.134 | 41.119 | 7.892 | 1.00 | 6.20 | 6 | C |
| ATOM | 1837 | C | THR | D | 10 | 35.214 | 43.436 | 4.976 | 1.00 | 10.07 | 6 | C |
| ATOM | 1838 | O | THR | D | 10 | 34.887 | 42.902 | 3.935 | 1.00 | 5.93 | 8 | O |
| ATOM | 1839 | N | ALA | D | 11 | 35.781 | 44.632 | 5.043 | 1.00 | 8.15 | 7 | N |
| ATOM | 1840 | CA | ALA | D | 11 | 36.193 | 45.341 | 3.862 | 1.00 | 10.45 | 6 | C |
| ATOM | 1841 | CB | ALA | D | 11 | 36.773 | 46.737 | 4.229 | 1.00 | 9.59 | 6 | C |
| ATOM | 1842 | C | ALA | D | 11 | 37.261 | 44.555 | 3.115 | 1.00 | 11.75 | 6 | C |
| ATOM | 1843 | O | ALA | D | 11 | 38.008 | 43.789 | 3.744 | 1.00 | 11.01 | 8 | O |
| ATOM | 1844 | N | ALA | D | 12 | 37.428 | 44.805 | 1.813 | 1.00 | 11.13 | 7 | N |
| ATOM | 1845 | CA | ALA | D | 12 | 38.439 | 44.106 | 1.030 | 1.00 | 11.80 | 6 | C |
| ATOM | 1846 | CB | ALA | D | 12 | 38.456 | 44.611 | −0.421 | 1.00 | 11.09 | 6 | C |
| ATOM | 1847 | C | ALA | D | 12 | 39.838 | 44.330 | 1.596 | 1.00 | 10.46 | 6 | C |
| ATOM | 1848 | O | ALA | D | 12 | 40.159 | 45.459 | 1.986 | 1.00 | 8.93 | 8 | O |
| ATOM | 1849 | N | GLY | D | 13 | 40.632 | 43.273 | 1.670 | 1.00 | 11.63 | 7 | N |
| ATOM | 1850 | CA | GLY | D | 13 | 42.018 | 43.352 | 2.113 | 1.00 | 9.40 | 6 | C |
| ATOM | 1851 | C | GLY | D | 13 | 42.048 | 43.563 | 3.634 | 1.00 | 10.22 | 6 | C |
| ATOM | 1852 | O | GLY | D | 13 | 43.122 | 43.919 | 4.107 | 1.00 | 7.69 | 8 | O |
| ATOM | 1853 | N | LYS | D | 14 | 40.965 | 43.319 | 4.365 | 1.00 | 8.00 | 7 | N |
| ATOM | 1854 | CA | LYS | D | 14 | 41.015 | 43.549 | 5.807 | 1.00 | 10.05 | 6 | C |
| ATOM | 1855 | CB | LYS | D | 14 | 39.991 | 44.631 | 6.218 | 1.00 | 11.34 | 6 | C |
| ATOM | 1856 | CG | LYS | D | 14 | 40.323 | 46.070 | 5.765 | 1.00 | 13.42 | 6 | C |
| ATOM | 1857 | CD | LYS | D | 14 | 41.685 | 46.502 | 6.343 | 1.00 | 15.94 | 6 | C |
| ATOM | 1858 | CE | LYS | D | 14 | 41.973 | 47.965 | 6.014 | 1.00 | 17.06 | 6 | C |
| ATOM | 1859 | NZ | LYS | D | 14 | 43.258 | 48.433 | 6.607 | 1.00 | 15.63 | 7 | N |
| ATOM | 1860 | C | LYS | D | 14 | 40.739 | 42.306 | 6.612 | 1.00 | 8.73 | 6 | C |
| ATOM | 1861 | O | LYS | D | 14 | 40.729 | 42.391 | 7.849 | 1.00 | 8.72 | 8 | O |
| ATOM | 1862 | N | GLU | D | 15 | 40.392 | 41.175 | 5.995 | 1.00 | 8.24 | 7 | N |
| ATOM | 1863 | CA | GLU | D | 15 | 40.019 | 40.018 | 6.817 | 1.00 | 11.30 | 6 | C |
| ATOM | 1864 | CB | GLU | D | 15 | 39.552 | 38.840 | 6.006 | 1.00 | 12.74 | 6 | C |
| ATOM | 1865 | CG | GLU | D | 15 | 40.569 | 38.210 | 5.063 | 1.00 | 13.46 | 6 | C |
| ATOM | 1866 | CD | GLU | D | 15 | 39.892 | 37.220 | 4.102 | 1.00 | 15.23 | 6 | C |
| ATOM | 1867 | OE1 | GLU | D | 15 | 38.702 | 36.868 | 4.120 | 1.00 | 10.38 | 8 | O |
| ATOM | 1868 | OE2 | GLU | D | 15 | 40.658 | 36.700 | 3.250 | 1.00 | 17.69 | 8 | O |
| ATOM | 1869 | C | GLU | D | 15 | 41.152 | 39.617 | 7.758 | 1.00 | 11.05 | 6 | C |
| ATOM | 1870 | O | GLU | D | 15 | 40.864 | 39.297 | 8.907 | 1.00 | 11.86 | 8 | O |
| ATOM | 1871 | N | ARG | D | 16 | 42.382 | 39.633 | 7.311 | 1.00 | 11.96 | 7 | N |
| ATOM | 1872 | CA | ARG | D | 16 | 43.472 | 39.262 | 8.208 | 1.00 | 14.89 | 6 | C |
| ATOM | 1873 | CB | ARG | D | 16 | 44.805 | 39.220 | 7.437 | 1.00 | 17.84 | 6 | C |
| ATOM | 1874 | CG | ARG | D | 16 | 45.966 | 39.007 | 8.388 | 1.00 | 25.50 | 6 | C |
| ATOM | 1875 | CD | ARG | D | 16 | 47.263 | 38.621 | 7.649 | 1.00 | 31.98 | 6 | C |
| ATOM | 1876 | NE | ARG | D | 16 | 48.220 | 38.168 | 8.650 | 1.00 | 37.59 | 7 | N |
| ATOM | 1877 | CZ | ARG | D | 16 | 49.449 | 37.704 | 8.576 | 1.00 | 40.87 | 6 | C |
| ATOM | 1878 | NH1 | ARG | D | 16 | 50.073 | 37.573 | 7.404 | 1.00 | 43.35 | 7 | N |
| ATOM | 1879 | NH2 | ARG | D | 16 | 50.078 | 37.359 | 9.694 | 1.00 | 41.61 | 7 | N |
| ATOM | 1880 | C | ARG | D | 16 | 43.580 | 40.265 | 9.363 | 1.00 | 11.57 | 6 | C |
| ATOM | 1881 | O | ARG | D | 16 | 43.846 | 39.856 | 10.471 | 1.00 | 7.54 | 8 | O |
| ATOM | 1882 | N | GLU | D | 17 | 43.509 | 41.574 | 9.058 | 1.00 | 8.70 | 7 | N |
| ATOM | 1883 | CA | GLU | D | 17 | 43.520 | 42.590 | 10.104 | 1.00 | 11.16 | 6 | C |
| ATOM | 1884 | CB | GLU | D | 17 | 43.356 | 43.975 | 9.464 | 1.00 | 10.95 | 6 | C |
| ATOM | 1885 | CG | GLU | D | 17 | 43.321 | 45.161 | 10.442 | 1.00 | 11.37 | 6 | C |
| ATOM | 1886 | CD | GLU | D | 17 | 43.336 | 46.423 | 9.614 | 1.00 | 14.08 | 6 | C |
| ATOM | 1887 | OE1 | GLU | D | 17 | 44.230 | 46.626 | 8.769 | 1.00 | 11.49 | 8 | O |
| ATOM | 1888 | OE2 | GLU | D | 17 | 42.391 | 47.252 | 9.756 | 1.00 | 14.65 | 8 | O |
| ATOM | 1889 | C | GLU | D | 17 | 42.415 | 42.345 | 11.131 | 1.00 | 10.84 | 6 | C |
| ATOM | 1890 | O | GLU | D | 17 | 42.610 | 42.536 | 12.360 | 1.00 | 9.09 | 8 | O |
| ATOM | 1891 | N | VAL | D | 18 | 41.222 | 41.917 | 10.678 | 1.00 | 9.00 | 7 | N |
| ATOM | 1892 | CA | VAL | D | 18 | 40.148 | 41.641 | 11.625 | 1.00 | 9.27 | 6 | C |
| ATOM | 1893 | CB | VAL | D | 18 | 38.804 | 41.488 | 10.876 | 1.00 | 9.98 | 6 | C |
| ATOM | 1894 | CG1 | VAL | D | 18 | 37.657 | 41.058 | 11.792 | 1.00 | 9.93 | 6 | C |
| ATOM | 1895 | CG2 | VAL | D | 18 | 38.445 | 42.837 | 10.240 | 1.00 | 8.46 | 6 | C |
| ATOM | 1896 | C | VAL | D | 18 | 40.467 | 40.420 | 12.475 | 1.00 | 8.32 | 6 | C |
| ATOM | 1897 | O | VAL | D | 18 | 40.313 | 40.366 | 13.706 | 1.00 | 6.46 | 8 | O |
| ATOM | 1898 | N | MET | D | 19 | 40.966 | 39.375 | 11.776 | 1.00 | 5.78 | 7 | N |
| ATOM | 1899 | CA | MET | D | 19 | 41.370 | 38.173 | 12.470 | 1.00 | 7.84 | 6 | C |
| ATOM | 1900 | CB | MET | D | 19 | 42.012 | 37.108 | 11.544 | 1.00 | 7.18 | 6 | C |
| ATOM | 1901 | CG | MET | D | 19 | 40.955 | 36.423 | 10.674 | 1.00 | 10.79 | 6 | C |
| ATOM | 1902 | SD | MET | D | 19 | 40.066 | 35.123 | 11.568 | 1.00 | 9.43 | 16 | S |
| ATOM | 1903 | CE | MET | D | 19 | 41.320 | 33.849 | 11.585 | 1.00 | 13.35 | 6 | C |
| ATOM | 1904 | C | MET | D | 19 | 42.380 | 38.513 | 13.580 | 1.00 | 8.79 | 6 | C |
| ATOM | 1905 | O | MET | D | 19 | 42.398 | 37.821 | 14.587 | 1.00 | 6.86 | 8 | O |
| ATOM | 1906 | N | GLU | D | 20 | 43.327 | 39.417 | 13.289 | 1.00 | 8.74 | 7 | N |
| ATOM | 1907 | CA | GLU | D | 20 | 44.322 | 39.799 | 14.331 | 1.00 | 12.83 | 6 | C |
| ATOM | 1908 | CB | GLU | D | 20 | 45.435 | 40.642 | 13.699 | 1.00 | 11.30 | 6 | C |
| ATOM | 1909 | CG | GLU | D | 20 | 46.357 | 39.657 | 12.930 | 1.00 | 13.63 | 6 | C |
| ATOM | 1910 | CD | GLU | D | 20 | 47.519 | 40.364 | 12.253 | 1.00 | 17.21 | 6 | C |
| ATOM | 1911 | OE1 | GLU | D | 20 | 47.597 | 41.620 | 12.258 | 1.00 | 17.52 | 8 | O |
| ATOM | 1912 | OE2 | GLU | D | 20 | 48.352 | 39.656 | 11.622 | 1.00 | 17.62 | 8 | O |
| ATOM | 1913 | C | GLU | D | 20 | 43.639 | 40.472 | 15.520 | 1.00 | 12.42 | 6 | C |
| ATOM | 1914 | O | GLU | D | 20 | 43.980 | 40.207 | 16.673 | 1.00 | 13.73 | 8 | O |
| ATOM | 1915 | N | LYS | D | 21 | 42.617 | 41.275 | 15.271 | 1.00 | 10.66 | 7 | N |
| ATOM | 1916 | CA | LYS | D | 21 | 41.854 | 41.867 | 16.365 | 1.00 | 11.11 | 6 | C |
| ATOM | 1917 | CB | LYS | D | 21 | 40.878 | 42.958 | 15.892 | 1.00 | 10.31 | 6 | C |
| ATOM | 1918 | CG | LYS | D | 21 | 41.568 | 44.254 | 15.563 | 1.00 | 11.75 | 6 | C |
| ATOM | 1919 | CD | LYS | D | 21 | 40.551 | 45.276 | 15.062 | 1.00 | 12.69 | 6 | C |
| ATOM | 1920 | CE | LYS | D | 21 | 41.190 | 46.632 | 14.741 | 1.00 | 14.75 | 6 | C |
| ATOM | 1921 | NZ | LYS | D | 21 | 40.107 | 47.556 | 14.250 | 1.00 | 12.59 | 7 | N |
| ATOM | 1922 | C | LYS | D | 21 | 41.115 | 40.792 | 17.136 | 1.00 | 8.78 | 6 | C |
| ATOM | 1923 | O | LYS | D | 21 | 41.229 | 40.694 | 18.372 | 1.00 | 10.75 | 8 | O |
| ATOM | 1924 | N | LEU | D | 22 | 40.465 | 39.846 | 16.472 | 1.00 | 10.46 | 7 | N |
| ATOM | 1925 | CA | LEU | D | 22 | 39.667 | 38.802 | 17.134 | 1.00 | 8.87 | 6 | C |
| ATOM | 1926 | CB | LEU | D | 22 | 38.920 | 37.980 | 16.061 | 1.00 | 8.01 | 6 | C |
| ATOM | 1927 | CG | LEU | D | 22 | 37.883 | 38.808 | 15.271 | 1.00 | 10.13 | 6 | C |
| ATOM | 1928 | CD1 | LEU | D | 22 | 37.464 | 38.033 | 14.021 | 1.00 | 9.62 | 6 | C |
| ATOM | 1929 | CD2 | LEU | D | 22 | 36.664 | 39.137 | 16.170 | 1.00 | 8.70 | 6 | C |
| ATOM | 1930 | C | LEU | D | 22 | 40.536 | 37.837 | 17.936 | 1.00 | 10.00 | 6 | C |
| ATOM | 1931 | O | LEU | D | 22 | 40.171 | 37.498 | 19.077 | 1.00 | 7.47 | 8 | O |
| ATOM | 1932 | N | LEU | D | 23 | 41.707 | 37.413 | 17.394 | 1.00 | 7.02 | 7 | N |
| ATOM | 1933 | CA | LEU | D | 23 | 42.521 | 36.497 | 18.203 | 1.00 | 10.23 | 6 | C |
| ATOM | 1934 | CB | LEU | D | 23 | 43.459 | 35.688 | 17.309 | 1.00 | 11.12 | 6 | C |
| ATOM | 1935 | CG | LEU | D | 23 | 42.767 | 34.912 | 16.180 | 1.00 | 13.04 | 6 | C |
| ATOM | 1936 | CD1 | LEU | D | 23 | 43.728 | 34.528 | 15.057 | 1.00 | 11.71 | 6 | C |
| ATOM | 1937 | CD2 | LEU | D | 23 | 42.107 | 33.666 | 16.753 | 1.00 | 12.37 | 6 | C |
| ATOM | 1938 | C | LEU | D | 23 | 43.234 | 37.115 | 19.399 | 1.00 | 10.47 | 6 | C |
| ATOM | 1939 | O | LEU | D | 23 | 43.943 | 36.413 | 20.157 | 1.00 | 10.67 | 8 | O |
| ATOM | 1940 | N | ALA | D | 24 | 43.032 | 38.376 | 19.741 | 1.00 | 8.71 | 7 | N |
| ATOM | 1941 | CA | ALA | D | 24 | 43.504 | 39.013 | 20.928 | 1.00 | 10.88 | 6 | C |
| ATOM | 1942 | CB | ALA | D | 24 | 44.146 | 40.395 | 20.663 | 1.00 | 10.61 | 6 | C |
| ATOM | 1943 | C | ALA | D | 24 | 42.298 | 39.250 | 21.861 | 1.00 | 12.75 | 6 | C |
| ATOM | 1944 | O | ALA | D | 24 | 42.581 | 39.689 | 22.961 | 1.00 | 11.80 | 8 | O |
| ATOM | 1945 | N | MET | D | 25 | 41.070 | 38.842 | 21.482 | 1.00 | 10.78 | 7 | N |
| ATOM | 1946 | CA | MET | D | 25 | 39.984 | 39.163 | 22.443 | 1.00 | 12.47 | 6 | C |
| ATOM | 1947 | CB | MET | D | 25 | 38.724 | 39.513 | 21.657 | 1.00 | 11.87 | 6 | C |
| ATOM | 1948 | CG | MET | D | 25 | 38.837 | 40.708 | 20.724 | 1.00 | 9.43 | 6 | C |
| ATOM | 1949 | SD | MET | D | 25 | 37.279 | 40.909 | 19.751 | 1.00 | 7.93 | 16 | S |
| ATOM | 1950 | CE | MET | D | 25 | 37.656 | 42.472 | 18.986 | 1.00 | 8.17 | 6 | C |
| ATOM | 1951 | C | MET | D | 25 | 39.756 | 37.981 | 23.364 | 1.00 | 10.74 | 6 | C |
| ATOM | 1952 | O | MET | D | 25 | 39.689 | 36.838 | 22.907 | 1.00 | 10.38 | 8 | O |
| ATOM | 1953 | N | PRO | D | 26 | 39.638 | 38.173 | 24.662 | 1.00 | 10.43 | 7 | N |
| ATOM | 1954 | CD | PRO | D | 26 | 39.570 | 39.495 | 25.327 | 1.00 | 11.37 | 6 | C |
| ATOM | 1955 | CA | PRO | D | 26 | 39.297 | 37.068 | 25.552 | 1.00 | 10.14 | 6 | C |
| ATOM | 1956 | CB | PRO | D | 26 | 38.977 | 37.760 | 26.908 | 1.00 | 11.47 | 6 | C |
| ATOM | 1957 | CG | PRO | D | 26 | 39.571 | 39.129 | 26.804 | 1.00 | 13.17 | 6 | C |
| ATOM | 1958 | C | PRO | D | 26 | 38.094 | 36.224 | 25.147 | 1.00 | 8.78 | 6 | C |
| ATOM | 1959 | O | PRO | D | 26 | 38.113 | 34.961 | 25.329 | 1.00 | 7.44 | 8 | O |
| ATOM | 1960 | N | GLU | D | 27 | 37.067 | 36.856 | 24.599 | 1.00 | 7.06 | 7 | N |
| ATOM | 1961 | CA | GLU | D | 27 | 35.809 | 36.171 | 24.302 | 1.00 | 9.61 | 6 | C |
| ATOM | 1962 | CB | GLU | D | 27 | 34.693 | 37.232 | 24.074 | 1.00 | 10.96 | 6 | C |
| ATOM | 1963 | CG | GLU | D | 27 | 34.496 | 38.142 | 25.290 | 1.00 | 13.96 | 6 | C |
| ATOM | 1964 | CD | GLU | D | 27 | 35.375 | 39.387 | 25.273 | 1.00 | 15.97 | 6 | C |
| ATOM | 1965 | OE1 | GLU | D | 27 | 36.379 | 39.456 | 24.532 | 1.00 | 13.92 | 8 | O |
| ATOM | 1966 | OE2 | GLU | D | 27 | 34.937 | 40.411 | 25.876 | 1.00 | 16.96 | 8 | O |
| ATOM | 1967 | C | GLU | D | 27 | 35.818 | 35.290 | 23.070 | 1.00 | 8.19 | 6 | C |
| ATOM | 1968 | O | GLU | D | 27 | 35.104 | 34.293 | 23.034 | 1.00 | 6.20 | 8 | O |
| ATOM | 1969 | N | VAL | D | 28 | 36.862 | 35.512 | 22.223 | 1.00 | 8.44 | 7 | N |
| ATOM | 1970 | CA | VAL | D | 28 | 36.933 | 34.688 | 20.998 | 1.00 | 7.24 | 6 | C |
| ATOM | 1971 | CB | VAL | D | 28 | 37.735 | 35.371 | 19.882 | 1.00 | 3.76 | 6 | C |
| ATOM | 1972 | CG1 | VAL | D | 28 | 38.115 | 34.476 | 18.758 | 1.00 | 3.75 | 6 | C |
| ATOM | 1973 | CG2 | VAL | D | 28 | 36.991 | 36.648 | 19.370 | 1.00 | 3.95 | 6 | C |
| ATOM | 1974 | C | VAL | D | 28 | 37.547 | 33.354 | 21.308 | 1.00 | 9.27 | 6 | C |
| ATOM | 1975 | O | VAL | D | 28 | 38.709 | 33.330 | 21.734 | 1.00 | 9.19 | 8 | O |
| ATOM | 1976 | N | LYS | D | 29 | 36.868 | 32.228 | 21.065 | 1.00 | 9.77 | 7 | N |
| ATOM | 1977 | CA | LYS | D | 29 | 37.439 | 30.911 | 21.290 | 1.00 | 10.82 | 6 | C |
| ATOM | 1978 | CB | LYS | D | 29 | 36.354 | 29.989 | 21.860 | 1.00 | 12.93 | 6 | C |
| ATOM | 1979 | CG | LYS | D | 29 | 35.708 | 30.606 | 23.097 | 1.00 | 15.22 | 6 | C |
| ATOM | 1980 | CD | LYS | D | 29 | 36.573 | 30.622 | 24.356 | 1.00 | 16.50 | 6 | C |
| ATOM | 1981 | CE | LYS | D | 29 | 36.919 | 32.063 | 24.674 | 1.00 | 18.62 | 6 | C |
| ATOM | 1982 | NZ | LYS | D | 29 | 37.533 | 32.336 | 26.009 | 1.00 | 20.84 | 7 | N |
| ATOM | 1983 | C | LYS | D | 29 | 38.023 | 30.242 | 20.062 | 1.00 | 11.20 | 6 | C |
| ATOM | 1984 | O | LYS | D | 29 | 38.928 | 29.399 | 20.213 | 1.00 | 10.74 | 8 | O |
| ATOM | 1985 | N | GLU | D | 30 | 37.527 | 30.591 | 18.876 | 1.00 | 10.26 | 7 | N |
| ATOM | 1986 | CA | GLU | D | 30 | 38.068 | 30.038 | 17.625 | 1.00 | 10.81 | 6 | C |
| ATOM | 1987 | CB | GLU | D | 30 | 37.655 | 28.615 | 17.277 | 1.00 | 13.90 | 6 | C |
| ATOM | 1988 | CG | GLU | D | 30 | 36.198 | 28.375 | 16.965 | 1.00 | 18.66 | 6 | C |
| ATOM | 1989 | CD | GLU | D | 30 | 35.861 | 26.876 | 16.789 | 1.00 | 20.56 | 6 | C |
| ATOM | 1990 | OE1 | GLU | D | 30 | 36.678 | 26.028 | 16.448 | 1.00 | 17.38 | 8 | O |
| ATOM | 1991 | OE2 | GLU | D | 30 | 34.684 | 26.508 | 16.956 | 1.00 | 21.70 | 8 | O |
| ATOM | 1992 | C | GLU | D | 30 | 37.675 | 31.011 | 16.512 | 1.00 | 10.06 | 6 | C |
| ATOM | 1993 | O | GLU | D | 30 | 36.609 | 31.646 | 16.617 | 1.00 | 7.37 | 8 | O |
| ATOM | 1994 | N | ALA | D | 31 | 38.502 | 31.177 | 15.507 | 1.00 | 8.02 | 7 | N |
| ATOM | 1995 | CA | ALA | D | 31 | 38.200 | 32.119 | 14.421 | 1.00 | 7.12 | 6 | C |
| ATOM | 1996 | CB | ALA | D | 31 | 38.807 | 33.489 | 14.683 | 1.00 | 8.42 | 6 | C |
| ATOM | 1997 | C | ALA | D | 31 | 38.745 | 31.565 | 13.117 | 1.00 | 8.88 | 6 | C |
| ATOM | 1998 | O | ALA | D | 31 | 39.884 | 31.020 | 13.144 | 1.00 | 8.65 | 8 | O |
| ATOM | 1999 | N | TYR | D | 32 | 37.999 | 31.613 | 12.001 | 1.00 | 5.43 | 7 | N |
| ATOM | 2000 | CA | TYR | D | 32 | 38.546 | 31.045 | 10.796 | 1.00 | 6.36 | 6 | C |
| ATOM | 2001 | CB | TYR | D | 32 | 37.990 | 29.674 | 10.391 | 1.00 | 8.15 | 6 | C |
| ATOM | 2002 | CG | TYR | D | 32 | 38.426 | 28.515 | 11.256 | 1.00 | 10.64 | 6 | C |
| ATOM | 2003 | CD1 | TYR | D | 32 | 39.545 | 27.765 | 10.930 | 1.00 | 13.77 | 6 | C |
| ATOM | 2004 | CE1 | TYR | D | 32 | 39.920 | 26.682 | 11.711 | 1.00 | 15.51 | 6 | C |
| ATOM | 2005 | CD2 | TYR | D | 32 | 37.754 | 28.227 | 12.419 | 1.00 | 11.24 | 6 | C |
| ATOM | 2006 | CE2 | TYR | D | 32 | 38.115 | 27.173 | 13.217 | 1.00 | 12.74 | 6 | C |
| ATOM | 2007 | CZ | TYR | D | 32 | 39.201 | 26.419 | 12.861 | 1.00 | 14.10 | 6 | C |
| ATOM | 2008 | OH | TYR | D | 32 | 39.533 | 25.377 | 13.684 | 1.00 | 16.30 | 8 | O |
| ATOM | 2009 | C | TYR | D | 32 | 38.154 | 31.873 | 9.560 | 1.00 | 7.98 | 6 | C |
| ATOM | 2010 | O | TYR | D | 32 | 37.040 | 32.416 | 9.586 | 1.00 | 7.94 | 8 | O |
| ATOM | 2011 | N | VAL | D | 33 | 39.074 | 31.877 | 8.611 | 1.00 | 6.38 | 7 | N |
| ATOM | 2012 | CA | VAL | D | 33 | 38.758 | 32.536 | 7.348 | 1.00 | 7.52 | 6 | C |
| ATOM | 2013 | CB | VAL | D | 33 | 39.981 | 32.990 | 6.536 | 1.00 | 8.07 | 6 | C |
| ATOM | 2014 | CG1 | VAL | D | 33 | 39.567 | 33.517 | 5.162 | 1.00 | 6.28 | 6 | C |
| ATOM | 2015 | CG2 | VAL | D | 33 | 40.723 | 34.076 | 7.330 | 1.00 | 7.68 | 6 | C |
| ATOM | 2016 | C | VAL | D | 33 | 37.968 | 31.475 | 6.595 | 1.00 | 7.23 | 6 | C |
| ATOM | 2017 | O | VAL | D | 33 | 38.398 | 30.299 | 6.681 | 1.00 | 10.00 | 8 | O |
| ATOM | 2018 | N | VAL | D | 34 | 36.784 | 31.773 | 6.055 | 1.00 | 6.32 | 7 | N |
| ATOM | 2019 | CA | VAL | D | 34 | 36.024 | 30.715 | 5.372 | 1.00 | 6.20 | 6 | C |
| ATOM | 2020 | CB | VAL | D | 34 | 34.708 | 30.380 | 6.088 | 1.00 | 8.45 | 6 | C |
| ATOM | 2021 | CG1 | VAL | D | 34 | 34.870 | 29.568 | 7.387 | 1.00 | 8.52 | 6 | C |
| ATOM | 2022 | CG2 | VAL | D | 34 | 33.920 | 31.673 | 6.419 | 1.00 | 8.05 | 6 | C |
| ATOM | 2023 | C | VAL | D | 34 | 35.687 | 31.195 | 3.959 | 1.00 | 8.31 | 6 | C |
| ATOM | 2024 | O | VAL | D | 34 | 35.649 | 32.430 | 3.697 | 1.00 | 8.44 | 8 | O |
| ATOM | 2025 | N | TYR | D | 35 | 35.506 | 30.287 | 3.043 | 1.00 | 7.60 | 7 | N |
| ATOM | 2026 | CA | TYR | D | 35 | 35.035 | 30.540 | 1.693 | 1.00 | 10.70 | 6 | C |
| ATOM | 2027 | CB | TYR | D | 35 | 34.992 | 29.145 | 0.966 | 1.00 | 13.94 | 6 | C |
| ATOM | 2028 | CG | TYR | D | 35 | 36.287 | 28.648 | 0.406 | 1.00 | 19.41 | 6 | C |
| ATOM | 2029 | CD1 | TYR | D | 35 | 36.500 | 28.632 | −0.976 | 1.00 | 21.68 | 6 | C |
| ATOM | 2030 | CE1 | TYR | D | 35 | 37.708 | 28.179 | −1.499 | 1.00 | 23.69 | 6 | C |
| ATOM | 2031 | CD2 | TYR | D | 35 | 37.334 | 28.196 | 1.232 | 1.00 | 21.93 | 6 | C |
| ATOM | 2032 | CE2 | TYR | D | 35 | 38.533 | 27.739 | 0.695 | 1.00 | 21.21 | 6 | C |
| ATOM | 2033 | CZ | TYR | D | 35 | 38.714 | 27.728 | −0.672 | 1.00 | 23.19 | 6 | C |
| ATOM | 2034 | OH | TYR | D | 35 | 39.907 | 27.295 | −1.250 | 1.00 | 23.55 | 8 | O |
| ATOM | 2035 | C | TYR | D | 35 | 33.570 | 31.004 | 1.702 | 1.00 | 9.18 | 6 | C |
| ATOM | 2036 | O | TYR | D | 35 | 32.887 | 30.788 | 2.719 | 1.00 | 7.05 | 8 | O |
| ATOM | 2037 | N | GLY | D | 36 | 33.114 | 31.550 | 0.583 | 1.00 | 9.23 | 7 | N |
| ATOM | 2038 | CA | GLY | D | 36 | 31.703 | 31.882 | 0.420 | 1.00 | 8.59 | 6 | C |
| ATOM | 2039 | C | GLY | D | 36 | 31.335 | 33.330 | 0.669 | 1.00 | 10.03 | 6 | C |
| ATOM | 2040 | O | GLY | D | 36 | 32.197 | 34.227 | 0.709 | 1.00 | 8.03 | 8 | O |
| ATOM | 2041 | N | GLU | D | 37 | 30.038 | 33.530 | 0.994 | 1.00 | 9.60 | 7 | N |
| ATOM | 2042 | CA | GLU | D | 37 | 29.553 | 34.874 | 1.190 | 1.00 | 13.84 | 6 | C |
| ATOM | 2043 | CB | GLU | D | 37 | 28.042 | 34.888 | 1.222 | 1.00 | 20.20 | 6 | C |
| ATOM | 2044 | CG | GLU | D | 37 | 27.302 | 34.244 | 2.338 | 1.00 | 26.34 | 6 | C |
| ATOM | 2045 | CD | GLU | D | 37 | 25.826 | 34.649 | 2.298 | 1.00 | 29.65 | 6 | C |
| ATOM | 2046 | OE1 | GLU | D | 37 | 24.966 | 33.760 | 2.335 | 1.00 | 33.05 | 8 | O |
| ATOM | 2047 | OE2 | GLU | D | 37 | 25.505 | 35.844 | 2.279 | 1.00 | 32.09 | 8 | O |
| ATOM | 2048 | C | GLU | D | 37 | 30.156 | 35.589 | 2.393 | 1.00 | 13.26 | 6 | C |
| ATOM | 2049 | O | GLU | D | 37 | 30.257 | 36.818 | 2.316 | 1.00 | 11.83 | 8 | O |
| ATOM | 2050 | N | TYR | D | 38 | 30.463 | 34.895 | 3.458 | 1.00 | 11.24 | 7 | N |
| ATOM | 2051 | CA | TYR | D | 38 | 31.113 | 35.525 | 4.609 | 1.00 | 13.22 | 6 | C |
| ATOM | 2052 | CB | TYR | D | 38 | 30.602 | 34.920 | 5.924 | 1.00 | 14.41 | 6 | C |
| ATOM | 2053 | CG | TYR | D | 38 | 29.125 | 35.241 | 6.086 | 1.00 | 17.44 | 6 | C |
| ATOM | 2054 | CD1 | TYR | D | 38 | 28.751 | 36.473 | 6.607 | 1.00 | 19.66 | 6 | C |
| ATOM | 2055 | CE1 | TYR | D | 38 | 27.404 | 36.793 | 6.741 | 1.00 | 22.87 | 6 | C |
| ATOM | 2056 | CD2 | TYR | D | 38 | 28.164 | 34.332 | 5.690 | 1.00 | 19.08 | 6 | C |
| ATOM | 2057 | CE2 | TYR | D | 38 | 26.806 | 34.627 | 5.817 | 1.00 | 21.26 | 6 | C |
| ATOM | 2058 | CZ | TYR | D | 38 | 26.460 | 35.851 | 6.360 | 1.00 | 23.15 | 6 | C |
| ATOM | 2059 | OH | TYR | D | 38 | 25.146 | 36.171 | 6.515 | 1.00 | 25.72 | 8 | O |
| ATOM | 2060 | C | TYR | D | 38 | 32.632 | 35.321 | 4.547 | 1.00 | 12.41 | 6 | C |
| ATOM | 2061 | O | TYR | D | 38 | 33.089 | 34.307 | 4.013 | 1.00 | 8.37 | 8 | O |
| ATOM | 2062 | N | ASP | D | 39 | 33.400 | 36.289 | 5.061 | 1.00 | 11.84 | 7 | N |
| ATOM | 2063 | CA | ASP | D | 39 | 34.846 | 36.127 | 5.064 | 1.00 | 10.07 | 6 | C |
| ATOM | 2064 | CB | ASP | D | 39 | 35.517 | 37.526 | 5.140 | 1.00 | 12.25 | 6 | C |
| ATOM | 2065 | CG | ASP | D | 39 | 35.115 | 38.291 | 3.866 | 1.00 | 12.99 | 6 | C |
| ATOM | 2066 | OD1 | ASP | D | 39 | 35.289 | 37.666 | 2.802 | 1.00 | 11.90 | 8 | O |
| ATOM | 2067 | OD2 | ASP | D | 39 | 34.602 | 39.408 | 3.976 | 1.00 | 12.15 | 8 | O |
| ATOM | 2068 | C | ASP | D | 39 | 35.285 | 35.299 | 6.253 | 1.00 | 8.98 | 6 | C |
| ATOM | 2069 | O | ASP | D | 39 | 36.213 | 34.516 | 6.139 | 1.00 | 9.58 | 8 | O |
| ATOM | 2070 | N | LEU | D | 40 | 34.658 | 35.524 | 7.416 | 1.00 | 5.50 | 7 | N |
| ATOM | 2071 | CA | LEU | D | 40 | 35.088 | 34.829 | 8.633 | 1.00 | 7.68 | 6 | C |
| ATOM | 2072 | CB | LEU | D | 40 | 35.715 | 35.942 | 9.546 | 1.00 | 8.52 | 6 | C |
| ATOM | 2073 | CG | LEU | D | 40 | 36.837 | 36.793 | 9.009 | 1.00 | 14.09 | 6 | C |
| ATOM | 2074 | CD1 | LEU | D | 40 | 37.168 | 37.996 | 9.919 | 1.00 | 13.28 | 6 | C |
| ATOM | 2075 | CD2 | LEU | D | 40 | 38.110 | 35.938 | 8.801 | 1.00 | 14.05 | 6 | C |
| ATOM | 2076 | C | LEU | D | 40 | 33.952 | 34.236 | 9.461 | 1.00 | 7.29 | 6 | C |
| ATOM | 2077 | O | LEU | D | 40 | 32.834 | 34.770 | 9.431 | 1.00 | 7.50 | 8 | O |
| ATOM | 2078 | N | ILE | D | 41 | 34.225 | 33.291 | 10.308 | 1.00 | 6.52 | 7 | N |
| ATOM | 2079 | CA | ILE | D | 41 | 33.347 | 32.659 | 11.251 | 1.00 | 8.65 | 6 | C |
| ATOM | 2080 | CB | ILE | D | 41 | 32.757 | 31.294 | 10.866 | 1.00 | 7.09 | 6 | C |
| ATOM | 2081 | CG2 | ILE | D | 41 | 33.763 | 30.136 | 10.738 | 1.00 | 7.76 | 6 | C |
| ATOM | 2082 | CG1 | ILE | D | 41 | 31.714 | 30.864 | 11.933 | 1.00 | 7.44 | 6 | C |
| ATOM | 2083 | CD1 | ILE | D | 41 | 30.882 | 29.692 | 11.430 | 1.00 | 8.47 | 6 | C |
| ATOM | 2084 | C | ILE | D | 41 | 34.095 | 32.701 | 12.586 | 1.00 | 10.10 | 6 | C |
| ATOM | 2085 | O | ILE | D | 41 | 35.293 | 32.408 | 12.685 | 1.00 | 9.03 | 8 | O |
| ATOM | 2086 | N | VAL | D | 42 | 33.391 | 33.059 | 13.669 | 1.00 | 10.09 | 7 | N |
| ATOM | 2087 | CA | VAL | D | 42 | 34.073 | 33.170 | 14.978 | 1.00 | 10.73 | 6 | C |
| ATOM | 2088 | CB | VAL | D | 42 | 34.404 | 34.614 | 15.388 | 1.00 | 13.00 | 6 | C |
| ATOM | 2089 | CG1 | VAL | D | 42 | 33.245 | 35.362 | 16.063 | 1.00 | 14.99 | 6 | C |
| ATOM | 2090 | CG2 | VAL | D | 42 | 35.471 | 34.830 | 16.425 | 1.00 | 12.34 | 6 | C |
| ATOM | 2091 | C | VAL | D | 42 | 33.159 | 32.540 | 16.008 | 1.00 | 9.22 | 6 | C |
| ATOM | 2092 | O | VAL | D | 42 | 31.939 | 32.782 | 15.940 | 1.00 | 9.43 | 8 | O |
| ATOM | 2093 | N | LYS | D | 43 | 33.739 | 31.804 | 16.934 | 1.00 | 6.57 | 7 | N |
| ATOM | 2094 | CA | LYS | D | 43 | 32.976 | 31.281 | 18.049 | 1.00 | 7.49 | 6 | C |
| ATOM | 2095 | CB | LYS | D | 43 | 33.242 | 29.814 | 18.298 | 1.00 | 8.66 | 6 | C |
| ATOM | 2096 | CG | LYS | D | 43 | 32.509 | 29.240 | 19.502 | 1.00 | 10.98 | 6 | C |
| ATOM | 2097 | CD | LYS | D | 43 | 32.987 | 27.756 | 19.527 | 1.00 | 12.07 | 6 | C |
| ATOM | 2098 | CE | LYS | D | 43 | 32.765 | 27.099 | 20.873 | 1.00 | 14.03 | 6 | C |
| ATOM | 2099 | NZ | LYS | D | 43 | 33.327 | 25.695 | 20.768 | 1.00 | 12.83 | 7 | N |
| ATOM | 2100 | C | LYS | D | 43 | 33.370 | 32.072 | 19.277 | 1.00 | 7.78 | 6 | C |
| ATOM | 2101 | O | LYS | D | 43 | 34.586 | 32.291 | 19.497 | 1.00 | 8.73 | 8 | O |
| ATOM | 2102 | N | VAL | D | 44 | 32.387 | 32.621 | 19.972 | 1.00 | 8.73 | 7 | N |
| ATOM | 2103 | CA | VAL | D | 44 | 32.629 | 33.468 | 21.129 | 1.00 | 7.44 | 6 | C |
| ATOM | 2104 | CB | VAL | D | 44 | 32.310 | 34.941 | 20.905 | 1.00 | 8.98 | 6 | C |
| ATOM | 2105 | CG1 | VAL | D | 44 | 33.072 | 35.522 | 19.705 | 1.00 | 7.87 | 6 | C |
| ATOM | 2106 | CG2 | VAL | D | 44 | 30.806 | 35.154 | 20.649 | 1.00 | 8.94 | 6 | C |
| ATOM | 2107 | C | VAL | D | 44 | 31.798 | 32.956 | 22.304 | 1.00 | 9.01 | 6 | C |
| ATOM | 2108 | O | VAL | D | 44 | 30.837 | 32.209 | 22.117 | 1.00 | 9.60 | 8 | O |
| ATOM | 2109 | N | GLU | D | 45 | 32.254 | 33.197 | 23.525 | 1.00 | 9.31 | 7 | N |
| ATOM | 2110 | CA | GLU | D | 45 | 31.678 | 32.770 | 24.753 | 1.00 | 12.52 | 6 | C |
| ATOM | 2111 | CB | GLU | D | 45 | 32.392 | 31.581 | 25.436 | 1.00 | 14.38 | 6 | C |
| ATOM | 2112 | CG | GLU | D | 45 | 32.127 | 30.265 | 24.685 | 1.00 | 18.54 | 6 | C |
| ATOM | 2113 | CD | GLU | D | 45 | 32.979 | 29.114 | 25.184 | 1.00 | 22.44 | 6 | C |
| ATOM | 2114 | OE1 | GLU | D | 45 | 33.025 | 28.070 | 24.514 | 1.00 | 23.21 | 8 | O |
| ATOM | 2115 | OE2 | GLU | D | 45 | 33.613 | 29.221 | 26.263 | 1.00 | 23.95 | 8 | O |
| ATOM | 2116 | C | GLU | D | 45 | 31.715 | 33.963 | 25.718 | 1.00 | 13.18 | 6 | C |
| ATOM | 2117 | O | GLU | D | 45 | 32.691 | 34.687 | 25.785 | 1.00 | 10.66 | 8 | O |
| ATOM | 2118 | N | THR | D | 46 | 30.602 | 34.241 | 26.380 | 1.00 | 13.36 | 7 | N |
| ATOM | 2119 | CA | THR | D | 46 | 30.527 | 35.298 | 27.381 | 1.00 | 13.78 | 6 | C |
| ATOM | 2120 | CB | THR | D | 46 | 29.805 | 36.570 | 26.915 | 1.00 | 15.75 | 6 | C |
| ATOM | 2121 | OG1 | THR | D | 46 | 28.450 | 36.171 | 26.648 | 1.00 | 14.48 | 8 | O |
| ATOM | 2122 | CG2 | THR | D | 46 | 30.488 | 37.200 | 25.710 | 1.00 | 15.69 | 6 | C |
| ATOM | 2123 | C | THR | D | 46 | 29.791 | 34.679 | 28.574 | 1.00 | 14.47 | 6 | C |
| ATOM | 2124 | O | THR | D | 46 | 29.069 | 33.663 | 28.418 | 1.00 | 13.33 | 8 | O |
| ATOM | 2125 | N | ASP | D | 47 | 29.938 | 35.295 | 29.735 | 1.00 | 13.95 | 7 | N |
| ATOM | 2126 | CA | ASP | D | 47 | 29.336 | 34.725 | 30.929 | 1.00 | 16.99 | 6 | C |
| ATOM | 2127 | CB | ASP | D | 47 | 29.734 | 35.409 | 32.247 | 1.00 | 22.34 | 6 | C |
| ATOM | 2128 | CG | ASP | D | 47 | 31.173 | 35.190 | 32.639 | 1.00 | 27.62 | 6 | C |
| ATOM | 2129 | OD1 | ASP | D | 47 | 31.825 | 34.359 | 31.947 | 1.00 | 31.21 | 8 | O |
| ATOM | 2130 | OD2 | ASP | D | 47 | 31.713 | 35.782 | 33.605 | 1.00 | 30.67 | 8 | O |
| ATOM | 2131 | C | ASP | D | 47 | 27.819 | 34.864 | 30.828 | 1.00 | 14.12 | 6 | C |
| ATOM | 2132 | O | ASP | D | 47 | 27.153 | 33.940 | 31.227 | 1.00 | 15.12 | 8 | O |
| ATOM | 2133 | N | THR | D | 48 | 27.297 | 35.969 | 30.327 | 1.00 | 11.97 | 7 | N |
| ATOM | 2134 | CA | THR | D | 48 | 25.875 | 36.189 | 30.244 | 1.00 | 13.15 | 6 | C |
| ATOM | 2135 | CB | THR | D | 48 | 25.373 | 37.236 | 31.240 | 1.00 | 13.15 | 6 | C |
| ATOM | 2136 | OG1 | THR | D | 48 | 26.017 | 38.511 | 30.973 | 1.00 | 15.41 | 8 | O |
| ATOM | 2137 | CG2 | THR | D | 48 | 25.612 | 36.804 | 32.692 | 1.00 | 14.04 | 6 | C |
| ATOM | 2138 | C | THR | D | 48 | 25.446 | 36.742 | 28.877 | 1.00 | 14.57 | 6 | C |
| ATOM | 2139 | O | THR | D | 48 | 26.291 | 37.155 | 28.065 | 1.00 | 12.38 | 8 | O |
| ATOM | 2140 | N | LEU | D | 49 | 24.125 | 36.733 | 28.685 | 1.00 | 11.23 | 7 | N |
| ATOM | 2141 | CA | LEU | D | 49 | 23.583 | 37.284 | 27.437 | 1.00 | 13.73 | 6 | C |
| ATOM | 2142 | CB | LEU | D | 49 | 22.065 | 37.080 | 27.339 | 1.00 | 12.51 | 6 | C |
| ATOM | 2143 | CG | LEU | D | 49 | 21.364 | 37.558 | 26.069 | 1.00 | 14.18 | 6 | C |
| ATOM | 2144 | CD1 | LEU | D | 49 | 22.014 | 37.002 | 24.817 | 1.00 | 11.03 | 6 | C |
| ATOM | 2145 | CD2 | LEU | D | 49 | 19.881 | 37.088 | 26.143 | 1.00 | 13.29 | 6 | C |
| ATOM | 2146 | C | LEU | D | 49 | 23.855 | 38.778 | 27.338 | 1.00 | 13.40 | 6 | C |
| ATOM | 2147 | O | LEU | D | 49 | 24.102 | 39.374 | 26.296 | 1.00 | 13.30 | 8 | O |
| ATOM | 2148 | N | LYS | D | 50 | 23.652 | 39.471 | 28.462 | 1.00 | 13.92 | 7 | N |
| ATOM | 2149 | CA | LYS | D | 50 | 23.916 | 40.892 | 28.544 | 1.00 | 16.34 | 6 | C |
| ATOM | 2150 | CB | LYS | D | 50 | 23.691 | 41.327 | 30.019 | 1.00 | 20.47 | 6 | C |
| ATOM | 2151 | CG | LYS | D | 50 | 23.302 | 42.793 | 30.072 | 1.00 | 26.88 | 6 | C |
| ATOM | 2152 | CD | LYS | D | 50 | 24.075 | 43.538 | 31.151 | 1.00 | 31.08 | 6 | C |
| ATOM | 2153 | CE | LYS | D | 50 | 23.804 | 45.046 | 31.108 | 1.00 | 32.48 | 6 | C |
| ATOM | 2154 | NZ | LYS | D | 50 | 23.836 | 45.593 | 32.509 | 1.00 | 34.98 | 7 | N |
| ATOM | 2155 | C | LYS | D | 50 | 25.348 | 41.230 | 28.148 | 1.00 | 13.33 | 6 | C |
| ATOM | 2156 | O | LYS | D | 50 | 25.614 | 42.240 | 27.468 | 1.00 | 13.39 | 8 | O |
| ATOM | 2157 | N | ASP | D | 51 | 26.299 | 40.411 | 28.563 | 1.00 | 12.48 | 7 | N |
| ATOM | 2158 | CA | ASP | D | 51 | 27.692 | 40.626 | 28.146 | 1.00 | 15.10 | 6 | C |
| ATOM | 2159 | CB | ASP | D | 51 | 28.663 | 39.759 | 28.947 | 1.00 | 16.35 | 6 | C |
| ATOM | 2160 | CG | ASP | D | 51 | 28.728 | 40.315 | 30.377 | 1.00 | 18.78 | 6 | C |
| ATOM | 2161 | OD1 | ASP | D | 51 | 28.465 | 41.527 | 30.553 | 1.00 | 19.84 | 8 | O |
| ATOM | 2162 | OD2 | ASP | D | 51 | 29.036 | 39.468 | 31.217 | 1.00 | 19.28 | 8 | O |
| ATOM | 2163 | C | ASP | D | 51 | 27.868 | 40.329 | 26.659 | 1.00 | 12.69 | 6 | C |
| ATOM | 2164 | O | ASP | D | 51 | 28.701 | 40.979 | 26.028 | 1.00 | 14.63 | 8 | O |
| ATOM | 2165 | N | LEU | D | 52 | 27.187 | 39.306 | 26.139 | 1.00 | 11.08 | 7 | N |
| ATOM | 2166 | CA | LEU | D | 52 | 27.246 | 39.107 | 24.685 | 1.00 | 10.83 | 6 | C |
| ATOM | 2167 | CB | LEU | D | 52 | 26.456 | 37.864 | 24.267 | 1.00 | 10.10 | 6 | C |
| ATOM | 2168 | CG | LEU | D | 52 | 26.605 | 37.462 | 22.793 | 1.00 | 9.50 | 6 | C |
| ATOM | 2169 | CD1 | LEU | D | 52 | 28.001 | 36.865 | 22.523 | 1.00 | 9.81 | 6 | C |
| ATOM | 2170 | CD2 | LEU | D | 52 | 25.529 | 36.464 | 22.405 | 1.00 | 7.72 | 6 | C |
| ATOM | 2171 | C | LEU | D | 52 | 26.704 | 40.331 | 23.976 | 1.00 | 12.03 | 6 | C |
| ATOM | 2172 | O | LEU | D | 52 | 27.289 | 40.837 | 22.985 | 1.00 | 10.16 | 8 | O |
| ATOM | 2173 | N | ASP | D | 53 | 25.546 | 40.859 | 24.479 | 1.00 | 11.64 | 7 | N |
| ATOM | 2174 | CA | ASP | D | 53 | 24.983 | 42.043 | 23.860 | 1.00 | 12.95 | 6 | C |
| ATOM | 2175 | CB | ASP | D | 53 | 23.750 | 42.582 | 24.595 | 1.00 | 14.34 | 6 | C |
| ATOM | 2176 | CG | ASP | D | 53 | 22.563 | 41.630 | 24.609 | 1.00 | 15.63 | 6 | C |
| ATOM | 2177 | OD1 | ASP | D | 53 | 22.449 | 40.684 | 23.791 | 1.00 | 15.62 | 8 | O |
| ATOM | 2178 | OD2 | ASP | D | 53 | 21.691 | 41.824 | 25.492 | 1.00 | 16.34 | 8 | O |
| ATOM | 2179 | C | ASP | D | 53 | 26.040 | 43.147 | 23.688 | 1.00 | 15.69 | 6 | C |
| ATOM | 2180 | O | ASP | D | 53 | 26.150 | 43.779 | 22.627 | 1.00 | 13.41 | 8 | O |
| ATOM | 2181 | N | GLN | D | 54 | 26.714 | 43.454 | 24.782 | 1.00 | 15.39 | 7 | N |
| ATOM | 2182 | CA | GLN | D | 54 | 27.750 | 44.490 | 24.826 | 1.00 | 19.04 | 6 | C |
| ATOM | 2183 | CB | GLN | D | 54 | 28.196 | 44.595 | 26.286 | 1.00 | 21.11 | 6 | C |
| ATOM | 2184 | CG | GLN | D | 54 | 28.911 | 45.886 | 26.619 | 1.00 | 30.34 | 6 | C |
| ATOM | 2185 | CD | GLN | D | 54 | 29.355 | 45.897 | 28.070 | 1.00 | 35.43 | 6 | C |
| ATOM | 2186 | OE1 | GLN | D | 54 | 29.038 | 45.001 | 28.873 | 1.00 | 38.74 | 8 | O |
| ATOM | 2187 | NE2 | GLN | D | 54 | 30.131 | 46.936 | 28.390 | 1.00 | 37.61 | 7 | N |
| ATOM | 2188 | C | GLN | D | 54 | 28.943 | 44.195 | 23.929 | 1.00 | 13.99 | 6 | C |
| ATOM | 2189 | O | GLN | D | 54 | 29.388 | 45.028 | 23.162 | 1.00 | 12.65 | 8 | O |
| ATOM | 2190 | N | PHE | D | 55 | 29.447 | 42.978 | 23.978 | 1.00 | 15.43 | 7 | N |
| ATOM | 2191 | CA | PHE | D | 55 | 30.586 | 42.554 | 23.134 | 1.00 | 13.50 | 6 | C |
| ATOM | 2192 | CB | PHE | D | 55 | 30.897 | 41.093 | 23.483 | 1.00 | 12.42 | 6 | C |
| ATOM | 2193 | CG | PHE | D | 55 | 32.002 | 40.549 | 22.602 | 1.00 | 11.54 | 6 | C |
| ATOM | 2194 | CD1 | PHE | D | 55 | 33.319 | 41.015 | 22.767 | 1.00 | 9.95 | 6 | C |
| ATOM | 2195 | CD2 | PHE | D | 55 | 31.713 | 39.571 | 21.668 | 1.00 | 9.64 | 6 | C |
| ATOM | 2196 | CE1 | PHE | D | 55 | 34.292 | 40.453 | 21.936 | 1.00 | 12.19 | 6 | C |
| ATOM | 2197 | CE2 | PHE | D | 55 | 32.705 | 39.010 | 20.874 | 1.00 | 10.13 | 6 | C |
| ATOM | 2198 | CZ | PHE | D | 55 | 34.002 | 39.465 | 21.026 | 1.00 | 9.99 | 6 | C |
| ATOM | 2199 | C | PHE | D | 55 | 30.255 | 42.714 | 21.653 | 1.00 | 13.58 | 6 | C |
| ATOM | 2200 | O | PHE | D | 55 | 30.888 | 43.510 | 20.915 | 1.00 | 10.92 | 8 | O |
| ATOM | 2201 | N | ILE | D | 56 | 29.107 | 42.119 | 21.255 | 1.00 | 10.76 | 7 | N |
| ATOM | 2202 | CA | ILE | D | 56 | 28.785 | 42.189 | 19.830 | 1.00 | 11.96 | 6 | C |
| ATOM | 2203 | CB | ILE | D | 56 | 27.579 | 41.278 | 19.498 | 1.00 | 13.95 | 6 | C |
| ATOM | 2204 | CG2 | ILE | D | 56 | 27.096 | 41.404 | 18.056 | 1.00 | 12.24 | 6 | C |
| ATOM | 2205 | CG1 | ILE | D | 56 | 28.011 | 39.898 | 19.947 | 1.00 | 14.49 | 6 | C |
| ATOM | 2206 | CD1 | ILE | D | 56 | 28.161 | 38.777 | 19.011 | 1.00 | 18.97 | 6 | C |
| ATOM | 2207 | C | ILE | D | 56 | 28.491 | 43.582 | 19.349 | 1.00 | 13.51 | 6 | C |
| ATOM | 2208 | O | ILE | D | 56 | 28.926 | 43.983 | 18.256 | 1.00 | 13.02 | 8 | O |
| ATOM | 2209 | N | THR | D | 57 | 27.710 | 44.341 | 20.110 | 1.00 | 15.60 | 7 | N |
| ATOM | 2210 | CA | THR | D | 57 | 27.326 | 45.675 | 19.621 | 1.00 | 17.80 | 6 | C |
| ATOM | 2211 | CB | THR | D | 57 | 26.048 | 46.210 | 20.297 | 1.00 | 19.23 | 6 | C |
| ATOM | 2212 | OG1 | THR | D | 57 | 26.180 | 46.125 | 21.711 | 1.00 | 22.03 | 8 | O |
| ATOM | 2213 | CG2 | THR | D | 57 | 24.810 | 45.352 | 20.028 | 1.00 | 20.73 | 6 | C |
| ATOM | 2214 | C | THR | D | 57 | 28.518 | 46.636 | 19.744 | 1.00 | 20.35 | 6 | C |
| ATOM | 2215 | O | THR | D | 57 | 28.824 | 47.295 | 18.729 | 1.00 | 22.00 | 8 | O |
| ATOM | 2216 | N | GLU | D | 58 | 29.389 | 46.530 | 20.744 | 1.00 | 20.17 | 7 | N |
| ATOM | 2217 | CA | GLU | D | 58 | 30.510 | 47.426 | 20.876 | 1.00 | 23.72 | 6 | C |
| ATOM | 2218 | CB | GLU | D | 58 | 30.773 | 47.819 | 22.355 | 1.00 | 27.15 | 6 | C |
| ATOM | 2219 | CG | GLU | D | 58 | 29.557 | 48.536 | 22.967 | 1.00 | 30.14 | 6 | C |
| ATOM | 2220 | CD | GLU | D | 58 | 29.380 | 49.919 | 22.337 | 1.00 | 33.67 | 6 | C |
| ATOM | 2221 | OE1 | GLU | D | 58 | 30.396 | 50.630 | 22.041 | 1.00 | 35.18 | 8 | O |
| ATOM | 2222 | OE2 | GLU | D | 58 | 28.204 | 50.285 | 22.109 | 1.00 | 34.26 | 8 | O |
| ATOM | 2223 | C | GLU | D | 58 | 31.818 | 46.930 | 20.286 | 1.00 | 22.92 | 6 | C |
| ATOM | 2224 | O | GLU | D | 58 | 32.507 | 47.785 | 19.723 | 1.00 | 22.33 | 8 | O |
| ATOM | 2225 | N | LYS | D | 59 | 32.204 | 45.660 | 20.404 | 1.00 | 20.11 | 7 | N |
| ATOM | 2226 | CA | LYS | D | 59 | 33.473 | 45.223 | 19.873 | 1.00 | 20.10 | 6 | C |
| ATOM | 2227 | CB | LYS | D | 59 | 34.111 | 44.113 | 20.719 | 1.00 | 21.48 | 6 | C |
| ATOM | 2228 | CG | LYS | D | 59 | 34.207 | 44.513 | 22.199 | 1.00 | 27.96 | 6 | C |
| ATOM | 2229 | CD | LYS | D | 59 | 35.424 | 45.373 | 22.500 | 1.00 | 30.31 | 6 | C |
| ATOM | 2230 | CE | LYS | D | 59 | 35.427 | 45.865 | 23.946 | 1.00 | 32.73 | 6 | C |
| ATOM | 2231 | NZ | LYS | D | 59 | 35.737 | 47.347 | 24.028 | 1.00 | 34.31 | 7 | N |
| ATOM | 2232 | C | LYS | D | 59 | 33.396 | 44.694 | 18.438 | 1.00 | 19.09 | 6 | C |
| ATOM | 2233 | O | LYS | D | 59 | 34.445 | 44.621 | 17.756 | 1.00 | 19.41 | 8 | O |
| ATOM | 2234 | N | ILE | D | 60 | 32.263 | 44.090 | 18.091 | 1.00 | 13.82 | 7 | N |
| ATOM | 2235 | CA | ILE | D | 60 | 32.200 | 43.489 | 16.762 | 1.00 | 12.95 | 6 | C |
| ATOM | 2236 | CB | ILE | D | 60 | 31.556 | 42.118 | 16.849 | 1.00 | 12.02 | 6 | C |
| ATOM | 2237 | CG2 | ILE | D | 60 | 31.412 | 41.544 | 15.429 | 1.00 | 11.69 | 6 | C |
| ATOM | 2238 | CG1 | ILE | D | 60 | 32.335 | 41.135 | 17.750 | 1.00 | 12.06 | 6 | C |
| ATOM | 2239 | CD1 | ILE | D | 60 | 33.810 | 41.039 | 17.546 | 1.00 | 12.30 | 6 | C |
| ATOM | 2240 | C | ILE | D | 60 | 31.545 | 44.412 | 15.729 | 1.00 | 14.62 | 6 | C |
| ATOM | 2241 | O | ILE | D | 60 | 32.126 | 44.822 | 14.727 | 1.00 | 10.09 | 8 | O |
| ATOM | 2242 | N | ARG | D | 61 | 30.278 | 44.774 | 15.969 | 1.00 | 14.65 | 7 | N |
| ATOM | 2243 | CA | ARG | D | 61 | 29.523 | 45.610 | 15.045 | 1.00 | 16.07 | 6 | C |
| ATOM | 2244 | CB | ARG | D | 61 | 28.038 | 45.579 | 15.500 | 1.00 | 16.92 | 6 | C |
| ATOM | 2245 | CG | ARG | D | 61 | 27.363 | 44.212 | 15.262 | 1.00 | 17.47 | 6 | C |
| ATOM | 2246 | CD | ARG | D | 61 | 25.893 | 44.275 | 15.640 | 1.00 | 22.16 | 6 | C |
| ATOM | 2247 | NE | ARG | D | 61 | 25.086 | 43.063 | 15.705 | 1.00 | 20.80 | 7 | N |
| ATOM | 2248 | CZ | ARG | D | 61 | 23.799 | 42.867 | 15.496 | 1.00 | 20.73 | 6 | C |
| ATOM | 2249 | NH1 | ARG | D | 61 | 22.940 | 43.817 | 15.183 | 1.00 | 21.08 | 7 | N |
| ATOM | 2250 | NH2 | ARG | D | 61 | 23.194 | 41.692 | 15.674 | 1.00 | 19.70 | 7 | N |
| ATOM | 2251 | C | ARG | D | 61 | 30.081 | 47.007 | 14.834 | 1.00 | 15.30 | 6 | C |
| ATOM | 2252 | O | ARG | D | 61 | 29.919 | 47.548 | 13.729 | 1.00 | 12.83 | 8 | O |
| ATOM | 2253 | N | LYS | D | 62 | 30.862 | 47.581 | 15.726 | 1.00 | 13.69 | 7 | N |
| ATOM | 2254 | CA | LYS | D | 62 | 31.537 | 48.845 | 15.612 | 1.00 | 16.28 | 6 | C |
| ATOM | 2255 | CB | LYS | D | 62 | 31.355 | 49.671 | 16.910 | 1.00 | 19.57 | 6 | C |
| ATOM | 2256 | CG | LYS | D | 62 | 29.965 | 50.343 | 16.732 | 1.00 | 21.09 | 6 | C |
| ATOM | 2257 | CD | LYS | D | 62 | 29.347 | 50.574 | 18.073 | 1.00 | 24.81 | 6 | C |
| ATOM | 2258 | CE | LYS | D | 62 | 28.160 | 51.539 | 17.965 | 1.00 | 26.49 | 6 | C |
| ATOM | 2259 | NZ | LYS | D | 62 | 27.744 | 51.698 | 19.399 | 1.00 | 32.17 | 7 | N |
| ATOM | 2260 | C | LYS | D | 62 | 32.991 | 48.777 | 15.185 | 1.00 | 15.48 | 6 | C |
| ATOM | 2261 | O | LYS | D | 62 | 33.619 | 49.794 | 14.910 | 1.00 | 13.01 | 8 | O |
| ATOM | 2262 | N | MET | D | 63 | 33.483 | 47.575 | 14.930 | 1.00 | 13.64 | 7 | N |
| ATOM | 2263 | CA | MET | D | 63 | 34.840 | 47.469 | 14.347 | 1.00 | 14.05 | 6 | C |
| ATOM | 2264 | CB | MET | D | 63 | 35.250 | 46.002 | 14.371 | 1.00 | 14.96 | 6 | C |
| ATOM | 2265 | CG | MET | D | 63 | 36.702 | 45.765 | 13.995 | 1.00 | 17.59 | 6 | C |
| ATOM | 2266 | SD | MET | D | 63 | 37.077 | 44.017 | 13.778 | 1.00 | 16.00 | 16 | S |
| ATOM | 2267 | CE | MET | D | 63 | 36.816 | 43.394 | 15.447 | 1.00 | 16.24 | 6 | C |
| ATOM | 2268 | C | MET | D | 63 | 34.798 | 48.084 | 12.950 | 1.00 | 12.08 | 6 | C |
| ATOM | 2269 | O | MET | D | 63 | 33.998 | 47.740 | 12.079 | 1.00 | 8.64 | 8 | O |
| ATOM | 2270 | N | PRO | D | 64 | 35.692 | 49.011 | 12.624 | 1.00 | 12.28 | 7 | N |
| ATOM | 2271 | CD | PRO | D | 64 | 36.723 | 49.571 | 13.578 | 1.00 | 13.06 | 6 | C |
| ATOM | 2272 | CA | PRO | D | 64 | 35.610 | 49.760 | 11.398 | 1.00 | 11.60 | 6 | C |
| ATOM | 2273 | CB | PRO | D | 64 | 36.749 | 50.767 | 11.450 | 1.00 | 14.09 | 6 | C |
| ATOM | 2274 | CG | PRO | D | 64 | 37.175 | 50.838 | 12.874 | 1.00 | 14.47 | 6 | C |
| ATOM | 2275 | C | PRO | D | 64 | 35.697 | 48.907 | 10.137 | 1.00 | 11.66 | 6 | C |
| ATOM | 2276 | O | PRO | D | 64 | 35.144 | 49.337 | 9.117 | 1.00 | 9.20 | 8 | O |
| ATOM | 2277 | N | GLU | D | 65 | 36.518 | 47.855 | 10.133 | 1.00 | 7.96 | 7 | N |
| ATOM | 2278 | CA | GLU | D | 65 | 36.670 | 46.978 | 8.990 | 1.00 | 10.89 | 6 | C |
| ATOM | 2279 | CB | GLU | D | 65 | 37.858 | 46.024 | 9.221 | 1.00 | 13.55 | 6 | C |
| ATOM | 2280 | CG | GLU | D | 65 | 39.187 | 46.824 | 9.538 | 1.00 | 13.78 | 6 | C |
| ATOM | 2281 | CD | GLU | D | 65 | 39.461 | 46.983 | 11.033 | 1.00 | 16.55 | 6 | C |
| ATOM | 2282 | OE1 | GLU | D | 65 | 38.484 | 47.049 | 11.844 | 1.00 | 12.89 | 8 | O |
| ATOM | 2283 | OE2 | GLU | D | 65 | 40.649 | 47.099 | 11.496 | 1.00 | 15.86 | 8 | O |
| ATOM | 2284 | C | GLU | D | 65 | 35.406 | 46.138 | 8.682 | 1.00 | 11.19 | 6 | C |
| ATOM | 2285 | O | GLU | D | 65 | 35.350 | 45.423 | 7.680 | 1.00 | 7.50 | 8 | O |
| ATOM | 2286 | N | ILE | D | 66 | 34.519 | 45.952 | 9.674 | 1.00 | 10.12 | 7 | N |
| ATOM | 2287 | CA | ILE | D | 66 | 33.361 | 45.070 | 9.550 | 1.00 | 11.60 | 6 | C |
| ATOM | 2288 | CB | ILE | D | 66 | 32.970 | 44.480 | 10.918 | 1.00 | 13.04 | 6 | C |
| ATOM | 2289 | CG2 | ILE | D | 66 | 31.591 | 43.825 | 10.916 | 1.00 | 13.13 | 6 | C |
| ATOM | 2290 | CG1 | ILE | D | 66 | 34.098 | 43.486 | 11.300 | 1.00 | 13.41 | 6 | C |
| ATOM | 2291 | CD1 | ILE | D | 66 | 33.797 | 42.450 | 12.339 | 1.00 | 16.26 | 6 | C |
| ATOM | 2292 | C | ILE | D | 66 | 32.247 | 45.760 | 8.785 | 1.00 | 12.88 | 6 | C |
| ATOM | 2293 | O | ILE | D | 66 | 31.842 | 46.893 | 9.084 | 1.00 | 11.94 | 8 | O |
| ATOM | 2294 | N | GLN | D | 67 | 31.690 | 45.048 | 7.820 | 1.00 | 13.32 | 7 | N |
| ATOM | 2295 | CA | GLN | D | 67 | 30.565 | 45.570 | 7.031 | 1.00 | 13.81 | 6 | C |
| ATOM | 2296 | CB | GLN | D | 67 | 30.876 | 45.313 | 5.537 | 1.00 | 17.32 | 6 | C |
| ATOM | 2297 | CG | GLN | D | 67 | 32.065 | 46.094 | 5.003 | 1.00 | 20.78 | 6 | C |
| ATOM | 2298 | CD | GLN | D | 67 | 32.258 | 45.972 | 3.503 | 1.00 | 25.96 | 6 | C |
| ATOM | 2299 | OE1 | GLN | D | 67 | 32.984 | 46.769 | 2.874 | 1.00 | 26.83 | 8 | O |
| ATOM | 2300 | NE2 | GLN | D | 67 | 31.666 | 44.957 | 2.880 | 1.00 | 26.96 | 7 | N |
| ATOM | 2301 | C | GLN | D | 67 | 29.249 | 44.883 | 7.366 | 1.00 | 14.11 | 6 | C |
| ATOM | 2302 | O | GLN | D | 67 | 28.204 | 45.520 | 7.260 | 1.00 | 12.99 | 8 | O |
| ATOM | 2303 | N | MET | D | 68 | 29.234 | 43.596 | 7.687 | 1.00 | 13.17 | 7 | N |
| ATOM | 2304 | CA | MET | D | 68 | 27.975 | 42.918 | 8.012 | 1.00 | 14.61 | 6 | C |
| ATOM | 2305 | CB | MET | D | 68 | 27.292 | 42.526 | 6.707 | 1.00 | 17.39 | 6 | C |
| ATOM | 2306 | CG | MET | D | 68 | 27.455 | 41.040 | 6.441 | 1.00 | 26.39 | 6 | C |
| ATOM | 2307 | SD | MET | D | 68 | 25.983 | 40.112 | 6.946 | 1.00 | 28.95 | 16 | S |
| ATOM | 2308 | CE | MET | D | 68 | 25.089 | 40.321 | 5.379 | 1.00 | 31.95 | 6 | C |
| ATOM | 2309 | C | MET | D | 68 | 28.269 | 41.745 | 8.924 | 1.00 | 12.16 | 6 | C |
| ATOM | 2310 | O | MET | D | 68 | 29.374 | 41.163 | 8.887 | 1.00 | 8.95 | 8 | O |
| ATOM | 2311 | N | THR | D | 69 | 27.352 | 41.409 | 9.826 | 1.00 | 10.00 | 7 | N |
| ATOM | 2312 | CA | THR | D | 69 | 27.548 | 40.286 | 10.750 | 1.00 | 9.18 | 6 | C |
| ATOM | 2313 | CB | THR | D | 69 | 27.973 | 40.679 | 12.180 | 1.00 | 10.80 | 6 | C |
| ATOM | 2314 | OG1 | THR | D | 69 | 26.965 | 41.517 | 12.738 | 1.00 | 10.14 | 8 | O |
| ATOM | 2315 | CG2 | THR | D | 69 | 29.266 | 41.533 | 12.207 | 1.00 | 8.79 | 6 | C |
| ATOM | 2316 | C | THR | D | 69 | 26.209 | 39.551 | 10.885 | 1.00 | 10.48 | 6 | C |
| ATOM | 2317 | O | THR | D | 69 | 25.177 | 40.204 | 10.825 | 1.00 | 9.21 | 8 | O |
| ATOM | 2318 | N | SER | D | 70 | 26.237 | 38.260 | 11.134 | 1.00 | 8.78 | 7 | N |
| ATOM | 2319 | CA | SER | D | 70 | 25.036 | 37.493 | 11.456 | 1.00 | 11.09 | 6 | C |
| ATOM | 2320 | CB | SER | D | 70 | 24.662 | 36.702 | 10.189 | 1.00 | 12.33 | 6 | C |
| ATOM | 2321 | OG | SER | D | 70 | 23.493 | 35.994 | 10.510 | 1.00 | 13.28 | 8 | O |
| ATOM | 2322 | C | SER | D | 70 | 25.401 | 36.622 | 12.648 | 1.00 | 12.05 | 6 | C |
| ATOM | 2323 | O | SER | D | 70 | 26.397 | 35.870 | 12.567 | 1.00 | 12.32 | 8 | O |
| ATOM | 2324 | N | THR | D | 71 | 24.700 | 36.673 | 13.769 | 1.00 | 10.51 | 7 | N |
| ATOM | 2325 | CA | THR | D | 71 | 25.097 | 35.890 | 14.952 | 1.00 | 9.85 | 6 | C |
| ATOM | 2326 | CB | THR | D | 71 | 25.203 | 36.844 | 16.156 | 1.00 | 11.18 | 6 | C |
| ATOM | 2327 | OG1 | THR | D | 71 | 26.203 | 37.821 | 15.911 | 1.00 | 8.76 | 8 | O |
| ATOM | 2328 | CG2 | THR | D | 71 | 25.517 | 36.186 | 17.508 | 1.00 | 12.67 | 6 | C |
| ATOM | 2329 | C | THR | D | 71 | 24.120 | 34.795 | 15.261 | 1.00 | 8.82 | 6 | C |
| ATOM | 2330 | O | THR | D | 71 | 22.916 | 35.051 | 15.240 | 1.00 | 9.45 | 8 | O |
| ATOM | 2331 | N | MET | D | 72 | 24.629 | 33.588 | 15.474 | 1.00 | 6.60 | 7 | N |
| ATOM | 2332 | CA | MET | D | 72 | 23.854 | 32.463 | 15.846 | 1.00 | 7.67 | 6 | C |
| ATOM | 2333 | CB | MET | D | 72 | 24.175 | 31.262 | 14.930 | 1.00 | 9.49 | 6 | C |
| ATOM | 2334 | CG | MET | D | 72 | 23.687 | 31.559 | 13.500 | 1.00 | 14.91 | 6 | C |
| ATOM | 2335 | SD | MET | D | 72 | 24.478 | 30.502 | 12.269 | 1.00 | 15.37 | 16 | S |
| ATOM | 2336 | CE | MET | D | 72 | 26.144 | 31.123 | 12.202 | 1.00 | 17.36 | 6 | C |
| ATOM | 2337 | C | MET | D | 72 | 24.124 | 32.078 | 17.306 | 1.00 | 7.83 | 6 | C |
| ATOM | 2338 | O | MET | D | 72 | 25.184 | 31.458 | 17.566 | 1.00 | 8.05 | 8 | O |
| ATOM | 2339 | N | ILE | D | 73 | 23.212 | 32.379 | 18.186 | 1.00 | 8.09 | 7 | N |
| ATOM | 2340 | CA | ILE | D | 73 | 23.332 | 32.016 | 19.610 | 1.00 | 8.99 | 6 | C |
| ATOM | 2341 | CB | ILE | D | 73 | 22.410 | 32.880 | 20.471 | 1.00 | 9.51 | 6 | C |
| ATOM | 2342 | CG2 | ILE | D | 73 | 22.288 | 32.354 | 21.920 | 1.00 | 11.36 | 6 | C |
| ATOM | 2343 | CG1 | ILE | D | 73 | 22.973 | 34.315 | 20.535 | 1.00 | 11.16 | 6 | C |
| ATOM | 2344 | CD1 | ILE | D | 73 | 21.936 | 35.363 | 20.896 | 1.00 | 11.78 | 6 | C |
| ATOM | 2345 | C | ILE | D | 73 | 23.038 | 30.542 | 19.775 | 1.00 | 11.33 | 6 | C |
| ATOM | 2346 | O | ILE | D | 73 | 22.036 | 30.000 | 19.267 | 1.00 | 11.92 | 8 | O |
| ATOM | 2347 | N | ALA | D | 74 | 23.949 | 29.824 | 20.414 | 1.00 | 12.81 | 7 | N |
| ATOM | 2348 | CA | ALA | D | 74 | 23.809 | 28.415 | 20.674 | 1.00 | 16.28 | 6 | C |
| ATOM | 2349 | CB | ALA | D | 74 | 25.122 | 27.728 | 21.084 | 1.00 | 17.94 | 6 | C |
| ATOM | 2350 | C | ALA | D | 74 | 22.875 | 28.195 | 21.886 | 1.00 | 18.40 | 6 | C |
| ATOM | 2351 | O | ALA | D | 74 | 22.936 | 28.914 | 22.879 | 1.00 | 16.19 | 8 | O |
| ATOM | 2352 | N | ILE | D | 75 | 22.208 | 27.070 | 21.826 | 1.00 | 20.41 | 7 | N |
| ATOM | 2353 | CA | ILE | D | 75 | 21.496 | 26.531 | 22.968 | 1.00 | 24.59 | 6 | C |
| ATOM | 2354 | CB | ILE | D | 75 | 20.391 | 25.581 | 22.489 | 1.00 | 25.97 | 6 | C |
| ATOM | 2355 | CG2 | ILE | D | 75 | 20.115 | 24.427 | 23.439 | 1.00 | 26.43 | 6 | C |
| ATOM | 2356 | CG1 | ILE | D | 75 | 19.126 | 26.385 | 22.194 | 1.00 | 24.83 | 6 | C |
| ATOM | 2357 | CD1 | ILE | D | 75 | 19.021 | 26.717 | 20.709 | 1.00 | 27.91 | 6 | C |
| ATOM | 2358 | C | ILE | D | 75 | 22.520 | 25.792 | 23.826 | 1.00 | 27.53 | 6 | C |
| ATOM | 2359 | O | ILE | D | 75 | 23.522 | 25.173 | 23.340 | 1.00 | 29.12 | 8 | O |
| ATOM | 2360 | OT | ILE | D | 75 | 22.263 | 25.858 | 25.044 | 1.00 | 29.73 | 8 | O |
| ATOM | 2361 | O | HOH | W | 1 | 10.122 | 48.309 | 2.632 | 1.00 | 71.67 | 8 | O |
| ATOM | 2362 | O | HOH | W | 2 | 43.515 | 40.335 | 4.275 | 1.00 | 2.02 | 8 | O |
| ATOM | 2363 | O | HOH | W | 3 | 25.096 | 40.046 | 14.391 | 1.00 | 4.72 | 8 | O |
| ATOM | 2364 | O | HOH | W | 4 | 14.309 | 48.257 | 1.287 | 1.00 | 11.98 | 8 | O |
| ATOM | 2365 | O | HOH | W | 5 | 39.903 | 40.707 | 3.072 | 1.00 | 4.59 | 8 | O |
| ATOM | 2366 | O | HOH | W | 6 | 13.993 | 28.927 | 16.050 | 1.00 | 8.16 | 8 | O |
| ATOM | 2367 | O | HOH | W | 7 | −2.824 | 44.331 | 8.615 | 1.00 | 6.77 | 8 | O |
| ATOM | 2368 | O | HOH | W | 8 | 46.758 | 44.137 | 12.543 | 1.00 | 9.80 | 8 | O |
| ATOM | 2369 | O | HOH | W | 9 | 22.057 | 38.363 | 30.808 | 1.00 | 10.53 | 8 | O |
| ATOM | 2370 | O | HOH | W | 10 | 41.588 | 30.809 | 8.960 | 1.00 | 14.97 | 8 | O |
| ATOM | 2371 | O | HOH | W | 11 | 32.061 | 36.737 | −0.438 | 1.00 | 13.10 | 8 | O |
| ATOM | 2372 | O | HOH | W | 12 | 25.621 | 43.699 | 12.042 | 1.00 | 12.21 | 8 | O |
| ATOM | 2373 | O | HOH | W | 13 | 41.643 | 43.045 | 19.819 | 1.00 | 12.14 | 8 | O |
| ATOM | 2374 | O | HOH | W | 14 | 18.598 | 35.090 | 17.431 | 1.00 | 7.05 | 8 | O |
| ATOM | 2375 | O | HOH | W | 15 | 35.642 | 46.870 | 0.589 | 1.00 | 14.25 | 8 | O |
| ATOM | 2376 | O | HOH | W | 16 | −2.309 | 45.120 | 15.349 | 1.00 | 5.19 | 8 | O |
| ATOM | 2377 | O | HOH | W | 17 | 21.148 | 34.153 | 17.144 | 1.00 | 9.28 | 8 | O |
| ATOM | 2378 | O | HOH | W | 18 | 10.042 | 49.433 | 21.052 | 1.00 | 16.66 | 8 | O |
| ATOM | 2379 | O | HOH | W | 19 | 24.907 | 43.442 | 9.509 | 1.00 | 8.06 | 8 | O |
| ATOM | 2380 | O | HOH | W | 20 | 40.340 | 29.254 | 22.526 | 1.00 | 15.14 | 8 | O |
| ATOM | 2381 | O | HOH | W | 21 | 24.110 | 53.078 | 16.016 | 1.00 | 13.14 | 8 | O |
| ATOM | 2382 | O | HOH | W | 22 | 30.704 | 31.905 | 3.919 | 1.00 | 15.56 | 8 | O |
| ATOM | 2383 | O | HOH | W | 23 | 16.095 | 40.414 | 25.005 | 1.00 | 19.01 | 8 | O |
| ATOM | 2384 | O | HOH | W | 24 | −4.341 | 51.429 | 0.089 | 1.00 | 10.32 | 8 | O |
| ATOM | 2385 | O | HOH | W | 25 | 34.577 | 38.997 | 0.926 | 1.00 | 10.61 | 8 | O |
| ATOM | 2386 | O | HOH | W | 26 | 31.432 | 48.786 | 11.676 | 1.00 | 11.01 | 8 | O |
| ATOM | 2387 | O | HOH | W | 27 | 32.097 | 37.715 | 29.475 | 1.00 | 10.30 | 8 | O |
| ATOM | 2388 | O | HOH | W | 28 | −2.898 | 39.485 | 16.073 | 1.00 | 12.22 | 8 | O |
| ATOM | 2389 | O | HOH | W | 29 | 22.459 | 34.856 | 30.472 | 1.00 | 17.04 | 8 | O |
| ATOM | 2390 | O | HOH | W | 30 | −10.084 | 35.455 | 15.693 | 1.00 | 11.27 | 8 | O |
| ATOM | 2391 | O | HOH | W | 31 | 44.404 | 44.372 | 13.687 | 1.00 | 7.67 | 8 | O |
| ATOM | 2392 | O | HOH | W | 32 | 30.987 | 42.125 | 27.230 | 1.00 | 18.75 | 8 | O |
| ATOM | 2393 | O | HOH | W | 33 | 24.805 | 29.509 | 24.797 | 1.00 | 10.67 | 8 | O |
| ATOM | 2394 | O | HOH | W | 34 | 13.345 | 25.150 | 11.364 | 1.00 | 12.88 | 8 | O |
| ATOM | 2395 | O | HOH | W | 35 | 44.404 | 42.640 | 6.498 | 1.00 | 6.66 | 8 | O |
| ATOM | 2396 | O | HOH | W | 36 | 13.422 | 56.153 | −1.749 | 1.00 | 15.97 | 8 | O |
| ATOM | 2397 | O | HOH | W | 37 | 19.476 | 35.360 | 30.724 | 1.00 | 11.73 | 8 | O |
| ATOM | 2398 | O | HOH | W | 38 | 9.109 | 42.563 | 1.635 | 1.00 | 6.86 | 8 | O |
| ATOM | 2399 | O | HOH | W | 39 | 40.432 | 23.485 | 5.437 | 1.00 | 12.80 | 8 | O |
| ATOM | 2400 | O | HOH | W | 40 | 42.096 | 30.240 | 11.776 | 1.00 | 14.42 | 8 | O |
| ATOM | 2401 | O | HOH | W | 41 | 42.873 | 37.972 | 25.858 | 1.00 | 11.47 | 8 | O |
| ATOM | 2402 | O | HOH | W | 42 | 9.770 | 32.553 | 31.637 | 1.00 | 15.13 | 8 | O |
| ATOM | 2403 | O | HOH | W | 43 | −6.902 | 49.549 | 6.190 | 1.00 | 9.47 | 8 | O |
| ATOM | 2404 | O | HOH | W | 44 | 28.555 | 30.858 | 2.221 | 1.00 | 13.41 | 8 | O |
| ATOM | 2405 | O | HOH | W | 45 | −1.387 | 38.899 | 22.433 | 1.00 | 11.37 | 8 | O |
| ATOM | 2406 | O | HOH | W | 46 | 37.404 | 40.600 | 2.609 | 1.00 | 14.02 | 8 | O |
| ATOM | 2407 | O | HOH | W | 47 | 22.531 | 31.795 | 29.779 | 1.00 | 17.69 | 8 | O |
| ATOM | 2408 | O | HOH | W | 48 | 23.048 | 38.797 | 14.218 | 1.00 | 14.69 | 8 | O |
| ATOM | 2409 | O | HOH | W | 49 | 17.943 | 35.402 | 10.729 | 1.00 | 20.01 | 8 | O |
| ATOM | 2410 | O | HOH | W | 50 | 5.174 | 54.657 | −2.822 | 1.00 | 17.90 | 8 | O |
| ATOM | 2411 | O | HOH | W | 51 | 13.246 | 20.750 | 19.283 | 1.00 | 29.29 | 8 | O |
| ATOM | 2412 | O | HOH | W | 52 | 48.032 | 37.211 | 11.489 | 1.00 | 16.87 | 8 | O |
| ATOM | 2413 | O | HOH | W | 53 | 30.209 | 20.178 | 20.611 | 1.00 | 18.81 | 8 | O |
| ATOM | 2414 | O | HOH | W | 54 | 23.513 | 22.824 | 21.491 | 1.00 | 20.26 | 8 | O |
| ATOM | 2415 | O | HOH | W | 55 | 21.561 | 29.530 | 25.223 | 1.00 | 20.52 | 8 | O |
| ATOM | 2416 | O | HOH | W | 56 | 42.111 | 36.443 | 28.208 | 1.00 | 19.47 | 8 | O |
| ATOM | 2417 | O | HOH | W | 57 | 40.876 | 29.661 | 15.978 | 1.00 | 16.06 | 8 | O |
| ATOM | 2418 | O | HOH | W | 58 | 16.494 | 34.453 | 13.028 | 1.00 | 25.55 | 8 | O |
| ATOM | 2419 | O | HOH | W | 59 | −0.857 | 46.408 | 17.858 | 1.00 | 16.37 | 8 | O |
| ATOM | 2420 | O | HOH | W | 60 | 32.821 | 25.971 | 0.795 | 1.00 | 16.71 | 8 | O |
| ATOM | 2421 | O | HOH | W | 61 | 45.627 | 45.117 | 6.519 | 1.00 | 18.94 | 8 | O |
| ATOM | 2422 | O | HOH | W | 62 | 21.976 | 40.997 | 13.661 | 1.00 | 17.37 | 8 | O |
| ATOM | 2423 | O | HOH | W | 63 | 15.836 | 30.099 | 15.388 | 1.00 | 15.61 | 8 | O |
| ATOM | 2424 | O | HOH | W | 64 | 30.469 | 26.207 | 24.253 | 1.00 | 22.51 | 8 | O |
| ATOM | 2425 | O | HOH | W | 65 | 16.718 | 46.859 | 20.929 | 1.00 | 14.88 | 8 | O |
| ATOM | 2426 | O | HOH | W | 66 | 10.425 | 43.686 | 25.115 | 1.00 | 19.64 | 8 | O |
| ATOM | 2427 | O | HOH | W | 67 | 21.112 | 30.369 | 11.432 | 1.00 | 24.23 | 8 | O |
| ATOM | 2428 | O | HOH | W | 68 | 17.191 | 27.886 | 14.604 | 1.00 | 16.40 | 8 | O |
| ATOM | 2429 | O | HOH | W | 69 | −1.313 | 55.974 | 8.469 | 1.00 | 20.48 | 8 | O |
| ATOM | 2430 | O | HOH | W | 70 | −5.616 | 49.292 | 2.546 | 1.00 | 14.23 | 8 | O |
| ATOM | 2431 | O | HOH | W | 71 | 18.410 | 59.447 | 0.811 | 1.00 | 16.23 | 8 | O |
| ATOM | 2432 | O | HOH | W | 72 | 4.679 | 43.284 | 24.130 | 1.00 | 26.97 | 8 | O |
| ATOM | 2433 | O | HOH | W | 73 | 28.210 | 48.003 | 7.105 | 1.00 | 25.13 | 8 | O |
| ATOM | 2434 | O | HOH | W | 74 | 24.472 | 50.097 | 17.032 | 1.00 | 18.43 | 8 | O |
| ATOM | 2435 | O | HOH | W | 75 | 44.769 | 47.165 | 4.712 | 1.00 | 20.42 | 8 | O |
| ATOM | 2436 | O | HOH | W | 76 | 20.456 | 33.034 | 10.801 | 1.00 | 21.21 | 8 | O |
| ATOM | 2437 | O | HOH | W | 77 | 27.096 | 24.676 | 0.583 | 1.00 | 16.78 | 8 | O |
| ATOM | 2438 | O | HOH | W | 78 | 45.463 | 43.772 | 2.754 | 1.00 | 19.58 | 8 | O |
| ATOM | 2439 | O | HOH | W | 79 | 36.902 | 42.152 | 24.314 | 1.00 | 19.89 | 8 | O |
| ATOM | 2440 | O | HOH | W | 80 | 25.028 | 31.904 | 31.035 | 1.00 | 25.70 | 8 | O |
| ATOM | 2441 | O | HOH | W | 81 | 19.951 | 31.779 | 31.151 | 1.00 | 20.25 | 8 | O |
| ATOM | 2442 | O | HOH | W | 82 | 2.821 | 56.599 | 5.110 | 1.00 | 24.51 | 8 | O |
| ATOM | 2443 | O | HOH | W | 83 | 0.240 | 38.318 | 26.532 | 1.00 | 20.61 | 8 | O |
| ATOM | 2444 | O | HOH | W | 84 | −5.501 | 28.962 | 8.643 | 1.00 | 25.67 | 8 | O |
| ATOM | 2445 | O | HOH | W | 85 | 15.080 | 16.024 | 18.827 | 1.00 | 23.79 | 8 | O |
| ATOM | 2446 | O | HOH | W | 86 | 17.453 | 52.249 | 2.615 | 1.00 | 24.26 | 8 | O |
| ATOM | 2447 | O | HOH | W | 87 | 10.408 | 41.106 | 29.551 | 1.00 | 25.17 | 8 | O |
| ATOM | 2448 | O | HOH | W | 88 | −1.008 | 36.087 | 24.249 | 1.00 | 22.03 | 8 | O |
| ATOM | 2449 | O | HOH | W | 89 | 28.715 | 28.420 | 1.496 | 1.00 | 33.15 | 8 | O |
| ATOM | 2450 | O | HOH | W | 90 | 26.479 | 48.310 | 17.176 | 1.00 | 14.80 | 8 | O |
| ATOM | 2451 | O | HOH | W | 91 | 4.544 | 21.028 | 22.060 | 1.00 | 23.39 | 8 | O |
| ATOM | 2452 | O | HOH | W | 92 | 7.062 | 20.090 | 15.336 | 1.00 | 17.95 | 8 | O |
| ATOM | 2453 | O | HOH | W | 93 | −8.087 | 52.015 | 8.198 | 1.00 | 13.31 | 8 | O |
| ATOM | 2454 | O | HOH | W | 94 | 13.718 | 42.096 | 26.908 | 1.00 | 15.23 | 8 | O |
| ATOM | 2455 | O | HOH | W | 95 | −5.925 | 31.260 | 22.875 | 1.00 | 18.50 | 8 | O |
| ATOM | 2456 | O | HOH | W | 96 | 2.262 | 47.977 | 19.728 | 1.00 | 22.03 | 8 | O |
| ATOM | 2457 | O | HOH | W | 97 | 39.222 | 48.045 | 1.791 | 1.00 | 23.00 | 8 | O |
| ATOM | 2458 | O | HOH | W | 98 | 13.202 | 60.207 | 12.694 | 1.00 | 18.82 | 8 | O |
| ATOM | 2459 | O | HOH | W | 99 | 35.592 | 47.975 | 18.124 | 1.00 | 21.60 | 8 | O |
| ATOM | 2460 | O | HOH | W | 100 | 32.405 | 49.718 | 9.091 | 1.00 | 23.23 | 8 | O |
| ATOM | 2461 | O | HOH | W | 101 | 32.530 | 40.013 | 27.354 | 1.00 | 26.88 | 8 | O |
| ATOM | 2462 | O | HOH | W | 102 | 15.214 | 19.032 | 19.403 | 1.00 | 31.35 | 8 | O |
| ATOM | 2463 | O | HOH | W | 103 | 29.693 | 49.421 | 9.197 | 1.00 | 18.95 | 8 | O |
| ATOM | 2464 | O | HOH | W | 104 | 34.770 | 34.970 | 27.712 | 1.00 | 36.84 | 8 | O |
| ATOM | 2465 | O | HOH | W | 105 | 13.525 | 57.916 | 19.061 | 1.00 | 24.00 | 8 | O |
| ATOM | 2466 | O | HOH | W | 106 | 22.201 | 34.397 | 12.453 | 1.00 | 27.96 | 8 | O |
| ATOM | 2467 | O | HOH | W | 107 | 35.580 | 49.792 | 6.450 | 1.00 | 17.81 | 8 | O |
| ATOM | 2468 | O | HOH | W | 108 | 28.722 | 38.669 | 0.882 | 1.00 | 28.33 | 8 | O |
| ATOM | 2469 | O | HOH | W | 109 | −1.552 | 26.414 | 23.825 | 1.00 | 25.54 | 8 | O |
| ATOM | 2470 | O | HOH | W | 110 | 6.586 | 43.932 | 26.846 | 1.00 | 30.40 | 8 | O |
| ATOM | 2471 | O | HOH | W | 111 | 26.222 | 21.733 | 1.726 | 1.00 | 27.89 | 8 | O |
| ATOM | 2472 | O | HOH | W | 112 | 38.816 | 11.853 | 6.953 | 1.00 | 31.38 | 8 | O |
| ATOM | 2473 | O | HOH | W | 113 | 40.804 | 10.936 | −0.338 | 1.00 | 43.84 | 8 | O |
| ATOM | 2474 | O | HOH | W | 114 | 14.465 | 23.160 | 5.012 | 1.00 | 35.94 | 8 | O |
| ATOM | 2475 | O | HOH | W | 115 | 29.274 | 51.396 | 12.634 | 1.00 | 31.23 | 8 | O |
| ATOM | 2476 | O | HOH | W | 116 | 36.111 | 26.053 | 21.704 | 1.00 | 27.88 | 8 | O |
| ATOM | 2477 | O | HOH | W | 117 | 48.720 | 40.919 | 8.861 | 1.00 | 27.14 | 8 | O |
| ATOM | 2478 | O | HOH | W | 118 | 16.857 | 35.240 | 8.265 | 1.00 | 29.17 | 8 | O |
| ATOM | 2479 | O | HOH | W | 119 | 40.272 | 15.140 | −2.430 | 1.00 | 28.86 | 8 | O |
| ATOM | 2480 | O | HOH | W | 120 | 8.464 | 39.395 | 3.469 | 1.00 | 26.29 | 8 | O |
| ATOM | 2481 | O | HOH | W | 121 | 24.991 | 50.953 | 19.868 | 1.00 | 38.39 | 8 | O |
| ATOM | 2482 | O | HOH | W | 122 | 32.494 | 43.852 | 25.475 | 1.00 | 20.94 | 8 | O |
| ATOM | 2483 | O | HOH | W | 123 | 42.907 | 20.569 | 5.948 | 1.00 | 20.98 | 8 | O |
| ATOM | 2484 | O | HOH | W | 124 | 11.409 | 60.427 | −4.212 | 1.00 | 22.37 | 8 | O |
| ATOM | 2485 | O | HOH | W | 125 | 41.932 | 17.910 | −0.484 | 1.00 | 24.91 | 8 | O |
| ATOM | 2486 | O | HOH | W | 126 | −6.114 | 27.302 | 10.662 | 1.00 | 25.66 | 8 | O |
| ATOM | 2487 | O | HOH | W | 127 | 26.137 | 21.371 | 21.978 | 1.00 | 29.82 | 8 | O |
| ATOM | 2488 | O | HOH | W | 128 | 8.216 | 61.214 | 10.723 | 1.00 | 30.53 | 8 | O |
| ATOM | 2489 | O | HOH | W | 129 | 26.118 | 11.522 | 20.391 | 1.00 | 26.63 | 8 | O |
| ATOM | 2490 | O | HOH | W | 130 | 38.986 | 15.328 | −4.568 | 1.00 | 35.28 | 8 | O |
| ATOM | 2491 | O | HOH | W | 131 | 32.545 | 51.686 | 12.997 | 1.00 | 34.45 | 8 | O |
| ATOM | 2492 | O | HOH | W | 132 | 10.691 | 28.090 | 29.628 | 1.00 | 23.73 | 8 | O |
| ATOM | 2493 | O | HOH | W | 133 | 42.534 | 51.095 | 7.222 | 1.00 | 27.35 | 8 | O |
| ATOM | 2494 | O | HOH | W | 134 | 24.319 | 44.661 | 27.426 | 1.00 | 29.68 | 8 | O |
| ATOM | 2495 | O | HOH | W | 135 | 27.184 | 27.908 | 26.119 | 1.00 | 29.01 | 8 | O |
| ATOM | 2496 | O | HOH | W | 136 | 12.482 | 23.286 | 9.718 | 1.00 | 25.90 | 8 | O |
| ATOM | 2497 | O | HOH | W | 137 | 13.725 | 58.650 | 6.382 | 1.00 | 30.09 | 8 | O |
| ATOM | 2498 | O | HOH | W | 138 | 16.824 | 30.085 | 33.861 | 1.00 | 29.63 | 8 | O |
| ATOM | 2499 | O | HOH | W | 139 | 25.538 | 40.408 | 32.871 | 1.00 | 25.50 | 8 | O |
| ATOM | 2500 | O | HOH | W | 140 | −4.601 | 42.598 | 6.460 | 1.00 | 28.50 | 8 | O |
| ATOM | 2501 | O | HOH | W | 141 | −4.314 | 55.800 | 1.473 | 1.00 | 23.98 | 8 | O |
| ATOM | 2502 | O | HOH | W | 142 | 4.180 | 40.729 | 2.968 | 1.00 | 38.79 | 8 | O |
| ATOM | 2503 | O | HOH | W | 143 | 2.880 | 34.275 | 3.975 | 1.00 | 35.32 | 8 | O |
| ATOM | 2504 | O | HOH | W | 144 | 22.685 | 41.116 | 9.871 | 1.00 | 37.58 | 8 | O |
| ATOM | 2505 | O | HOH | W | 145 | 5.911 | 42.222 | 1.293 | 1.00 | 38.18 | 8 | O |
| ATOM | 2506 | O | HOH | W | 146 | −0.037 | 32.781 | 6.946 | 1.00 | 36.95 | 8 | O |
| ATOM | 2507 | O | HOH | W | 147 | 9.637 | 20.349 | 10.139 | 1.00 | 25.04 | 8 | O |
| ATOM | 2508 | O | HOH | W | 148 | 28.382 | 43.815 | 3.179 | 1.00 | 43.71 | 8 | O |
| ATOM | 2509 | O | HOH | W | 149 | 22.090 | 59.094 | 6.799 | 1.00 | 36.67 | 8 | O |
| ATOM | 2510 | O | HOH | W | 150 | 10.612 | 40.037 | 0.986 | 1.00 | 30.45 | 8 | O |
| ATOM | 2511 | O | HOH | W | 151 | 33.822 | 49.094 | 4.672 | 1.00 | 31.89 | 8 | O |
| ATOM | 2512 | O | HOH | W | 152 | 10.343 | 15.118 | 17.380 | 1.00 | 41.68 | 8 | O |
| ATOM | 2513 | O | HOH | W | 153 | 17.037 | 38.046 | 31.684 | 1.00 | 36.09 | 8 | O |
| ATOM | 2514 | O | HOH | W | 154 | −4.211 | 57.650 | 5.730 | 1.00 | 37.59 | 8 | O |
| ATOM | 2515 | O | HOH | W | 155 | 10.360 | 30.679 | 5.056 | 1.00 | 35.82 | 8 | O |
| ATOM | 2516 | O | HOH | W | 156 | 9.319 | 30.377 | 30.150 | 1.00 | 45.13 | 8 | O |
| ATOM | 2517 | O | HOH | W | 157 | 32.537 | 46.738 | 25.670 | 1.00 | 42.21 | 8 | O |
| ATOM | 2518 | O | HOH | W | 158 | −7.040 | 33.392 | 21.564 | 1.00 | 29.97 | 8 | O |
| ATOM | 2519 | O | HOH | W | 159 | 25.487 | 57.687 | 13.423 | 1.00 | 56.41 | 8 | O |
| ATOM | 2520 | O | HOH | W | 160 | 22.390 | 27.493 | 27.703 | 1.00 | 51.26 | 8 | O |
| ATOM | 2521 | O | HOH | W | 161 | −1.944 | 32.594 | 29.002 | 1.00 | 35.60 | 8 | O |
| ATOM | 2522 | O | HOH | W | 162 | 19.494 | 57.986 | 15.703 | 1.00 | 27.66 | 8 | O |
| ATOM | 2523 | O | HOH | W | 163 | 4.218 | 34.783 | −0.031 | 1.00 | 37.95 | 8 | O |
| ATOM | 2524 | O | HOH | W | 164 | 10.878 | 61.676 | 2.439 | 1.00 | 40.13 | 8 | O |
| ATOM | 2525 | O | HOH | W | 165 | 32.378 | 32.684 | 29.566 | 1.00 | 39.10 | 8 | O |
| ATOM | 2526 | O | HOH | W | 166 | 40.742 | 27.829 | 18.701 | 1.00 | 32.58 | 8 | O |
| ATOM | 2527 | O | HOH | W | 167 | 15.407 | 53.314 | 22.674 | 1.00 | 35.33 | 8 | O |
| ATOM | 2528 | O | HOH | W | 168 | 24.525 | 50.894 | 4.209 | 1.00 | 30.85 | 8 | O |
| ATOM | 2529 | O | HOH | W | 169 | 24.227 | 47.558 | 13.850 | 1.00 | 13.12 | 8 | O |
| ATOM | 2530 | O | HOH | W | 170 | 19.469 | 40.382 | 25.052 | 1.00 | 14.83 | 8 | O |
| ATOM | 2531 | O | HOH | W | 171 | 25.449 | 45.435 | 13.288 | 1.00 | 34.42 | 8 | O |
| ATOM | 2532 | O | HOH | W | 172 | 36.869 | 45.896 | 18.115 | 1.00 | 21.20 | 8 | O |
| ATOM | 2533 | O | HOH | W | 173 | 12.012 | 28.477 | 9.138 | 1.00 | 29.45 | 8 | O |
| ATOM | 2534 | O | HOH | W | 174 | 16.359 | 41.479 | 3.585 | 1.00 | 38.60 | 8 | O |
| ATOM | 2535 | O | HOH | W | 175 | 34.326 | 31.763 | −2.161 | 1.00 | 2.02 | 8 | O |
| ATOM | 2536 | O | HOH | W | 176 | 17.655 | 52.366 | −0.174 | 1.00 | 22.73 | 8 | O |
| ATOM | 2537 | O | HOH | W | 177 | 14.404 | 48.517 | 21.739 | 1.00 | 25.45 | 8 | O |
| ATOM | 2538 | O | HOH | W | 178 | 43.749 | 32.200 | 8.020 | 1.00 | 29.02 | 8 | O |
| ATOM | 2539 | O | HOH | W | 179 | 15.592 | 26.829 | 16.174 | 1.00 | 48.16 | 8 | O |
| ATOM | 2540 | O | HOH | W | 180 | 10.525 | 56.368 | −4.081 | 1.00 | 30.23 | 8 | O |
| ATOM | 2541 | O | HOH | W | 181 | 8.713 | 25.942 | 28.348 | 1.00 | 25.77 | 8 | O |
| ATOM | 2542 | O | HOH | W | 182 | 29.737 | 7.237 | 11.850 | 1.00 | 33.68 | 8 | O |
| ATOM | 2543 | O | HOH | W | 183 | 21.164 | 33.439 | 7.966 | 1.00 | 38.28 | 8 | O |
| ATOM | 2544 | O | HOH | W | 184 | 24.119 | 46.149 | 24.377 | 1.00 | 37.23 | 8 | O |
| ATOM | 2545 | O | HOH | W | 185 | 21.753 | 56.692 | 10.121 | 1.00 | 33.11 | 8 | O |
| ATOM | 2546 | O | HOH | W | 186 | 17.585 | 38.448 | 12.402 | 1.00 | 39.35 | 8 | O |
| ATOM | 2547 | O | HOH | W | 187 | −2.754 | 38.361 | 11.916 | 1.00 | 44.68 | 8 | O |
| ATOM | 2548 | O | HOH | W | 188 | 16.639 | 27.328 | 18.235 | 1.00 | 24.46 | 8 | O |
| ATOM | 2549 | O | HOH | W | 189 | 14.615 | 34.256 | 8.534 | 1.00 | 41.84 | 8 | O |
| ATOM | 2550 | O | HOH | W | 190 | 14.514 | 43.801 | 25.157 | 1.00 | 33.67 | 8 | O |
| ATOM | 2551 | O | HOH | W | 191 | 26.469 | 30.002 | 3.866 | 1.00 | 53.62 | 8 | O |
| ATOM | 2552 | O | HOH | W | 192 | 3.416 | 28.107 | 4.131 | 1.00 | 47.37 | 8 | O |
| ATOM | 2553 | O | HOH | W | 193 | 15.414 | 27.531 | 11.247 | 1.00 | 40.29 | 8 | O |
| ATOM | 2554 | O | HOH | W | 194 | 24.628 | 58.221 | 6.032 | 1.00 | 37.02 | 8 | O |
| ATOM | 2555 | O | HOH | W | 195 | −1.971 | 51.690 | 17.147 | 1.00 | 35.28 | 8 | O |
| ATOM | 2556 | O | HOH | W | 196 | 16.659 | 23.680 | 26.303 | 1.00 | 37.61 | 8 | O |
| END | | | | | | | | | | | | |

[Advantages]

By using the present invention, it becomes possible to identify accurately and efficiently FFRPs regulating metabolism, growth, and infectivity of bacteria. Targeting the identified FFRPs, antimicrobial agents can be searched by using a known ligand screening method, or by a virtual screening method using 3D structure information.

### Sequence Listing

<110> National Institute of Advanced Industrial Science and Technology(AIST)
<120> Method of Screening for Antimicrobial Agents
<130> 334-03166
<140>
<141>
<160> 33
<170> PatentIn Ver. 2.1
<210> 1
<211> 157
<212> PRT
<213> Pseudomonas aeruginosa
<220>
<223> Inventor: Suzuki, Masashi; Koike, Hideaki

FIG. 1 shows correlation between amino acid sequences of different FFRPs (i.e., a multiple alignment) coded in the genomes of various bacteria. Hydrophobic phase inside α-helices are marked with asterisks (*), with types of residues, isoleucine, valine, leucine, and methionine in these phases highlighted in bold. Outside the α-helices at each position, the type of residues most frequently found is highlighted in bold.

[FIG. 2]

FIG. 2 is a diagram showing phylogenetic relations between FFRPs.

In this diagram, nodes characterized with bootstrap values of 500 or higher are colored in red. Nodes characterized with smaller bootstrap values, 350 or higher but less than 500, are indicated by closed circles colored in black. Four demi-FFRPs are colored in green, and the two subgroups together covering the eight P. aeruginosa FFRPs are colored in red and blue, respectively.

[FIG. 3]

FIG. 3 shows the thirty-two amino acid positions where pharmaceutical agents will bind. In this figure along the line, “(iii) Modeling of leucine binding”, residues producing conflicts (X) or no conflict (O) when leucine is modeled to fit pot1216151 are indicated.

[FIG. 4]

FIG. 4 show a wire model of the crystal structure of an FFRP (pot1216151) derived from an archaeon (Pyrococcus OT3). In this figure, the electron densities of two molecules of an unidentified ligand derived from E. coli are colored in red.

[FIG. 5]

FIG. 5 shows an enlargement of the electron density of the ligand shown in FIG. 4, to which a valine molecule is best fit. In this figure, the valine molecule is represented by a wire model, and the atoms are differentiated: oxygen (red), nitrogen (blue), and carbon (yellow). Hydrogen atoms are omitted.

[FIG. 6]

FIG. 6 shows residues of pot1216151 positioned within 6 Å from the unidentified assembly promotion factor derived from E. coli, and residues of other FFRP molecules occupying the identical positions. Residues found in E. coli Lrp but not found in EcO468065 at the same positions are colored in red, residues found in Ec0468065 but not found in E. coli Lrp at the same positions are colored in blue, and residues found in both at the same positions are colored in green.

[FIG. 7]

FIG. 7 shows positions potentially important for leucine-binding by E. coli Lrp, and the residues of archaeal FFRPs found at the same positions. In this figure, residues found in E. coli Lrp but not found in Ec0468065 at the same positions are colored in red, residues found in Ec0468065 but not found in E. coli Lrp are colored in blue, and residues found in both are colored in green.

[FIG. 8]

FIG. 8 shows a ribbon diagram of the crystal structure of pot1216151 with coloring seven positions important for leucine-binding by E. coli Lrp in blue.

[FIG. 9]

FIG. 9 shows a ribbon diagram of leucine (yellow) binding to pot1216151. FIG. 9 shows a view looked along the arrow shown in FIG. 10: two dimers closest to the arrow and a leucine molecule are shown. In the upper half of this model, positions the same as those colored in blue in FIG. 8 are highlighted in the same color, i.e., in blue. Meanwhile, residues forming the gap between the two dimers are highlighted in green. In the lower half of this model, residues contacting the modeled leucine molecule with no conflict are colored in blue, and three other residues positioned conflicting with the leucine molecule are colored in red.

[FIG. 10]

FIG. 10 is a ribbon diagram of a model of leucine-binding to pot1216151.

[FIG. 11]

FIG. 11 shows residues of pot1216151 positioned close to the leucine molecule modeled to fit the protein, and residues of other FFRP molecules occupying the same positions as those in pot1216151. In this figure, residues found in E. coli Lrp but not found in Ec0468065 at the same positions are colored in red, residues found in Ec0468065 but not found in E. coli Lrp at the same positions are colored in blue, and residues found in both at the same positions are colored in green.

[FIG. 12]

FIG. 12 shows residues of pot1216151 whose side chains are facing the gaps formed between the dimers, and residues of other FFRPs occupying the same positions. In this figure, residues found in E. coli Lrp but not found in Ec0468065 at the same positions are colored in red, residues found in Ec0468065 but not found in E. coli Lrp at the same positions are colored in blue, and residues found in both at the same positions are colored in green.

[FIG. 13]

FIG. 13 shows a partial rearrangement of the thirty-two positions shown in FIG. 3.

[FIG. 14]

FIG. 14 shows positions of FFRPs identified as important for DNA recognition. In this figure, residues found in E. coli Lrp but not found in Ec0468065 at the same positions are colored in red, residues found in Ec0468065 but not found in E. coli Lrp at the same positions are colored in blue, and residues found in both at the same positions are colored in green.

[FIG. 15]

FIG. 15 shows a ribbon diagram of the 3D structure of a dimer of the FFRP (pot1216151) in two views.

[FIG. 16]

FIG. 16 shows profiles of gel filtration of the FFRP pot1216151 purified using different methods: mild (black, purification 1) and rigorous (red, purification 2).

[FIG. 17]

FIG. 17 shows profiles of gel filtration of the FFRP pot1216151. In this diagram, profiles shown are those obtained in the absence (black) or presence of chemical compounds: amino acids (a, b) and other metabolic intermediates (c), malic acid (red), 2-oxoglutaric acid (green), and oxaloacetic acid (blue).

1. A method of identifying a target protein for its use in screening for an antimicrobial agent, which comprises the steps of:
1) from the amino acid sequences of all open reading frames identified using the genomic sequence of a target bacterium, selecting amino acid sequences having homologies of 20% or higher to one of feast/famine regulatory proteins (FFRPs) having amino acid sequences set forth in SEQ ID NOS. 9 to 31; and
2) from the amino acid sequences selected in step 1), excluding any amino acid sequence that does not match with a multiple alignment by forming the same secondary structural elements, the multiple alignment being shown in FIG. 1; and
3) identifying a protein, which has an amino acid sequence obtained through steps 1) and 2), as the target protein for its use in the screening for the antimicrobial agent.

2. The method according to claim 1, further comprising the step of excluding any amino acid sequence phylogenetically related with one of E. coli FFRPs having amino acid sequences set forth in SEQ ID NOS. 9 to 11, by a bootstrap value of 900/1,000 or higher.

3. The method according to claim 1 or 2, wherein, in step 1), from the amino acid sequences of all open reading frames identified using the genomic sequence of the target bacterium, amino acid sequences having FASTA Z scores of 180 or higher to one of FFRPs having amino acid sequences set forth in SEQ ID NOS. 9 to 31 are selected.

4. The method according to any one of claims 1 to 3, wherein, in step 2), any amino acid sequence that is unlikely to form α helices in five regions 34 to 43, 59 to 66, 70 to 82, 126 to 135, and 170 to 180 or β strands in five regions 90 to 96, 109 to 117, 142 to 148, 154 to 160, and 193 to 205 is excluded from the amino acid sequences selected by step 1), wherein these positions are identified using the numbering scheme shown in the multiple alignment in FIG. 1.

5. A method of screening for an antimicrobial agent, which comprises the step of selecting a chemical compound as a candidate of the agent, wherein the chemical compound is able to bind specifically to a protein identified by the method according to any one of claims 1 to 4 or to its assembly, thereby altering the three-dimensional structure or the assembly form of the protein.

6. A method of screening for an antimicrobial agent acting against Pseudomonas aeruginosa, which comprises the step of selecting a chemical compound as a candidate of the antimicrobial agent, wherein said chemical compound is able to bind specifically to a target protein or its assembly, thereby altering the three-dimensional structure or the assembly form of the protein, wherein the target protein is either
i) a protein having an amino acid sequence set forth in one of SEQ ID NOS. 1 to 8; or
ii) a protein which is derived from Pseudomonas aeruginosa, which is related to an amino acid sequence set forth in one of SEQ ID NOS. 1 to 8 by deletion, substitution, or insertion of up to several amino acid residues, and which is able to act as an FFRP.

7. A method of screening for an antimicrobial agent acting against Pseudomonas aeruginosa, which comprises the step of selecting a chemical compound as a candidate of the antimicrobial agent, said chemical compound being able to bind specifically to a target protein having an amino acid sequence set forth in one of SEQ ID NOS. 1 to 8 or able to bind to its assembly, thereby altering the three-dimensional structure or the assembly form of the target protein.

8. A method of screening for an antimicrobial agent acting against Pseudomonas aeruginosa, which comprises the step of selecting a chemical compound as a candidate of the antimicrobial agent, said chemical compound being able to bind specifically to a target protein having an amino acid sequence set forth in one of SEQ ID NOS. 1 to 7 or its assembly, thereby altering the three-dimensional structure or the assembly form of the target protein.

9. The method according to any one of claims 5 to 8, wherein the chemical compound is selected on the basis of interaction between the chemical compound and at least one of thirty-two amino acid residues in the target protein, said thirty-two amino acid residues being found at positions 113, 116, 119, 120, 126, 144, 145, 147, 148, 149, 150, 152, 153, 154, 155, 166, 169, 173, 176, 177, 181, 183, 185, 193, 195, 196, 197, 198, 199, 200, 201, and 202, respectively, wherein these positions are identified using the numbering scheme shown in the multiple alignment in FIG. 1.

10. The method according to any one of claims 5 to 8, wherein the chemical compound is selected on the basis of interaction between the chemical compound and at least one of eighteen amino acid residues in the target protein, said eighteen amino acid residues being found at positions 113, 126, 147, 148, 149, 150, 152, 153, 154, 155, 176, 177, 181, 196, 197, 198, 199, and 202, respectively, wherein these positions are identified using the numbering scheme shown in the multiple alignment in FIG. 1.

11. The method according to any one of claims 5 to 8, wherein the chemical compound is selected on the basis of interaction between the chemical compound and at least one of seven amino acid residues in the target protein, said seven amino acid residues being found at positions 147, 154, 169, 181, 199, 200, and 201, respectively, wherein these positions are identified using the numbering scheme shown in the multiple alignment in FIG. 1.

12. The method according to any one of claims 5 to 8, wherein the chemical compound is selected on the basis of interaction between the chemical compound and at least one of seven amino acid residues in the target protein, said seven amino acid residues being found at positions 147, 149, 154, 169, 173, 200, and 202, respectively, wherein these positions are identified using the numbering scheme shown in the multiple alignment in FIG. 1.

13. The method according to any one of claims 5 to 8, wherein the chemical compound is selected on the basis of interaction between the chemical compound and at least one of twenty-eight amino acid residues in the target protein, said twenty-eight amino acid residues being found at positions 116, 119, 120, 126, 144, 145, 147, 148, 149, 150, 152, 153, 154, 166, 169, 173, 177, 181, 183, 185, 193, 195, 196, 197, 198, 199, 200, and 201, respectively, wherein these positions are identified using the numbering scheme shown in the multiple alignment in FIG. 1.
